SEQUENCE LISTING (220 sequences)

SEQ ID NO: 1 - DNA, 1233 bp, Escherichia coli
atgcaaaataaattagcttccggtgccaggcttggacgtcaggcgttacttttccctctctgtctggtgctttacgaattttcaacctatatcggcaacgatatgattcaacccggtatgttggccgtggtggaacaatatcaggcgggcattgattgggttcctacttcgatgaccgcgtatctggcgggcgggatgtttttacaatggctgctggggccgctgtcggatcgtattggtcgccgtccggtgatgctggcgggagtggtgtggtttatcgtcacctgtctggcaatattgctggcgcaaaacattgaacaattcaccctgttgcgcttcttgcagggcataagcctctgtttcattggcgctgtgggatacgccgcaattcaggaatccttcgaagaggcggtttgtatcaagatcaccgcgctgatggcgaacgtggcgctgattgctccgctacttggtccgctggtgggcgcggcgtggatccatgtgctgccctgggaggggatgtttgttttgtttgccgcattggcagcgatctcctttttcggtctgcaacgagccatgcctgaaaccgccacgcgtataggcgagaaactgtcactgaaagaactcggtcgtgactataagctggtgctgaagaacggccgctttgtggcgggggcgctggcgctgggattcgttagtctgccgttgctggcgtggatcgcccagtcgccgattatcatcattaccggcgagcagttgagcagctatgaatatggcttgctgcaagtgcctattttcggggcgttaattgcgggtaacttgctgttagcgcgtctgacctcgcgccgcaccgtacgttcgctgattattatgggcggctggccgattatgattggtctattggtcgctgctgcggcaacggttatctcatcgcacgcgtatttatggatgactgccgggttaagtatttatgctttcggtattggtctggcgaatgcgggactggtgcgattaaccctgtttgccagcgatatgagtaaaggtacggtttctgccgcgatgggaatgctgcaaatgctgatctttaccgttggtattgaaatcagcaaacatgcctggctgaacgggggcaacggactgtttaatctcttcaaccttgtcaacggaattttgtggctgtcgctgatggttatctttttaaaagataaacagatgggaaattctcacgaagggtaa

SEQ ID NO: 2 - protein, 410 aa, Escherichia coli
MQNKLASGARLGRQALLFPLCLVLYEFSTYIGNDMIQPGMLAVVEQYQAGIDWVPTSMTAYLAGGMFLQWLLGPLSDRIGRRPVMLAGVVWFIVTCLAILLAQNIEQFTLLRFLQGISLCFIGAVGYAAIQESFEEAVCIKITALMANVALIAPLLGPLVGAAWIHVLPWEGMFVLFAALAAISFFGLQRAMPETATRIGEKLSLKELGRDYKLVLKNGRFVAGALALGFVSLPLLAWIAQSPIIIITGEQLSSYEYGLLQVPIFGALIAGNLLLARLTSRRTVRSLIIMGGWPIMIGLLVAAAATVISSHAYLWMTAGLSIYAFGIGLANAGLVRLTLFASDMSKGTVSAAMGMLQMLIFTVGIEISKHAWLNGGNGLFNLFNLVNGILWLSLMVIFLKDKQMGNSHEG

SEQ ID NO: 3 - DNA, 1254 bp, Escherichia coli
atgagccgtactacaactgttgatggcgctccggcaagcgacactgacaagcaaagcatttctcagccaaatcaatttattaaacgcggtacgccgcaatttatgcgcgtcaccctggcgctgttctctgccggactggcaacatttgcacttctctattgtgtgcagcctatccttccggtgctttcgcaggagtttggcttaacccccgcgaacagtagtatttcactgtccatttccacggcgatgttggctattggtttgctgtttactggcccgctatccgatgccattggtcgcaaaccagtgatggtcacggcgctactgttggcctccatttgtacgttactttcgacaatgatgaccagctggcacggcattttgattatgcgcgccttgattgggctttcgttaagtggcgtggcagctgttggcatgacttatcttagcgaggaaatccatcccagtttcgtggccttttcaatggggttgtatatcagcggcaactcaattggcggcatgagcggacgcttaattagcggtgtcttcacggactttttcaactggcgaattgctctggcggcaatcggttgtttcgcgctggcctcggcgttgatgttctggaaaatcctccctgaatcacgccattttcgcccgacttcgctgcgccctaagacgttgtttatcaactttcgtctgcactggcgtgaccggggattaccgttattgttcgcagaaggctttttgctgatggggtcgttcgtcacgctgtttaattacatcggctatcggttgatgctctccccctggcatgtcagtcaggccgtggttggcttattatcgctggcttatttgaccggtacatggagctcacccaaagccggaaccatgaccacccgctatgggcgtggtccagtgatgttgttttcgacgggggttatgctgtttggtttactgatgaccttattcagctcgctgtggctgatctttgccggaatgttactcttctcagcaggattcttcgcagcccactcagtagccagcagctggatcggcccccgcgcaaaacgcgctaaaggccaggcctcctcgctgtatctgttcagttactatctggggtcgagtattgccgggacgctgggtggtgttttctggcataactatggctggaacggcgtcggcgcatttattgctctgatgctggtcattgctctgctggtcgggacgcgtttgcatcgtcgtctgcacgcctaa

SEQ ID NO: 4 - protein, 417 aa, Escherichia coli
MSRTTTVDGAPASDTDKQSISQPNQFIKRGTPQFMRVTLALFSAGLATFALLYCVQPILPVLSQEFGLTPANSSISLSISTAMLAIGLLFTGPLSDAIGRKPVMVTALLLASICTLLSTMMTSWHGILIMRALIGLSLSGVAAVGMTYLSEEIHPSFVAFSMGLYISGNSIGGMSGRLISGVFTDFFNWRIALAAIGCFALASALMFWKILPESRHFRPTSLRPKTLFINFRLHWRDRGLPLLFAEGFLLMGSFVTLFNYIGYRLMLSPWHVSQAVVGLLSLAYLTGTWSSPKAGTMTTRYGRGPVMLFSTGVMLFGLLMTLFSSLWLIFAGMLLFSAGFFAAHSVASSWIGPRAKRAKGQASSLYLFSYYLGSSIAGTLGGVFWHNYGWNGVGAFIALMLVIALLVGTRLHRRLHA

SEQ ID NO: 5 - DNA, 1416 bp, Escherichia coli
atgacagatcttcccgacagcacccgttggcaattgtggattgtggctttcggcttctttatgcagtcgctggacaccaccatcgtaaacaccgcccttccctcaatggcgcaaagcctcggggaaagtccgttgcatatgcacatggtcattgtctcttatgtgctgaccgtggcggtgatgctgcccgccagcggctggctggcggacaaagtcggcgtgcgcaatattttctttaccgccatcgtgctgtttactctcggttcactgttttgcgcgctttccggcacgctgaacgaactgttgctggcacgcgcgttacagggcgttggcggcgcgatgatggtgccggtcggcagattgacggtgatgaaaatcgtaccgcgcgagcaatatatggcggcgatgacctttgtcacgttacccggtcaggtcggtccgctgctcggtccggcgctcggcggtctgctggtggagtacgcatcgtggcactggatctttttgatcaacattccggtggggattatcggtgcgatcgccacattgctgttaatgccgaactacaccatgcagacgcggcgctttgatctctccggatttttattgctggcggttggcatggcggtattaaccctggcgctggacggcagtaaaggtacaggtttatcgccgctgacgattgcaggcctggtcgcagttggcgtggtggcactggtgctttatctgctgcacgccagaaataacaaccgtgccctgttcagtctgaaactgttccgtactcgtaccttttcgctgggcctggcggggagctttgccggacgtattggcagtggcatgttgccctttatgacaccggttttcctgcaaattggcctcggtttctcgccgtttcatgccggactgatgatgatcccgatggtgcttggcagcatgggaatgaagcgaattgtggtacaggtggtgaatcgctttggttatcgtcgggtactggtagcgaccacgctgggtctgtcgctggtcaccctgttgtttatgactaccgccctgctgggctggtactacgttttgccgttcgtcctgtttttacaagggatggtcaactcgacgcgtttctcctccatgaacaccctgacgctgaaagatctcccggacaatctggcgagcagcggcaacagcctgctgtcgatgattatgcaattgtcgatgagtatcggcgtcactatcgccgggctgttgctgggactttttggttcacagcatgtcagcgtcgacagcggcaccacacaaaccgtctttatgtacacctggcttagcatggcgttgatcatcgcccttccggcgttcatctttgccagagtgccgaacgatacgcatcaaaatgtagctatttcgcggcgaaaaaggagcgcgcaataa

SEQ ID NO: 6 - protein, 471 aa, Escherichia coli
MTDLPDSTRWQLWIVAFGFFMQSLDTTIVNTALPSMAQSLGESPLHMHMVIVSYVLTVAVMLPASGWLADKVGVRNIFFTAIVLFTLGSLFCALSGTLNELLLARALQGVGGAMMVPVGRLTVMKIVPREQYMAAMTFVTLPGQVGPLLGPALGGLLVEYASWHWIFLINIPVGIIGAIATLLLMPNYTMQTRRFDLSGFLLLAVGMAVLTLALDGSKGTGLSPLTIAGLVAVGVVALVLYLLHARNNNRALFSLKLFRTRTFSLGLAGSFAGRIGSGMLPFMTPVFLQIGLGFSPFHAGLMMIPMVLGSMGMKRIVVQVVNRFGYRRVLVATTLGLSLVTLLFMTTALLGWYYVLPFVLFLQGMVNSTRFSSMNTLTLKDLPDNLASSGNSLLSMIMQLSMSIGVTIAGLLLGLFGSQHVSVDSGTTQTVFMYTWLSMALIIALPAFIFARVPNDTHQNVAISRRKRSAQ

SEQ ID NO: 7 - DNA, 1218 bp, Escherichia coli
atgcccgaacccgtagccgaacccgcgctaaacggattgcgcctgaatttgcgcattgtctctatagtcatgtttaacttcgccagctacctcaccatcgggttgccgctcgctgtattaccgggctatgtccatgatgtgatgggctttagcgccttctgggcaggattggttatcagcctgcaatatttcgccaccttgctgagccgccctcatgccggacgttacgccgattcgctgggacccaaaaagattgtcgtcttcggtttatgcggctgctttttgagcggtctggggtatctgacggcaggattaaccgccagtctgcctgtcatcagcctgttattactttgcctggggcgcgtcatccttgggattgggcaaagttttgccggaacgggatcgaccctatggggcgttggcgtggttggctcgctgcatatcgggcgggtgatttcgtggaacggcattgtcacttacggggcgatggcgatgggtgcgccgttaggcgtcgtgttttatcactggggcggcttgcaggcgttagcgttaatcattatgggcgtggcgctggtggccattttgttggcgatcccgcgtccgacggtaaaagccagtaaaggcaaaccgctgccgtttcgcgcggtgcttgggcgcgtctggctgtacggtatggcgctggcactggcttccgccggatttggcgtcatcgccacctttatcacgctgttttatgacgctaaaggttgggacggtgcggctttcgcgctgacgctgtttagctgtgcgtttgtcggtacgcgtttgttattccctaacggcattaaccgtatcggtggcttaaacgtagcgatgatttgctttagcgttgagataatcggcctgctactggttggcgtggcgactatgccgtggatggcgaaaatcggcgtcttactggcgggggccgggttttcgctggtgttcccggcattgggtgtagtggcggtaaaagcggttccgcagcaaaatcagggggcggcgctggcaacttacaccgtatttatggatttatcgcttggcgtgactggaccactggctgggctggtgatgagctgggcgggcgtaccggtgatttatctggcggcggcgggactggtcgcaatcgcgttattactgacgtggcgattaaaaaaacggcctccggaacacgtccctgaggccgcctcatcatcttaa

SEQ ID NO: 8 - protein, 405 aa, Escherichia coli
MPEPVAEPALNGLRLNLRIVSIVMFNFASYLTIGLPLAVLPGYVHDVMGFSAFWAGLVISLQYFATLLSRPHAGRYADSLGPKKIVVFGLCGCFLSGLGYLTAGLTASLPVISLLLLCLGRVILGIGQSFAGTGSTLWGVGVVGSLHIGRVISWNGIVTYGAMAMGAPLGVVFYHWGGLQALALIIMGVALVAILLAIPRPTVKASKGKPLPFRAVLGRVWLYGMALALASAGFGVIATFITLFYDAKGWDGAAFALTLFSCAFVGTRLLFPNGINRIGGLNVAMICFSVEIIGLLLVGVATMPWMAKIGVLLAGAGFSLVFPALGVVAVKAVPQQNQGAALATYTVFMDLSLGVTGPLAGLVMSWAGVPVIYLAAAGLVAIALLLTWRLKKRPPEHVPEAASSS

SEQ ID NO: 9 - DNA, 1185 bp, Escherichia coli
atgaaaaggcaaagaaacgtcaatttgttattgatgttggtattactcgtggccgtcggtcagatggcgcaaaccatttatattccagctattgccgatatggcgcgcgatctcaacgtccgtgaaggggcggtgcagagcgtaatgggcgcttatctgctgacttacggtgtctcacagctgttttatggcccgatttccgaccgcgtgggccgccgaccggtgatcctcgtcggaatgtccatttttatgctggcaacgctggtcgcggtcacgacctccagtttgacggtgttgattgccgccagcgcgatgcaggggatgggcaccggcgttggcggcgtaatggcgcgtactttaccgcgagatttatatgaacggacacagttgcgccatgctaacagcctgttaaacatggggattctcgtcagtccgttgctcgcaccgctaatcggcggtctgctggatacgatgtggaactggcgcgcctgttatctctttttgttggttctttgtgctggtgtgaccttcagtatggcccgctggatgccggaaacgcgtccggtcgatgcaccgcgcacgcgcctgcttaccagttataaaacgcttttcggtaacagcggttttaactgttatttgctgatgctgattggcggtctggccgggattgccgcctttgaagcctgctccggcgtgctgatgggcgcggtgttagggctgagcagtatgacggtcagtattttgtttattctgccgattccggcagcgttttttggcgcatggtttgccggacgtcccaataaacgcttctccacgttaatgtggcagtcggttatctgctgcctgctggctggcttgctgatgtggatccccgactggtttggcgtgatgaatgtctggacgctgctcgttcccgccgcgctgttctttttcggtgccgggatgctgtttccgctggcgaccagcggcgcgatggagccgttccccttcctggcgggcacggctggcgcgctggtcggcggtctgcaaaacattggttccggcgtgctggcgtcgctctctgcgatgttgccgcaaaccggtcagggcagcctggggttgttgatgaccttaatgggattgttgatcgtgctgtgctggctgccgctggcgacgcggatgtcgcatcaggggcagcccgtttaa

SEQ ID NO: 10 - protein, 394 aa, Escherichia coli
MKRQRNVNLLLMLVLLVAVGQMAQTIYIPAIADMARDLNVREGAVQSVMGAYLLTYGVSQLFYGPISDRVGRRPVILVGMSIFMLATLVAVTTSSLTVLIAASAMQGMGTGVGGVMARTLPRDLYERTQLRHANSLLNMGILVSPLLAPLIGGLLDTMWNWRACYLFLLVLCAGVTFSMARWMPETRPVDAPRTRLLTSYKTLFGNSGFNCYLLMLIGGLAGIAAFEACSGVLMGAVLGLSSMTVSILFILPIPAAFFGAWFAGRPNKRFSTLMWQSVICCLLAGLLMWIPDWFGVMNVWTLLVPAALFFFGAGMLFPLATSGAMEPFPFLAGTAGALVGGLQNIGSGVLASLSAMLPQTGQGSLGLLMTLMGLLIVLCWLPLATRMSHQGQPV

SEQ ID NO: 11 - DNA, 1212 bp, Escherichia coli
atgcaacctgggaaaagatttttagtctggctggcgggtttgagcgtactcggttttctggcaaccgatatgtatctgcctgctttcgccgccatacaggccgacctgcaaacgcctgcgtctgctgtcagtgccagccttagtctgttccttgccggttttgccgcagcccagcttctgtgggggccgctctccgaccgttatggtcgtaaaccggtattattaatcggcctgacaatttttgcgttaggtagtctggggatgctgtgggtagaaaacgccgctacgctgctggtattgcgttttgtacaggctgtgggtgtctgcgccgcggcggttatctggcaagcattagtgacagattattatccttcacagaaagttaaccgtatttttgcggccatcatgccgctggtgggtctatctccggcactggctcctctgttaggaagctggctgctggtccatttttcctggcaggcgattttcgccaccctgtttgccattaccgtggtgctgattctgcctattttctggctcaaacccacgacgaaggcccgtaacaatagtcaggatggtctgacctttaccgacctgctacgttctaaaacctatcgcggcaacgtgctgatatacgcagcctgttcagccagtttttttgcatggctgaccggttcaccgttcatccttagtgaaatgggctacagcccggcagttattggtttaagttatgtcccgcaaactatcgcgtttctgattggtggttatggctgtcgcgccgcgctgcagaaatggcaaggcaagcagttattaccgtggttgctggtgctgtttgctgtcagcgtcattgcgacctgggctgcgggcttcattagccatgtgtcgctggtcgaaatcctgatcccattctgtgtgatggcgattgccaatggcgcgatctaccctattgttgtcgcccaggcgctgcgtcccttcccacacgcaactggtcgcgccgcagcgttgcagaacactcttcaactgggtctgtgcttcctcgcaagtctggtagtttcctggctgatcagtatcagcacgccattgctcaccaccaccagcgtgatgttatcaacagtagtgctggtcgcgctgggttacatgatgcaacgttgtgaagaagttggctgccagaatcatggcaatgccgaagtcgctcatagcgaatcacactaa

SEQ ID NO: 12 - protein, 403 aa, Escherichia coli
MQPGKRFLVWLAGLSVLGFLATDMYLPAFAAIQADLQTPASAVSASLSLFLAGFAAAQLLWGPLSDRYGRKPVLLIGLTIFALGSLGMLWVENAATLLVLRFVQAVGVCAAAVIWQALVTDYYPSQKVNRIFAAIMPLVGLSPALAPLLGSWLLVHFSWQAIFATLFAITVVLILPIFWLKPTTKARNNSQDGLTFTDLLRSKTYRGNVLIYAACSASFFAWLTGSPFILSEMGYSPAVIGLSYVPQTIAFLIGGYGCRAALQKWQGKQLLPWLLVLFAVSVIATWAAGFISHVSLVEILIPFCVMAIANGAIYPIVVAQALRPFPHATGRAAALQNTLQLGLCFLASLVVSWLISISTPLLTTTSVMLSTVVLVALGYMMQRCEEVGCQNHGNAEVAHSESH

SEQ ID NO: 13 - DNA, 1251 bp, Escherichia coli
atgaataaacaatcctggctgcttaacctcagcctgttgaaaacgcacccggcgtttcgcgcagtattcctcgctcgtttcatctcaattgtgtctctgggtttgctcggcgtcgcggtgccggtgcagatccagatgatgacacattccacctggcaggtggggctttcggtgacgctgaccggcggcgcgatgtttgttggcctgatggtcggcggtgtgctggcggatcgctatgagcgcaaaaaagtgattttgctggcgcgcggcacctgtggcattggcttcattggactgtgccttaatgcactgctgccggagccgtcattgctggcaatctatttacttggtttatgggatggttttttcgcatcgcttggcgttacggcgctattggcggcgacaccagcactggtagggcgtgaaaacttaatgcaggccggggcgatcaccatgttgaccgtgcgtctggggtcggtgatttcgcccatgattggcggtttattgctggcgaccggtggcgtagcctggaactacgggctggcggcggcgggcacgtttattaccttgctaccgttgttaagccttccggcgttgccaccgccaccgcagccgcgtgagcatccgttgaaatcattactggcaggatttcgttttctgcttgccagcccgctggtgggcgggattgcgctgctgggtggtttattgacgatggcgagcgcggtgcgggtactgtatccggcgctggctgacaactggcagatgtcagcggcacagattggttttctctacgcggcgatcccgctcggcgcggctattggtgcgttaaccagcgggaagctggcacatagtgcgcgaccagggttattgatgctgctctccacgctgggatcgttcctcgccattggtctgtttggcctgatgccgatgtggattttaggcgtggtttgtctggcgctgttcggctggttgagtgcggtcagctcgttgctgcaatacacaatgctgcaaacgcaaaccccggaagcgatgttagggcggattaacggtttgtggacggcgcagaacgtgacgggcgatgccataggcgcggcgctgctgggtggtttgggcgcgatgatgacaccggttgcttccgcaagcgcgagcggttttggtttgttgattatcggcgtgttgttattgctggtgctggtggagttgcgacattttcgccagacgccgccgcaggtgacagcgtccgacagttaa

SEQ ID NO: 14 - protein, 416 aa, Escherichia coli
MNKQSWLLNLSLLKTHPAFRAVFLARFISIVSLGLLGVAVPVQIQMMTHSTWQVGLSVTLTGGAMFVGLMVGGVLADRYERKKVILLARGTCGIGFIGLCLNALLPEPSLLAIYLLGLWDGFFASLGVTALLAATPALVGRENLMQAGAITMLTVRLGSVISPMIGGLLLATGGVAWNYGLAAAGTFITLLPLLSLPALPPPPQPREHPLKSLLAGFRFLLASPLVGGIALLGGLLTMASAVRVLYPALADNWQMSAAQIGFLYAAIPLGAAIGALTSGKLAHSARPGLLMLLSTLGSFLAIGLFGLMPMWILGVVCLALFGWLSAVSSLLQYTMLQTQTPEAMLGRINGLWTAQNVTGDAIGAALLGGLGAMMTPVASASASGFGLLIIGVLLLLVLVELRHFRQTPPQVTASDS

SEQ ID NO: 15 - DNA, 1188 bp, Escherichia coli
atgaacttatccctacgacgctctaccagcgcccttcttgcctcgtcgttgttattaaccatcggacgcggcgctacgctgccatttatgaccatttacttgagtcgccagtacagcctgagtgtcgatctaatcggttatgcgatgacaattgcgctcactattggcgtcgtttttagcctcggttttggtatcctggcggataagttcgacaagaaacgctatatgttactggcaattaccgccttcgccagcggttttattgccattactttagtgaataacgtgacgctggttgtgctcttttttgccctcattaactgcgcctattctgtttttgctaccgtgctgaaagcctggtttgccgacaatctttcgtccaccagcaaaacgaaaatcttctcaatcaactacaccatgctaaacattggctggaccatcggtccgccgctcggcacgctgttggtaatgcagagcatcaatctgcccttctggctggcagctatctgttccgcgtttcccatgcttttcattcaaatttgggtaaagcgcagcgagaaaatcatcgccacggaaacaggcagtgtctggtcgccgaaagttttattacaagataaagcactgttgtggtttacctgctctggttttctggcttcttttgtaagcggcgcatttgcttcatgcatttcacaatatgtgatggtgattgctgatggggattttgccgaaaaggtggtcgcggttgttcttccggtgaatgctgccatggtggttacgttgcaatattccgtgggccgccgacttaacccggctaacatccgcgcgctgatgacagcaggcaccctctgtttcgtcatcggtctggtcggttttattttttccggcaacagcctgctattgtggggtatgtcagctgcggtatttactgtcggtgaaatcatttatgcgccgggcgagtatatgttgattgaccatattgcgccgccagaaatgaaagccagctatttttccgcccagtctttaggctggcttggtgccgcgattaacccattagtgagtggcgtagtgctaaccagcctgccgccttcctcgctgtttgtcatcttagcgttggtgatcattgctgcgtgggtgctgatgttaaaagggattcgagcaagaccgtgggggcagcccgcgctttgttaa

SEQ ID NO: 16 - protein, 395 aa, Escherichia coli
MNLSLRRSTSALLASSLLLTIGRGATLPFMTIYLSRQYSLSVDLIGYAMTIALTIGVVFSLGFGILADKFDKKRYMLLAITAFASGFIAITLVNNVTLVVLFFALINCAYSVFATVLKAWFADNLSSTSKTKIFSINYTMLNIGWTIGPPLGTLLVMQSINLPFWLAAICSAFPMLFIQIWVKRSEKIIATETGSVWSPKVLLQDKALLWFTCSGFLASFVSGAFASCISQYVMVIADGDFAEKVVAVVLPVNAAMVVTLQYSVGRRLNPANIRALMTAGTLCFVIGLVGFIFSGNSLLLWGMSAAVFTVGEIIYAPGEYMLIDHIAPPEMKASYFSAQSLGWLGAAINPLVSGVVLTSLPPSSLFVILALVIIAAWVLMLKGIRARPWGQPALC

SEQ ID NO: 17 - DNA, 1212 bp, Escherichia coli
atgtcgactcgtaccccttcatcatcttcatcccgcctgatgctgaccatcgggctttgttttttggtcgctctgatggaagggctggatcttcaggcggctggcattgcggcgggtggcatcgcccaggctttcgcactcgataaaatgcaaatgggctggatatttagcgccggaatactcggtttgctacccggcgcgttggttggcggaatgctggcggaccgttatggtcgcaagcgcattttgattggctcagttgcgctgtttggtttgttctcactggcaacggcgattgcctgggatttcccctcactggtctttgcgcggctgatgaccggtgtcgggctgggggcggcgttgccgaatcttatcgccctgacgtctgaagccgcgggtccacgttttcgtgggacggcagtgagcctgatgtattgcggtgttcccattggcgcggcgctggcggcgacactgggtttcgcgggggcaaacttagcatggcaaacggtgttttgggtaggtggtgtggtgccgttgattctggtgccgctattaatgcgctggctgccggagtcggcggttttcgctggcgaaaaacagtctgcgccaccactgcgtgccttatttgcgccagaaacggcaaccgcgacgctgctgctgtggttgtgttatttcttcactctgctggtggtctacatgttgatcaactggctaccgctacttttggtggagcaaggattccagccatcgcaggcggcaggggtgatgtttgccctgcaaatgggggcggcaagcgggacgttaatgttgggcgcattgatggataagctgcgtccagtaaccatgtcgctactgatttatagcggcatgttagcttcgctgctggcgcttggaacggtgtcgtcatttaacggtatgttgctggcgggatttgtcgcggggttgtttgcgacaggtgggcaaagcgttttgtatgccctggcaccgttgttttacagttcgcagatccgcgcaacaggtgtgggaacagccgtggcggtagggcgtctgggggctatgagcggtccgttactggccgggaaaatgctggcattaggcactggcacggtcggcgtaatggccgcttctgcaccgggtattcttgttgctgggttggcggtgtttattttgatgagccggagatcacgaatacagccgtgcgccgatgcctaa

SEQ ID NO: 18 - protein, 403 aa, Escherichia coli
MSTRTPSSSSSRLMLTIGLCFLVALMEGLDLQAAGIAAGGIAQAFALDKMQMGWIFSAGILGLLPGALVGGMLADRYGRKRILIGSVALFGLFSLATAIAWDFPSLVFARLMTGVGLGAALPNLIALTSEAAGPRFRGTAVSLMYCGVPIGAALAATLGFAGANLAWQTVFWVGGVVPLILVPLLMRWLPESAVFAGEKQSAPPLRALFAPETATATLLLWLCYFFTLLVVYMLINWLPLLLVEQGFQPSQAAGVMFALQMGAASGTLMLGALMDKLRPVTMSLLIYSGMLASLLALGTVSSFNGMLLAGFVAGLFATGGQSVLYALAPLFYSSQIRATGVGTAVAVGRLGAMSGPLLAGKMLALGTGTVGVMAASAPGILVAGLAVFILMSRRSRIQPCADA

SEQ ID NO: 19 - DNA, 1374 bp, Escherichia coli
atgccaaaagttcaggccgacggcctgccattgccccagcgatacggtgcgatattaaccattgtgattggtatttcgatggccgtccttgacggcgcaatcgccaacgtcgccctgccaacaatcgccacggaccttcatgccacgccagccagttccatctgggtagtgaacgcctatcaaatcgccattgtcatctccctgctctcgttttcgtttctgggcgatatgtttggctatcgacgtatttataaatgcggtctggtcgtttttctgttgtcttcactgttctgcgccctttctgattcgctgcaaatgctcacccttgcgcgtgtcatacaaggtttcggcggtgcagcgttgatgagcgttaataccgcacttatccgcctgatctatccacaacgttttctgggtagagggatgggcataaactcgtttattgttgccgtctcttctgctgccgggccgacaattgctgcagcaatcctctccatcgcatcctggaaatggttatttttaatcaacgtaccgttaggtattatcgccctgcttctggcgatgcgttttctgccacccaatggttctcgcgccagtaaaccccgtttcgacctgcccagcgccgtgatgaacgcgttaaccttcggcctgcttatcactgcgttgagtggtttcgctcaggggcaatcgctgacgttaattgctgcggaactggtggtaatggttgttgttggtattttctttattcgccgccagctttctcttcccgtaccgctgctaccggtggatttactgcgtatcccgctgttttcactttctatttgcacatctgtttgctctttctgcgcacaaatgctggcaatggtttccctgcccttttacctgcaaaccgtgctcgggcgtagtgaagtcgaaacaggtttacttctgacaccgtggccgttagcaacgatggtgatggctccgctggcaggctatttgattgaacgcgtacatgcaggattgctgggggctttagggttgttcatcatggctgcggggcttttttccctggttctgctgcccgcgtcacctgcggatatcaatattatctggccgatgatcttatgtggtgctggatttggcttattccagtcacccaataaccacaccattattacctccgcgcctcgcgaacgtagcggtggagccagtggcatgttaggaacggctcgtctactgggtcagagtagcggcgcggcgctggtggcgctgatgctaaatcagtttggagataatggtacacacgtctcgctgatggctgcggctattctggcagtgattgctgcctgtgtcagtggtttacgtatcactcagccacgatccagggcataa

SEQ ID NO: 20 - protein, 457 aa, Escherichia coli
MPKVQADGLPLPQRYGAILTIVIGISMAVLDGAIANVALPTIATDLHATPASSIWVVNAYQIAIVISLLSFSFLGDMFGYRRIYKCGLVVFLLSSLFCALSDSLQMLTLARVIQGFGGAALMSVNTALIRLIYPQRFLGRGMGINSFIVAVSSAAGPTIAAAILSIASWKWLFLINVPLGIIALLLAMRFLPPNGSRASKPRFDLPSAVMNALTFGLLITALSGFAQGQSLTLIAAELVVMVVVGIFFIRRQLSLPVPLLPVDLLRIPLFSLSICTSVCSFCAQMLAMVSLPFYLQTVLGRSEVETGLLLTPWPLATMVMAPLAGYLIERVHAGLLGALGLFIMAAGLFSLVLLPASPADINIIWPMILCGAGFGLFQSPNNHTIITSAPRERSGGASGMLGTARLLGQSSGAALVALMLNQFGDNGTHVSLMAAAILAVIAACVSGLRITQPRSRA

SEQ ID NO: 21 - DNA, 1218 bp, Escherichia coli
atggcaacagcatggtataaacaagttaatccaccacaacggaaagctcttttttccgcatggcttggatatgtatttgatggctttgattttatgatgatattttacattcttcatattataaaagcagatcttggcattacggatattcaggctactttaatagggacagtggccttcatagccagacctattggaggtggtttttttggtgccatggctgataaatatggtcgtaagccaatgatgatgtgggcaattttcatttactcagtcggaacaggccttagcggtattgctacaaacttatatatgctcgcagtttgccgttttattgttggcttagggatgtctggtgaatatgcatgtgcttcaacttatgcggtagaaagttggcctaaaaatcttcaatctaaagctagtgcttttttggtaagtggtttttctgttggaaatattattgcggcacaaataatccctcagtttgctgaagtatatggatggagaaactctttttttataggcctgttaccagttttactagttctttggatcagaaaaagtgctccagaaagtcaggagtggattgaagataaatataaggataaatcaacatttttgtctgtcttcagaaaaccacatctttcaatctctatgatcgttttcctcgtctgtttttgtctatttggtgcaaactggccgataaacggactacttccttcctacctggcagataatggagttaatacagtggtcatttcaactctgatgacaatagcaggtttaggaacactgacaggtacaatattttttggttttgttggtgataagattggtgtaaaaaaagcctttgtagtcggtctaataacttcatttattttcctttgtcctcttttttttatttctgtgaaaaactcttctcttataggattatgtctctttggattaatgtttacaaatttaggtattgcagggttggttccaaaatttatatatgattactttccaacaaaattaagaggattagggaccggtcttatttataacttaggggcaactggaggaatggccgcacctgtattagctacatacatttcaggatattatggcttaggtgtttcattattcattgttacggttgcattctctgccttattaattttgttagttggttttgatattccaggtaaaatttataaactatccgtggctaaataa

SEQ ID NO: 22 - protein, 405 aa, Escherichia coli
MATAWYKQVNPPQRKALFSAWLGYVFDGFDFMMIFYILHIIKADLGITDIQATLIGTVAFIARPIGGGFFGAMADKYGRKPMMMWAIFIYSVGTGLSGIATNLYMLAVCRFIVGLGMSGEYACASTYAVESWPKNLQSKASAFLVSGFSVGNIIAAQIIPQFAEVYGWRNSFFIGLLPVLLVLWIRKSAPESQEWIEDKYKDKSTFLSVFRKPHLSISMIVFLVCFCLFGANWPINGLLPSYLADNGVNTVVISTLMTIAGLGTLTGTIFFGFVGDKIGVKKAFVVGLITSFIFLCPLFFISVKNSSLIGLCLFGLMFTNLGIAGLVPKFIYDYFPTKLRGLGTGLIYNLGATGGMAAPVLATYISGYYGLGVSLFIVTVAFSALLILLVGFDIPGKIYKLSVAK

SEQ ID NO: 23 - DNA, 1191 bp, Escherichia coli
atgaccacccgacagcattcgtcgtttgctattgtttttatccttggcctgctggccatgttgatgccgctgtcgattgatatgtatctgcccgcgctaccggtaatttcagcgcagtttggcgtaccggcgggcagtacgcagatgaccctcagtacttatattctgggctttgcgttggggcagttaatctacgggccgatggcagacagcttcgggcgtaagccggtggtgctcggcggtacgctggtgtttgccgccgccgcggtggcgtgtgcgttggcaaacaccatcgatcagctgattgtgatgcgtttcttccacgggctggctgcggctgcggccagcgtggtcattaacgccctgatgcgcgatatttacccgaaagaagagttctcgcggatgatgtcgtttgtcatgctggtgacaaccattgcaccgctgatggcaccgatagttggcggctgggtgctggtgtggctgagctggcattacatcttctggatcctggcattagcggcgattctggcttcggcaatgattttcttcctgattaaagaaaccttaccaccggagcgtcgtcagccatttcacattcgtaccactattggtaactttgcggcgctgttccgccataaacgtgtcctgagctacatgcttgccagtggtttcagctttgccgggatgttctcattcttaagcgccggaccgtttgtttatattgaaattaaccacgtcgcgccggaaaactttggttattactttgcgctaaacattgtttttctgttcgtgatgaccatctttaacagccgcttcgtccgccgcattggcgcgttaaatatgttccgctcggggttgtggatacaatttattatggcagcgtggatggtcatcagtgcgctgctggggctgggattttggtcgctggtggttggcgttgcggcgtttgtgggctgcgtgtcgatggtgtcatccaatgcgatggcggtcattcttgatgagtttccccatatggcgggaacggcatcttcgctggcaggaaccttccgttttggcataggggcaattgttggcgcattgctttctcttgcgacctttaactctgcatggccgatgatttggtcaattgcattctgcgcaaccagctccattctcttctgtctgtacgccagtcggccgaaaaaacggtaa

SEQ ID NO: 24 - protein, 396 aa, Escherichia coli
MTTRQHSSFAIVFILGLLAMLMPLSIDMYLPALPVISAQFGVPAGSTQMTLSTYILGFALGQLIYGPMADSFGRKPVVLGGTLVFAAAAVACALANTIDQLIVMRFFHGLAAAAASVVINALMRDIYPKEEFSRMMSFVMLVTTIAPLMAPIVGGWVLVWLSWHYIFWILALAAILASAMIFFLIKETLPPERRQPFHIRTTIGNFAALFRHKRVLSYMLASGFSFAGMFSFLSAGPFVYIEINHVAPENFGYYFALNIVFLFVMTIFNSRFVRRIGALNMFRSGLWIQFIMAAWMVISALLGLGFWSLVVGVAAFVGCVSMVSSNAMAVILDEFPHMAGTASSLAGTFRFGIGAIVGALLSLATFNSAWPMIWSIAFCATSSILFCLYASRPKKR

SEQ ID NO: 25 - DNA, 1317 bp, Escherichia coli
atgggaaacacatcaatacaaacgcagagttaccgtgcggtagataaagatgcagggcaaagcagaagttacattattccattcgcgctgctgtgctcactgttttttctttgggcggtagccaataaccttaacgacattttattacctcaattccagcaggcttttacgctgacaaatttccaggctggcctgatccaatcggccttttactttggttatttcattatcccaatccctgctgggatattgatgaaaaaactcagttataaagcagggattattaccgggttatttttatatgccttgggtgctgcattattctggcccgccgcagaaataatgaactacaccttgtttttagttggcctatttattattgcagccggattaggttgtctggaaactgccgcaaacccttttgttacggtattagggccggaaagtagtggtcacttccgcttaaatcttgcgcaaacatttaactcgtttggcgcaattatcgcggttgtctttgggcaaagtcttattttgtctaacgtgccacatcaatcgcaagacgttctcgataaaatgtctccagagcaattgagtgcgtataaacacagcctggtattatcggtacagacaccttatatgatcatcgtggctatcgtgttactggtcgccctgctgatcatgctgacgaaattcccggcattgcagagtgataatcacagtgacgccaaacaaggatcgttctccgcatcgctttctcgcctggcgcgtattcgccactggcgctgggcggtattagcgcaattctgctatgtcggcgcacaaacggcctgctggagctatttgattcgctacgctgtagaagaaattccaggtatgactgcaggctttgccgctaactatttaaccggaaccatggtgtgcttctttattggtcgtttcaccggtacctggctcatcagtcgcttcgcaccacacaaagtcctggccgcctacgcattaatcgctatggcactgtgcctgatctcagccttcgctggcggtcatgtgggcttaatagccctgactttatgcagcgcctttatgtcgattcagtacccaacaatcttctcgctgggcattaagaatctcggccaggacaccaaatatggttcgtccttcatcgttatgaccattattggcggcggtattgtcactccggtcatgggttttgtcagtgacgcggcgggcaacatccccactgctgaactgatccccgcactctgcttcgcggtcatctttatctttgcccgtttccgttctcaaacggcaactaactaa

SEQ ID NO: 26 - protein, 438 aa, Escherichia coli
MGNTSIQTQSYRAVDKDAGQSRSYIIPFALLCSLFFLWAVANNLNDILLPQFQQAFTLTNFQAGLIQSAFYFGYFIIPIPAGILMKKLSYKAGIITGLFLYALGAALFWPAAEIMNYTLFLVGLFIIAAGLGCLETAANPFVTVLGPESSGHFRLNLAQTFNSFGAIIAVVFGQSLILSNVPHQSQDVLDKMSPEQLSAYKHSLVLSVQTPYMIIVAIVLLVALLIMLTKFPALQSDNHSDAKQGSFSASLSRLARIRHWRWAVLAQFCYVGAQTACWSYLIRYAVEEIPGMTAGFAANYLTGTMVCFFIGRFTGTWLISRFAPHKVLAAYALIAMALCLISAFAGGHVGLIALTLCSAFMSIQYPTIFSLGIKNLGQDTKYGSSFIVMTIIGGGIVTPVMGFVSDAAGNIPTAELIPALCFAVIFIFARFRSQTATN

SEQ ID NO: 27 - DNA, 1755 bp, Lactococcus lactis strain SRCM103457
atggctaaccgcatagaaggcaaagctgtggacaaaacctcaattaaacatttcataaaattaattcgcgcggcgaagccgcgctatttattctttataattggaattcttgcgggcatagtgggcacccttattcagctgcaggtacccaaaatggtgcagccgttagtgaacagctttggccatggcgtcaatggcggaaaagtggctctggtgatagcgttatatattggcagcgcggcggtgtcagcgattgcggcgattgtattaggaatatttggcgaaagcgtggtcaaaaaccttagaacacgcgtatgggataaaatgattcatctaccggtcaaatattttgatgaagtgaaaaccggcgaaatgagcagccgcctggctaatgatacaacacaggtgaaaaacttaattgcgaatagcattccgcaggcgtttacaagcattctgttattagtgggcagcattgtattcatgttacaaatgcagtggcgcttaacattagctatgattattgcggtgccggtcgtgatgcttattatgttccccattatgacctttggacagaaaattggacggacccggcaggatagcctggcgaacttccagggaattgcgagcgaaagcctgagcgaaattcggctggtcaaaagcagcaacgcggaaaaacaggctagcaaaaaagcggaaaacgatgtaaacgctttatataaaattggagtcaaagaagctatatttgatggcttaatgagcccggtgatgatgctgagcatgatgctaatgatatttggcttattagcgtacggcatttatcttataagcaccggcgtaatgagcttaggaaccttattaggcatgatgatgtatttaatgaatctgataggagctgtcccgaccgtagcgaccttctttaccgaattagcgaaagcgagcggcagcaccggccgcttaacagaactgttagatgaagaacaggaagtcttacatcagggcgaaagcttagatttagaaggcaaaacacttagcgcgcggcatgtggactttgcgtatgatgatagcgaacagattctgcgcgatataagctttgaagcgcagccgaacagcattattgcttttgctggcccgagcggcggcggcaaaagcaccattttcagcttactggaacgcttttatcagccgacagcgggcgaaattaccattgatggccagcccattgataacattagcttagaaaactggcgaagccagattggctttgtaagccaggatagcgcgattatggcgggcacaattcgcgaaaacctgacctatggcttagaaggcgactataccgatgaagacttatggcaggtgttagacctggcgtttgcgcggtcatttgtggaaaacatgccggatcagttaaacacagaagtgggcgaacggggcgtaaaaataagcggcggccagcggcagcgcctggcgattgcgcgcgcgttcttacggaacccgaaaatactaatgttagatgaagcgacagcgagcctggatagcgaaagcgaaagcatggtgcagaaggcgctggatagcttaatgaaaggccggaccacacttgtcattgctcatcggttaagcaccattgtggatgctgataagatttacttcattgaaaaaggccagataaccggaagcggaaaacataacgaattagtggctacccatccgctttatgcgaagtatgtgagcgaacagttaaccgtgggccagtaa

SEQ ID NO: 28 - protein, 584 aa, Lactococcus lactis strain SRCM103457
MANRIEGKAVDKTSIKHFIKLIRAAKPRYLFFIIGILAGIVGTLIQLQVPKMVQPLVNSFGHGVNGGKVALVIALYIGSAAVSAIAAIVLGIFGESVVKNLRTRVWDKMIHLPVKYFDEVKTGEMSSRLANDTTQVKNLIANSIPQAFTSILLLVGSIVFMLQMQWRLTLAMIIAVPVVMLIMFPIMTFGQKIGRTRQDSLANFQGIASESLSEIRLVKSSNAEKQASKKAENDVNALYKIGVKEAIFDGLMSPVMMLSMMLMIFGLLAYGIYLISTGVMSLGTLLGMMMYLMNLIGAVPTVATFFTELAKASGSTGRLTELLDEEQEVLHQGESLDLEGKTLSARHVDFAYDDSEQILRDISFEAQPNSIIAFAGPSGGGKSTIFSLLERFYQPTAGEITIDGQPIDNISLENWRSQIGFVSQDSAIMAGTIRENLTYGLEGDYTDEDLWQVLDLAFARSFVENMPDQLNTEVGERGVKISGGQRQRLAIARAFLRNPKILMLDEATASLDSESESMVQKALDSLMKGRTTLVIAHRLSTIVDADKIYFIEKGQITGSGKHNELVATHPLYAKYVSEQLTVGQ

SEQ ID NO: 29 - DNA, 1005 bp, Escherichia coli
atgaatgctgtaactgaaggaagaaaagtcctccttgaaatcgccgatcttaaagtgcactttgaaatcaaagatggcaaacagtggttctggcaaccgccgaaaacgctcaaagccgtcgatggtgtcactcttcgcctgtatgaaggggaaacattaggtgtggtaggggaatcgggatgcggtaagtccacctttgctcgcgccatcatcggtttggtcaaggcgaccgacggtcatgttgcctggttaggtaaagagttgctgggcatgaaacccgatgaatggcgtgccgttcgcagtgatattcagatgattttccaggatccgttggcatcgctaaacccgcgtatgaccatcggcgagatcatcgctgaaccactgcgtacttatcatccgaaaatgtcacgccaggaagttcgcgagcgcgtgaaggcgatgatgctgaaagtcgggttattgcctaacctgattaaccgctatccgcatgagttctctggtgggcagtgccagcgtatcgggattgcgcgtgctcttattcttgaaccgaagctgattatctgtgatgagccggtgtcggcgctggacgtgtcaattcaggcacaggtggtcaacctgctccagcaattgcaacgtgagatgggattgtcattaatttttatcgctcatgacctggccgtggtaaaacacatttccgatcgtgtgttggtgatgtatctcggccatgcggtagaactggggacctatgatgaggtctaccacaatccactacatccttacaccagggcattgatgtcggcagtccccatacctgatccggatctggagaagaacaaaaccatccagttactggaaggggaattaccgtcgccgatcaacccgccttccggttgtgttttccgtacccgttgcccgattgccggtccagagtgcgccaaaacacgtcctgttctggaggggagtttcagacactccgtttcttgcctgaaagtcgatccgctttaa

SEQ ID NO: 30 - protein, 334 aa, Escherichia coli
MNAVTEGRKVLLEIADLKVHFEIKDGKQWFWQPPKTLKAVDGVTLRLYEGETLGVVGESGCGKSTFARAIIGLVKATDGHVAWLGKELLGMKPDEWRAVRSDIQMIFQDPLASLNPRMTIGEIIAEPLRTYHPKMSRQEVRERVKAMMLKVGLLPNLINRYPHEFSGGQCQRIGIARALILEPKLIICDEPVSALDVSIQAQVVNLLQQLQREMGLSLIFIAHDLAVVKHISDRVLVMYLGHAVELGTYDEVYHNPLHPYTRALMSAVPIPDPDLEKNKTIQLLEGELPSPINPPSGCVFRTRCPIAGPECAKTRPVLEGSFRHSVSCLKVDPL

SEQ ID NO: 31 - DNA, 1251 bp, Escherichia coli
atgtcgttggcaaaagcgtccttgtggacggcggccagtacactggtcaagattggtgccgggttactggtcggtaagttgctggcggtgtcatttggtccggcggggcttgggctggcggcaaatttccgccagttgattaccgtgctcggcgtgcttgccggggctggcatctttaacggtgtaaccaaatacgttgcccagtaccatgataatccgcaacagctgcgccgcgtggtcggcacttcatcagcgatggtacttggtttctctacgctgatggcgctggtttttgtgctggcagctgcgccaatcagccagggattgtttggtaataccgactatcaggggctggtgcgtttagtggcgctggtgcaaatggggatcgcctggggcaacctgttactggcgctgatgaaaggctttcgcgatgccgcaggtaatgcgttatcgctgattgtcggcagcttgattggcgttctcgcgtactacgtcagttaccgtttgggcggttatgaaggggcgttgctgggtctggcgctgattcccgcgctggtggtaattcctgccgccatcatgttgatcaaacgtggtgtcatcccgttaagctatctgaaacccagctgggataacggtctggcagggcagttgagcaaatttacgctcatggcgttgattacgtcggtgaccttgcctgttgcttacatcatgatgcgtaaactgctggcggcgcagtatagctgggatgaggtggggatctggcaaggggtgagcagtatttccgatgcctacctgcaatttattacggcatcgttcagcgtatatttgctgcccacgttgtcgcggctaacggaaaagcgcgatatcacccgggaagtggttaaatcgctgaaattcgtcttaccggcagtggcggcggcgagttttaccgtctggctgctgcgtgattttgctatctggctgctgttgtcgaataaatttaccgctatgcgcgatctctttgcctggcagttagtgggtgatgtgttaaaagtgggcgcttatgtctttggttatctggtgatcgccaaagcgtcactgcggttttatattctggcggaagtcagccagttcactttattgatggtatttgcccactggctaatccctgcgcatggtgcactgggcgcggcgcaggcatatatggcaacttatatcgtctatttttctctttgttgtggcgtgtttttactctggcgtaggcgggcatga

SEQ ID NO: 32 - protein, 416 aa, Escherichia coli
MSLAKASLWTAASTLVKIGAGLLVGKLLAVSFGPAGLGLAANFRQLITVLGVLAGAGIFNGVTKYVAQYHDNPQQLRRVVGTSSAMVLGFSTLMALVFVLAAAPISQGLFGNTDYQGLVRLVALVQMGIAWGNLLLALMKGFRDAAGNALSLIVGSLIGVLAYYVSYRLGGYEGALLGLALIPALVVIPAAIMLIKRGVIPLSYLKPSWDNGLAGQLSKFTLMALITSVTLPVAYIMMRKLLAAQYSWDEVGIWQGVSSISDAYLQFITASFSVYLLPTLSRLTEKRDITREVVKSLKFVLPAVAAASFTVWLLRDFAIWLLLSNKFTAMRDLFAWQLVGDVLKVGAYVFGYLVIAKASLRFYILAEVSQFTLLMVFAHWLIPAHGALGAAQAYMATYIVYFSLCCGVFLLWRRRA

SEQ ID NO: 33 - DNA, 1140 bp, Escherichia coli
atgatgaaatccaaaatgaaattgatgccattattggtgtcagtaaccttgataagcggttgcacagtacttccgggcagcaatatgtcgacgatgggcaaagacgtcatcaaacagcaggacgctgatttcgatctcgacaaaatggtgaatgtttatccgctgaccccgcgcctgattgaccaattacgcccacgcccgaatgtagcgcgccccaatatgacgctggaaagtgagatcgcgaattaccagtatcgcgtcgggccgggggacgttcttaatgtcaccgtctgggatcacccggaactcaccacgccagccggtcagtaccgcagctccagcgacaccggcaactgggtacagcctgacggcactatgttttacccgtatatcggcaaggtccacgtagtcgggaaaacgctcgctgaaatccgcagtgatattaccgggcgcttagcgacgtacatcgctgacccgcaggtggacgttaatatcgccgccttccgctcacaaaaggcctatatctccggtcaggtgaataaatccggtcaacaggcgatcaccaacgtgccactgactattctcgacgccatcaacgccgcaggtggcctgaccgacaccgctgactggcgcaacgtggtgctaacacacaatggtcgtgaagagcgcatttctttgcaggcgctgatgcaaaacggcgacctcaaccagaatcgcctgctttaccccggcgatattctctacgtgccacgtaatgatgatctgaaagtatttgtgatgggtgaagtgaagaaacagagcaccctgaaaatggactttagcggcatgaccctgactgaagccctgggcaatgctgaaggcatcgacatgaccacctccaacgccagcggcatctttgtcattcgtccgctgaaaggcgagggcgggcgtaacggcaagattgccaatatctaccagctggatatgtccgatgccacgtcgctggtgatggcgacagaattccgcctgcaaccttatgacgtggtgtatgtcaccaccgccccggtttcccgctggaaccgtctgatcaatcagttgctgccaactattagcggtgtccgttacatgacggatacagccagcgacattcataactggtaa

SEQ ID NO: 34 - protein, 379 aa, Escherichia coli
MMKSKMKLMPLLVSVTLISGCTVLPGSNMSTMGKDVIKQQDADFDLDKMVNVYPLTPRLIDQLRPRPNVARPNMTLESEIANYQYRVGPGDVLNVTVWDHPELTTPAGQYRSSSDTGNWVQPDGTMFYPYIGKVHVVGKTLAEIRSDITGRLATYIADPQVDVNIAAFRSQKAYISGQVNKSGQQAITNVPLTILDAINAAGGLTDTADWRNVVLTHNGREERISLQALMQNGDLNQNRLLYPGDILYVPRNDDLKVFVMGEVKKQSTLKMDFSGMTLTEALGNAEGIDMTTSNASGIFVIRPLKGEGGRNGKIANIYQLDMSDATSLVMATEFRLQPYDVVYVTTAPVSRWNRLINQLLPTISGVRYMTDTASDIHNW

SEQ ID NO: 35 - DNA, 1737 bp, Helicobacter pylori strain ATCC 700392 / 26695
atggctaaaaagaaacataaaattagcacgctgaaatactttttacgtagcctgaagcagatctatatgcttatcacgttcaaggagaaaatggtttttttcctattagtgctaatggccgtttttagcagctttgttgaagtgatgagccttacgcttctaatgccgtttatcacgcttgcgtcagatccgaaccgtgcgctggacgataaagattggaaaatggtttatgattttttccatttttcaagcccagttcggttaatgtatttcttttctttttgcttagtgggcatttatttattccgtatgttttatggcgtgagcttcacgtatttaaaaggccggttttcaaataagaaagcatatcagatcaagcagcagttatttctgcaacatattaaaagcaattacctttctcacttaaatcataatttagatagcctgcgtgacattatcaataataaagctgaaggcatgtttatgagctttaatgctttcctgagcttacttacggaaataacggtgatcgtttttttctactcaacgcttatactgacgaattggaaaataacgcttgtttttacgatgatccttgcactgcagatttttttaattgtaaagaaagtaacggttcttatcaagaaaaagggagagatggccgcaaaatcaaaagcccagacgttaaaagttttttcaaaatttttcagcaacttcaaaatcacgaaacttaaagacaatcacgaagaagctcataagctttttggagaaaatagccggaaagctcatgacactgagattatttacgcgacgttgcaagtagtaccacgttattcaatagaaacggttggttttagcttattaattctggccgtagcgtatattcttttcaaatacggtgaagcgaaaatggtgcttccgacgattagcatgtatgcgcttgccttatatcgtatacttccgagcgtgacgggaatgatcaattactataacgaaatcgcgtataatcaattagccacgaatatggtttttaaaagcttaagcaaaacgatcgttgaagaggatctggtaccgctggactttaacgagaaaatcacgttgcagaatatttcattcgcgtataagtcaaagcatcccgttttaaaagattttaatcttacgattcagcgtgggcaaaaagtagcgcttataggtcattctggctgcggcaaatcaacgcttgccgatattattatgggcttaacgtacccgaaaagcggcgaaatttttattgataatacgttactgacgaataaaaatcgtcgttcatggcgaaaaaagataggttatatccctcagaatatttacttatttgatggtacggttggcgataatatcgcgtttggctctgcaatagatgaaaaacgtttaattaaggtgtgcaaaatggcgcatatttatgattttctgtgcgagcatgaaggcttaaaaacgcaagtgggcgaaggcggggcgaagttaagcggtgggcagaaacaacgtataggtattgctcgtgcactgtacgataatccggaaattttagttctggatgaagcaacgtcagcacttgacaatgaaacggagtctaaaatcatggatgaaatctatcagatcgcgaaaaataaaacgctaatcgttatcgctcaccgtctgagcacgattgaacgttgtgaagtaatcattgacatgagccagcataaagacaaccttggttaa

SEQ ID NO: 36 - protein, 578 aa, Helicobacter pylori strain ATCC 700392 / 26695
MAKKKHKISTLKYFLRSLKQIYMLITFKEKMVFFLLVLMAVFSSFVEVMSLTLLMPFITLASDPNRALDDKDWKMVYDFFHFSSPVRLMYFFSFCLVGIYLFRMFYGVSFTYLKGRFSNKKAYQIKQQLFLQHIKSNYLSHLNHNLDSLRDIINNKAEGMFMSFNAFLSLLTEITVIVFFYSTLILTNWKITLVFTMILALQIFLIVKKVTVLIKKKGEMAAKSKAQTLKVFSKFFSNFKITKLKDNHEEAHKLFGENSRKAHDTEIIYATLQVVPRYSIETVGFSLLILAVAYILFKYGEAKMVLPTISMYALALYRILPSVTGMINYYNEIAYNQLATNMVFKSLSKTIVEEDLVPLDFNEKITLQNISFAYKSKHPVLKDFNLTIQRGQKVALIGHSGCGKSTLADIIMGLTYPKSGEIFIDNTLLTNKNRRSWRKKIGYIPQNIYLFDGTVGDNIAFGSAIDEKRLIKVCKMAHIYDFLCEHEGLKTQVGEGGAKLSGGQKQRIGIARALYDNPEILVLDEATSALDNETESKIMDEIYQIAKNKTLIVIAHRLSTIERCEVIIDMSQHKDNLG

SEQ ID NO: 37 - DNA, 333 bp, Escherichia coli
atgaacccttatatttatcttggtggtgcaatacttgcagaggtcattggtacaaccttaatgaagttttcagaaggttttacacggttatggccatctgttggtacaattatttgttattgtgcatcattctggttattagctcagacgctggcttatattcctacagggattgcttatgctatctggtcaggagtcggtattgtcctgattagcttactgtcatggggatttttcggccaacggctggacctgccagccattataggcatgatgttgatttgtgccggtgtgttgattattaatttattgtcacgaagcacaccacattaa

SEQ ID NO: 38 - protein, 110 aa, Escherichia coli
MNPYIYLGGAILAEVIGTTLMKFSEGFTRLWPSVGTIICYCASFWLLAQTLAYIPTGIAYAIWSGVGIVLISLLSWGFFGQRLDLPAIIGMMLICAGVLIINLLSRSTPH

SEQ ID NO: 39 - DNA, 1524 bp, Bifidobacterium longum subsp. infantis (strain ATCC 15697)
atgtcgaacgagaataccgccgtgggcgatgtgcgcaagaaaggcggattaggccagcgcattgcgtatgcttgcggcaacttagggcaggccgcgttttataacgctatgtcgacgtattttgtgacgtatgtgacctcgtgcctgtttgtgtcgtattcgaaagctttagcggcgcagatgattgcggtgattaccggcctgattgtagtaattcgcattgcggaaatttttattgacccgttactgggcaacttagtggataataccaccaccaaatggggccggtttcgcccgtggcagtttattggcgggctggtgagcagcgtgctgattatgttaattttttcaggaatgtttggactggtgaacgtgaataccacgctgtttattgtgctgtttgtgattacgtttattgtgcttgatgtgttttatagcctgcgggatattagctattggggcatgattccggccttaagcagcgatagccatgagcggagcacctataccgcgttaggcacctttaccggcagcattggctataacggcattaccgtgattgtgattccgattgtgtcgtattttacctggacttttaccggcgcgaaaggccagggccaggccggatggaccagctttggctttattgtggccttattaggcctgattaccgcgtgggccgtggcgtttggcaccaaagagtcgaccaacgccctgcgcgcgaaagcgcagaaaaacggcaacccgtttgaagcctttaaagcgctgtttcagaacgatcagctgttatgggttgcgctgagctatctgctgtatgcgattgctaacgtgattacgaccggagtgatgtattatttatttgtgtttgtgcttgatgaaccggccgcgtttagcgtgaccgggattattccgctgattgcgggatttattatggccccgctgtatccgatattaaaccgctggataccacggcgctatctgtttgcgggcggcatggtgagcatgattattggctatacgatgttagcgctgtttagctcgaacctgccggttgtgattgtggcgctgatttttttttacgtgcccgcccagtttattcagatgaccgccattctgagcctgaccgatagcattgaatatggccagctgaaaaacggcaaacgcaacgaagccgtgaccctgagcgtgcggccaatgttagataaaattggcggcgccatgagcaacggagtggttggagcggtagcgttagccgctggcatgaccgggcacgcgacggctgcggatatgaccgccagcaatattaccacgtttaaaacctttgcgttttatattccgcttgtattaattattttatcgttagtggtgttttggtttaaagtgaaaattgatgagaaaatgcatgcccagattgtagatgaattagaggcgaaattagctagcggcgaaattgtggatgacgaagcccagaccgtggaagcggttgaagcgattaacgaagaaacgccggccgcgaaaaactaa

SEQ ID NO: 40 - protein, 507 aa, Bifidobacterium longum subsp. infantis (strain ATCC 15697)
MSNENTAVGDVRKKGGLGQRIAYACGNLGQAAFYNAMSTYFVTYVTSCLFVSYSKALAAQMIAVITGLIVVIRIAEIFIDPLLGNLVDNTTTKWGRFRPWQFIGGLVSSVLIMLIFSGMFGLVNVNTTLFIVLFVITFIVLDVFYSLRDISYWGMIPALSSDSHERSTYTALGTFTGSIGYNGITVIVIPIVSYFTWTFTGAKGQGQAGWTSFGFIVALLGLITAWAVAFGTKESTNALRAKAQKNGNPFEAFKALFQNDQLLWVALSYLLYAIANVITTGVMYYLFVFVLDEPAAFSVTGIIPLIAGFIMAPLYPILNRWIPRRYLFAGGMVSMIIGYTMLALFSSNLPVVIVALIFFYVPAQFIQMTAILSLTDSIEYGQLKNGKRNEAVTLSVRPMLDKIGGAMSNGVVGAVALAAGMTGHATAADMTASNITTFKTFAFYIPLVLIILSLVVFWFKVKIDEKMHAQIVDELEAKLASGEIVDDEAQTVEAVEAINEETPAAKN

SEQ ID NO: 41 - DNA, 1533 bp, Bifidobacterium longum subsp. infantis (strain ATCC 15697)
atgtcgaacgagaatgtggccgatggtgatgtgcgccgaaaaggcggactaggccagcgcattgcgtatgcctttggcaacttagggcaggccgcgttttataacgccatgtcgacgtattttattgtgtatgtgacctcgtgcctgtttagcggcgtagataaagcccttgccgcgaaactgattggcgtgattacctcgttagtggtgattattcgcattgcggaaatttttgtggacccgctgctgggcaacctggttgataataccaataccaaatggggccggtttcgcccgtggcagttttttggcggactggtgagcagcgtgctgcttgccgtggtattttcgggcatgttcggcctggtgaacgtaaatagcactttatttattgtggtgtttgtgattacgtttattgtgcttgatgtgttttatagcctgcgggatattagctattggggcatgattccggccttaagcagcgatagccatgaacggagcacctatactgcgttaggcagctttaccggcagcattggcggcaacgcgattaccattttagtgataccggttgtgacctatttttcctgggtttttaccggcgaaaacgcggaacatcagagcggatggaccgcttttggcattattgtggccgtgttaggcattatgaccgcgtggaccgtggcgtttggcacccgagagaaccaatcggcgcttcgcgcgaacgccgaggataacgggggaccgttgcaggcctttaaagcggtgtttcagaacgatcagctgttatgggttgcgctgagctatctgctgtatgcgattgccaacgtgaccaccaccggagtgcttttatttctgtttaaatttgtgcttgataatcaggccgcttttagcgccaccggagtgattgcgatgattgccggcctggtgatgagcccgctgtatccgatactgaaccgatatataccgcgccgctatctgtatattggcggcatggtgagcatgattgtggcttacgtgatgttagcgctgtttagctcgaatattgtgctggtgtttattgccctggtgttattttatgtaccaggcacgttaattatgatgacggtgattttaagcctgaccgatagcattgaatatggccagctgaaaaacggcaaacgcaacgaagcggtaacgcttagcattcgcccgatgttagacaaaattgggggcgctctgagcaacggcatagtgggctttattgcgcttgccgcgggcatgacgggcgacgctaccgccgcggacatgaccccgacgaatattcatgtgtttaaaatttgcgccttttacgcgccgttaattctgattgtgttatcgctggttgtgtttgtgagcaaagtgaaaattaccgagaaaatgcatgcccagattgtagatgaattagaggcgaaattagctagcggcgaaattgtggatgacgaagcccagccagtggaagccgcggaagccgcggctgcggttggcgacgaaacgcagactgtgaaaaactaa

SEQ ID NO: 42 - protein, 510 aa, Bifidobacterium longum subsp. infantis (strain ATCC 15697)
MSNENVADGDVRRKGGLGQRIAYAFGNLGQAAFYNAMSTYFIVYVTSCLFSGVDKALAAKLIGVITSLVVIIRIAEIFVDPLLGNLVDNTNTKWGRFRPWQFFGGLVSSVLLAVVFSGMFGLVNVNSTLFIVVFVITFIVLDVFYSLRDISYWGMIPALSSDSHERSTYTALGSFTGSIGGNAITILVIPVVTYFSWVFTGENAEHQSGWTAFGIIVAVLGIMTAWTVAFGTRENQSALRANAEDNGGPLQAFKAVFQNDQLLWVALSYLLYAIANVTTTGVLLFLFKFVLDNQAAFSATGVIAMIAGLVMSPLYPILNRYIPRRYLYIGGMVSMIVAYVMLALFSSNIVLVFIALVLFYVPGTLIMMTVILSLTDSIEYGQLKNGKRNEAVTLSIRPMLDKIGGALSNGIVGFIALAAGMTGDATAADMTPTNIHVFKICAFYAPLILIVLSLVVFVSKVKITEKMHAQIVDELEAKLASGEIVDDEAQPVEAAEAAAAVGDETQTVKN

SEQ ID NO: 43 - DNA, 1128 bp, Bifidobacterium longum subsp. infantis (strain ATCC 15697)
atggctgaagtggtatttgatcatgtgactcggatttatccgggcaacgacaaaccaagcgtggacgacctgaatcttgacattaaagacggcgaatttttagtgttagtgggccccagcggctgtgggaaatcaacgacccttcgaatgttagcgggcctggaagaagtgaataaaggccgcattttaattgggggcaaagacgtgaccacgatgcagccgaaagatcgcgatattgctatggtgtttcagaactatgctctgtatccgcatatgaccgtagcggataatatgggctttgcgctgaaaattgcgggcactccgaaagatgaaattcgcaaacgcgtggaaaaagcggctgaaattttagatctgaccgaatatttagatcgcaaaccgaaagctctgagcggtgggcagcggcagcgagtggcgatggggcgagctattgtgcgggaaccgaaagtgtttttaatggacgaaccgctgagcaacttagatgcgaaattacgggtgcagacccggacccagattgcggctctgcagcggcagttaggcgtgaccaccctgtatgtgacccatgaccagaccgaagcgctgacgatgggcgaccgcattgcggtgattaaattaggcattctgcaacaggttggcgcgccgaccgagctgtacgaccggccggcgaacgtgtttgtggccggctttattggcagcccgagcatgaacctgaatacccatccggttgtgaacggcaaagccaaaattggggaagataccgtggacctgccggccgaagcggtgaataaattaaccgccgaggataacggccagattgttgtgggctttcggccagaagatgccggcctggccccggttgacgatccgaacgcgtttagcctgaaagtagtgaacgtggaagatctgggcagcgacggctatatttatgggaccattgtgaccgacggaagcgcggccgaagcgagccaggtgatgagcgatcagaataaattaaccacgatacgggtgaatccgcgggccctgccgaaagtaggcgccaccgtgaaaattaaaattgatccggcgaaaatgcatctgtttgctccgagcactgaactgcggctgaactga

SEQ ID NO: 44 - protein, 375 aa, Bifidobacterium longum subsp. infantis (strain ATCC 15697)
MAEVVFDHVTRIYPGNDKPSVDDLNLDIKDGEFLVLVGPSGCGKSTTLRMLAGLEEVNKGRILIGGKDVTTMQPKDRDIAMVFQNYALYPHMTVADNMGFALKIAGTPKDEIRKRVEKAAEILDLTEYLDRKPKALSGGQRQRVAMGRAIVREPKVFLMDEPLSNLDAKLRVQTRTQIAALQRQLGVTTLYVTHDQTEALTMGDRIAVIKLGILQQVGAPTELYDRPANVFVAGFIGSPSMNLNTHPVVNGKAKIGEDTVDLPAEAVNKLTAEDNGQIVVGFRPEDAGLAPVDDPNAFSLKVVNVEDLGSDGYIYGTIVTDGSAAEASQVMSDQNKLTTIRVNPRALPKVGATVKIKIDPAKMHLFAPSTELRLN

SEQ ID NO: 45 - DNA, 1218 bp, Bifidobacterium longum subsp. infantis (strain ATCC 15697)
atggctctagacgtaggcaaggccttaaagagcaaaaccttagtggttggcgtgttaagcatgagcttactgatgagcgcgtcgaatgctgtgtcgggcaccataccggccatgaaagaagcctttagcgattatagcgccgccaacgttgagctgctgaccacggtgccgacgattggcagcatggttggcacggctttaaccggcctatttgcgaatgcgattggccgaaagaagattgccatggccggatttttaataagcgcggtgactggcgtgataccggccttttttccgtattactggccgatattaataagccgaatgttttttggctttggaagcgccctgtttgtgaccttatcagtgagctatattaccgatctgtacgatggcgacatgcaacgcaaactgcttggatggagacaggccgtgggcaatcttggcgatgtggtgctgctgtttgtggcgtcactgctgattaccattaactggcaaagcacgtatttaattttttttctgttatttgtgccaatggtgttagtgggcatgtttataccgaaagaatttgacaacttttcgatacgctcggccttagtggatgacgaaggccatgttgtggataaatcggcgagccagaaacagacgacgaactggcaagtgctgtggctggcctttatttttctggttgtgagcatgctttataacgtgatgagcattaaattagcgtcgtacgtggttgacgagggcattgggagcgctagcttagcgactttaattttttcgtttttagttgtggcgaccattttaagcggcgtgttatttgataaagtggcgaaagtgaccaaacgcctgacggtgaccattagcgaagtggtgattggcatttgttttattgcgactgccctgacgaaaaacgtgccgctgatgtttgcgctggtgctgatagcgggctttgcttggggcattattaacccagccctgaccgctcggtttgtggattatagcccagcccatagcatgaacctgaccacgagcattgtgataattggcattaatattggctgtctgattagcccgtatttttttgccctgacggcttcgatttttggcaatagcagcgcgggatttgcgattattgtgggaggggccttatatttagtgatggttgtgattgaactgattaccttaaaagtggataagaaattaacggtgtaa

SEQ ID NO: 46 - protein, 405 aa, Bifidobacterium longum subsp. infantis (strain ATCC 15697)
MALDVGKALKSKTLVVGVLSMSLLMSASNAVSGTIPAMKEAFSDYSAANVELLTTVPTIGSMVGTALTGLFANAIGRKKIAMAGFLISAVTGVIPAFFPYYWPILISRMFFGFGSALFVTLSVSYITDLYDGDMQRKLLGWRQAVGNLGDVVLLFVASLLITINWQSTYLIFFLLFVPMVLVGMFIPKEFDNFSIRSALVDDEGHVVDKSASQKQTTNWQVLWLAFIFLVVSMLYNVMSIKLASYVVDEGIGSASLATLIFSFLVVATILSGVLFDKVAKVTKRLTVTISEVVIGICFIATALTKNVPLMFALVLIAGFAWGIINPALTARFVDYSPAHSMNLTTSIVIIGINIGCLISPYFFALTASIFGNSSAGFAIIVGGALYLVMVVIELITLKVDKKLTV

SEQ ID NO: 47 - DNA, 1206 bp, Bifidobacterium longum subsp. infantis (strain ATCC 15697)
atgatacagcgctactctcaaggaacgtcagctagaagaatttttccaatattactgatagccgagggactatgcctaagcggcatgtccgtggacctgactattacctcactgaccgggagcagcttagcccccgctccctggatggctacttttccgatagcctgcatatttattgggacggtgattggcactccggctatgggacgcttagtgggacggtttggctaccggcaagtatttgtgtttggcgcgcttctagcggtgttagggggaataagctcagcgacggctttacggtttcatcagtttcccttattatgtataggaacgttttgtgtgggcatatatcagagcggaacgaactattatcggtacgcggctgcggacagcatgccaggcaaggagtcgaaagctattaacggaattctaagcgctggagcgatagcttcgattattggcccggtactggccactttttttggcactgccaccaatattgagtatgaaggatcatactatgtggtgtcggtgttagcggcgtgcgccgtaattgtgctgctggccttaccgaagatacagagccccactccagcccatatgcgcgataaggctaccgcctcgactaccccaaattacccggctttactgacccggccacggtttgtgctgggcgccaccatttcgtttgtggcttcatttgtgatggtgttagtgatgtcgggcgctccaatagtgttagaatcgacgtttagccaaaacgcccaaaccagaatggttgcgatgcaactgcatatggttggcatgtatctgcccaccctgtttgcgatttttatatttcataaaaagaactccgagatggcccaggccttaacgggattaattattggcatgttaggcactgtggttgctttagcggccagcgctagctacgcggtgactctggacctgctgctgattggaataagctggagcctgtgctatgcggctggctcggcgctgttaactaagagctatacggaaagcgagcgaagctgggcccgaggcgtgggcgagttagctccagtattagggcaagtgctgggatcagtgttagctggcgtgctgctggccgccggatggaaaaccgtatttgtgaccgtgctggccatgattgtatgcgccttaattgcgatggctggataccgcaataagattaaatcataa

SEQ ID NO: 48 - protein, 401 aa, Bifidobacterium longum subsp. infantis (strain ATCC 15697)
MIQRYSQGTSARRIFPILLIAEGLCLSGMSVDLTITSLTGSSLAPAPWMATFPIACIFIGTVIGTPAMGRLVGRFGYRQVFVFGALLAVLGGISSATALRFHQFPLLCIGTFCVGIYQSGTNYYRYAAADSMPGKESKAINGILSAGAIASIIGPVLATFFGTATNIEYEGSYYVVSVLAACAVIVLLALPKIQSPTPAHMRDKATASTTPNYPALLTRPRFVLGATISFVASFVMVLVMSGAPIVLESTFSQNAQTRMVAMQLHMVGMYLPTLFAIFIFHKKNSEMAQALTGLIIGMLGTVVALAASASYAVTLDLLLIGISWSLCYAAGSALLTKSYTESERSWARGVGELAPVLGQVLGSVLAGVLLAAGWKTVFVTVLAMIVCALIAMAGYRNKIKS

SEQ ID NO: 49 - DNA, 1224 bp, Bifidobacterium longum subsp. infantis (strain ATCC 15697)
atgtcaaagataataaattactcagaagtactggagagcaagcggctgatggtaggcgtgttaagcgtgtcatttctattaagcgctgggaatgctatatcgggaacgataccagccatggaagaggcctttagcaatataagcaaggcgaatatagaaactctaacgaccataccgactgccggaataatgttaggaaccgtgttaagcggagtatttagcaattacctaggaaagaaaaagagcgtgttagccgggttaataattgctttagtaggcggagtaattccagcctttctgccccaatattggcccattttcataagccggtttttattcggcgtgggcatgggaattttcaatccgctttcagtgagctatattaccgacctgtacgtgggcgaccggcagcggtcacttcttggatatcgcaatgccgtatcgaatctgggagacaccattatgctttttgtagctgggattttaataaccttcggatggaatattacctacctggtgttttttgcgttactgataccgatagtactaataatattatttgtacccaaagagtttgacaattttgacattcgaaactcagctctggacgaaaatgggcagatatcagattcagtgtcagacgtgaagccctcaacgaatttaaaagtgattgaagtaggcgtagtattcatggtaattactatgctatataacgccattccgctgaagttcgcctcgtatatagtgacggaacatattggcactgcgagcaccgcgacttggattttttcgtttctggtgttagccggcatatttagcggcgtgctgtttgagaaaataagcaaagtatttaaacggttaacggtgtttgtatttgaaatagtaataggcgtggcgtatattacgatagcctttacctataatataccgttactgacggctttagtgttaatttcaggcttcggatggggaataattaacccggctttaactgcccggttagtagacgtgagcccgataaactcaatgaatctgtcaacttcaataatagtaattttcatttcagtagggtcacttataagcccgtatttttttgctatgtttgccggcttatttgggaacgacagcgctgcctttgctatagtagttgggggagcgctgtacgtggtgatggccgtgcttgactttattaagataaagaagaataaagaactgtcgatttaa

SEQ ID NO: 50 - protein, 407 aa, Bifidobacterium longum subsp. infantis (strain ATCC 15697)
MSKIINYSEVLESKRLMVGVLSVSFLLSAGNAISGTIPAMEEAFSNISKANIETLTTIPTAGIMLGTVLSGVFSNYLGKKKSVLAGLIIALVGGVIPAFLPQYWPIFISRFLFGVGMGIFNPLSVSYITDLYVGDRQRSLLGYRNAVSNLGDTIMLFVAGILITFGWNITYLVFFALLIPIVLIILFVPKEFDNFDIRNSALDENGQISDSVSDVKPSTNLKVIEVGVVFMVITMLYNAIPLKFASYIVTEHIGTASTATWIFSFLVLAGIFSGVLFEKISKVFKRLTVFVFEIVIGVAYITIAFTYNIPLLTALVLISGFGWGIINPALTARLVDVSPINSMNLSTSIIVIFISVGSLISPYFFAMFAGLFGNDSAAFAIVVGGALYVVMAVLDFIKIKKNKELSI

SEQ ID NO: 51 - DNA, 1578 bp, Neurospora crassa OR74A
atgggcatttttaataaaaaaccagttgcccaggccgtggatttaaatcagatacaggaagaagcccctcagttcgaacgggttgattggaaaaaagatccaggtttacgtaaattatatttttatgcgtttatcttatgcatcgcctcggcgaccaccggttatgacggcatgttttttaactcggttcagaactttgaaacctggatcaaatatttcggcgatccacggggaagcgaattaggtttattaggtgccttatatcagatcggtagcatcggcagcattccattcgtgccattattaaccgataacttcggccgtaaaaccccaattattattggctgcgttattatgattgttggtgcggttttgcaggcgacggcgaaaaacttagacacttttatgggcggccggaccatgttaggcttcggcaatagcttagcgcagattgcgagcccaatgttattaaccgaattagcgcaccctcaacatcgtgcccggttaaccaccatttataactgcttatggaacgttggtgcgttagtggtgtcgtggttagcgttcggcaccaactatattaataacgattggtcatggcgtatcccagcgttattgcaggcctttccaagcattatccagttattaggtatttggtgggttccagaatccccacggtttttaattgcgaaagataaacatgatgaagcgttgcatattttagcgaaatatcatgcgaacggcgatccaaaccatccaaccgtgcagttcgaatttcgtgaaattaaagaaaccattcgtttagaaatggagtcgaccaaaaactcgtcgtatttagatttctttaaatcgcgtggcaaccgttatcgtttagcgattttattatcgttaggcttttttagccaatggagcggcaacgcgattattagcaactatagcagcaaattatatgaaaccgccggcgtgaccgatagcaccgcgaagttaggtttaagcgcggggcagaccggtttagcattaattgttagcgtgaccatggcattattagtggataaattaggtcggcgattagcctttttagccagcacgggcggcatgtgcggcaccttcgtgatctggactttaactgcgggcttatatggcgaacatcgtttaaaaggcgcggataaagcgatgattttcttcatttgggtttttggcattttttattcgttagcgtggtccgggttattagtgggctatgcgattgagattttaccatatcggttacgtggcaaagggttaatggtgatgaatatgtcggttcagtgcgcattaacgttaaatacttacgcaaacccagttgcattcgactatttcggtccagaccattcgtggaaattatacttaatctatacttgctggattgcggccgaatttgtgtttgtgttttttatgtatgtggaaaccaaaggcccaacgttagaagaattagcgaaagtgatcgacggcgacgaagccgacgttgcgcatattgatatccatcaggttgaaaaagaagttgaaatccatgaacacgaaggcaaatccgttgcctga

SEQ ID NO: 52 - protein, 525 aa, Neurospora crassa OR74A
MGIFNKKPVAQAVDLNQIQEEAPQFERVDWKKDPGLRKLYFYAFILCIASATTGYDGMFFNSVQNFETWIKYFGDPRGSELGLLGALYQIGSIGSIPFVPLLTDNFGRKTPIIIGCVIMIVGAVLQATAKNLDTFMGGRTMLGFGNSLAQIASPMLLTELAHPQHRARLTTIYNCLWNVGALVVSWLAFGTNYINNDWSWRIPALLQAFPSIIQLLGIWWVPESPRFLIAKDKHDEALHILAKYHANGDPNHPTVQFEFREIKETIRLEMESTKNSSYLDFFKSRGNRYRLAILLSLGFFSQWSGNAIISNYSSKLYETAGVTDSTAKLGLSAGQTGLALIVSVTMALLVDKLGRRLAFLASTGGMCGTFVIWTLTAGLYGEHRLKGADKAMIFFIWVFGIFYSLAWSGLLVGYAIEILPYRLRGKGLMVMNMSVQCALTLNTYANPVAFDYFGPDHSWKLYLIYTCWIAAEFVFVFFMYVETKGPTLEELAKVIDGDEADVAHIDIHQVEKEVEIHEHEGKSVA

SEQ ID NO: 53 - DNA, 1590 bp, Aspergillus oryzae RIB40
atgatcgctgtaggcaaacaacgggaagaagatgtgtctgatccagtgttagccaacttattagcggaagatcggaccccatggtataaaaaaccaaacttacggcgattatacttaattttatttccagcttgcatgggtatcgaaattacctctgggttcgattctcagattattaatactgtgcagattgtgtatacctggaataagtactttgggcgacttacgggagacaccgtggacgggatgccagaatacgaagtggagcctaatttaaagggtttccttggtgcagcttatagccttggtgcgattctttcgttaccattcgtgccatgggtaaatcaacggttcggtcggcgatggacggtaatgtttgggtcgtgcatttcgttagttggggcacttcttcaagggttttctaacggtgttggtatgtatatagtagcgcggatgctacttggtttcgggatcccatactgcattgtggccgggtcgtgcttaattggggaattagggtacccaaaagagcggccaattttaacgagcttattcaactctagctactttatcgggcaaattgttgcggccgccgttggtttaggtacggtgactattgcttcgaactgggcatggcgaattccatctttattgcagttagcgccagcgatggtgcaagtggtgttcgtatttttcttaccagaatcgccacggtacttaatttcgaaggatcggcatgaagaggcctttggtatcttagccaagtaccacgccgaaggcgatcggaatagcgtaattgtgcgggcagaaatcgcccagatcgagcggaccattaaattagaattagaggaagcaaagcaatcgtggtgggatatgtttcggaccgcagggatgcggcgacggttattaattagcgcattcttaggtttattcacccaatggagcggtaataccttaatctcttactacttatcggatttattagacatggttggtattaccgattcagtgaccaaaagcaaaattaatattgggatcgcgtgctggggactcgtgtcgggtaccgccctagcgcttacggcaccattatttaaacggcgaacgatgtacctaacctgtgcaacttctttactttgcgtgtatatcggttggactatcagcatggaacggtttatgactactgaagtgcgggcagccgctattttaactattttcttcattttcgcctatagccctgcttataaccttggttataatgcgttaacttatacttacttaatcgagatttttccatactttgggcgatcgcggggcctcagctggtttcagttctacggtcggggcagtgctttcttcgcgacttacgtgaatccagttgggttagaccggatttcgtggcgatggttattagtatactgctgttggcttgcctttgaattagtattcatttactttctttttcctgagacttcgggtcggactttagaagaattatctttcatgtttgaaggcaaggagaaggccaatgaagtggccgcggctgtgcataaacagattgaagtggacgggaaaactgagggccaggcgtaa

SEQ ID NO: 54 - protein, 529 aa, Aspergillus oryzae RIB40
MIAVGKQREEDVSDPVLANLLAEDRTPWYKKPNLRRLYLILFPACMGIEITSGFDSQIINTVQIVYTWNKYFGRLTGDTVDGMPEYEVEPNLKGFLGAAYSLGAILSLPFVPWVNQRFGRRWTVMFGSCISLVGALLQGFSNGVGMYIVARMLLGFGIPYCIVAGSCLIGELGYPKERPILTSLFNSSYFIGQIVAAAVGLGTVTIASNWAWRIPSLLQLAPAMVQVVFVFFLPESPRYLISKDRHEEAFGILAKYHAEGDRNSVIVRAEIAQIERTIKLELEEAKQSWWDMFRTAGMRRRLLISAFLGLFTQWSGNTLISYYLSDLLDMVGITDSVTKSKINIGIACWGLVSGTALALTAPLFKRRTMYLTCATSLLCVYIGWTISMERFMTTEVRAAAILTIFFIFAYSPAYNLGYNALTYTYLIEIFPYFGRSRGLSWFQFYGRGSAFFATYVNPVGLDRISWRWLLVYCCWLAFELVFIYFLFPETSGRTLEELSFMFEGKEKANEVAAAVHKQIEVDGKTEGQA

SEQ ID NO: 55 - DNA, 2811 bp, Helicobacter pylori (Campylobacter pylori)
atgaagaaccttaaaagcctattaagctttttactagcgagcttttgggtagcgatcccgctgatcgccctttacgcgtgtgcctgtgccatagcaacgtttatagaaaatgattacggcacgagcacgtcaaaggcgattgtgtataatacgccgtggtttaacttcttgcatgcctatttattagtggttctgataggcactttcattaaaagcaaagcgctggagcgtaaacggtatgccagcttatttttccatagctcattaattttcatcattctgggcgctgccatcacgcgtttctttgggatagaaggcttaatgcatgtgcgagaaaatagcgcccagagcagctttgaaagcgccgacacgtacttaaatatcacgttaaacgacacgacgaaattaagcctgaaaacgccgctgacgttttattattcaaaacgactgcgtccaattcatgcaacgctggatcataagccgctgattctggaaccgttagagatctataagcagaatgcaattaaaaaagatgacgcgacgattctggtgctgaaagccacgtataatggcgtgagccgtaaattcaatcttattaaaaataatcggaacgaaggcatagaagaaagcgaagtgtttaaagacgacaagcttagcctgtcatttggatcagcgtatattgaattaccatttcagatcaaactaaagcggtttgaactggagcggtacgcaggcagcatgtcaccgtcaagctacgcgtcagaagttgaagttttaaaattagataatacgctgatcaagccgtatcggatttttatgaatcatgttctggactatgaaggctatcgttttttccagagctcatatgacacggatgaaaaaggcacgatcttaagcgtaaataaagaccccgggaaaatcccaacgtatctgggctatgccatgttaattttaggcgcgttatggttattattagataagaatggccggtttctgaagttatcacgttttctgaaatctcaacaggttgcgtcattcctgcttgcgctgattcttgtaagcccgtttgcgagctcattcgcaaacgaaggccagattgacatgcatggcggcaaaagcgcgaaaattgagcggcagagcgtagaaaacccagcgaataaagaagatagcaaaagcgccattctggagcggctaaagcatttacgtgagtattcaaaagaccacctgaaagcatttcagcggctccaggtccaggattttgatggccggatcaagccgctggacacgattagcattgaatatattcataagattctgaaaaaagatgattttcagggccttaatgcaatgcaagtgttactgggcatcatgtttttcccaaacgattggcgaagcgttaaaatgatttatacgagcaataaagcactgcgtaagttaatcggcacgccgctggacgaaagccggatcgcgtttcgtgatgcctttgatagccgaggctataaactgaaaaacctagttgaagaagtaaaccagaaatcaccaaacgcccgtaatgagttagataaagatgtactgaaagtagatgaacgtatcaatctggtatatacgttatttagcgcgcagttcctgcgtattttcccgagcgataaaacgacggcgtggctttcaccaattgaagccatcaatagcccaaataaagaaatttcaagcgtggctacggaatttctgaaaaatatttttagcggctttgatgacgcgctgaaagcaaaccagtgggataaagtagaaaaaacgcttaaaaacctgagcgtatatcaacaggagcatgcaaaaaatctttatctgagcagcagcaaagtggatagcgaaatttttctgaatcatacgaacttttttaatagcctaacgttaccgtatatcttacttggcctgttattatttatcgttgtgatcagcagcttagttaaaaatacgattcctaatatttggctaacgaaaatcttatatgcagcgatcttattatgcgccatcgcgcatagcatgggccttattttacgttggtatgtgagcggccattcaccgtggagcaatgcgtatgagtcaatgttatatatcgcttgggcgagcgttatcgctggctttattctgcgttcaaaacttgccctttcagcgagcagctttctggcagggatcgccctttttgtggcgcatctgggctttatggacccgcagatcggccatctggtgcccgtattaaaatcatattggcttaatatccatgtaagcgtaatcacggcgtcatatgggtttctaggcttatgttttgtgcttggcattttatcattagttttatttatcttacgtaaacagggccggttcaacctggacaaaacgattctttcaattagcgcgatcaacgaaatgagcatgattctgggcctattcatgcttactgcaggcaacttcctgggcggcgtgtgggccaacgaaagctggggacgctattggggctgggacccgaaagaaacgtgggccttaattagcatttgcgtatatgcgctgatcttgcatttacggtttctggggagccataactggccatttattctggccagcagcagcgtgcttggcttttattcagttctgatgacgtattttggcgtgaactactacttaagcggcttgcatagctatgcagctggcgatccattacccatcccgacgtttctgtactttttagtagccataccgtttgcgcttgtgatcttagcctatttcaaacgtcatttatcattaccgaaactggtttaa

SEQ ID NO: 56 - protein, 936 aa, Helicobacter pylori (Campylobacter pylori)
MKNLKSLLSFLLASFWVAIPLIALYACACAIATFIENDYGTSTSKAIVYNTPWFNFLHAYLLVVLIGTFIKSKALERKRYASLFFHSSLIFIILGAAITRFFGIEGLMHVRENSAQSSFESADTYLNITLNDTTKLSLKTPLTFYYSKRLRPIHATLDHKPLILEPLEIYKQNAIKKDDATILVLKATYNGVSRKFNLIKNNRNEGIEESEVFKDDKLSLSFGSAYIELPFQIKLKRFELERYAGSMSPSSYASEVEVLKLDNTLIKPYRIFMNHVLDYEGYRFFQSSYDTDEKGTILSVNKDPGKIPTYLGYAMLILGALWLLLDKNGRFLKLSRFLKSQQVASFLLALILVSPFASSFANEGQIDMHGGKSAKIERQSVENPANKEDSKSAILERLKHLREYSKDHLKAFQRLQVQDFDGRIKPLDTISIEYIHKILKKDDFQGLNAMQVLLGIMFFPNDWRSVKMIYTSNKALRKLIGTPLDESRIAFRDAFDSRGYKLKNLVEEVNQKSPNARNELDKDVLKVDERINLVYTLFSAQFLRIFPSDKTTAWLSPIEAINSPNKEISSVATEFLKNIFSGFDDALKANQWDKVEKTLKNLSVYQQEHAKNLYLSSSKVDSEIFLNHTNFFNSLTLPYILLGLLLFIVVISSLVKNTIPNIWLTKILYAAILLCAIAHSMGLILRWYVSGHSPWSNAYESMLYIAWASVIAGFILRSKLALSASSFLAGIALFVAHLGFMDPQIGHLVPVLKSYWLNIHVSVITASYGFLGLCFVLGILSLVLFILRKQGRFNLDKTILSISAINEMSMILGLFMLTAGNFLGGVWANESWGRYWGWDPKETWALISICVYALILHLRFLGSHNWPFILASSSVLGFYSVLMTYFGVNYYLSGLHSYAAGDPLPIPTFLYFLVAIPFALVILAYFKRHLSLPKLV

SEQ ID NO: 57 - DNA, 1290 bp, Chitinophaga sp. CF118
atgagctttaacgtaaaaaaccggatcgcggaattattttcgaaacggatcttccgtgatagtttttattatactatcggtgcattcttagtcaaagggatcaacttcttcttagtcccagtgtatacgtcgtacctgtcgccatcggaatatggtatcttagatttatacctgacgtttacgaatatcttaacgatcttttgctcgttaggtttatctcaattaatcttcgtcgaattttataagatcaaaggtgaggaacggaagcggttcttctcggtcatcttttttgggtatgccttatttgggttaatcatctgcgctatcggtggagtgatcttccattttgcgagcgaatatttcactaaagttgccctggcgagcatgctaagcgtcatcttatcgtcggcaatcgcgtatatcacgttctatcaaacgaacttctttaactatttacggctagccggtcggtcgaagttatttgtttacttcagtatcatcactggtgtcgttaacgccggtctgaatatcttatttgtcgtcaaaggtaatatgggttataacggtatccttttaggtaatatcatcgttttattcgcgtcggttgtgtttggtttcttcgtgatcaaagataagatcgactttaagtttaatatcgacttaaaggttttttcgggttatacgaaaatcggtttagcctttatcttatcgtcgttatgccagtggttaatcaacggtgcggatagatggatcgtgctgaacgctttaaatacgactgagttaggtatctatgctttagcgtttaagttttcgtcatttgtcgatccattaatcatcacgccaatcacgtatgcctatcttccatatatctttaaaaagtatgctgaaaacgattatagcgaaaagttaatgaaaatcgccggtttagtgtttctgatctttctgccaatcgctttattagtaccatatttactaccaatggtaatcagtaataaggattattatatggcagtccaactgatcccattcctgatcatcgcttattatttttatttcatcagtcaattagccgggaacgtcttagtatatttaaagaagatccggtgcctggtgcagaatatcatcatcagcgggatcagcaatatcatcttaaattatatcttagttagaaagatgggaatcatcgggtgcgcgtatgcctatttaatctcgaatatcatctgggcaatcttaaatatctattaccgtaatatgtatttaagtagccttaagaacgaaaaatcgaactaa

SEQ ID NO: 58 - protein, 429 aa, Chitinophaga sp. CF118
MSFNVKNRIAELFSKRIFRDSFYYTIGAFLVKGINFFLVPVYTSYLSPSEYGILDLYLTFTNILTIFCSLGLSQLIFVEFYKIKGEERKRFFSVIFFGYALFGLIICAIGGVIFHFASEYFTKVALASMLSVILSSAIAYITFYQTNFFNYLRLAGRSKLFVYFSIITGVVNAGLNILFVVKGNMGYNGILLGNIIVLFASVVFGFFVIKDKIDFKFNIDLKVFSGYTKIGLAFILSSLCQWLINGADRWIVLNALNTTELGIYALAFKFSSFVDPLIITPITYAYLPYIFKKYAENDYSEKLMKIAGLVFLIFLPIALLVPYLLPMVISNKDYYMAVQLIPFLIIAYYFYFISQLAGNVLVYLKKIRCLVQNIIISGISNIILNYILVRKMGIIGCAYAYLISNIIWAILNIYYRNMYLSSLKNEKSN

SEQ ID NO: 59 - DNA, 1413 bp, Eubacterium sp. CAG:581
atgaaccgaaataaaaagttaggcattaacgtggtgttaatgagcataagcagctttggcagcaaattattaaccttcttcttagtgccgttatatactagctatctcaccaccgcggaatatggcgtgagcgatctcataaccaccaccaccagcctcttagcgccgatttttaccgcgaccataggcgaagcggtattacgctatgcgctggaaaaagatatagataagtatcaagtgtttcgcattggattatttatacatatggttgggtttattgctttaatgtgctttagcccgttactcctgatgataaagttactcaaaccgtattatgtgttctttatattatattattttagctttaccttttatagcttctttagcctattttgccgcgggataaataaagtggttgcgttcaccattagcgggattatacagaccctagtgatggttgggaccaatatagtaagcctcattttcttagatatgggaatatatgggtacctcctgagctttattgtgagcaactttgcgggcatgattctcttagtgactttaggcaaaatgcatgtatattttaaaataggcaaaattgataagaaattaatgaagaaaatgttattttatagcctgccgatgataaccaactcaattagctggtggattagcaatagcagcgataagtatattttaaccttcttatgcggcgcgaccataaacggcatttatagcgtggcgtataaaattccgaccatattaagcgtatgctatggcgtgtttatgagcgcgtggcgcctcagcgctgtggatgattttgggagcgaagaaaccgatcgcttttatagccaaatattaactaagatgaccaaggcgctgataattgtgggcgcgggcattgtgttatttaataaaattttagcgaaatttttatatgcgaaggatttctttagcgcgcgcgaatttgtgcccgtgttagtgattgcgtttttattgcatgggttaggcgaattctatggcagcgtgtataccagcgcgaaatgcaccaagatgctcttttatagcagcctcgcgggcgcggtgtgcaatatagtactcaactttgcgttaataccggaatttcaggcgatgggcgcggctattgcgaccatgattagctatggcgtgatactcgcgatacgcgcgattcatagccgcaccattatgaagatgaccattccggtgaccaatattaccattagcagcgtgatttttataaccatggcgattattcagaccatagatttccaaggcagctttattattagcgcgattttattctgtataattgcgtttttcaaccgcgatatggtagttgagattataaataccgtgttaaaaaagaaaccgaagaaaaccgcgaaacagtaa

SEQ ID NO: 60 - protein, 470 aa, Eubacterium sp. CAG:581
MNRNKKLGINVVLMSISSFGSKLLTFFLVPLYTSYLTTAEYGVSDLITTTTSLLAPIFTATIGEAVLRYALEKDIDKYQVFRIGLFIHMVGFIALMCFSPLLLMIKLLKPYYVFFILYYFSFTFYSFFSLFCRGINKVVAFTISGIIQTLVMVGTNIVSLIFLDMGIYGYLLSFIVSNFAGMILLVTLGKMHVYFKIGKIDKKLMKKMLFYSLPMITNSISWWISNSSDKYILTFLCGATINGIYSVAYKIPTILSVCYGVFMSAWRLSAVDDFGSEETDRFYSQILTKMTKALIIVGAGIVLFNKILAKFLYAKDFFSAREFVPVLVIAFLLHGLGEFYGSVYTSAKCTKMLFYSSLAGAVCNIVLNFALIPEFQAMGAAIATMISYGVILAIRAIHSRTIMKMTIPVTNITISSVIFITMAIIQTIDFQGSFIISAILFCIIAFFNRDMVVEIINTVLKKKPKKTAKQ

SEQ ID NO: 61 - DNA, 1350 bp, Dyadobacter soli DSM 25329 contig Ga0069981_102
atggctttgcagttacttcagaaattacggaataaacatttcttaagcctggccgggaacggcatcatgagcgtgttagggatgctcaatatgattattctgtatcgtgcattaccagtagcgagcatcggaatgtgggtcttttttctgagcatattattactggtagacacctttcggagcggctttttaacgacggcgtttattaaattctatgcaggcgccagcgatgcgcggaagcgtgaagtcgtcgggagcgcgtggttcattggcggggcgatcacgggcatcttagccctgattaatatcccggcctttctgttcagcgactggtttaagaacccatccgtggtgctgtttgtagaatggtttggcatcatttatatcgcgagcctgccgtactttattgctagctgcgtggtgcaggccgagcagcggtttgatcagttactatgcattcgttttttaagccagggattctttatcttattcgtcatggtaatggcttttaccaaaaccgcgaatttgcagaatattatttatgcatatttaggcggcgccgcattaacgagcatctttacgatcgtaatgggctggtcacggttaggcaactttaaggaccgtacttcagaaagcatcaaggaaatttttcacttcgggaaattctcagtcgggaccactttatcatcgaatttattcgggacttcgaatacgatgattattaactttatgttaggcccggccgcattagcggtgtttaacttagggcagcggctgatggaaattattgaaataccattacgaagctttgcggcgaccggcatgccagaattatccgcggcgtataatgaaggcaaccgtccaaaagtgattgaaaccatgaagcggtatgccggcttaattaccatggcattactaccggcgtgcatcggcgccgtgattttagccgacgtcgcgatacatattatagggggagaaaaatatatcaattcggaagcggccaatattatgcgtttatatatgacctttgccctgctgtatccgttagatcgtttctttgcattaactttagatgtcattcaccagccaaaaattaattttgtcaaagtactgattatgttagcagggagcgtcattgcttcagtgacggcgatctatattaccggctcaatctatggcgtagcgatcgccggcgtggtcccgacgttaattggcgtgggcattgggtactggggactcaaccggttccagccattttcaattctgagcgtctttacgacgggctacgccgaggccgtgtcactggtgcggctatggtggggcaagctgatggtccataagtctcaataa

SEQ ID NO: 62 - protein, 449 aa, Dyadobacter soli DSM 25329 contig Ga0069981_102
MALQLLQKLRNKHFLSLAGNGIMSVLGMLNMIILYRALPVASIGMWVFFLSILLLVDTFRSGFLTTAFIKFYAGASDARKREVVGSAWFIGGAITGILALINIPAFLFSDWFKNPSVVLFVEWFGIIYIASLPYFIASCVVQAEQRFDQLLCIRFLSQGFFILFVMVMAFTKTANLQNIIYAYLGGAALTSIFTIVMGWSRLGNFKDRTSESIKEIFHFGKFSVGTTLSSNLFGTSNTMIINFMLGPAALAVFNLGQRLMEIIEIPLRSFAATGMPELSAAYNEGNRPKVIETMKRYAGLITMALLPACIGAVILADVAIHIIGGEKYINSEAANIMRLYMTFALLYPLDRFFALTLDVIHQPKINFVKVLIMLAGSVIASVTAIYITGSIYGVAIAGVVPTLIGVGIGYWGLNRFQPFSILSVFTTGYAEAVSLVRLWWGKLMVHKSQ

SEQ ID NO: 63 - DNA, 1233 bp, Lactococcus raffinolactis (ATCC 43920)
atgtttttagccgtatgggaaggcattttacggttcggcttaaacgaaaataaccgggaaaaattaacgagcttaattagcaccaccgtattttttgggttaggggcaacgatattttgggctattattttaccttttccatatttacggatctttaatacgttaccgtatgttcacttattcattattagcttattattgcagcctattttatcaacgttgcagattgcggtccgagggattaaggagtcgaagaactatgtgatatcaggcattatcagcacttttgttaacgtgggcttattaattttcttagtggtgttcttacggttagggttattaggcttactcttatcatttttaattggcaaccttgtgaacgtgttatatttattatttggctcatcactcttaaaatatatttcatttaaagccatcgataaaaacctcttaaagaagttattaaaattcagctggccattaatttttaacttagtatttgtgtggtttctcggtggctatgttatgttttatttaggctcgtttgtaggcagcgatgaaaccggctttttcagctttgcgaataaatttgcctcaattgtggcgatgtttgggagcgtgttaggcatggccactatagaagatactattattacttcggaaaacgataactttattgaaagctttgctaagaagaatacggaaatgtttaagatgtttttaaacgtgggcatattactcttaccagtaattggcatgtactatttcaccttaggcaatagcgcgtatcagaatactcttataattgtcccgttcttattaatggcgtcaatttttcaagccatggctaccaacgtgggcaatattatgattgtgcatcagaaaactaaggctattgccgtggcgtcgttattagtgggcatatttaacgccatcttctgctttgtagggtaccactttctcggcttaaccggcgtgtgcattgcctatctctttgcttcatttttattatttttattccggtataagatggggcaaaaaattcagaactataacttaaaatggcgctttattgtagtgctcgcggtcatattcattatgttaggcttattaatacagtttgaaaacgtgattatcaatagcattttagtcttagtcactgcggtgtttgagatatttatttatcggcatattatattaaaaatactcaaccgcattattgggcgagcgtaa

SEQ ID NO: 64 - protein, 410 aa, Lactococcus raffinolactis (ATCC 43920)
MFLAVWEGILRFGLNENNREKLTSLISTTVFFGLGATIFWAIILPFPYLRIFNTLPYVHLFIISLLLQPILSTLQIAVRGIKESKNYVISGIISTFVNVGLLIFLVVFLRLGLLGLLLSFLIGNLVNVLYLLFGSSLLKYISFKAIDKNLLKKLLKFSWPLIFNLVFVWFLGGYVMFYLGSFVGSDETGFFSFANKFASIVAMFGSVLGMATIEDTIITSENDNFIESFAKKNTEMFKMFLNVGILLLPVIGMYYFTLGNSAYQNTLIIVPFLLMASIFQAMATNVGNIMIVHQKTKAIAVASLLVGIFNAIFCFVGYHFLGLTGVCIAYLFASFLLFLFRYKMGQKIQNYNLKWRFIVVLAVIFIMLGLLIQFENVIINSILVLVTAVFEIFIYRHIILKILNRIIGRA

SEQ ID NO: 65 - DNA, 1305 bp, Prevotella ruminicola (AR32)
atgactgaaagccggatgaagcggtcgctgactggtacgttttggtcgatgacggagcgattcgctaaaatgggaatacagatagtttgcacgtttatcatcgcccagtttgttgcaccctcggaatttgggctggtgagcatgatgagcatctttttagcgttttcgacgatcttaatagatagcggcttttcgcaggcactgatccatgagcagaacgtaactcctcaagatgaaagctcgatattttggttcaatatcggtctagggtgcgctgtctatggcatattttggctgatcgctccattaatcgcgaacttttataacgaacctcagctaactctactaatccgagtggcctttttagcactgatatttcaatcgttaatcgttgttcagcaaggactattatttaaaagcgttgactttaaagcggtgtcgaagatctcgttttgggctgttttattatcgggtatcgccggtatcgtggtgagctatatccggaaagatgtgtggggcctaatcgttcaaaacctgctgttcgccttacttcaaactgttttatactgggtttattcgcgttggcgtccaagcttaatctttaaaatgcaatgtgtgcgaaagtacttacgattttcgatgaatctgctgggtagcaatatgctggcagcgataacggataatctggcgaacctgttcgtcgggaaggcgtataatgcgacgatattagggcattatactatggcgaataaaataccatatttaacgtcggggacggtttgttatggtatcaagcgtgtgagctattcgatcatgtcgacgttccagaacgataacgagcaactagcgcggtattcgcaacgtgtggttgggacggcgttctggatcttatcgccagttatgatcttaatgctggttctggcagagcccttcatctcgtggctatttccagaggagtgggctccagccgcaatatatctgcgttatttttgtgtgatagggctggtgtactgctttgccgatgttaatcaggatatcctgctagtgaaaggacgcacggatatcctatttcgtttagatatcgtgcgacggacggtgttagttgcactgctgatcatcggtatacaatactcgatggaaacgtttttactgctgttagtgatatataatatactaaacgcgatcatcgtgagctatatagcaggccgactgatagattgttcgttatggcgacagatacgactggttgggtcgacgccactatatttcctaactaatatcaaccaacggcgagagaaacggtga

SEQ ID NO: 66 - protein, 434 aa, Prevotella ruminicola (AR32)
MTESRMKRSLTGTFWSMTERFAKMGIQIVCTFIIAQFVAPSEFGLVSMMSIFLAFSTILIDSGFSQALIHEQNVTPQDESSIFWFNIGLGCAVYGIFWLIAPLIANFYNEPQLTLLIRVAFLALIFQSLIVVQQGLLFKSVDFKAVSKISFWAVLLSGIAGIVVSYIRKDVWGLIVQNLLFALLQTVLYWVYSRWRPSLIFKMQCVRKYLRFSMNLLGSNMLAAITDNLANLFVGKAYNATILGHYTMANKIPYLTSGTVCYGIKRVSYSIMSTFQNDNEQLARYSQRVVGTAFWILSPVMILMLVLAEPFISWLFPEEWAPAAIYLRYFCVIGLVYCFADVNQDILLVKGRTDILFRLDIVRRTVLVALLIIGIQYSMETFLLLLVIYNILNAIIVSYIAGRLIDCSLWRQIRLVGSTPLYFLTNINQRREKR

SEQ ID NO: 67 - DNA, 1284 bp, Flavobacterium spartansii
atggaaaaagatataatactgaaggcagaaaatattagcaaacagtatcgtctgggccaggttggtactggcactctgtcgcatgacattaaccgttggtggcataagattcgcggtaaagagaatccgtatctgaaaattggagacacgaacgatcgttcaacgaaaggcaatagcgattatgtatgggccttgcaggacattaactttgaagtggaacggggcgaagtgctaggcattattggtaaaaatggcgctggcaaaagcacgttactgaagatattaagcaaagtgacggcgccaacgactggctcgataaaatcacgtggccgtattgcgagcttattagaagtaggcacgggtttcaacggcgaaatgactggccgcgaaaatatttttcttaacggtgcaattttaggcatgactaaaaaagaaattagcagcaagttagatgaaattatagaatttagcggttgcgaacgttatatagatacgcccgtgaaacgctactcaagcggcatgacggtacgtctggcgtttgctgtggctgcgtttttagaacccgaaattttagttgtggatgaagtgttagctgtgggcgatgcagaatttcagaagaaggcgattggtaaaatgcaagatattagcaaaggtgaaggccgtactgtgctgtttgtgtctcataatatggccgcggtgaagtcgctgtgcacgcgcggcatagtgctagaaaacggaaaaattaaatttaacggcgatattgatgaggcgttagatgtgtatagcaataacggtaatagcacggatcatacggttgtgtttgatagcgatacgaaacggacgggatcacgcaatatagagtttataagcgttgagatacgcgatcagaatcagaagccatcgcatgaattcagcattggcgatgatctgattattaaattccgccttcagaataatacgaatgaagcgaaaagcgaagtaggcatacaaatacgcagcagcgacgaaatgccgatttttcatattatgggtcgcgatagcaactttgaactgactcatgtggaaaaacaggaagagtatgtggtgtcgataaaagacatacgcttatttcccggaatgtatactatttcatttacgtcgaatacgacgactggccaccaaatatttgactgcattgaaaacgcgttaagctttcagattatagatggcggcaaatatactattcgctcgttaccacgcgcggcaggcttattctttctgaacccagaatggaaaaaattataa

SEQ ID NO: 68 - protein, 427 aa, Flavobacterium spartansii
MEKDIILKAENISKQYRLGQVGTGTLSHDINRWWHKIRGKENPYLKIGDTNDRSTKGNSDYVWALQDINFEVERGEVLGIIGKNGAGKSTLLKILSKVTAPTTGSIKSRGRIASLLEVGTGFNGEMTGRENIFLNGAILGMTKKEISSKLDEIIEFSGCERYIDTPVKRYSSGMTVRLAFAVAAFLEPEILVVDEVLAVGDAEFQKKAIGKMQDISKGEGRTVLFVSHNMAAVKSLCTRGIVLENGKIKFNGDIDEALDVYSNNGNSTDHTVVFDSDTKRTGSRNIEFISVEIRDQNQKPSHEFSIGDDLIIKFRLQNNTNEAKSEVGIQIRSSDEMPIFHIMGRDSNFELTHVEKQEEYVVSIKDIRLFPGMYTISFTSNTTTGHQIFDCIENALSFQIIDGGKYTIRSLPRAAGLFFLNPEWKKL

SEQ ID NO: 69 - DNA, 1305 bp, Sporomusa sphaeroides DSM 2875
atgtcagatactgttataaaggttgagaacttatcaaagcaatatcaattaggtactattggttcgggtactttatatcgtgatttgcagtcgtggtgggctcggttaaagggtaaagaagatccgaattcaaagctaggtcaatgggatgtttctcagataggcgaagataatagcttttgggctttacgagatgtatcatttgagattaaccaaggggacattgttggtattattggtcgtaacggggcaggcaaatcaactttattaaagatattatcacgggttactgggccaacttcggggcagataaaaataaagggtcgggttgcatcgttattagaagttggtactggttttcatccggaactgactgggcgagaaaatgtttatctaaacggggccattctaggtatgcggaaggccgagatagatcgtaaatttgacgagatagttgacttcgctgaaatatcaaagtttatagatactccagttaaacggtattcatcaggaatgtatgttcggttagcctttgccgttgccgctcatctagatccagaaattttaattgtagatgaagttctggccgttggggacatagagtttcaaaagaagtgcttaggcaagatgaaagatgttggccaacaggggcgaactgttttatttgtttcgcataatatggttgccattgaacaattatgctcatcggctattctgatcgaaaatgggcaaataaaaagcaaatcgtttactgttcgggatattattaaggattatttatataaaaacgaatatagccaatttactctatgggagaataaggagaaagaatttagcaacccatatttttatccagttaaaatgtatattggtgataaagatggtaagttactacctaccccaatacgtaacgatcaagatgccttcatttttatagaagcccagattaagaagattgataaagcattaactgttggttatgctatatatactgattcgggtgagtgcttatattggtcatatcagactgacgagagcgaagaagaatggcctactctaatagttggctcaaacgttttatattcaaacctgccaaaacggttattaaacgaagggaaatatcggattgaattaattggtggagtccattttcgaaagtggttatttcagccattaaaatcaccgccttcgatttcgttagaaattgttggtggcttatcggattcaccatatctattatataagcggccaactgttttaaaccctataattcgatggaactcatttaatcgaaaggttgagtaa

SEQ ID NO: 70 - protein, 434 aa, Sporomusa sphaeroides DSM 2875
MSDTVIKVENLSKQYQLGTIGSGTLYRDLQSWWARLKGKEDPNSKLGQWDVSQIGEDNSFWALRDVSFEINQGDIVGIIGRNGAGKSTLLKILSRVTGPTSGQIKIKGRVASLLEVGTGFHPELTGRENVYLNGAILGMRKAEIDRKFDEIVDFAEISKFIDTPVKRYSSGMYVRLAFAVAAHLDPEILIVDEVLAVGDIEFQKKCLGKMKDVGQQGRTVLFVSHNMVAIEQLCSSAILIENGQIKSKSFTVRDIIKDYLYKNEYSQFTLWENKEKEFSNPYFYPVKMYIGDKDGKLLPTPIRNDQDAFIFIEAQIKKIDKALTVGYAIYTDSGECLYWSYQTDESEEEWPTLIVGSNVLYSNLPKRLLNEGKYRIELIGGVHFRKWLFQPLKSPPSISLEIVGGLSDSPYLLYKRPTVLNPIIRWNSFNRKVE

SEQ ID NO: 71 - DNA, 864 bp, Flavobacterium spartansii
atgaataataactcacccaacgattggttatttgaaataacgccgaaaaataagttcttctcactgaatctgaaagaagtgtggcaatatcgtgacctgctgtttttattcgtgaaacgtgacgtgataacggtgtataagcagacggtgctgggaccattatggtatttgattcaaccattatttacgagcgtgacgtttacgataatattcaataacgtggcaggcattgagactggcacggtgccgccatttctgtttaacttggctggcataacggtgtggaactattttacggcatgtttgacgggaacgagcgacacgtttaaggcgaacgcagctatttttggcaaagtgtattttccacgaataattacgccgttgtcagtggtgattagcaacttagtgaagtttggcatacagtttctgatttttattggcttctatattttctactattttcagggcgcggatctgtcgcttaacggtttagtgctgttttttccggtgctgataataataatgggcattctgggcttaggactaggtatgttaataagcgctatggtgacgaaatatcgtgacttttcgcatctgattggttttggaatacagctgttaatgtatctgagcgcggtgatgtatccgatggaattaattaaagataaactgccaagctacggatggttagtggtgtataacccactggcgtatattattgagactgcacgttttatgctgttaaacgttggtgatatttcagtgttaggcttaacgtatacgctgattgtgacgataacggtgttttttataggcctgttggtgtttaataaaactgagaaatcgtttatagatacggtgtaa

SEQ ID NO: 72 - protein, 287 aa, Flavobacterium spartansii
MNNNSPNDWLFEITPKNKFFSLNLKEVWQYRDLLFLFVKRDVITVYKQTVLGPLWYLIQPLFTSVTFTIIFNNVAGIETGTVPPFLFNLAGITVWNYFTACLTGTSDTFKANAAIFGKVYFPRIITPLSVVISNLVKFGIQFLIFIGFYIFYYFQGADLSLNGLVLFFPVLIIIMGILGLGLGMLISAMVTKYRDFSHLIGFGIQLLMYLSAVMYPMELIKDKLPSYGWLVVYNPLAYIIETARFMLLNVGDISVLGLTYTLIVTITVFFIGLLVFNKTEKSFIDTV

SEQ ID NO: 73 - DNA, 852 bp, Sporomusa sphaeroides DSM 2875
atgcatctagaacaggaacaatggacttcgattattaaaccttcgaacggatggtttgatattgattttaaagggttatggcactatcgggatttaattgcaatgatggttaaacgggattttgttgccttttataaacaaaccatcttagggcctctatggtttttactgcagccgttattaactactattgtttttactattgtttttggccagattgccaaattacctactaacgggttacctcaagtattattttatttatcagggactgttatgtggaactactttgctgcctgcttaaatactacctcaaatacctttgttgcgaacgccggtatttttggtaaagtttattttccacggctagttataccaattggcgttgttattaccaatatgttatcattcgggattcaatttgcactatttttagctctactaatcttctttatgcttcaaggggcagcagttgccccaaacttatggatcttagttactcctttattagttctgcaaatgggctgcttagggctaggttgtggtattatagtttcatcaatgactaccaaataccgggacttatcattattagttacttttggtgcccagttatggatgtatgcgactccggttgtttatcctgtttcgatggcatccgggttcatgagctgggttttagtattaaacccaatgactgctattatcgaaatctttcgttacgcgtttttaggtgggcagatgattccattatggtactggttagttagcggtgcaattaccgttattttattcatggttggtattattctattctcgaaagttgagaaaaattttatggatactgtttga

SEQ ID NO: 74 - protein, 283 aa, Sporomusa sphaeroides DSM 2875
MHLEQEQWTSIIKPSNGWFDIDFKGLWHYRDLIAMMVKRDFVAFYKQTILGPLWFLLQPLLTTIVFTIVFGQIAKLPTNGLPQVLFYLSGTVMWNYFAACLNTTSNTFVANAGIFGKVYFPRLVIPIGVVITNMLSFGIQFALFLALLIFFMLQGAAVAPNLWILVTPLLVLQMGCLGLGCGIIVSSMTTKYRDLSLLVTFGAQLWMYATPVVYPVSMASGFMSWVLVLNPMTAIIEIFRYAFLGGQMIPLWYWLVSGAITVILFMVGIILFSKVEKNFMDTV

SEQ ID NO: 75 - DNA, 1851 bp, Candidatus Planktophila sulfonica
atgattgaattccttagcaaaccaaagttcagcatacttactaaatccccgatttatcgagccagccgcgtattatccgttaaggaccggcgaaagattagccttgtgattgtggttcaggtttgccttggtctgtttgatttagccggtgtagcggtgtttggtgtaatgggtagcttagcggtaagcggagtgcagagccagagcccgaactcacgggtaggccaggttcttgatgcgattggaatagccaacctgccgttccaaaagcaggccgcgattctggcgattctggctacttgcttccttatggcccggaccttccttactgtagtggtaacgaaacggactatgcttttcctgtcccgacgaggcgccgctattagctcagatttagtagctcgactgctgagcaagtcattacttttcattcaggagcgaacgactcaacagacgcttttcgctctgacgcaaggtgtgagcacgcttactataggtgtacttgctaccgtggtaacgatgattagcgatacgagccttcttattttacttactgcgggccttttcgtagtggatcccgcgatagctatatccacgttagtggtattcggtagcattattctgttcatgtataaaaaccttcataataaagctaaaacgctgggtttcgcggaaagcactcttcatataaagtccaacgaaaaggttcttgaagtgttaaactcataccgggaaagcgtagttcgaaatcgcaggaactattatagcgagcaaattggtatgttacgattcgatcttgctaagacccaggcgcagttatcctttatgccctatattggtaagtatgtattagagactagcgtagtattaggttcacttttaattgcggcgattcagttcgcgttgcacgatgctgttaccgccgtggccacgctttcaatatttcttgtggctggtacgcgcattagcccagctgctttaaggctgcaacaggcgcttattcagattaagagctcactgggtggctcagaaccgactcttgatttaattgatgcccttcgtgacacccttccagcgacgtccgttaatcaagagctggaccttacgcatgaacagttcgtaccccgaattcagattcaggatatatcatttacgtaccccaactcagatcggcaggccttaagcggtgttaaccttgatattcagtccggttccgtggttgccctggtaggcccatcaggtggcggcaagacgacgcttgttgatgtaattttaggtattattgaacccaatagcggtaaagtattaatttcaaatcttacccccattgatgctattcagatttcaccgggtgcgatttcatacgtgccccaagatgtgtcaattattgatggtacggtacgagaaaatattgcgatgggtttccccgttgaagtggctaccgatgagcttgtgcataatgctattgaattagctggattaaaagaattcgttgctacgctgccgaatggtcttgacacgtacgtaggcgaaaaaggtgctcggatttcaggtggccaacggcagcgcctgggaattgctagggcgctcttcacgaacccgaaattactggtactggatgaagctacttccgccttagatggtgaaacggagagccgaattacggatagcatactgtccttaaagggtaaagttacggtattaatggttgctcataggctgtccacggtacgcaacgccgatcaagttgtgtatatgtcagaaggcagcattaaagctgtaggcactttcgagagcgtgaggaatacggtgccggatttcgatacgcaggcccaacttatgggattatga

SEQ ID NO: 76 - protein, 616 aa, Candidatus Planktophila sulfonica
MIEFLSKPKFSILTKSPIYRASRVLSVKDRRKISLVIVVQVCLGLFDLAGVAVFGVMGSLAVSGVQSQSPNSRVGQVLDAIGIANLPFQKQAAILAILATCFLMARTFLTVVVTKRTMLFLSRRGAAISSDLVARLLSKSLLFIQERTTQQTLFALTQGVSTLTIGVLATVVTMISDTSLLILLTAGLFVVDPAIAISTLVVFGSIILFMYKNLHNKAKTLGFAESTLHIKSNEKVLEVLNSYRESVVRNRRNYYSEQIGMLRFDLAKTQAQLSFMPYIGKYVLETSVVLGSLLIAAIQFALHDAVTAVATLSIFLVAGTRISPAALRLQQALIQIKSSLGGSEPTLDLIDALRDTLPATSVNQELDLTHEQFVPRIQIQDISFTYPNSDRQALSGVNLDIQSGSVVALVGPSGGGKTTLVDVILGIIEPNSGKVLISNLTPIDAIQISPGAISYVPQDVSIIDGTVRENIAMGFPVEVATDELVHNAIELAGLKEFVATLPNGLDTYVGEKGARISGGQRQRLGIARALFTNPKLLVLDEATSALDGETESRITDSILSLKGKVTVLMVAHRLSTVRNADQVVYMSEGSIKAVGTFESVRNTVPDFDTQAQLMGL

SEQ ID NO: 77 - DNA, 1809 bp, Butyrivibrio hungatei XBD2006
atgtcgaatactgataataagaaagtatcgctttttagcaagttgcgctatatctttgatcgcaaacagaaaggccagctcgttatcctcgccgtactgatcttgtttgggggaatctttgaaaccttttcgatcagcatgatgatcccggtgatggccgggatcatcaaccaggataaattgcaatcggctatcgaggataataaggtgctacgcatcatcgcggaatcgctgaacttggggactggctcagaactcgccgccaagctcactgtgtgtctcatcgtgctcttcctcgttaagaacgcgtatcagatctttctgatctaccggcagaatacttttatcacccgcgcgcgcaatgacatgatctcgcgcgtaatgcgggaatttctcaatcgcccgtatgaggattatttaggggccgatatcccaacggtgtttcgcatcaccgactcggatatcccccgcacctttactctgatgctttctctgctttctctgtcgactgaactcgtagtgtcaatgggcctgggcatggtactgatcatcactgatccaatcctcactgtgatctgcgtatttgtgtttgtggcgcttacgctgtttaatactaaggtgcttaaaccgaaactgaataaaactggcaaggagaaccaggaaactcagagccggatcgcgaaatggcgcctccaggcgatctatgggctgaaagatgttaaggtgctcaatcgccaggatttctttatccgcaactattatgagagcggaaaggtaggggcagatctcgcgcgggattactcagttctgaataatatgccacgcacgctgatcgagactgtatttgttgccgtggtattgctctatgtgttgttttttaaactcaatagcaatggtgttcaaaacgccggtgatgtggataagctcatcatcgcattgggcttagtggccacccggctgatgccagcagtgaatcggatcaatacgtatatcgctgagatcgcttataatcagtactcgctggattttgtttataataacttgactgatagcatgaaaatggataaagcaatgcgcgcagagcgcgccgcgatcgcggggccagagctgcatctcgaaaaagagatcgagatcaaggatatcacgtttgcctatccagacgccgagactaatatctttactggcgcgaatatggtgatcccgaaaggcaagagcgtagggatcatcggcccgtctggggcagggaagagcacggttgtagatatcatcttagggctgctccatgtccagtcgggagagatcttgtgcgacggctcgaatatcttctcgaactacgatagctggctggctcagatcgggtatatcccgcagaccatctatatggttgatgagtcgatccgcgagaatatcgcgttcgggatcgatgcggacaaaatcgatgaggaccgcatctgggaagtgatggaagaagctcagctggccgagtttgttaaatcgctgccagaaggcctggacactaaaatcggggaccgcggagtacgcctgagcgggggccagcgccagcggatcggcatcgcacgcgccctgtatcataacccagagatcctggtatttgatgaagccacctcggccctggataatgagactgaggccgctgtgatggaagccgtaaattcgttccacggcaagaagaccatgatcatcatcgctcaccgcttgaatacgatcgagaattgtgacatcatctatgaagtgaaagacgagaaaatcacccagacgacgttggaagggcgcaatgtgatccactga

SEQ ID NO: 78 - protein, 602 aa, Butyrivibrio hungatei XBD2006
MSNTDNKKVSLFSKLRYIFDRKQKGQLVILAVLILFGGIFETFSISMMIPVMAGIINQDKLQSAIEDNKVLRIIAESLNLGTGSELAAKLTVCLIVLFLVKNAYQIFLIYRQNTFITRARNDMISRVMREFLNRPYEDYLGADIPTVFRITDSDIPRTFTLMLSLLSLSTELVVSMGLGMVLIITDPILTVICVFVFVALTLFNTKVLKPKLNKTGKENQETQSRIAKWRLQAIYGLKDVKVLNRQDFFIRNYYESGKVGADLARDYSVLNNMPRTLIETVFVAVVLLYVLFFKLNSNGVQNAGDVDKLIIALGLVATRLMPAVNRINTYIAEIAYNQYSLDFVYNNLTDSMKMDKAMRAERAAIAGPELHLEKEIEIKDITFAYPDAETNIFTGANMVIPKGKSVGIIGPSGAGKSTVVDIILGLLHVQSGEILCDGSNIFSNYDSWLAQIGYIPQTIYMVDESIRENIAFGIDADKIDEDRIWEVMEEAQLAEFVKSLPEGLDTKIGDRGVRLSGGQRQRIGIARALYHNPEILVFDEATSALDNETEAAVMEAVNSFHGKKTMIIIAHRLNTIENCDIIYEVKDEKITQTTLEGRNVIH

SEQ ID NO: 79 - DNA, 1746 bp, Roseburia intestinalis CAG:13
atgaaagatcttgttaaatcggttctaaaagtctttgatggcaagcagaagcggaagctcgtaggcatgatgtttctgatactgatcaactccggagtatcgctgttaggagtctcagtgttatcgccgtttatcaccgcagtgatgaacccggaagagttacttaaaaacgacatcatacgttctgtctatgatggctttcatatgcagaatacgaaccagctgatcacgctgctttctgtgctgatcatcatcgtgtatatcgcgaaaaacgcatttatcatctttatcaataatatgctgtactgtttctcgtattatggcaaacgagagatgcaggaccggatgatgaaatattatatctcgcgtgattacacctttttcttaaatcataattcggctgagctgatgcgtgatatcaatacggatccagagatgttttatgcagcagtgttaaatatgcttcagttagcgtcggaactttgtgtatcgctgatcttagttggctatttattagttaaagatgcactgcttacgttaggagtcgcatttgcaatggtagtcatggtttttatctttatgaaaaaactgcggagaacgttagcacgtttcggtgatgaccgtcgtaaatacaatgctaatatcctgcagtgcatgcagcaggcattcggtgggatcaaagagatcaagatcgcgaatcgagaggcgtattttgaaggggaatttgttaagcagaacgggttatacacgtatgtaatcaaacagaattctttcttatcgtcgatcccgaaaccgatcatggaagcgctttgcatcacgggcttaatggcagcgatcatcgtacgtatcaatgcaacctcgacctcgccggagcagtttgtaggcaccttagcggtttttgcagccgcggcgtttgctctgttaccctctgcgaataagatgtctgaatatttaggctcaatcatccataatggagtagtgatccataagatcggggaagagtatgctgcaatccgggatatggaaatccagaccgaaaaagaagaaaactacaaaaaagttaccttagacaaagaaatcaaagtagaggatatgacgttccattacccggatactgaggatgcagttttagctcatgtaaacgtgaccatcccgaaaaataagtcggtcgcgtttatcggcccgtccggggcaggcaagacgacgatggtagacctgatcttaggagtcttaaaaccgcaggctggcaagatcaccgttgatgggatggatatcaaagaatcttaccgtggctggcatgataagatcgggtatatccctcagactatctacatgcttgacgataccatccggaataatatcgcgtttggaaagacggaagggatcgatgatgcggatatctgggaagcgttaaaacaggcgcaattagatgagtttgttaaatcgttagatgagggattagatacgatgatcggcgaggctggagtccggttatccgggggccagcgtcagcgtatcgggatcgcgcgtgcactataccggcgtccagaagtgttagtgctggatgaggcgacttcggcgctggatacggaaaccgaagctgctgtcatggaagcgatcgactcgttgcaggggaagatgaccatgctgatcatcgcgcaccggctgtctacgatcaaaaactgcgacatggtttatcaggttgagggaggatcggtgacccggaaagaaaaggaatcggtttga

SEQ ID NO: 80 - protein, 581 aa, Roseburia intestinalis CAG:13
MKDLVKSVLKVFDGKQKRKLVGMMFLILINSGVSLLGVSVLSPFITAVMNPEELLKNDIIRSVYDGFHMQNTNQLITLLSVLIIIVYIAKNAFIIFINNMLYCFSYYGKREMQDRMMKYYISRDYTFFLNHNSAELMRDINTDPEMFYAAVLNMLQLASELCVSLILVGYLLVKDALLTLGVAFAMVVMVFIFMKKLRRTLARFGDDRRKYNANILQCMQQAFGGIKEIKIANREAYFEGEFVKQNGLYTYVIKQNSFLSSIPKPIMEALCITGLMAAIIVRINATSTSPEQFVGTLAVFAAAAFALLPSANKMSEYLGSIIHNGVVIHKIGEEYAAIRDMEIQTEKEENYKKVTLDKEIKVEDMTFHYPDTEDAVLAHVNVTIPKNKSVAFIGPSGAGKTTMVDLILGVLKPQAGKITVDGMDIKESYRGWHDKIGYIPQTIYMLDDTIRNNIAFGKTEGIDDADIWEALKQAQLDEFVKSLDEGLDTMIGEAGVRLSGGQRQRIGIARALYRRPEVLVLDEATSALDTETEAAVMEAIDSLQGKMTMLIIAHRLSTIKNCDMVYQVEGGSVTRKEKESV

SEQ ID NO: 81 - DNA, 1827 bp, Pedobacter ginsengisoli
atgaaaatatatttccgtttattatcattcgcgaaaccaatcgaaaagtttgcgattccatatgtgatcacgacggtactatcagtgatttttagcgtgttaaatttaacgttattagcgccattattcgaaacgttaatcagcgatgagccgacgaaaagctcaggtttagcggataacgcagctagcgcgtttaatattacggcgcagtttaaagagtttgtgaattcgtcgatcataacgaatggtcaggagaaaacgcttacgtatatatgcctaattattgtactaagcgtattattatcgaatatcttccgttattttagccagcgttcgatggaagatcttcgggtgcatacgttattaaatttacggaaaactgtgttcgaaaacgtgatgaacttgcatgttggttattttaataacgaacggaaaggtgacattatctcgaaagtgtcggccgatgtgcaagtggtgcagtttacggtaacgaatacgcttcaagtggtgtttaaagagccactaacgttaattttctatgtgttagtgctattatcaattagcatcaagcttacgttattttcgttattagtgattccgatatcggcgtttgtgatcagcaaaattgtgaaacgtatcaaacatcaagcaaaggaagctcatgagtcgttcgcgaaaatgattggttttttagatgaagcattagggggcatcaaaataattaaagcatttaacgcgtcagagcgaatcaaagaacgttttcataacgaaaatgtattttatagcaatatcaatcggaagatggtgcggcgacaacagttaggtagcccgttaagcgagtttttaagcattgtgatggtgtcattcattgtgtggtatggtggccggttaataataagccatcaacccggtgcgctatcgacgagccagtttatcgcgtatatcgcgatcttttcccaggtgatgcggccggcgaaggcattaacggatagcttttcgggtatacatactggtatcgcggcaggcgaacgggtgctagatttaattgacacgaaagcggaacaaataaataagcccgatgcgaaagaaatcagcagcttcaataactcgttagtgtttgaaaacgtgtcattttcgtacgaatcgaaagaagtgttaaaaaatattaatttaacgattcagaaagggaaaacgatcgcattagtgggaccaagcggtggcggcaaaagcactctaatggatttaatcccacgtttccacgatccaaaatcgggttcgatcaaaattgatggtttagattatgcggatttaacggttgagtcgattcggagccagcttggtatggtgaatcaagaaagcattttatttaatgatacgatctttaataatatcgcatttgcgaagccggatgcgacggaagccgaagtgattgccgcggcgaaaattgcgaacgcgcatgacttcatacttcaaacggaaaacggttatgaaacgtatacgggtgaccgtggtaataaacttagcgggggccagaaacaacggttatgtatcgcgcgtgcgattttagcgaatccaccgataatgttattagacgaagcgacgagcgcgttagatacggagtcggaaaagttagtgcaagatgcgcttaataaattaatggaaaaccggacttcattagtgatcgcgcaccgtttaagcacgatccatggtgcggatttaatagtggtgattgataagggtgagataattgaaacgggtacgcatcaagagttattatcgcataacggtctatataaaaaacttattgaattgcagacgttttcgtaa

SEQ ID NO: 82 - protein, 608 aa, Pedobacter ginsengisoli
MKIYFRLLSFAKPIEKFAIPYVITTVLSVIFSVLNLTLLAPLFETLISDEPTKSSGLADNAASAFNITAQFKEFVNSSIITNGQEKTLTYICLIIVLSVLLSNIFRYFSQRSMEDLRVHTLLNLRKTVFENVMNLHVGYFNNERKGDIISKVSADVQVVQFTVTNTLQVVFKEPLTLIFYVLVLLSISIKLTLFSLLVIPISAFVISKIVKRIKHQAKEAHESFAKMIGFLDEALGGIKIIKAFNASERIKERFHNENVFYSNINRKMVRRQQLGSPLSEFLSIVMVSFIVWYGGRLIISHQPGALSTSQFIAYIAIFSQVMRPAKALTDSFSGIHTGIAAGERVLDLIDTKAEQINKPDAKEISSFNNSLVFENVSFSYESKEVLKNINLTIQKGKTIALVGPSGGGKSTLMDLIPRFHDPKSGSIKIDGLDYADLTVESIRSQLGMVNQESILFNDTIFNNIAFAKPDATEAEVIAAAKIANAHDFILQTENGYETYTGDRGNKLSGGQKQRLCIARAILANPPIMLLDEATSALDTESEKLVQDALNKLMENRTSLVIAHRLSTIHGADLIVVIDKGEIIETGTHQELLSHNGLYKKLIELQTFS

SEQ ID NO: 83 - DNA, 1731 bp, Verrucomicrobia bacterium CG1_02_43_26
atgaaaaaattatttccatacttagtgtatttaaaagatgtcaaaatggcatttatcttagcaatcttagccggtatcttttatggtgcaagcacgggtttaggtatcccagtgttaatccgttacttatacccaaaaatcttttcgaatgaagcaatctcggttatgacgttaactttaatctgcacgttaccagtgatcgtatcggccttccgggctggtgggaacttcttaaattcgtattatttatcatattgcgggcagtatatcctccaacaattacggatcaaggttttcgccaagatccagaacttaccaatcgcatttttccataaacggcaaccaggggacttaatctcacgtgtgagcaacgatacgacgatcttgcagcaaacgatcatcaatgcatctcaagagatcctcaagcagccaatcacgttattaggggcagtatcgtatatcgtttatatgtgttttcagcaaagcgatgtagtatttttaatcatctttatcttagcaatcccattatgcatcgttccaatccgttatatcgggatcaaaatgcgtaataaagcacgggcaatgcaggaacaaacgtccgagttaactcaccggttagcccagaatctctcggcagttaaagaaatccggtcgttttgtttagaggaatacgagttatcgcgttacgaagcagtttgccatgttttacgggaacgttttttaaaagtggttaagtatacttcaatcttaagcccatcgatcgaagtcatcgcctcgtttggagtcgcaatggcattttggtattcgtataaatcgaagatcgagccagatgtgtttatctcgatcgtcggtgccttatatttatcgtatgaaccaatcaagaaattaggtcgattaaactccgagatgcaacaggggctcgcatcgttagagcgcctcgaaaatatcttagaagaaccaatcacgatccatgacccagcagtacctcacccagtcggtaaattagagggcaatatccagtttaagaacgtaagctttgcatatgagatggccccagtcttacgggacgtgacggtccatttaacggccaaaaagacgtatgcattagtcggtccatcgggtgctggtaagacgacttttgctcatttaatcccacgtttttacgatttagcagaagggaacggttcgatcacgatcgatggtatcaatatcaaagacatgcgattaaaagacttacggcagaatatcgcattagtgagccaggatccagtgttatttaatgatacgatctataataatatcttagttggtaaaccatcggcaagccatgaggaattaatgactgcagccaaacgggcatttgctcaccatttcatccttgagctcgagaacggttatgatacgttagttggtgaaaatggttcaatgttatcgggtggccaaaaacagcgtatcgccttagcacgtgcatttttaaaggatgccccaatcttaatcttagatgaagcaacgagcgcattagatgcaaattcggagcagttaatccagcaagccttagaggaattatttaaagggaaaacggtgatcatcatcgctcatcggttttcgacgatcaagcacgccgaccgtatctttgtatttaaatcgggtgagatcatcgaagaaggcacgcatgacttactctgcgaagggaaaggtttatacgaagaactctacgccaaacagagctag

SEQ ID NO: 84 - protein, 576 aa, Verrucomicrobia bacterium CG1_02_43_26
MKKLFPYLVYLKDVKMAFILAILAGIFYGASTGLGIPVLIRYLYPKIFSNEAISVMTLTLICTLPVIVSAFRAGGNFLNSYYLSYCGQYILQQLRIKVFAKIQNLPIAFFHKRQPGDLISRVSNDTTILQQTIINASQEILKQPITLLGAVSYIVYMCFQQSDVVFLIIFILAIPLCIVPIRYIGIKMRNKARAMQEQTSELTHRLAQNLSAVKEIRSFCLEEYELSRYEAVCHVLRERFLKVVKYTSILSPSIEVIASFGVAMAFWYSYKSKIEPDVFISIVGALYLSYEPIKKLGRLNSEMQQGLASLERLENILEEPITIHDPAVPHPVGKLEGNIQFKNVSFAYEMAPVLRDVTVHLTAKKTYALVGPSGAGKTTFAHLIPRFYDLAEGNGSITIDGINIKDMRLKDLRQNIALVSQDPVLFNDTIYNNILVGKPSASHEELMTAAKRAFAHHFILELENGYDTLVGENGSMLSGGQKQRIALARAFLKDAPILILDEATSALDANSEQLIQQALEELFKGKTVIIIAHRFSTIKHADRIFVFKSGEIIEEGTHDLLCEGKGLYEELYAKQS

SEQ ID NO: 85 - DNA, 1671 bp, Brachyspira hampsonii P280/1
atgctcttgactctgtctttctaccttgttggcggttttggactgttcatgtatgggcttaaggtattctcggatggactgcaggagtcgacggagaatgctctcaaggacatcctgcacaaggtcacgcagaacaagatcttagggatctcgttgggcttcctgatcaccgcgatcgtacagtcaagttcggcggtcaccgtaatgacggtttcgttcgttaatgccaatttgctgactttgtcgcaggcaattaacatcatccttggggctaacatcggcacgacggtcacggggtggatcatctcactgaacatcgacgtcttagctctgccatccctcggaatcgggtccatcatcgtcatctttggcagtgaaaatcgtaagttgcgtttctttggcgagatcctcatgggattcggaatgatcttttacgggctgatcctgatgaaaacagcgttcgagggtgttcgtggctctgaggacttcgagaaggtgttcttaatcgccaatgccgacacaatgtatggtcgtttcttatgtgttgttatcgggatggtcgtaactgctatcatccaatcgagctccgcggcccttggcgtaacgatctcgctcgcatccgtgggccttatcgattaccccactggcgtcgctctcatcctcggacaaaacatcggcacgactattacggccgtattggcaacactgggtgctagcacaaatgcaaagcgtgctgctttggtgcactgtttgttcaacatcttcggcgttatctacatgttcttcctgtttccgtactacatcaagctggtcgacatcattgtgggtttcatgaacatcggggaccccaatctggtcgtaaacaacaaatacgttaacatctcattctacatcgcagccgcgcacaccatgttcaacattatcaatgtcatcgttttctactttctcacagagaagctcgagaaaatcgtttgttttattattaaggacaaagaggacgagaaacacgtgtccgtcttgtcagacaagctgcttaacatgccagtttctgctgagatcgaggtccgtaaggaagtaacatacatgggagacattgcaaagaaaatgctggcgcgtatcgaacaacttttcgacaatccttccgagcgtctgctgactaaaatccgtgaccacgagaagatgctggacaacacggaccaggagattcacgccttcttgctcaagttactgggcaagaacacgttaaactccgcaaacattgcttcactcattaacatctcaacatactacgaaaatcttggcgacaatcttaaggacttgggtaaagcaattatcaagggtaatgagaagaaaacgagtttcaatgagactcaaaaggaagatatcatcaagatgctgcacaacaacaaggacttcatcgactacctggcgggtctgatcctggagtactactcacttaacaaggagaaaacttacgacgaagcaatggagaagtaccaccagattaagggtttctactacgaggcccgtgagcgtcactacgacaatgtcgacaagtcgttgatcccagcattgaatgcacacctgtatggcgacgttctggtctactttaatcgctccatcggtaatctggtcaacattgtcgaggcaatcacgggtaaggacaagtga

SEQ ID NO: 86 - protein, 556 aa, Brachyspira hampsonii P280/1
MLLTLSFYLVGGFGLFMYGLKVFSDGLQESTENALKDILHKVTQNKILGISLGFLITAIVQSSSAVTVMTVSFVNANLLTLSQAINIILGANIGTTVTGWIISLNIDVLALPSLGIGSIIVIFGSENRKLRFFGEILMGFGMIFYGLILMKTAFEGVRGSEDFEKVFLIANADTMYGRFLCVVIGMVVTAIIQSSSAALGVTISLASVGLIDYPTGVALILGQNIGTTITAVLATLGASTNAKRAALVHCLFNIFGVIYMFFLFPYYIKLVDIIVGFMNIGDPNLVVNNKYVNISFYIAAAHTMFNIINVIVFYFLTEKLEKIVCFIIKDKEDEKHVSVLSDKLLNMPVSAEIEVRKEVTYMGDIAKKMLARIEQLFDNPSERLLTKIRDHEKMLDNTDQEIHAFLLKLLGKNTLNSANIASLINISTYYENLGDNLKDLGKAIIKGNEKKTSFNETQKEDIIKMLHNNKDFIDYLAGLILEYYSLNKEKTYDEAMEKYHQIKGFYYEARERHYDNVDKSLIPALNAHLYGDVLVYFNRSIGNLVNIVEAITGKDK

SEQ ID NO: 87 - DNA, 1923 bp, Thermotoga maritima
atggaagaacgcgaactgacgctgagcgacatattattgatgtttaaacgccgttcaaagctgttttggttagttttagttttaacggtattcgccacgggaatctacttatttttagccacgcctcagtatgaagcatatgcccgtgtgaaagtctctacgcagaagggtatgcgcctaggcttatcgatagaaggcttattaggcggcctgtcgtcgctgttaggaggcggcggctctcaggaagatgagatccagatcatgctgtcacgccgaaatatcatcgccgtgatcgacgaactggatttagttcataaactgctggacgaaaaagacatcgagaaggccaagaaaaacggactgacggaagatgatttaaaactgagcctgtattcttatatggttgaaaaactgatcacggtagagccagtgaaaaactcttcgatcttagaagtcaaggttacgtggcatgaaccaaaattagccgccgaaatcgccaataaaatcgttgaaaactatacgaagatctctgaaaatatcgccaagtcgcagttaggcgccaagatggacttcctggaagagcaaatcccaaaggttgagcaagaactgaaagaagccgaatcgaaattaaaggtctttaaagagcagaaccgcatatacagcgttgaagcccagactcaggttttaatcgaccgttatgcaagcttattgcaaaagatggaagaagcccgcatcgccatggaagccacgcagaaacagagcgagttcttctctaaagaactgaaagatctggacatcgaaatcgagcagatcaaagactcgatcacgtttgatccaatcataagccaactgaaatcgaatatgatcaatatacagatcgagttagcaggcctgatggaacgatatacggagactaacccagcagtggtgcagaaaaaagcagaactgcaggagactcagaaacagctggaaatcgagctgaaacgcttactgacgtcccaggtgaagaaaacgggaaacccagtctatgaagaagccctgtcatctctgatcaaggccgagtctgagaaaatcttataccagagccagtatgaagcctttaagaaactgtatgaggacatggaaaaagaattatcaaaattaccagaactggaacagaaattaatcgaactggaacgcgactataaggtgaaagaaacgatctatacgacgttattgcaggccaagtatgagagcctgatctcggaagccgccatcacggccaacgccaacgtcatcgactgggccgtgccaccattagaaccatcgaaaccaaataaaaaactgacgctggccataggcggcgttttaggaatctttttaggcatcctggcagtgttcttcgccgagttttcagaccgccgtatcaagagcgaatctgaagccgagtacttattaggattagaaaagatcgtcgcccgcgttccactaacgcagaacgaagaagtgatggaaaaagccttaggcatcccagccgtgaaatcaggcaaggttacgtttatcacggccctggaagatggcgccggcgtgtcgacgctggccaaacatatggccaaattactgtcaaagaaggaaaaggtactactgttaacggaagaaaaagtggaaggaacttttgaccgcaaagacgtttttgacttattaaaaaatccagacgttctggaagaattaaaagagagatatgacaaaatcatcgttgacgcaccatcgttaaagcgtacgccagacttcctgccaatagccgaaaagtcggacacggtctatatcgtgatccgattagaacatacgctgtcggaagatttaaaaacgctgcatactttgaagaagatcgacggttttatcttaaacggattaacgaagaaaaattcaacgtacgtggaataa

SEQ ID NO: 88 - protein, 640 aa, Thermotoga maritima
MEERELTLSDILLMFKRRSKLFWLVLVLTVFATGIYLFLATPQYEAYARVKVSTQKGMRLGLSIEGLLGGLSSLLGGGGSQEDEIQIMLSRRNIIAVIDELDLVHKLLDEKDIEKAKKNGLTEDDLKLSLYSYMVEKLITVEPVKNSSILEVKVTWHEPKLAAEIANKIVENYTKISENIAKSQLGAKMDFLEEQIPKVEQELKEAESKLKVFKEQNRIYSVEAQTQVLIDRYASLLQKMEEARIAMEATQKQSEFFSKELKDLDIEIEQIKDSITFDPIISQLKSNMINIQIELAGLMERYTETNPAVVQKKAELQETQKQLEIELKRLLTSQVKKTGNPVYEEALSSLIKAESEKILYQSQYEAFKKLYEDMEKELSKLPELEQKLIELERDYKVKETIYTTLLQAKYESLISEAAITANANVIDWAVPPLEPSKPNKKLTLAIGGVLGIFLGILAVFFAEFSDRRIKSESEAEYLLGLEKIVARVPLTQNEEVMEKALGIPAVKSGKVTFITALEDGAGVSTLAKHMAKLLSKKEKVLLLTEEKVEGTFDRKDVFDLLKNPDVLEELKERYDKIIVDAPSLKRTPDFLPIAEKSDTVYIVIRLEHTLSEDLKTLHTLKKIDGFILNGLTKKNSTYVE

SEQ ID NO: 89 - DNA, 747 bp, Clostridium sp. CAG:1013
atgtatgtgttagaggcgtgtgtagatagcctggatagcgcactggctgcaaaggaagggggagcgacccgcctggaattgtgcgcgaatttaattattgggggcactaccccggcgctgagcttagtggaatgggtgaaaagagacactgcactgccagtccatgtactgctgcgcccacgctttggtgattttttgtataccgaagaagagttcagcatgatgctggaagatgccgcggcgctgctggaccgaggggcagatgcgattgtgagcggatttttgactccagagggagatctggatctgttgcggatggaaaagatggttgaactgtgccataagagagggaaacggtttactcttcatcgagcgtttgacgcatgccgcgacccgtttctggcattagagcagtgccgcgagttaggagtggacactgtgttgaccagcggccagaagaatacctgcctggaagggcttccgctgctgaaggaactgtgggaaaagagaggagatgtggaactgttgataggggcaggagtggacgcagaggcgatacggcgcattcgggaagagctgccgcaggcgagaagctttcatatgagcggcaagaaagttgtggagagcgcaatgacttatcggaaggacggagtaagcatgggactgccggcgttttcagagtatactttgtggcgaactgacccagagaaattgcgggcagcagggaaagagttaatgcggcagtaa

SEQ ID NO: 90 - protein, 248 aa, Clostridium sp. CAG:1013
MYVLEACVDSLDSALAAKEGGATRLELCANLIIGGTTPALSLVEWVKRDTALPVHVLLRPRFGDFLYTEEEFSMMLEDAAALLDRGADAIVSGFLTPEGDLDLLRMEKMVELCHKRGKRFTLHRAFDACRDPFLALEQCRELGVDTVLTSGQKNTCLEGLPLLKELWEKRGDVELLIGAGVDAEAIRRIREELPQARSFHMSGKKVVESAMTYRKDGVSMGLPAFSEYTLWRTDPEKLRAAGKELMRQ

SEQ ID NO: 91 - DNA, 750 bp, Odoribacter splanchnicus DSM 20712
atgcgacgtcaggattttaaaatagaagtctgcacgaactcggttgagtcggttcgagcggcgctcgctggtggggcagatcgtatagagctctgcgcgggcatgccagagggcggcacgacgccatcgtatggtgagatctgcttagttcgagagttaatgccagcagggatgcatgttatcgttcgtccccgagggggcgatttcctataccgtgaggatgaactcgaagttatgtaccgggatatagaaatggcacgaaaattaggtgtcgatggcgtcgtcttaggttgcttaacgcgtgaaggcgaagttgacgaaggagtcatgcggaaattaatggccgcgtgtggcgaaatgtcggttacgtttcaccgtgcgttcgatatgtgtcgtgatccatttcaggcgttgcagacgatcgaaaagcttgggtgcgcacgaatcttaacgtcgggccaatcgaactcggccgaaactggcgttccattattaaaagaattagttgcgcgtgcgcgtaatgttatcatcatgccagggtgcggagtcaacgctgcgaacgttcgtaagatcgcggaatctacgggtgcgttcgaattccatctctcggcgcgtatccggcttgggtctgatatggtctatcggaacccggccgtatcaatgggcggcacggttcaagttgatgagtacggccgtgaagtttcgtcgtctgagaaagttgaagaagttatccgtcggttagagtactga

SEQ ID NO: 92 - protein, 249 aa, Odoribacter splanchnicus DSM 20712
MRRQDFKIEVCTNSVESVRAALAGGADRIELCAGMPEGGTTPSYGEICLVRELMPAGMHVIVRPRGGDFLYREDELEVMYRDIEMARKLGVDGVVLGCLTREGEVDEGVMRKLMAACGEMSVTFHRAFDMCRDPFQALQTIEKLGCARILTSGQSNSAETGVPLLKELVARARNVIIMPGCGVNAANVRKIAESTGAFEFHLSARIRLGSDMVYRNPAVSMGGTVQVDEYGREVSSSEKVEEVIRRLEY

SEQ ID NO: 93 - DNA, 702 bp, Mitsuaria sp. PDC51
atgactggggctccgttattcatcgaagttatcgccttgactgctcgcgacgcctgtgaggcagaggcagccggggcagaccgtatcgagttagtcagtgacatggcacaaggcggcctgactccgtctgcccaggttgtggacgaggtagtacgtcaatgtcgcttgcctgtcatggtaatggtccgtccgcacgcccgcagcttctgttacgacgaggcagacatgcgtcaagtacgtgaaggggttgcaatggtacgtgacgctggtgcccatggacttgtctttggggcgttaacagctgatggggacatcgaccgtgcagctctcgaccaagtcttacgttgggcggatgggcttcccttaacgttccatcgtgcattcgacgaggcccgtgatcccgtacgtgccttctcagagctctcagcttatcgtggtgccgtcacgcagctgttgtcatctggcggagcacctatcgcagaagagggagcggaattacttgcacagctggtaacgcgttggcgccttggtgagggtgtagagcctctggttggggcgggagttcacgcaggaaatttagcagcattacatcgtcgtatcggtgcccgtcaataccacgttgggagtggagctcgcgccggcggaagtttcgcctcagggatcgacgcagctcgtatcgctgcactccgccaagccttatga

SEQ ID NO: 94 - protein, 233 aa, Mitsuaria sp. PDC51
MTGAPLFIEVIALTARDACEAEAAGADRIELVSDMAQGGLTPSAQVVDEVVRQCRLPVMVMVRPHARSFCYDEADMRQVREGVAMVRDAGAHGLVFGALTADGDIDRAALDQVLRWADGLPLTFHRAFDEARDPVRAFSELSAYRGAVTQLLSSGGAPIAEEGAELLAQLVTRWRLGEGVEPLVGAGVHAGNLAALHRRIGARQYHVGSGARAGGSFASGIDAARIAALRQAL

SEQ ID NO: 95 - DNA, 678 bp, Prevotella intermedia ATCC 25611 (DSM 20706)
atgcccattctagaagtgtgcacgggcagcttgcagagcgtgatgaacgcagttaaaggcggcgcgcaacgtattgaactgtgttcagcattaagcctggatggcttaacgccgagcttgggcctgattaaaactgttcgtacgttatttccggaattaaccatccatgcgctgatacgtgttcgtgaaggcgacttctgttataccgaagaagaagtaaaagcgatggaaaccgatatcaaagcagttctgccgtatgcagacgctatcgtgtgcggcgcgcttaccgcggacggcgatattgatatcgcgaccacgcgtcggttaatcgatgcatgcgaaggcaaaccgtttacttttcatcgtgctttcgacgtgtgccgtaacccgttaaaagcgttagatgaattagcggcgcttcattgcacgcgtgttttaacgagcggccagaccccgactgcagaagcgggcattccggcgttgaaagaatacgttaaacatactgaaggccgtatgaccatattgccgggcggcggcgtgactccgttaaatgctaagaagattttagcggaaacgggagcaaccgagattcatggaagcgcgagcggcattgcggaaaacggccgtaaagaaactctgaccgaagttgttgcgcaaatcctgcaggaaattaaataa

SEQ ID NO: 96 - protein, 225 aa, Prevotella intermedia ATCC 25611 (DSM 20706)
MPILEVCTGSLQSVINAVKGGAQRIELCSALSLDGLTPSLGLIKTVRTLFPELTIHALIRVREGDFCYTEEEVKAMETDIKAVLPYADAIVCGALTTDGDIDIATTRRLIDACEGKPFTFHRAFDVCRNPLKALDELAVLNCTRVLTSGQATTAEAGIPALKEYIKHTEGHMTILPGGGVTPLNAKKIIAETGATEIHGSASGMTESGRKETLTEVVVQILQEIK

SEQ ID NO: 97 - DNA, 1719 bp, Actinobaculum suis DSM 20639
atgggcaaatctcaagaaagcattagctttaaagagaaattcgcttacggtatgggtgacgctgccactgcctttagtgcagtaagcgttgccagcttctcaatgttttatttcactgattacgtaggcgtgagtgcgaccgctctcggcacggtgctgatcttcacgcgcgttttcgatgcaatcaccaacattattatgggttacgttgtggaccagactcgtaccaaagagggtaaagcacgtccctgggttaaatgggcaatcctgcccatgtttgcagctctggttgcggtctttgcaattccaacgggaatgtcctctaccgcaaccattgtatggattacgattgtggtgaacatctactacttggtctacactattagcaacatcccctatgggacccttgggaccctgatctcacgtgatccacaggttcgctccgagctcacgctgctgcgtatgattggataccacggagtgagcatcctgctgtcctttatcacggtcggtttggttgcctacctgggtggtgggagcaaccgtggctggatctacaccatggttatctatggtgccattatgtcgattatcttctactttacctacagttgtacacgtgagcgcgtccgctcaacgagtgagatgcgccgtgatgccgaacgtgctgcggccgcagagtccggtgccgcaaatagtacaccacctaatggtggtcctgaggctggtacgggcacggcacctgcgaagtctgccggtgctactggaactgcgccggcaaaacctgctccggcaaaacgtctgggaatctttgccagcattggtttgctgttccgtaacaaatactggatccttattttcttgatcatgattctgacttggatcttggttaacctgtttggtggtgtaaatgtctattacgctcgtcacatccttggtgacgccaactacgtcggtgttcttaacgcagcatttacgggagcgcaaatcgtaggtttctttctggtgagtattcctatccgccgctttggtcgccgccgtactattgtattcgggcttgggatgatcatcgtgggctctttactggtcctgacggggccccagaacatctacgtcctggctgtagcaagcatcatccgtggactcggcttttctcctctcatgggtactgcatacgccatgctcgcggatgtgattgactacggtgagtggaagaatggtgtccgcaatgaggggatctcttattctggtggtaccttttctaccactgtggggagtggattggccagctcggggatcgtctggatcatgggtgcggcgggatacatctcagcaaaggacgcagtccagcctgcatctgtactggagtccattcagttcctgttctcgtgggcacccgttatcatcgcagcattgctgctcgtactgttctacttctacgaccttgacaccttctaccccgagatcgcgcgtgaccttgaacaaggcattaccctgaaagatcgtgagaaagatgccgcacgtcaggcgcaccacgtgtcgggagttgagccggcgcaccctgatcaccaccaaaacaatcccgagagccctgcgggaagtactcagactagcgcacagacaggaacgcagacttctaccaaataccgtactgagaagggcgggcagaatcgtacaaattacagtaccgagagtgaagccggcaacgcggttgaataa

SEQ ID NO: 98 - protein, 572 aa, Actinobaculum suis DSM 20639
MGKSQESISFKEKFAYGMGDAATAFSAVSVASFSMFYFTDYVGVSATALGTVLIFTRVFDAITNIIMGYVVDQTRTKEGKARPWVKWAILPMFAALVAVFAIPTGMSSTATIVWITIVVNIYYLVYTISNIPYGTLGTLISRDPQVRSELTLLRMIGYHGVSILLSFITVGLVAYLGGGSNRGWIYTMVIYGAIMSIIFYFTYSCTRERVRSTSEMRRDAERAAAAESGAANSTPPNGGPEAGTGTAPAKSAGATGTAPAKPAPAKRLGIFASIGLLFRNKYWILIFLIMILTWILVNLFGGVNVYYARHILGDANYVGVLNAAFTGAQIVGFFLVSIPIRRFGRRRTIVFGLGMIIVGSLLVLTGPQNIYVLAVASIIRGLGFSPLMGTAYAMLADVIDYGEWKNGVRNEGISYSGGTFSTTVGSGLASSGIVWIMGAAGYISAKDAVQPASVLESIQFLFSWAPVIIAALLLVLFYFYDLDTFYPEIARDLEQGITLKDREKDAARQAHHVSGVEPAHPDHHQNNPESPAGSTQTSAQTGTQTSTKYRTEKGGQNRTNYSTESEAGNAVE

SEQ ID NO: 99 - DNA, 1362 bp, Ruminococcus gnavus
atggagaagaaacgttatctcaagtggtacaacaaagtgggctacggctctggtgacctggcagggaatgtcgtttacgctttcttatcttcttttgttatgctgtacttgacgaacactgtcggccttaatccgggcattattggaaccctgattatggtgtctaaacttttcgatgggattagtgacatgttcttcggaaccatgattgataagacaaagagtaagttgggtaaggcccgtccgtggatgttatacgcgtacattggctgtgcggttacactggttgccaacttcgcaattcctgattccttggggacgacggcgcaatatgcctggttcttcattgcctatacacttttgaatgcggttttcttcaccgctaacaacattgcctacgcatcactggtaactttctgtacgaagaactctcgtgagcgtgtggaaatgggtagttggcgcttcattttcgcattctctacatcattacttattcaatccgtaacggtacaattcgttcgtgcagctggtggtggggcagcggcatggcgtacggttgctgtcgtttacgcaattattggactgattgtcaacacgatttcggttttctcaattaaggaacttccggaagaggagttgaaggcgggaaaggatcatacggaagagaagtactcgctggtagaagcagcaaagctgttattcagtaacaagtactacctgatgatttgtgcgacctacatctgtcagcaaatttacagcgcgatgttaaacatgggtatctactatatgatttacattctgaagaatgagaatttgtacagcgtgttctcgtgggccattaacattccggtgattattgccatgtgcattacgccgatgctcgtcgagaaaatgaagggcttgtaccgtatgaatctggccggttacattctgggtacagcaggacgtgtcggcgtaattttcgcagggtacatgggctcagtaccgcttatgttggcctttacggcagtcgctgccctgggtatggcaccgtggcaaggtgatatgggagcagtggtagcatcatgtagtgagtacacgtatctgacgaagcacaagcatattgatggtactatgtacagctgtacgtctttcggaacgaagattggtggtggtattggggttgctctgtgtggatggttgctggacgcctcagggttcgttaaggaagccactattcaacccgagtcatgccttaacatgcttcacgttatgtacttgtggattccgatggtactcagtctgattattacgttcattatgagtcgtatgaatgtcgaggacgccaatgagaaactgcgtaagcaactggaagaatgttga

SEQ ID NO: 100 - protein, 453 aa, Ruminococcus gnavus
MEKKRYLKWYNKVGYGSGDLAGNVVYAFLSSFVMLYLTNTVGLNPGIIGTLIMVSKLFDGISDMFFGTMIDKTKSKLGKARPWMLYAYIGCAVTLVANFAIPDSLGTTAQYAWFFIAYTLLNAVFFTANNIAYASLVTFCTKNSRERVEMGSWRFIFAFSTSLLIQSVTVQFVRAAGGGAAAWRTVAVVYAIIGLIVNTISVFSIKELPEEELKAGKDHTEEKYSLVEAAKLLFSNKYYLMICATYICQQIYSAMLNMGIYYMIYILKNENLYSVFSWAINIPVIIAMCITPMLVEKMKGLYRMNLAGYILGTAGRVGVIFAGYMGSVPLMLAFTAVAALGMAPWQGDMGAVVASCSEYTYLTKHKHIDGTMYSCTSFGTKIGGGIGVALCGWLLDASGFVKEATIQPESCLNMLHVMYLWIPMVLSLIITFIMSRMNVEDANEKLRKQLEEC

SEQ ID NO: 101 - DNA, 2061 bp, Curtobacterium sp. 314Chir4.1
atgtcaactgcgaccgctccagttgaagtgcagcgcggacggcagtcgagcccggccggccagccagggaaaccgattatgagccatcgccagattctgctggtgatttatggcctgatggccggcatgtttctgagcagcctggatcagaccattgtggggaccgcgattcgcaccattggtgatgatctgcatggcctggatcagcaggcctgggtgaccaccgcgtatctgattgcgtcgaccattaccaccccgatttatggcaaactgagcgatgtgtttgggcgccggccgctgtatatttttggcattgcggtgtttattctgggcagcctgctgagcaccatgagcaccagcatgattatgctggccgcgtttcgagcgtttcagggcattggcgccggggcgctgatgagcctgccgctggcgattatgggcgatattctggcgccgcgggaacgcgcgaaatatcaagggtattttttagcggtgtttggcattagcagcgtgattggcccgctgattggcgggctgtttgcgggcgcgagctcgattctgggcattaccggctggcgctgggtttttctgattaacgtgccgattggcgtggctgctctgctgatggtgattgcgtttctgcatctgccgaaatttggcgatgcgaaagcgaaaccgcgcattgattggtggggcgcgaccagcgtgattgtgaccctggtgccgctgctgttagtggccgagcagggccgcacctggggctggggcagcgcgggcgcgattgcgtgctatgtgattggcgcgctgggcctggttgcgtttctggtgattgaaaccctgatgaaagatgatgcgattattccgctgaaactgtttcgcagcggagtgtttagcatggcgaccattctgggctttttagtgggctttgcgatgtttggcgcgatgctgaccattccgctgtatctgcagattgtgaccgggctgaccccgaccgaaagcggctttgcgaccctgccgatgattggtgggctgatgattgcgagcattgcgagcgggcagattgtggcgcgcaccggcaaatatcgtatttttccggtgattggcaccgcgctggtgagcattggctatgtggtgctgacctttatgaccattgataaaccgctgtggtttctgatgattggcatgtttctgattggcctgggcctgggccagctgatgcagtcgattaccctggcgagccagaatagcgtgcagccgcgcgatatgggagtggcgacctcgagcgcgaccttttttcgccagattgggggaaccctgggcaccgcggtgctgctgtcggtgctgtttagcattatgccggcgaatattctgcatgcgaccgcggatcagaaaaacctgtcgtcagcgctggatgcggcgctgaacccgaccgtggcgtcggcgaaagcgaaccaaggagtgatggatcagatttggaacccgattgtgaccccgattaaacagcaagtgcagagcgggctggatgcggcgaccgcccaggtgactcaggcgaccgccggtgctccggaagcggtgcagcagcaggcgctgaccgcggccgcggataaagcgcatgcgtcggtgcaggatggcaaactggttgtggattggagcgatagcgcgcagcgcacctattgggttgatgatctggcgccgaccctgagcaaacagattgataaaggcacctcggataaagctagcagctcgtcggaaacctcggataccagctatctgaacggcgcggatagcgcgctaacccgtccgtttatgaccggctttaatagcagcgcggtgaccgtgtattgggttggctttgcggtgattctgctggcgtttattctgtcgtggttttttaaagcgccgccgctgcgcaaatcgagcgctctgcaggaacaggccgataacgatcgcaccgcggatgatttagaagtgcaggccgtgaccgcggctgataccatgggcagcccaaccgcgccagtgaccgggagcattgcggtgcagcgtggcagcagcgatgattga

SEQ ID NO: 102 - protein, 686 aa, Curtobacterium sp. 314Chir4.1
MSTATAPVEVQRGRQSSPAGQPGKPIMSHRQILLVIYGLMAGMFLSSLDQTIVGTAIRTIGDDLHGLDQQAWVTTAYLIASTITTPIYGKLSDVFGRRPLYIFGIAVFILGSLLSTMSTSMIMLAAFRAFQGIGAGALMSLPLAIMGDILAPRERAKYQGYFLAVFGISSVIGPLIGGLFAGASSILGITGWRWVFLINVPIGVAALLMVIAFLHLPKFGDAKAKPRIDWWGATSVIVTLVPLLLVAEQGRTWGWGSAGAIACYVIGALGLVAFLVIETLMKDDAIIPLKLFRSGVFSMATILGFLVGFAMFGAMLTIPLYLQIVTGLTPTESGFATLPMIGGLMIASIASGQIVARTGKYRIFPVIGTALVSIGYVVLTFMTIDKPLWFLMIGMFLIGLGLGQLMQSITLASQNSVQPRDMGVATSSATFFRQIGGTLGTAVLLSVLFSIMPANILHATADQKNLSSALDAALNPTVASAKANQGVMDQIWNPIVTPIKQQVQSGLDAATAQVTQATAGAPEAVQQQALTAAADKAHASVQDGKLVVDWSDSAQRTYWVDDLAPTLSKQIDKGTSDKASSSSETSDTSYLNGADSALTRPFMTGFNSSAVTVYWVGFAVILLAFILSWFFKAPPLRKSSALQEQADNDRTADDLEVQAVTAADTMGSPTAPVTGSIAVQRGSSDD

SEQ ID NO: 103 - DNA, 1665 bp, Niabella drilacis DSM25811
atgtcgaaattagactttatagtgatctgcgtttttgcgatcttaatggtaatcatcgggctggtttttacggctcaaagctcgaaaagctcgaaatcgttttttgaggcaggcggctcgactccatggtggatcaacggtctgtcgttatttatctcgtatttctctgccgccacctttgtggtgtggggcagtatcgcgtataaacatggtttagtggcgaataccatacaatttaccatgtgtttatcgggtatcgtgaccgcgctgtggatagccggtcgttggaagaaaacgggtgcgacgacggccgcggaatacctgggtaaacgatacgggaaatcagcgaaacagtactttagctatatggtgctgttatatagcctggtttcgacggccgcgatactttatgccgttggtaagatcgtttatgtggcgaccccgttttctttagaaacctgtatcatcgcgatcggtatcactatcatcgtttatacgacgggtggaggcttatggggagtgttagttactgatgttgtgcaatttgtgatcttatcagctgcggtaatcatcatcatcccattatctttatccgaagttggtgggatgactcagtttctgcaaaaaacgccagaccgtttctttgacccagtgaacgatgaatatacgttcggttttatgtgctcgttctttatataccaggttgtttatatagcgggcaattggtcgtatgtgcaacggtatacttcggtgtcctccgcgcggaacgcgaaaaaagtggcgtggcttttctccgggttatacctggttgcaccaatcatctggatgctccccccgatgatctaccgtgtaatcaatccgggtctggatggtgccggtgccgaaggggcatatatgatgttatgccaaaaagtgttaccggccgggttaataggtctggtgttatcagggatgatcgctgcgacgtcaagcaaagccaatacgaccatcaataccgctgccatcatctttgctcaggatatctataaaggtgttttctttaagcatacctctgataaaaagcaaatactggttgcgcggatctttactatattattcggtgctgctacggtgtttctggctatcttagttccagctgccgggggaatcgttgaagtggtgttaagcactgcggctatcgccgggggcagtttatttgggccggtaatattttcattattctcgcggcgacaaacggaatcgtcgcttatcgttatctcaatcgtatcagttgttgtgtcgttattctttaaagccctggctccatcgctgctggacatcaaattatcgcgtactgtggaaactatccttggtgttggtctgccactggtgctgttagctttcttcgaattatatgctgccataaaaggtataaaagtgccatattttatcaccgaagccggtgaaagcgggctgtcaccggatatcgaatctgccaaacaaaatatattcggtacccgggtgatcgcgtggtcggcagggtttgttggtgttggtatcagcttattaggttttctggccgaaaatggttcggttgcggttgttgttggtgccggtatcgtaatcatctcagcgttaatcttaatccggcaaaaacgggctgcggcagttgccgggacctcgtga

SEQ ID NO: 104 - protein, 554 aa, Niabella drilacis DSM25811
MSKLDFIVICVFAILMVIIGLVFTAQSSKSSKSFFEAGGSTPWWINGLSLFISYFSAATFVVWGSIAYKHGLVANTIQFTMCLSGIVTALWIAGRWKKTGATTAAEYLGKRYGKSAKQYFSYMVLLYSLVSTAAILYAVGKIVYVATPFSLETCIIAIGITIIVYTTGGGLWGVLVTDVVQFVILSAAVIIIIPLSLSEVGGMTQFLQKTPDRFFDPVNDEYTFGFMCSFFIYQVVYIAGNWSYVQRYTSVSSARNAKKVAWLFSGLYLVAPIIWMLPPMIYRVINPGLDGAGAEGAYMMLCQKVLPAGLIGLVLSGMIAATSSKANTTINTAAIIFAQDIYKGVFFKHTSDKKQILVARIFTILFGAATVFLAILVPAAGGIVEVVLSTAAIAGGSLFGPVIFSLFSRRQTESSLIVISIVSVVVSLFFKALAPSLLDIKLSRTVETILGVGLPLVLLAFFELYAAIKGIKVPYFITEAGESGLSPDIESAKQNIFGTRVIAWSAGFVGVGISLLGFLAENGSVAVVVGAGIVIISALILIRQKRAAAVAGTS

SEQ ID NO: 105 - DNA, 1539 bp, Saccharicrinis fermentans DSM 9555 = JCM 21142
atgaaaaaagttattacgtcaatcgggttaattgtgttatgcttaccggttttatttgcgcaaagacccacgattgcgtcagggcattttagctcgcttaactggattgtgctggttgtttttctggttggtacgacggtttttggagagctggttaaaaataaagataagggcttaaacggcttctttcgtggcggcaataacttaccatggtggacggttagcttatcattaatcgctacgaaaacgagcgttgcgacgtttattgctgttcctgcctttgttttttcacttcatggcgatctgtcatatctgcaaatgacgtttggctttgctttaggtaatatcttaatggtgtttgttttacttaaagagtattatgaggatcatatctacagcccttatgattttattcagaaccgtctgggcattcgtgtgagccaattaagccgtacgttctttatggttggggccacgatgagccaaggcgtgcgtttacttggcacggcgcttgtgttaagcgttattacgggtcagagcacgatattatgcattggcattattgcggcgtttgcggttgtttggtcgtatattggtggcataacgacggtagtttggacggatgctctgcaatttgttatctttatctttggcgctcttttcgcccttttttatgctatcggtgatattccgggcggactgagcgagatgatcgtaatcgccgatcagaaagcgaagcttacgctgcttgatttatcattagatcctcataaaacgtatactttatgggttggcattttaggttgcacgttttttgagtttggctcaaacgccgtggatcaagtggtgacgcagagagcgctttgctgtcggaacctaaaagaagctcgtaaagctgttggatttagcgttatcggagtagcgacgacgtggatcatggcctttgttggcattggcctggttgcgtactatcatataaacccgttaagcccagaaatcaacgcgagcatggttcaggaaccagatcgtatttttccgtattatgtggtgaattcattaccgaacggcatttcgggtttaattattgcggccatgtttgcggccggcatcagcacgctagatagcgccctgacggcgttatcgcaaacgagcgttatgggtgttggtagcattctgtttccccgtctgaagttcatgaatgaagataaagttgtgaaaattagcaagatggcgattattgtgtggggagggatcttagccctgttagcgtatggttttagcttctttcaagatggcggcttattagcgctgggttttaaagttcctgggtatgcgtacggtacgttaataggtattgcgtttcttgctctgatgcgacgtggcagctttatgggcattttaatggggagcctgctggcgattgcgtgcatcggatggatgcatatggaaggcataagcttcttctggtggttcccagtgggcgccttcgttgtgattatgatggcgatcttttatgatatgatcgtgccgaaaaaataa

SEQ ID NO: 106 - protein, 512 aa, Saccharicrinis fermentans DSM 9555 = JCM 21142
MKKVITSIGLIVLCLPVLFAQRPTIASGHFSSLNWIVLVVFLVGTTVFGELVKNKDKGLNGFFRGGNNLPWWTVSLSLIATKTSVATFIAVPAFVFSLHGDLSYLQMTFGFALGNILMVFVLLKEYYEDHIYSPYDFIQNRLGIRVSQLSRTFFMVGATMSQGVRLLGTALVLSVITGQSTILCIGIIAAFAVVWSYIGGITTVVWTDALQFVIFIFGALFALFYAIGDIPGGLSEMIVIADQKAKLTLLDLSLDPHKTYTLWVGILGCTFFEFGSNAVDQVVTQRALCCRNLKEARKAVGFSVIGVATTWIMAFVGIGLVAYYHINPLSPEINASMVQEPDRIFPYYVVNSLPNGISGLIIAAMFAAGISTLDSALTALSQTSVMGVGSILFPRLKFMNEDKVVKISKMAIIVWGGILALLAYGFSFFQDGGLLALGFKVPGYAYGTLIGIAFLALMRRGSFMGILMGSLLAIACIGWMHMEGISFFWWFPVGAFVVIMMAIFYDMIVPKK

SEQ ID NO: 107 - DNA, 1413 bp, Citrobacter freundii MGH152
atgactgaacttcctgacagcacccgctggcaactttggattgtggcatttggcttttttatgcagtctctggataccaccatcgtcaataccgcgcttccttctatggccaaaagccttggggaaagcccactgcatatgcatatggtcattgtgtcttatgtgcttaccgttgccgtaatgcttcctgccagcggctggctggctgataaaatcggtgtgcgcaatatattcttcaccgctatcattctgtttacgctgggatctcttttttgcgcctggtcgggaacgctgaatgaactggtgatggctcgcgtgctccaaggtgtaggcggcgcgatgatggtgccggtcggcagattgacggtgatgaaaatcgtcccacgcgagcaatatatggccgcaatgactttcgtcaccttgcctggtcagattggtccacttctgggcccggcactcggcggcattctggttgagtacgcctcgtggcactggatctttttgattaatattccggtaggcattgtcggggcaatagcgacgctcatgctgatgcctaactataccatgcaaactcgccgtttcgacctttctggattcatgatgctggctgtcggcatggccgtacttaccctcgcgcttgatggttcaaagggaaccggcatttcgcatatcacgctggcagcactggttatctgtggagtactggcgatcctgctgtatctgaaacacgcccataataaccctcgtgcactgtttagcctgcacctgtttcgtactcataccttctctctcggcctgtttggcagctttgccggacggattggtagcgggatgctcccgttcatgaccccggtattcctgcagattggattgggcttttctccatttcatgccggtttgatgatgatcccgatggtgctgggctcaatgggaatgaaacgcatcgtggttcaggtcgtcaaccggtttggttaccgtcgcgtactggtcaccaccacgcttggcctgtctatggtcagcttgttgctgatgagcaccgcgctccttggttggtattacgccctgccgttcatactgttcctccagggaatggtcaattctacccgcttctcgtcaatgaatacgctgacgctaaaagacctgccggatgaccttgcgagcagcggcaatagcctgctgtctatgatcatgcaactgtctatgtcaatcggagtaaccatcgccggtctgttgctcggcatgttcgggcagcaacatatcgccatcgacagcgcgtctacgcataccgtctttatgtatacctggctgtgcattgcgttccttatcgcactgcctgccatcatttttgcccgcgtgccggacgacacgcagaccaacgcggtgatttcacggcgcaaaaggtcaacctga

SEQ ID NO: 108 - protein, 470 aa, Citrobacter freundii MGH152
MTELPDSTRWQLWIVAFGFFMQSLDTTIVNTALPSMAKSLGESPLHMHMVIVSYVLTVAVMLPASGWLADKIGVRNIFFTAIILFTLGSLFCAWSGTLNELVMARVLQGVGGAMMVPVGRLTVMKIVPREQYMAAMTFVTLPGQIGPLLGPALGGILVEYASWHWIFLINIPVGIVGAIATLMLMPNYTMQTRRFDLSGFMMLAVGMAVLTLALDGSKGTGISHITLAALVICGVLAILLYLKHAHNNPRALFSLHLFRTHTFSLGLFGSFAGRIGSGMLPFMTPVFLQIGLGFSPFHAGLMMIPMVLGSMGMKRIVVQVVNRFGYRRVLVTTTLGLSMVSLLLMSTALLGWYYALPFILFLQGMVNSTRFSSMNTLTLKDLPDDLASSGNSLLSMIMQLSMSIGVTIAGLLLGMFGQQHIAIDSASTHTVFMYTWLCIAFLIALPAIIFARVPDDTQTNAVISRRKRST

SEQ ID NO: 109 - DNA, 1413 bp, Citrobacter werkmanii NBRC 105721
atgactgaacttcctgacagcacccgctggcaactttggattgtggctttcggcttttttatgcagagtctggataccaccatcgtcaataccgcgcttcctagtatggccgccagccttggggaaagcccgttgcatatgcatatggtcattgtgtcatatgtattgaccgttgccgtaatgctgcctgccagcggatggttagcggacaaagttggcgtgcgcaatatcttcttcaccgccatcatcctgtttacgcttggcagtctgttttgcgcctggtcgagcacgcttaacgagctggtgatggcgcgcgtcttgcaaggcgttggtggcgcgatgatggtgccggttggcagactaacggtgatgaaaatcgtcccgcgtgagcaatatatggccgcgatgacgtttgtcacgttgcctggccagattggcccattgctgggtccggcgttgggcggcgttctggttgagtatgcctcgtggcactggatatttttgctgaatattccggttggcattgtcggcgcgatagctacgttgatgctgatgcctaactataccatgcaaacgcgacgcttcgatcttagtggattcgcgatgctggccgtcggcatggccgtgctaacgctggcgcttgatggctctaaagggaccggcatctcgggtatcacgttagcggtgctggttatctgcggagcgttggctattgcgctatatctgaaacacgcccatcgtaatccacgcgcgctgttttctctgaatctgtttcatactccaaccttcagtttgggtctgcttggctcttttgccgggcgcatcggcagcggtatgctgccgtttatgacgccagtgtttttgcagattgggcttggtttttcaccttttcatgccgggctgatgatggtcccgatggtgcttggctctatgggcatgaagcgcatcgtggtccaggtcgttaaccgctttggctaccgccgcgtactggtcactactacgcttggcctgtctctggtcagcctgttgctgatgaccaccgcgctgcttggctggtattacgccctgccttttgtgctgttcttgcaagggatggtcaactcaacgcgtttttcatcgatgaatacccttactcttaaagacctgccagacgatcttgccagcagcggcaatagcctgctgagtatgatcatgcaattgagtatgtctatcggagtgactatcgctggcctgttgctaggcatgtttggtcagcagcatatcgctatcgacagcggctctacgcataccgtctttatgtatacctggctgtgcattgctttcattattgctctgcctgccattatttttgcccgcgtgccgaacgacacgcaaactaacgcggtgatctcacggcgcaaaaggagcacctga

SEQ ID NO: 110 - protein, 470 aa, Citrobacter werkmanii NBRC 105721
MTELPDSTRWQLWIVAFGFFMQSLDTTIVNTALPSMAASLGESPLHMHMVIVSYVLTVAVMLPASGWLADKVGVRNIFFTAIILFTLGSLFCAWSSTLNELVMARVLQGVGGAMMVPVGRLTVMKIVPREQYMAAMTFVTLPGQIGPLLGPALGGVLVEYASWHWIFLLNIPVGIVGAIATLMLMPNYTMQTRRFDLSGFAMLAVGMAVLTLALDGSKGTGISGITLAVLVICGALAIALYLKHAHRNPRALFSLNLFHTPTFSLGLLGSFAGRIGSGMLPFMTPVFLQIGLGFSPFHAGLMMVPMVLGSMGMKRIVVQVVNRFGYRRVLVTTTLGLSLVSLLLMTTALLGWYYALPFVLFLQGMVNSTRFSSMNTLTLKDLPDDLASSGNSLLSMIMQLSMSIGVTIAGLLLGMFGQQHIAIDSGSTHTVFMYTWLCIAFIIALPAIIFARVPNDTQTNAVISRRKRST

SEQ ID NO: 111 - DNA, 1413 bp, Citrobacter amalonaticus
atgactgaattgcctaatagcacccgctggcaactgtggatcgtggctttcggcttctttatgcagtcgctggataccaccattgttaatactgcgcttccttcgatggcgaaaagcttaggggaaagcccgctgcatatgcatatggttgtggtttcgtatgtgctgaccgtggccgttatgctgcccgcgagcggttggctggccgacaaaattggcgtgcggaatatcttctttaccgcgatcgtactgtttaccctgggctcgctgttttgcgctggttcgggcacgctaaatgaactggttatggcccgggtgcttcaaggcgtcggcggcgcgatgatggtaccggttggccggctgacggtgatgaagatcgttccgcgcgagcagtatatggccgcgatgacgtttgttacgctgccgggccagataggcccgcttctgggcccggcgctgggcgggattttagtggaatatgcgtcgtggcactggatcttcctgattaaccttccggttggtatcgttggcgcgattgcgacgctgatgctgatgcctaactataccatgcaaacccgacgcttcgatttgtcgggttttctgctgctggccgctgggatggccgtcctgaccattgcgctggacggcagcaaagggatgggaatgtcgtcgttcgcgttgggtgcgttagtggtgtgcggcgtggccgcgatccttttgtacctgaagcacgcgaacgataacccgcgggcgctgttctcattgcacctgttccgcacgcccaccttctcgctgggactgtttggcagctttgcgggacgcattggcagcggcatgcttcccttcatgacgccggtttttcttcaaatcggtttgggcttttcgccgtttcacgccgggctgatgatgattcctatggtgttgggctcaatgggaatgaaacggatcgttgtccaggttgtcaaccgttttggctatcgacgggtgctggttttcaccacgttgggactgtccgtggtaaccctgctgtttatgtcgactgcgctgcttggctggtattacgtgctgccgctggtgctgtttgtccagggcatggttaactcgacgcgtttctcgtcgatgaataccctgaccctgaaagatctgccggatgagcttgcgagcagcgggaatagcctgctgtcgatgatcatgcaattgtcgatgtcaattggcgttaccgtcgccggtctgctgctaggaatgttcgcgcagcagcatatgactgtggatagcggaacgtcgcataccgtttttatgtatacctggctgtgtatggcttttattattgcgcttcctgctgtgatttttgcgcgcgttcccaacgatacccataaaaatgttgttatcgcgcgacgcaaaaggagaacctga

SEQ ID NO: 112 - protein, 470 aa, Citrobacter amalonaticus
MTELPNSTRWQLWIVAFGFFMQSLDTTIVNTALPSMAKSLGESPLHMHMVVVSYVLTVAVMLPASGWLADKIGVRNIFFTAIVLFTLGSLFCAGSGTLNELVMARVLQGVGGAMMVPVGRLTVMKIVPREQYMAAMTFVTLPGQIGPLLGPALGGILVEYASWHWIFLINLPVGIVGAIATLMLMPNYTMQTRRFDLSGFLLLAAGMAVLTIALDGSKGMGMSSFALGALVVCGVAAILLYLKHANDNPRALFSLHLFRTPTFSLGLFGSFAGRIGSGMLPFMTPVFLQIGLGFSPFHAGLMMIPMVLGSMGMKRIVVQVVNRFGYRRVLVFTTLGLSVVTLLFMSTALLGWYYVLPLVLFVQGMVNSTRFSSMNTLTLKDLPDELASSGNSLLSMIMQLSMSIGVTVAGLLLGMFAQQHMTVDSGTSHTVFMYTWLCMAFIIALPAVIFARVPNDTHKNVVIARRKRRT

SEQ ID NO: 113 - DNA, 1416 bp, Klebsiella oxytoca
atgactgatcttcccgacagcacccgctggcaattgtggattgtggctttcggcttctttatgcagtcactggacaccaccatcgttaataccgcgcttccctcaatggcgcaaagcctcggggaaagcccgctgaatatgcatatggtcattgtctcttatgtactgaccgttgcggtgatgctgcccgccagcggctggctggccgacaaagtcggcgtacgcaatatcttctttaccgccatcgtgctgtttaccctcggttcgctgttttgcgcactttctgccacgttaaacgaactgttgcttgctcgcgcgttgcaaggcgttggcggcgcgatgatggtgccggtcggcagattaactgtgatgaaaattgttccgcgtgagcaatatatggccgcgatgacctttgttacgctgcccggtcaggtcggccctctattgggtccggcgctcggcggcctgttagtggaatacgcgtcttggcactggatctttttgatcaatataccggtagggattatcggtgcgattgccacgctgatgttaatgcctaactataccatgcagacgcggcgtttcgatctctctggttttttgttgttagcggtcggcatggccgtattaacgctggcgctggatggctcgaaaggcacgggactttcaccgctggcgattgccggactggcagcgattggcgttgtggcactggtgctttatctgctgcacgccagaaataataaccgcgccctgttcagcctgaaactgttccgcacccgtaccttttcgctgggcctggccgggtcgtttgccggacgcatcggctcgggcatgttgccctttatgactccggttttcttgcagattggcctcggtttctcgccgttccatgccggactgatgatgatcccgatggtgctcggcagcatgggaatgaagcgaattgtagttcaggttgtgaatcgctttggctaccgtcgggtattagtggcgaccacgctgggcctgtcgctggtcaccctgctgtttatgactaccgcgctgctgggctggtactatgttttgccgttcgtcctgtttttgcaagggatggtcaactcgacgcgtttctcttctatgaataccctgacgctgaaagatctcccggacaatctggcgagcagcggcaatagcctgctatcgatgattatgcaattatcgatgtcgatcggcgtcactatcgccgggctgttgctgggactttttggttctcagcatgtcagcgtcgacagcggcaccactcaaaccgtctttatgtatacctggcttagcatggcgtttatcatcgcccttccggcgttcatctttgccagagtgtcgaacgatacgcatcaaaatgtcgctatttcgcggcgaaaaaggagcgcgcaatga

SEQ ID NO: 114 - protein, 471 aa, Klebsiella oxytoca
MTDLPDSTRWQLWIVAFGFFMQSLDTTIVNTALPSMAQSLGESPLNMHMVIVSYVLTVAVMLPASGWLADKVGVRNIFFTAIVLFTLGSLFCALSATLNELLLARALQGVGGAMMVPVGRLTVMKIVPREQYMAAMTFVTLPGQVGPLLGPALGGLLVEYASWHWIFLINIPVGIIGAIATLMLMPNYTMQTRRFDLSGFLLLAVGMAVLTLALDGSKGTGLSPLAIAGLAAIGVVALVLYLLHARNNNRALFSLKLFRTRTFSLGLAGSFAGRIGSGMLPFMTPVFLQIGLGFSPFHAGLMMIPMVLGSMGMKRIVVQVVNRFGYRRVLVATTLGLSLVTLLFMTTALLGWYYVLPFVLFLQGMVNSTRFSSMNTLTLKDLPDNLASSGNSLLSMIMQLSMSIGVTIAGLLLGLFGSQHVSVDSGTTQTVFMYTWLSMAFIIALPAFIFARVSNDTHQNVAISRRKRSAQ

SEQ ID NO: 115 - DNA, 1407 bp, Escherichia albertii B156
atgactgaactccccgacagcacccgttggcaactgtggattgtggctttcggcttctttatgcagtctctggataccactatcgttaataccgcgctcccatctatggcgcaaagcctgggagaatcgccactgcatatgcatatggtcattgtctcgtatgtgctgaccgttgcggtgatgctgccagccagcggctggctggcagacaaagtcggcgtacgtaatattttctttaccgccatcgtgctgtttacccttggttctctgttttgcgcgctctctggcacgctgaacgaactgttactggcgcgtgcgttgcaaggcgtcggcggcgcgatgatggtaccggtcggcagattaacggtgatgaaaattgttccgcgtgaacaatatatggccgcgatgacctttgtcacgctgcccggtcagattggtccgctgcttggcccggcacttggcggcctgctggtagaatacgcatcttggcactggatctttttaatcaatattccggtagggattatcggtgcgatcgctactctgatgttaatgccgaactataccatgcagactcggcgtttcgatctttctggctttctgttactggccgtcggcatggccgtactaacgctggcgctggatggctcgaaaggcaccgggctgtctccgctagccatcaccgtgctggttgctgttggcgtgctagccattgcgctctatctgcttcatgccagaaataatcaccgtgcgctgttctcgctgaaactgttccatactcgtaccttttctctgggcttagcgggaagcttcgccggtcgtgtcggcagcggcatgctaccatttatgacccccgttttccttcagattggccttggcttttctccgtttcacgccgggttaatgatgatcccgatggtgcttggcagcatgggaatgaagcgaattgtggtccaggtagtgaatcggtttggttaccgtcgagtgttagtggcaaccacgctgggcctgtctctggtcaccctactgtttatgactaccgctctgcttggctggtactatattttaccgttcgtcctgtttttgcaagggatggtcaactctacccgtttctcttctatgaataccctgacgctgaaggatctgccggaccgactggcgagcagcggcaatagcctgctgtctatgattatgcaattatctatgtcgatcggcgtcaccattgccgggctcttactgggattttttggttctcagcatatcagcggcgccactcaaaccgtctttatgtatacctggctcagcatggcgtttatcatcgccctcccggcacttgtctttgccagagttccgaatgatacgcatcaaaatgtggctattagtcggcgaaaaaggagcgcgcaatga

SEQ ID NO: 116 - protein, 468 aa, Escherichia albertii B156
MTELPDSTRWQLWIVAFGFFMQSLDTTIVNTALPSMAQSLGESPLHMHMVIVSYVLTVAVMLPASGWLADKVGVRNIFFTAIVLFTLGSLFCALSGTLNELLLARALQGVGGAMMVPVGRLTVMKIVPREQYMAAMTFVTLPGQIGPLLGPALGGLLVEYASWHWIFLINIPVGIIGAIATLMLMPNYTMQTRRFDLSGFLLLAVGMAVLTLALDGSKGTGLSPLAITVLVAVGVLAIALYLLHARNNHRALFSLKLFHTRTFSLGLAGSFAGRVGSGMLPFMTPVFLQIGLGFSPFHAGLMMIPMVLGSMGMKRIVVQVVNRFGYRRVLVATTLGLSLVTLLFMTTALLGWYYILPFVLFLQGMVNSTRFSSMNTLTLKDLPDRLASSGNSLLSMIMQLSMSIGVTIAGLLLGFFGSQHISGATQTVFMYTWLSMAFIIALPALVFARVPNDTHQNVAISRRKRSAQ

SEQ ID NO: 117 - DNA, 1413 bp, Salmonella enterica subsp. Salamae
atgactgaactccctgacagcactcgctggcaactttggattgtggctttcggcttttttatgcagtcgttggataccaccatcgttaatactgcgctgccttcaatggcgaaaagcctgggagaaagcccgctgcatatgcatatggtagtggtgtcatatgtccttaccgttgccgtaatgcttcctgccagcggctggctggctgacaaaatcggcgtgcgtaatattttctttgccgccatcgtgctgttcactctgggatcgctgttttgcgccctttcgggcacgctcaatcaactggtattagcgcgagtcttgcaaggcgtaggcggcgcgatgatggtgccggttggacgcctgacggttatgaaaatcgtgccgcgtgcgcaatatatggccgcgatgaccttcgttaccctgccgggacaggtagggcccttacttggccctgcgctgggcggagtgttagtggaatatgcttcatggcactggatctttctgatcaatatcccagtgggaattgttggcgctatcgcgacctttatgctgatgcccaactataccatagaaacccggcgctttgatctgccagggttcctgctgttagccataggcatggccgtacttacgctggcgctggacggcagcaaaagcatgggaatctcgccttggactttagccggactcgctgcgggcggcgcggctgctattctgctttatcttcttcatgccaagaagaactcaggcgcgttattcagcctgcgattgttccgcacgcctacgttttcactgggattgctgggctcatttgccgggcgcatcggcagcgggatgctgccgtttattacgccggtgttcttgcaaattgggcttgggttttcaccgtttcatgccgggttaatgatgatccctatggtgctgggcagcatgggaatgaagcggattgtggtgcagatagtcaaccgttttggctatcgtcgggttctggttgcgaccacgcttggactggctttggtcagcctgttgtttatgtcggttgcgctgcttggctggtattatcttctgccgctggtactgctcttgcagggaatggtgaactcggcgcgtttttcatcgatgaataccctaacgctaaaagacctccctgatacgctggccagcagcggcaatagcttattatcaatgattatgcagctttcgatgtcaatcggcgttactattgccggaatgttgctgggcatgtttggccaacagcatatcgggatagactcaagcgccactcatcacgtgttcttgtatacctggctatgtatggccgtcattatcgcgctaccggcgataatttttgcccgcgtgccgaacgatactcagcaaaatatggttatttcacggcgtaaaagaagcctgtaa

SEQ ID NO: 118 - protein, 470 aa, Salmonella enterica subsp. Salamae
MTELPDSTRWQLWIVAFGFFMQSLDTTIVNTALPSMAKSLGESPLHMHMVVVSYVLTVAVMLPASGWLADKIGVRNIFFAAIVLFTLGSLFCALSGTLNQLVLARVLQGVGGAMMVPVGRLTVMKIVPRAQYMAAMTFVTLPGQVGPLLGPALGGVLVEYASWHWIFLINIPVGIVGAIATFMLMPNYTIETRRFDLPGFLLLAIGMAVLTLALDGSKSMGISPWTLAGLAAGGAAAILLYLLHAKKNSGALFSLRLFRTPTFSLGLLGSFAGRIGSGMLPFITPVFLQIGLGFSPFHAGLMMIPMVLGSMGMKRIVVQIVNRFGYRRVLVATTLGLALVSLLFMSVALLGWYYLLPLVLLLQGMVNSARFSSMNTLTLKDLPDTLASSGNSLLSMIMQLSMSIGVTIAGMLLGMFGQQHIGIDSSATHHVFLYTWLCMAVIIALPAIIFARVPNDTQQNMVISRRKRSL

SEQ ID NO: 119 - DNA, 1416 bp, Klebsiella pneumoniae 30684/NJST258_2
atgactgacttaccggcgagcgtgcgctggcagctatggatagtggcgtttggctttttcatgcagtcgctggacaccaccattgtaaataccgcgttaccttcgatggcgaaaagcttaggggaatcaccgctgcatatgcatatgatcattgtttcgtatgtgctaacggttgcggtgatgctgccggcgagcggctggctggccgaccgggttggagtgcgcaatatcttctttaccgcgattgtgctgttcaccgcgggatcgctgttctgcgcgcaggcgagcaccttagaccagctggtgatggcgcgagtgctgcaaggagtcggcggcgcgatgatggtgccggttggccgcctgacggtgatgaagattgttccgcgcgatcagtatatggccgcgatgacctttgttaccctgccgggccaggttggcccgctgttaggcccggcgctgggcggagtgctggttgagtacgcgtcgtggcattggatcttcttaattaatattccggttgggattgtaggcgcgattgcgaccttatgtctgatgcctaactataccatgcagacccggcgctttgatttatcgggcttcctgctgctggccgcgggaatggcgaccctgaccctggcgttagacggccagaaagggttaggcatctcgcccgcgtggttagcgggactggttgcggttggcttatgcgcgctgctgttatacctgtggcatgcgcgcggcaacgcgcgggcgctgttcagccttaacctgttccgcaaccgcactttttcattaggcttaggcggcagctttgcgggacgcattggcagcggcatgctgccgttcatgaccccggtgttcctgcagattggcttaggcttttcgcctttccatgccgggctgatgatgattccgatggtgttaggcagcatgggcatgaagcggattgtggttcaggttgttaaccgcttcggctaccgacgggtgctggttgcgagcaccctgggcctggccgcggttagcctgctgttcatgttcagcgcgctggccggctggtattacgcgctgccgctggttctgttcctgcagggaatgattaacgcgagccgcttttcgtcaatgaataccctgacgctgaaagatttaccggacgatctggcgagcagcggcaatagcctgctgtcgatggtgatgcagctgtcgatgtcaattggcgtgaccattgccgggctgctgctgggactgtatggccagcagcatatgagcttagacgcggcgagcacccaccaggttttcttatatacctatctgagcatggccgcgattattgcgctgccggcgttaattttctcccgggtaccggatgacgttggcagcaataccgttttacggaggcgcaataggtcaggatcgtga

SEQ ID NO: 120 - protein, 471 aa, Klebsiella pneumoniae 30684/NJST258_2
MTDLPASVRWQLWIVAFGFFMQSLDTTIVNTALPSMAKSLGESPLHMHMIIVSYVLTVAVMLPASGWLADRVGVRNIFFTAIVLFTAGSLFCAQASTLDQLVMARVLQGVGGAMMVPVGRLTVMKIVPRDQYMAAMTFVTLPGQVGPLLGPALGGVLVEYASWHWIFLINIPVGIVGAIATLCLMPNYTMQTRRFDLSGFLLLAAGMATLTLALDGQKGLGISPAWLAGLVAVGLCALLLYLWHARGNARALFSLNLFRNRTFSLGLGGSFAGRIGSGMLPFMTPVFLQIGLGFSPFHAGLMMIPMVLGSMGMKRIVVQVVNRFGYRRVLVASTLGLAAVSLLFMFSALAGWYYALPLVLFLQGMINASRFSSMNTLTLKDLPDDLASSGNSLLSMVMQLSMSIGVTIAGLLLGLYGQQHMSLDAASTHQVFLYTYLSMAAIIALPALIFSRVPDDVGSNTVLRRRNRSGS

SEQ ID NO: 121 - DNA, 1416 bp, Klebsiella pneumoniae
atgactgacttaccgaccagcgtgcgctggcagctatggatagtggcgtttggctttttcatgcagtcgctggacaccaccattgtaaataccgcgttaccttcgatggcgaaaagcttaggggagagtccgctgcatatgcatatgatcattgtttcgtatgtgctaacggttgcggtgatgctgccggcgagcggctggctggccgaccgggttggagtgcgcaatatcttctttaccgcgattgtgctgttcaccgcgggatcgctgttctgcgcgcaggcgagcaccttagaccagctggtgatggcgcgagtgctgcaaggagtcggcggcgcgatgatggtgccggttggccgcctgacggtgatgaagattgttccgcgcgatcagtatatggccgcgatgacctttgtaaccctgccgggccaggttggcccgctgttaggcccggcgctgggcggagtgctggttgagtacgcgtcatggcactggatcttcttaattaatattccggttgggattgttggcgcgattgcgaccttatgtctgatgcctaactataccatgcagacccggcgctttgatttatcgggcttcctgctgctggccgccgggatggcgactctgaccctggcgttagacggccagaaagggttaggcatctcgcccgcgtggttagcgggattagtggccgttggcttatgcgcgctgctgttatacctgtggcacgcgcgcggcaacgcgcgggcgctgttcagccttaacctgttccgcaaccgcaccttttcgttaggcttaggcggcagctttgcgggacgcattggcagcggcatgctgccgttcatgaccccggtgttccttcagattggcttaggcttttcgcctttccacgccgggctgatgatgattccgatggtgttaggcagcatgggcatgaagcggattgtggttcaggttgttaaccgcttcggctaccgacgggtgctggttgcgagcaccctgggcttagcggccgttagcctgctgttcatgttcagcgcgttagcgggatggtattacgcgctgccgctggttctgttcctgcagggaatgattaactcgagccgcttttcgtcaatgaataccctgacgctgaaagatttaccggacgatctggcgagcagcggcaatagcctgctgtcgatggtgatgcagctgtcgatgtcaattggcgtgaccattgccgggctgctgctgggactgtatggccagcagcatatgtcattagacgcggcgagcacccatcaggttttcttatatacctatctgagcatggccgcgattattgcgctgccggcgttaattttctcccgggtaccggatgacgttggcagcaataccgttttacggcgacgcaataggtcaggatcgtga

SEQ ID NO: 122 - protein, 471 aa, Klebsiella pneumoniae
MTDLPTSVRWQLWIVAFGFFMQSLDTTIVNTALPSMAKSLGESPLHMHMIIVSYVLTVAVMLPASGWLADRVGVRNIFFTAIVLFTAGSLFCAQASTLDQLVMARVLQGVGGAMMVPVGRLTVMKIVPRDQYMAAMTFVTLPGQVGPLLGPALGGVLVEYASWHWIFLINIPVGIVGAIATLCLMPNYTMQTRRFDLSGFLLLAAGMATLTLALDGQKGLGISPAWLAGLVAVGLCALLLYLWHARGNARALFSLNLFRNRTFSLGLGGSFAGRIGSGMLPFMTPVFLQIGLGFSPFHAGLMMIPMVLGSMGMKRIVVQVVNRFGYRRVLVASTLGLAAVSLLFMFSALAGWYYALPLVLFLQGMINSSRFSSMNTLTLKDLPDDLASSGNSLLSMVMQLSMSIGVTIAGLLLGLYGQQHMSLDAASTHQVFLYTYLSMAAIIALPALIFSRVPDDVGSNTVLRRRNRSGS

SEQ ID NO: 123 - DNA, 1416 bp, Pseudocitrobacter faecalis
atgactgaattgcctgccagcgtgcgctggcaattgtggatagtggcgttcggcttctttatgcaatcgctggacaccactatcgtcaataccgccttgccttcaatggccgcttcactgggagagagcccgctgcatatgcatatggttgtggtatcatacgtgctgactgtggccgtgatgctccctgcctcgggctggctggccgaccgagttggcgtacgcaatatcttcttcaccgccattgtcctgttcaccgccggctcgctgttttgttcgctggcgagcacgttgaatgagctgctgatggcgcgtgtacttcaaggcgtcggcggcgcgatgatggtgcctgtcgggcgcctgactgtgatgaaaatcgtcccgcgctctcagtatatggccgcgatgaccttcgtcaccctgcctggtcaggtcggcccgctcctcggcccggcgttgggcggcgtgctggtagaatacgcctcgtggcactggatcttcctgatcaatattccggtcggcattatcggcgggatcgccacgctgatgctgatgcctaactatacgctgcaaacccgccgttttgacctgagcggatttatcgtgctggcgctgggcatggccgtgttgactctggccttggacgggcagaaagggctgggtatatcaagcctcacgctggcgattctggtcgccgtcggtgtcgcctcaatactgttttatttgtggcacgccaggggcaatgacaacgcgctgtttagcctgaagctctttcgcacgcccactttctcgttgggcctggctggctcattcgccggacgcatcggtagcggcatgctgccttttatgacgccggtctttctccagattgggctcggattttcgccgtttcacgccgggctgatgatgatcccgatggtgttgggcagcatgggaatgaaacggattgtggttcaggttgtcaaccgttttggttaccggaatgtgctggtcaccaccacgattggcttggcgctggtgagcctgctgtttatggcgacggcgctggctggctggtattacgccctgccgctggtgctgtttattcagggaatggtcaactcgagccgcttctcgtcaatgaatacgctgacgctgaaagatctccctgatgagcatgccagcagcggcaatagcctgctgtcgatgatcatgcagttgtcaatgtcaattggcgtcaccgtagcgggccttctgctgggcatgttcggccagcagcatgtcgccgccgatagcgccgtcacgcaccatgtctttatgtatacttatttgtgcatggcgctcattatcgccctgcctgccctgatttttgcccgcgtacccgacgacgtcaccaaaaacgtggtcatcgcccgacgaaaaaggtcagaacagtga

SEQ ID NO: 124 - protein, 471 aa, Pseudocitrobacter faecalis
MTELPASVRWQLWIVAFGFFMQSLDTTIVNTALPSMAASLGESPLHMHMVVVSYVLTVAVMLPASGWLADRVGVRNIFFTAIVLFTAGSLFCSLASTLNELLMARVLQGVGGAMMVPVGRLTVMKIVPRSQYMAAMTFVTLPGQVGPLLGPALGGVLVEYASWHWIFLINIPVGIIGGIATLMLMPNYTLQTRRFDLSGFIVLALGMAVLTLALDGQKGLGISSLTLAILVAVGVASILFYLWHARGNDNALFSLKLFRTPTFSLGLAGSFAGRIGSGMLPFMTPVFLQIGLGFSPFHAGLMMIPMVLGSMGMKRIVVQVVNRFGYRNVLVTTTIGLALVSLLFMATALAGWYYALPLVLFIQGMVNSSRFSSMNTLTLKDLPDEHASSGNSLLSMIMQLSMSIGVTVAGLLLGMFGQQHVAADSAVTHHVFMYTYLCMALIIALPALIFARVPDDVTKNVVIARRKRSEQ

SEQ ID NO: 125 - DNA, 1236 bp, Yokenella regensburgei ATCC43003
atgcaaaatcataccctctcgggcaaacgtctgggtcgtcaggcgctactctttccgctctgcttagtactgtacgaattttcgacctatattggaaacgacatgattcagccgggcatgttagccgtggttgagcagtaccaggctggtatcgaatgggtccccacttcgatgaccgcttacttagcgggcgggatattcctgcagtggttactggggccgctgtcggatcgtatcggccgtcgtccggtgatgctaaccggagtggtctggtttatcgtgacctgcttagcgacgctgctggcgcagaatatcgagcagttcacgtttctgcgttttctgcaaggagtgagcctgtgctttatcggtgctgtgggctatgcggcgattcaggagtcgttcgaagaggccgtgtgtattaaaattaccgcgctgatggcgaacgtggcgctaatcgcgccgctgttaggtccgttagtgggcgcggcgtggattcattttgcgccgtgggaaaccatgtttgtgctgttcgcggcgctggccgcgatctcgttttttggcctgcagcgtgctatgccggaaacggcgacccgcataggcgaaaagctgtcgttaaaagagctaggtcgtgactattcagcggtgctgaaaaacctgcgctttgtcgcgggcgcgctggctacgggcttcgtcagcctgccgctcctggcgtggatcgcgcagtcaccggtcatcattatgagcggtgaacacgcgagcagctacgagtatggtatgctgcaagtgccgatcttcggtgctctgatcattggtaatctggtgctggcgcgtttaacttcgcgccgttcggtgcgctcgctaatcatcatgggcggctggccgatcgtggctgggctcattctcgcggctgcggctactgtggtatcaagccatgcgtatctgtggatgacggccgggttaagcgtgtacgctttcggtattggtctggcgaacgcgggactggtgcgcctgaccttattcgcgagcgacatgtcaaaaggcacggtagccgcggcgatgggcatgctgcagatgatgatcttcaccgtcgggatcgaagtcagcaagcacgcgtatctgctcggcggcaacggcctctttagcctgtttaatctggccggtggattagtctggctggtgctgatcgtctactttctgaaagacaaaacggttggtgcttcgactaagcccgactag

SEQ ID NO: 126 - protein, 411 aa, Yokenella regensburgei ATCC43003
MQNHTLSGKRLGRQALLFPLCLVLYEFSTYIGNDMIQPGMLAVVEQYQAGIEWVPTSMTAYLAGGIFLQWLLGPLSDRIGRRPVMLTGVVWFIVTCLATLLAQNIEQFTFLRFLQGVSLCFIGAVGYAAIQESFEEAVCIKITALMANVALIAPLLGPLVGAAWIHFAPWETMFVLFAALAAISFFGLQRAMPETATRIGEKLSLKELGRDYSAVLKNLRFVAGALATGFVSLPLLAWIAQSPVIIMSGEHASSYEYGMLQVPIFGALIIGNLVLARLTSRRSVRSLIIMGGWPIVAGLILAAAATVVSSHAYLWMTAGLSVYAFGIGLANAGLVRLTLFASDMSKGTVAAAMGMLQMMIFTVGIEVSKHAYLLGGNGLFSLFNLAGGLVWLVLIVYFLKDKTVGASTKPD

SEQ ID NO: 127 - DNA, 1233 bp, Cronobacter muytjensii
atgcaaactcatgctaaccgaaccggacggttaggacgccaggcgctgctgtttccgttatgtctggtgttatacgagttctcaacttatattggcaacgatatgattcagccgggaatgttagcggttgttgaacagtatcaggccggcgtggaatgggtgccgaccagcatgaccgcgtatttagcgggcggcatgttccttcagtggctgctcggcccgttaagcgaccgcattggccggcgcccggtgatgctggccggcgtggtgtggttcattgtgacctgcctcgcgaccctgctggcgcagactattgagcagtttactgtgctacggtttcttcagggcattagcctgtgcttcattggcgccgtgggatatgcggctattcaggaaagcttcgaggaagcggtgtgcattaaaattaccgcgctaatggcgaacgtggcgctgatcgcgccgctgttaggcccgctggttggcgcggcctgggtgcacgcggcgccttgggaaatgatgttcgtgctgtttgcggcgctggccgcgatctcattttttgggctgtggcgcgcgatgccggaaaccgcgacccggctcggcgaaaaactgagcctgcgcgagctgggacgggattataaagcggtgctgaaaaacctgcggttcgtttcaggcgcgctggcgattggattcgttagcctgccgctgctggcgtggatcgctcagagcccggtaatcattattagcggcgaacagatgagcacctatgagtatgggctgcttcaggtgccgatctttggcgcgctgattttaggcaacctggtgctggcgaagttaaccgctcggcgcagcgtgcgcagcctgatcattatgggcggctggccgatcatggccgggctggcgttagcggcgtttgcgaccctgttatcgtcgcatgcgtatttatggatgaccgccgggttatcaatctacgcgtttggcattggcattgcgaacgcgggcctggtgcgcctgaccctgttcgcgagcgatattagcaaaggcaccgtatccgcggcgatgggcatgcttcagatgaccatctttactgttggcattgaagtgagcaaacatgcgtggattggcggcggcaacggcctgtttaacctgttcaatttagctaacggactgctgtggctgggcctgatggtgatctttctgaaagacaaaaccgttggcgcgagccgggaaggatga

SEQ ID NO: 128 - protein, 410 aa, Cronobacter muytjensii
MQTHANRTGRLGRQALLFPLCLVLYEFSTYIGNDMIQPGMLAVVEQYQAGVEWVPTSMTAYLAGGMFLQWLLGPLSDRIGRRPVMLAGVVWFIVTCLATLLAQTIEQFTVLRFLQGISLCFIGAVGYAAIQESFEEAVCIKITALMANVALIAPLLGPLVGAAWVHAAPWEMMFVLFAALAAISFFGLWRAMPETATRLGEKLSLRELGRDYKAVLKNLRFVSGALAIGFVSLPLLAWIAQSPVIIISGEQMSTYEYGLLQVPIFGALILGNLVLAKLTARRSVRSLIIMGGWPIMAGLALAAFATLLSSHAYLWMTAGLSIYAFGIGIANAGLVRLTLFASDISKGTVSAAMGMLQMTIFTVGIEVSKHAWIGGGNGLFNLFNLANGLLWLGLMVIFLKDKTVGASREG

SEQ ID NO: 129 - DNA, 1236 bp, Klebsiella oxytoca
atgcagaattattcgctttcaggcaaacgcttaggacgccaggcgctgttatttccgctatgcctggtgctatacgagttctcgacctatattggcaatgacatgattcagccgggcatgttatcggttgtggctgaatttggcgtgggcaacgaatgggtgccgacctcaatgaccgcgtatctggccggcggaatgtttcttcagtggctgctggggccgctttcggaccgcattggccgtcgtccggtgatgctgaccggagtagtgtggtttattgtaacctgcctggcgacgctgctggcgcaaactatcgagcagttcacgctgctgcgctttcttcagggaattagcctgtgctttattggcgcggttggctatgcggcgatacaggagtcatttgaagaggccgtgtgcattaagattaccgcgctgatggcgaacgttgcgctgatcgcgccgctgttagggccgttagtgggcgcggcctgggtccacttactgccgtgggaaatgatgtttgttctgtttgcggtgttagcggcgattgcgtttttcgggcttcagaaggcgatgccggaaaccgcgacccgcatgggcgagaagctatcggtaaaagagctggggcgcgattatcgtgaagtgctgaaaaacctgcgctttgttgcgggcgcgctggcgaccgggtttgttagcctgccgctgctggcgtggatcgcgcaatcgccggtgattattattagcggcgagcaggcgaccagctacgaatatggcctgctgcaagtgccgatctttggcgcgctgatcgcggggaacctggtgctggcgcgccagacttcacgcaaaacggtgcgctcgctgatcatcttaggcggctggccgatcatgattggcctgctgatttccgcggttgcgacggttgcgtcgacgcatgcgtatctgtggatgaccgccgggctgagcgtctacgcgttcgggatcgggttagcgaacgcgggactggtgcgtctgacgctgtttgcgagcgagatgtcaaaggggacggtgtccgcggcgatgggaatgctgcaaatgctgatttttaccgttgggattgaagtttcaaagcatgcgtatgagtcaggcgggagcggattattcagcctattaaacctgctgagcggagtgttatggctggcgatgacggtttacttcctgaaggataaacgcgtgggcaacgcgcatgaaccgcagtaa

SEQ ID NO: 130 - protein, 411 aa, Klebsiella oxytoca
MQNYSLSGKRLGRQALLFPLCLVLYEFSTYIGNDMIQPGMLSVVAEFGVGNEWVPTSMTAYLAGGMFLQWLLGPLSDRIGRRPVMLTGVVWFIVTCLATLLAQTIEQFTLLRFLQGISLCFIGAVGYAAIQESFEEAVCIKITALMANVALIAPLLGPLVGAAWVHLLPWEMMFVLFAVLAAIAFFGLQKAMPETATRMGEKLSVKELGRDYREVLKNLRFVAGALATGFVSLPLLAWIAQSPVIIISGEQATSYEYGLLQVPIFGALIAGNLVLARQTSRKTVRSLIILGGWPIMIGLLISAVATVASTHAYLWMTAGLSVYAFGIGLANAGLVRLTLFASEMSKGTVSAAMGMLQMLIFTVGIEVSKHAYESGGSGLFSLLNLLSGVLWLAMTVYFLKDKRVGNAHEPQ

SEQ ID NO: 131 - DNA, 1233 bp, Citrobacter koseri (Citrobacter diversus)
atgcaaaacctttctcaaactggcgttcggctcggacgccaggctctgcttttccctctctgtctggtgctttacgaattttcaacctatattggcaacgatatgattcagccagggatgctggccgttgtggaacagtatcaggccgggattgactgggtcccgacctcgatgactgcctacctggccggcggaatgtttcttcagtggctgcttgggccgctgtcggatcgtattggacgccgcccggtgatgcttgccggcgtgatttggtttattgttacctgccttgccactctgctggcgcaaaatattgagcagttcaccctgctgcgttttcttcaaggagtaagcctgtgctttattggcgccgtgggatacgccgccattcaggaatcgtttgaagaagcggtgtgtattaaaattaccgcgctaatggctaacgtcgccctgattgcgccgcttctggggccgttagtgggcgccgcctgggtgcatgtcctgccgtgggaaggaatgtttgtcctgtttgccgtactggccgcgattgcttttgttggccttcagcgcgcgatgccggagactgcgacccgccttggcgagaagctgtcgcttaaagaactgggacgggattataccctggtgctgaaaaacgtgcggtttgtcgccggcgcgctggcgctgggattcgttagcctgccgctgctggcgtggattgcccagtcgccgattataattattagcggggagcagctcagcagctatgaatatggccttcttcaggtccctgtttttggcgcgctgattgccgggaatctggtgctggcgcggcttacctcgcggcgaaccgtgcgcgctcttattattatgggcggctggccgattgttgcgggacttctgattgccgctgccgcgaccgtggtttcatcgcatgcgtatctatggatgaccgccgggctgagcgtttacgcctttggcattggcgtggcgaacgccgggctggtgcgtctgaccctgtttgccagcgatatgagcaaaggcaccgtatcagcggcgatgggaatgcttcagatgctgatttttaccgtgggaattgaagtgagcaagcatgcttatctgagcggcggcaacggccttttcagcctcttcaacctggcgaacggcattctctggctgctgcttatggtcattttcctgaaagacaaacgggtaggggactcacgggaagggtga

SEQ ID NO: 132 - protein, 410 aa, Citrobacter koseri (Citrobacter diversus)
MQNLSQTGVRLGRQALLFPLCLVLYEFSTYIGNDMIQPGMLAVVEQYQAGIDWVPTSMTAYLAGGMFLQWLLGPLSDRIGRRPVMLAGVIWFIVTCLATLLAQNIEQFTLLRFLQGVSLCFIGAVGYAAIQESFEEAVCIKITALMANVALIAPLLGPLVGAAWVHVLPWEGMFVLFAVLAAIAFVGLQRAMPETATRLGEKLSLKELGRDYTLVLKNVRFVAGALALGFVSLPLLAWIAQSPIIIISGEQLSSYEYGLLQVPVFGALIAGNLVLARLTSRRTVRALIIMGGWPIVAGLLIAAAATVVSSHAYLWMTAGLSVYAFGIGVANAGLVRLTLFASDMSKGTVSAAMGMLQMLIFTVGIEVSKHAYLSGGNGLFSLFNLANGILWLLLMVIFLKDKRVGDSREG

SEQ ID NO: 133 - DNA, 1233 bp, Escherichia marmotae
atgcaaaataaattagcaactggtgccagacttgggcgccaggcattactttttcctctttgtctggtgctttacgaattctcgacctatatcggcaacgatatgatccaacctggtatgcttgccgtggttgagcagtatcaggctggtattgactgggttcctacttcgatgaccgcttacctggccggaggaatgtttttgcagtggctgttaggaccactgtcggatcgtattggtcgccgtccggtaatgttagctggcgtggtgtggttcataatcacctgcctggcgatattactggcgcaaaatattgaacaattcacgctgttacgctttcttcaagggataagcctttgtttcattggcgctgttggttacgccgcgattcaggaatcttttgaagaagcggtttgtatcaagatcaccgcgctgatggcgaatgtggcgctgattgctccgttacttgggccattagttggtgccgcgtggattcatgtgttaccttgggaaggaatgtttgtgttatttgctgcattagcggcgatctcattcttcggtctgcaacgtgcgatgccagaaacggctacgcgcattggcgagaaactgtcgctgaaagagcttggtcgagactataagctggtactgaagaatagccgctttgttgcaggagcgctggcgctgggatttgtctcgctgccgttactggcgtggattgcccagtcaccgattatcatcatcaccggtgaacagttatcgagctatgaatatggtttactgcaagtgcctattttcggcgcgttaattgcgggcaatttactgctggcacgtctgacctcgcgccgtaccgtacgttcgctgattattatgggtggctggccgattatgattggtctgctggtcgccgcagcggccacggtcatctcatcgcacgcgtatttatggatgaccgccgggttatcgatttatgcttttggtattggtctggcgaatgcagggctggtgcgtttaacgttattcgcctcggatatgtcgaaagggacggtatccgcggcgatgggaatgttgcaaatgctgatctttaccgtaggcattgagatctcgaaacatgcctggctgaatggcggcaatggattatttaatttatttaatcttgccaacggtgttttatggctgctgctgatgtttatctttttaaaagataaacaaactggaaattctaacgacggataa

SEQ ID NO: 134 - protein, 410 aa, Escherichia marmotae
MQNKLATGARLGRQALLFPLCLVLYEFSTYIGNDMIQPGMLAVVEQYQAGIDWVPTSMTAYLAGGMFLQWLLGPLSDRIGRRPVMLAGVVWFIITCLAILLAQNIEQFTLLRFLQGISLCFIGAVGYAAIQESFEEAVCIKITALMANVALIAPLLGPLVGAAWIHVLPWEGMFVLFAALAAISFFGLQRAMPETATRIGEKLSLKELGRDYKLVLKNSRFVAGALALGFVSLPLLAWIAQSPIIIITGEQLSSYEYGLLQVPIFGALIAGNLLLARLTSRRTVRSLIIMGGWPIMIGLLVAAAATVISSHAYLWMTAGLSIYAFGIGLANAGLVRLTLFASDMSKGTVSAAMGMLQMLIFTVGIEISKHAWLNGGNGLFNLFNLANGVLWLLLMFIFLKDKQTGNSNDG

SEQ ID NO: 135 - DNA, 1227 bp, Shigella flexneri
atgcaaaataaattggctagtggtgccagactcggtcgtcaggcgttgctcttccctctctgtctggtgctctacgaattttcaacctatatcggcaacgatatgatccaacccggtatgttggccgttgtggaacaatatcaggccggcattgattgggttcctacttcgatgaccgcgtatctggccggcggaatgtttttgcaatggctcctggggccgctgtcggatcgtattggtcgtcgtccggtgatgctggccggtgtggtgtggtttatcgtcacctgtctggcaatattgctggcgcaaaatattgaacaattcaccctgttgcgtttcttgcagggcataagcttctgtttcattggcgctgtgggatacgccgcaattcaggaaagtttcgaagaagcggtttgtatcaagatcaccgcgctgatggcgaacgtggcgctgattgctccgctactcggtccgctggttggcgcggcgtggatccatgtgctgccttgggaaggaatgtttgttttgtttgccgcattggcagcgatcagttttttcggtctgcaacgagctatgcctgaaaccgccatgcgtataggcgagaaactgtcactgaaagaactcggtcgtgactataagctggtgctgaagaacggccgttttgtggccggggcgctgggattcgttagcctgccattgctggcgtggatcgcccagtcgccgattatcatcattaccggggagcagttgagcagctatgaatatggcttgctgcaagtgcctattttcggggcgctcattgccggtaacttgctgttggcgcgtctgacctcgcgtcgtaccgtacgttcgctgattattatgggcggctggccgattatgattggtctgttggtcgctgctgcggcaacggttatctcatcgcacgcgtatttgtggatgaccgccgggttgagtatttatgctttcggtattggtctggcgaatgcgggactggtgcgattgaccctgtttgccagcgatatgagtaaaggtacggtttctgcggcgatgggaatgctgcaaatgctgatctttaccgtcggtattgaaatcagcaaacatgcctggctgaacgggggcaacggactgtttaatctcttcaacctcgtcaacggaattttgtggctgtcgctgatggttatctttttgaaagataaacagatgggaaattctcacggaggataa

SEQ ID NO: 136 - protein, 408 aa, Shigella flexneri
MQNKLASGARLGRQALLFPLCLVLYEFSTYIGNDMIQPGMLAVVEQYQAGIDWVPTSMTAYLAGGMFLQWLLGPLSDRIGRRPVMLAGVVWFIVTCLAILLAQNIEQFTLLRFLQGISFCFIGAVGYAAIQESFEEAVCIKITALMANVALIAPLLGPLVGAAWIHVLPWEGMFVLFAALAAISFFGLQRAMPETAMRIGEKLSLKELGRDYKLVLKNGRFVAGALGFVSLPLLAWIAQSPIIIITGEQLSSYEYGLLQVPIFGALIAGNLLLARLTSRRTVRSLIIMGGWPIMIGLLVAAAATVISSHAYLWMTAGLSIYAFGIGLANAGLVRLTLFASDMSKGTVSAAMGMLQMLIFTVGIEISKHAWLNGGNGLFNLFNLVNGILWLSLMVIFLKDKQMGNSHGG

SEQ ID NO: 137 - DNA, 1233 bp, Salmonella enterica subsp. Salamae
atgcataatcgtttgcaatcaggcggcagactgggtcgtcaggcgctgctctttcctctttgtctggtgctgtacgaattttcaacttatattggcaacgacatgatccagccgggaatgctggccgtggtcgcgcagtatcaggcgagcctggactgggtcccgacgtcgatgaccgcttatctggccggcggaatgttcttgcagtggctgctcggcccgttgtcggaccgtattggccgtcgtcctgtgatgctggccggcgttgtgtggtttattgttacctgcctggcgacgctactggcgaaaaatatcgaacaatttacgttcctgcgttttttgcaaggaattagcttgtgttttatcggcgctgtagggtatgccgcgattcaggaatcgtttgaggaagcggtatgtataaaaattaccgcgctgatggcgaacgtggcgttaatcgcgccgctattgggacccctggttggggcagcttgggtgcatgttctgccgtgggaaggaatgtttattttatttgccgtactggccgctatcgcctttttcgggcttcaacgcgctatgccggagactgccacgcgacggggcgaaacgttatcattcaaagtgctgggccgggattatcggctggtgataaaaaaccggcgatttgttgctggagcgttagcgctgggatttgttagcctgccgctgttggcctggattgcgcagtcgccgattatcatcattagcggcgaacagcttagcagctatgaatatgggctgcttcaggtgcctgtctttggcgcgctgattgccgggaatctggtgttggcgcgtttaacctcgcgacgtacggtccgctcgctgatcgtaatgggcggatggcctattgttgccgggctgattatcgccgccgcggcgactgtggtatcatcgcatgcttatttatggatgactgcgggattaagcgtatacgcttttggcattgggctggcgaatgccggactggtgcggctcacgctattctcgagcgatatgtcaaaaggaacggtttccgcggcgatgggaatgcttcaaatgctgatttttaccgtcggcattgaagtatcaaaacacgcctggttaagcggcggcaatgggctatttagcctgtttaatctggctaacgggattctctggctactgctgatgctggttttcttaaaagataagcggactggggactcgcaaactggctaa

SEQ ID NO: 138 - protein, 410 aa, Salmonella enterica subsp. Salamae
MHNRLQSGGRLGRQALLFPLCLVLYEFSTYIGNDMIQPGMLAVVAQYQASLDWVPTSMTAYLAGGMFLQWLLGPLSDRIGRRPVMLAGVVWFIVTCLATLLAKNIEQFTFLRFLQGISLCFIGAVGYAAIQESFEEAVCIKITALMANVALIAPLLGPLVGAAWVHVLPWEGMFILFAVLAAIAFFGLQRAMPETATRRGETLSFKVLGRDYRLVIKNRRFVAGALALGFVSLPLLAWIAQSPIIIISGEQLSSYEYGLLQVPVFGALIAGNLVLARLTSRRTVRSLIVMGGWPIVAGLIIAAAATVVSSHAYLWMTAGLSVYAFGIGLANAGLVRLTLFSSDMSKGTVSAAMGMLQMLIFTVGIEVSKHAWLSGGNGLFSLFNLANGILWLLLMLVFLKDKRTGDSQTG

SEQ ID NO: 139 - DNA, 1233 bp, Citrobacter youngae ATCC 29220
atgcagaaccgtctctcatcaggcgcccggctgggtcgtcaggctctcctttttcctctttgtctggtgctttacgaattctcgacttatatcggcaacgatatgatccaacctggcatgctcgcggttgtggagcaatataacgccggactcgattgggtacctacttcgatgaccgcttatctggccgggggcatgtttctccagtggctgctcggaccgctgtcggatcgtattgggcgccgcccggtgatgctcaccggcgtgctttggttcattgtgacctgccttgcgaccctgcttgcgcagaatattgagcaatttacctttctgcgtttcctccaagggataagcctgtgctttatcggcgcggttggttacgccgcgattcaggaatcgtttgaagaagcggtgtgcataaaaattaccgcgctgatggctaacgtagcgctgattgcgccgctgctgggaccgctcgtcggcgctgcctgggtgcatattctgccgtgggaaggaatgtttatcctgtttgctgcccttgcgtcgatctcgttttttggtctgcaacgcgcgatgccggaaacggctacccggctcggcgaaaagctgtcgattaaagagctgggtaaagattataagctggtgctaaaaaatgggcgctttgttgccggtgcgctggctctcggttttgtcagcctgccgctcctggcgtggattgcgcaatcgccgattattatcatcagcggtgaacatcttagcagctacgaatatggcctgctccaggtgccgatttttggcgctctcattgccggtaatctggcgctggcgcgactgacttcgcgcaagaccgtgcgttcgctgattatcatgggcggctggcctatcgccgtcgggctggtgattgccgcggccgccacggttgtgtcatcgcatgcgtatctctggatgacggccggtcttagcgtctatgcgttcggcattggtctggcgaatgccgggctggttcgcctgacgctgtttgcctcagaaatgtcaaaggggacggtttcagcagcaatgggcatgctccagatgctgatctttaccgtcggtattgagctgagcaaacacgcttatctgctgggaggaaacggcctcttcagcctgtttaatctggctagcggcgtgctgtggctgatactcatggttatcttcctgaaagataaacgggtagggaattcgcggaaaatttaa

SEQ ID NO: 140 - protein, 410 aa, Citrobacter youngae ATCC 29220
MQNRLSSGARLGRQALLFPLCLVLYEFSTYIGNDMIQPGMLAVVEQYNAGLDWVPTSMTAYLAGGMFLQWLLGPLSDRIGRRPVMLTGVLWFIVTCLATLLAQNIEQFTFLRFLQGISLCFIGAVGYAAIQESFEEAVCIKITALMANVALIAPLLGPLVGAAWVHILPWEGMFILFAALASISFFGLQRAMPETATRLGEKLSIKELGKDYKLVLKNGRFVAGALALGFVSLPLLAWIAQSPIIIISGEHLSSYEYGLLQVPIFGALIAGNLALARLTSRKTVRSLIIMGGWPIAVGLVIAAAATVVSSHAYLWMTAGLSVYAFGIGLANAGLVRLTLFASEMSKGTVSAAMGMLQMLIFTVGIELSKHAYLLGGNGLFSLFNLASGVLWLILMVIFLKDKRVGNSRKI

SEQ ID NO: 141 - DNA, 1233 bp, Citrobacter freundii
atgcagaaccgtctttcttcaggcgcccgcttggggcgccaggcactccttttccctctttgtctggtgctttacgaattctctacttatatcggtaacgatatgatccaacctggaatgttagcggttgtggagcaatatcaggccggtcttgattgggttccaacgtcgatgaccgcttatctggcaggcggcatgtttctccagtggttgctcgggccgctgtctgaccgtgttggtcgtcgtccggtgatgctcaccggagtggtttggttcattgtgacctgcctagcaacgctgtttgctcaaaatattgagcaatttactttcctgcgttttctccaagggattagcctgtgctttattggcgcggtcggttacgccgcaattcaggagtcgtttgaagaagcggtgtgcattaaaatcactgcgctaatggcaaacgtagcattgattgcaccgcttttgggaccgttggttggggccgcctgggtccatgtccttccgtgggaaggcatgtttgtcctgtttgccgcgctggctgcaattgccttttttggcctgcagcgcgcgatgccggaaacggctacccgtctgggcgagaagctttctatcaaagagttgggcaaagattataagctggtgctaaagaatgtgcgttttgtcgcaggcgcactggcattgggctttgtctcactgccattgctggcgtggattgcgcagtcgccgatcattattatctcaggcgaacatcttagcagctacgaatatggcctgctccaggtgccgatttttggtgcactgattgcaggcaacctggtgctggcgcgtctgacttcgcgccgcacggtgcgctctctgatcatcatgggtggctggccaatctccgtgggattgatcattgccgccgcggccacggttgtttcttctcatgcgtatctgtggatgacggctggtctttcactttatgcatttggcattggcgtggcgaatgccgggttggtgcgcctgacgctgtttgcctcagaaatgagcaaagggactgtttcagcagcaatgggtatgctgcaaatgctgatatttaccgttggtattgagctgagcaaacatgcttatctgctgggtggaaacggcttgttctcactgtttaatctggccagcggtgtgctgtggctgattctgatggttatctttctgaaagataagcgagtagggaattcgcgggaaggctaa

SEQ ID NO: 142 - protein, 410 aa, Citrobacter freundii
MQNRLSSGARLGRQALLFPLCLVLYEFSTYIGNDMIQPGMLAVVEQYQAGLDWVPTSMTAYLAGGMFLQWLLGPLSDRVGRRPVMLTGVVWFIVTCLATLFAQNIEQFTFLRFLQGISLCFIGAVGYAAIQESFEEAVCIKITALMANVALIAPLLGPLVGAAWVHVLPWEGMFVLFAALAAIAFFGLQRAMPETATRLGEKLSIKELGKDYKLVLKNVRFVAGALALGFVSLPLLAWIAQSPIIIISGEHLSSYEYGLLQVPIFGALIAGNLVLARLTSRRTVRSLIIMGGWPISVGLIIAAAATVVSSHAYLWMTAGLSLYAFGIGVANAGLVRLTLFASEMSKGTVSAAMGMLQMLIFTVGIELSKHAYLLGGNGLFSLFNLASGVLWLILMVIFLKDKRVGNSREG

SEQ ID NO: 143 - DNA, 1230 bp, Enterobacter kobei
atgcaaaaccattcacttcccggccgccgtcttggacgccaggcgcttctttttccgttgtgtctggtcctgtacgaattctcaacttatattgcgaacgacatgattcagccgggcatgttagcggttgtagagcagtataacgcgggcattgaatgggtaccgacctcgatgaccgcgtatttagcgggcggaatgtttcttcagtggctgctggggccgctgtcggatcgtatcggccgtcgcccggtcatgcttaccggcgtggtgtggtttattgtcacctgtctggctacccttctggcgcaggacattgaacagttcactctgctgcgctttcttcaaggcgtgagcctgtgctttatcggcgccgtgggctatgcggcgatccaggagtcgtttgacgaggcgacctgtattaagattactgctcttatggcgaacgtggcgctgattgcgcctctgctggggccgctggttggtgcggcctgggtgcatgctgcgccttgggaaggcatgtttgtgctgttcgcggtactggccgcgatcgcgtttttcggcctgcatcgcgcgatgccggaaaccgcgacgagattaggggagcccctttcgcttaacgcgctgggccgggattataaagcggtgctgaaaaatggccgcttcgttgcgggcgcgctggcgacgggctttgtcagcctgccgctgctggcgtggattgcgcagtcgccgatcattattattagcgcggaaggcatgtcgagctatgagtacggtctgcttcaggtgcctatctttggcgcgctgatcattggtaacctggtgctggcgcgtctgacctcacgccgtaccgtgagatcgctgatcatcatgggcggcgggccgatcgtcgcgggactgctggtcgcggccgtggctaccgtggcgtcatcgcatgcgtatctttggatgaccgcgggcctgtcaatctatgcttttggtatcggccttgcgaacgccgggctggtgcgcctgacgctgttcgcgagcgatatgtcaaaaggtaccgtgtcagcggctatgggcatgctgcaaatggcgatcttcaccgtcggcattgaagtctcaaaacatgcgtttctggccggcggcaatggcctgttcagcctgtttaatctggcgaacgggcttatttggctggcgctgatggttgtattcctgaaggataaaacggtcggcaacgcgctttcataa

SEQ ID NO: 144 - protein, 409 aa, Enterobacter kobei
MQNHSLPGRRLGRQALLFPLCLVLYEFSTYIANDMIQPGMLAVVEQYNAGIEWVPTSMTAYLAGGMFLQWLLGPLSDRIGRRPVMLTGVVWFIVTCLATLLAQDIEQFTLLRFLQGVSLCFIGAVGYAAIQESFDEATCIKITALMANVALIAPLLGPLVGAAWVHAAPWEGMFVLFAVLAAIAFFGLHRAMPETATRLGEPLSLNALGRDYKAVLKNGRFVAGALATGFVSLPLLAWIAQSPIIIISAEGMSSYEYGLLQVPIFGALIIGNLVLARLTSRRTVRSLIIMGGGPIVAGLLVAAVATVASSHAYLWMTAGLSIYAFGIGLANAGLVRLTLFASDMSKGTVSAAMGMLQMAIFTVGIEVSKHAFLAGGNGLFSLFNLANGLIWLALMVVFLKDKTVGNALS

SEQ ID NO: 145 - DNA, 1236 bp, Enterobacter sp.
atgctaaaccggtcatcatcaggcactcgcctgggtcggcaggcgctactattcccactgtgcctggtgctatacgagttctcgacctatattgggaacgatatgattcagccaggcatgttagccgtggttgcgcagtataacgcgggcattgaatgggtgccaacctcgatgaccgcttacctggccggtggcatgtttcttcagtggctgttaggcccgctgagcgaccggatcggccgacggccggtgatgttaaccggtgtggcgtggtttattgttacctgtctggcgaccctgctggcgcaaaccattgaacagtttatggtgctgcggtttcttcaaggtgttagcctgtgctttattggcgcggttggttacgcggcgattcaggaaagctttgaagaagcggtgtgcattaaaattaccgcgctgatggctaacgtggcgctgatcgcgccgctgctgggcccgctggtaggcgcggcctgggtgcatgttgcgccctgggaaggcatgtttgtgctgtttgcggtgctagcggcgattagcttttatgggctgcatcgggcgatgccagagactgctacccgcattggcgagaagttatcactgcaggaactgggtcgcgactataaagaagtgctgaaaaacgggcgctttgtggccggtgcgctggcgatcggctttgtttgtctgccgctgctggcgtggattgcgcagagcccggtgattattattagcggcgagaacttaagcagctacgagtatggcctgctccaggtgccgatctttggggcgctgatcgtggggaatattgtgctggcgcggctaaccagccgccggaccgtgcgcagcctgatcatcatgggcggctggccgattgtgatcggtctggttgttgcggccgtggcgactgttgtttcaagccatgcgtacctgtggatgaccgccgggttaagcatttacgcgtttgggatcggtctggctaacgcgggcctggtgcgcctgactctgtttgcgagcgaagtgagcaaaggtactgtgagcgcggcgatgggcatgcttcagatgctgatttttaccgtgggcatcgaagtgagcaaacatgcgttcagcagcggcggcaacggtctgttcagcctgtttaatctggtaaacggcctgctgtggctggcgctgatgtttgttttcctgaaagacaagcgagtgggctcaagccttcaaccgggataa

SEQ ID NO: 146 - protein, 411 aa, Enterobacter sp.
MLNRSSSGTRLGRQALLFPLCLVLYEFSTYIGNDMIQPGMLAVVAQYNAGIEWVPTSMTAYLAGGMFLQWLLGPLSDRIGRRPVMLTGVAWFIVTCLATLLAQTIEQFMVLRFLQGVSLCFIGAVGYAAIQESFEEAVCIKITALMANVALIAPLLGPLVGAAWVHVAPWEGMFVLFAVLAAISFYGLHRAMPETATRIGEKLSLQELGRDYKEVLKNGRFVAGALAIGFVCLPLLAWIAQSPVIIISGENLSSYEYGLLQVPIFGALIVGNIVLARLTSRRTVRSLIIMGGWPIVIGLVVAAVATVVSSHAYLWMTAGLSIYAFGIGLANAGLVRLTLFASEVSKGTVSAAMGMLQMLIFTVGIEVSKHAFSSGGNGLFSLFNLVNGLLWLALMFVFLKDKRVGSSLQPG

SEQ ID NO: 147 - DNA, 1230 bp, Lelliottia sp. WB101
atgctaaaccgttcatcatcgggtaaccgtctgggtcgtcaggcgctactttttcctctgtgtctggtgctttatgaattttcgacctatattggcaatgatatgattcagccgggtatgttagcggttgtggagcagtataacgccgggattgaatgggtaccgacttcgatgaccgcgtacctggctggcggcatgttccttcagtggctgctggggccgctgtcggatcgcatcggccgtcgcccggtgatgctgactggcgtggtgtggttcattgttacctgcttagcgatcctgctggctcaaacgatcgaacaatttactctgctgcgatttcttcaaggagtgagcctgtgcttcattggcgccgtgggatacgcggctatccaggagtcgttcgaggaagcggtgtgtattaaaattaccgcgctgatggcgaacgtggcgctgatcgcgccgctgttaggcccgttagtgggcgcggcctgggtgcatgtagcgccgtgggaaggaatgttcgtgctgtttgcggctcttgcggcgatctcgttttttggccttcatcgcgctatgcctgaaaccgcgactcgactgggcgaaaaactgtcgcttaaagagctggggcgcgactataaagaagtgttaaggaatggccgcttcgtggccggcgcgctggcgaccgggttcgttagcctgccgctgctggcgtggatcgcgcagtcaccggttatcatcatcagcggtgagaagttatcaagctatgaatatggtctactccaggtgcctgtattcggcgcgctgatcattggtaacctggtgctggctcgcttaacttcgcgtcgtagcgtgcgttcgctgattatcatgggcggctggccgatcgtggccgggctggttgtagctgcggtagcgaccgttgcgtcatctcatgcgtacctgtggatgactgccgggctgtcaatctacgcgttcgggatcgggctggcgaatgccgggctggtgcgcctgacgctgttcgcgagcgagatgtcaaaaggcaccgtatcggccgctatgggaatgctgcagatgctgattttcaccgtgggcatcgaagtttcaaaacatgcgtatagcttcggcggcaacgggctgtttagcctgttcaacttagcgaatggcgtgctgtggttagggctgatggtgatgtttctgaaagataaacgcgttggtagcgcgcttcagtaa

SEQ ID NO: 148 - protein, 409 aa, Lelliottia sp. WB101
MLNRSSSGNRLGRQALLFPLCLVLYEFSTYIGNDMIQPGMLAVVEQYNAGIEWVPTSMTAYLAGGMFLQWLLGPLSDRIGRRPVMLTGVVWFIVTCLAILLAQTIEQFTLLRFLQGVSLCFIGAVGYAAIQESFEEAVCIKITALMANVALIAPLLGPLVGAAWVHVAPWEGMFVLFAALAAISFFGLHRAMPETATRLGEKLSLKELGRDYKEVLRNGRFVAGALATGFVSLPLLAWIAQSPVIIISGEKLSSYEYGLLQVPVFGALIIGNLVLARLTSRRSVRSLIIMGGWPIVAGLVVAAVATVASSHAYLWMTAGLSIYAFGIGLANAGLVRLTLFASEMSKGTVSAAMGMLQMLIFTVGIEVSKHAYSFGGNGLFSLFNLANGVLWLGLMVMFLKDKRVGSALQ

SEQ ID NO: 149 - DNA, 1233 bp, Enterobacter ludwigii EcWSU1
atgcttaaccgttcatcatcaggttcacgtctgggtcgtcaggcgcttctctttcctctttgtctggtgttatatgaattttcaacttatattggcaacgatatgattcagcctggcatgctggccgtcgttgaacagtataacgctgggattgaatgggtaccgacttcgatgaccgcgtacctggctggcggcatgttccttcagtggctgcttgggccgctgtcggatcgtatcggccgtcgtccggtgatgctgactggcgtggtgtggttcattgtgacctgcttagcgacgctgctggcgcaaaatatcgagcaattcaccctgctgcgctttctgcaaggagtgagcttatgctttattggtgcggttgggtacgcggcgattcaggaatcgttcgaggaagcggtgtgtattaagatcaccgcgctgatggcgaacgtggcgctgattgcgcctctgctggggccgctggttggcgctgcctgggtccatgtcgctccgtgggaaggaatgtttgtgctgttcgcggtgctggccgcggttgcttttttcggcctgcatcgcgcgatgccggaaaccgctacccgtctgggtgagaaactgtcgctaaaagagctgggccgggattataaagaagtgctgaagaacggccgcttcgttgcgggcgcgctagcgactgggttcgtcagcctgcctctgctggcgtggatcgcgcagtcgccggttatcattattagcggcgagcagttaagcagctacgagtacggcctgctccaggtgcctatcttcggcgcgctgatcattggtaacctggtgctggcgcgtttaacttcgcgccgtacggtgcgtgcgcttattatcatgggcggctggcctatcgctgccgggctggtcttagcggccgttgcgactgtggtgtcgtcccacgcgtacctgtggatgaccgctggacttagcgtctatgcgtttggtattggtgtggcgaatgccgggctggtacgcctgacgctgttcgcgagcgagatgagcaaaggcacggtgtcagcggcgatgggcatgcttcaaatgctgatctttaccgtcggaattgaagtgagcaaacatgcgtatgctttcggcggcaacggtctgtttagcctgttcaatctggctaacggcgtgttatgggtcgggctcattgtggtgtttctgaaggataaacgcgttgggaatgctcttcagccgtaa

SEQ ID NO: 150 - protein, 410 aa, Enterobacter ludwigii EcWSU1
MLNRSSSGSRLGRQALLFPLCLVLYEFSTYIGNDMIQPGMLAVVEQYNAGIEWVPTSMTAYLAGGMFLQWLLGPLSDRIGRRPVMLTGVVWFIVTCLATLLAQNIEQFTLLRFLQGVSLCFIGAVGYAAIQESFEEAVCIKITALMANVALIAPLLGPLVGAAWVHVAPWEGMFVLFAVLAAVAFFGLHRAMPETATRLGEKLSLKELGRDYKEVLKNGRFVAGALATGFVSLPLLAWIAQSPVIIISGEQLSSYEYGLLQVPIFGALIIGNLVLARLTSRRTVRALIIMGGWPIAAGLVLAAVATVVSSHAYLWMTAGLSVYAFGIGVANAGLVRLTLFASEMSKGTVSAAMGMLQMLIFTVGIEVSKHAYAFGGNGLFSLFNLANGVLWVGLIVVFLKDKRVGNALQP

SEQ ID NO: 151 - DNA, 654 bp, Actinoplanes utahensis
atgctaacccatgtgtttggcctattaggcgcggcgctgagcatgagcattgcgtggcctcaggtgtatcgcagctgcgtgcgtcgtcgtaccggcggcctgagcgcgaccgcgtgcatgctggccgtggcgatgccgctgggctgggtgacctatgggctgctgattggagaccgatttcaagtggtgacgaacgcggtgagcgcgagcaccggagtggcgattctgattgcgctgctggtgacccgaccagcggtgcgcagcggccgcgcgctgctggcgagcgtgggcgcggccgccggagtgctgctggccgtgacggcgattgcggcgagcgcggcgctgcctcaggtgagcgggccgcgcgccgcggcgatgctgggcaccgtgctggccgcgattagctttgtgagcgcgataccgcagccgttagcgctgctgcgtgatcgcgatcaggatattagcgggctgagcccggtgcgttggacgctcgcggcgagcgcgtgcgcgagctggtttgcgtatggcttaggagtggggcagccagcggtgtgggcgagcgcgttagtgggcctgaccagcgcgctgaccgtgtgcggcgtgctgtttacccgtcgtgcgggcgccggcgtgcgcgtgctggcgaccgcgtaa

SEQ ID NO: 152 - protein, 217 aa, Actinoplanes utahensis
MLTHVFGLLGAALSMSIAWPQVYRSCVRRRTGGLSATACMLAVAMPLGWVTYGLLIGDRFQVVTNAVSASTGVAILIALLVTRPAVRSGRALLASVGAAAGVLLAVTAIAASAALPQVSGPRAAAMLGTVLAAISFVSAIPQPLALLRDRDQDISGLSPVRWTLAASACASWFAYGLGVGQPAVWASALVGLTSALTVCGVLFTRRAGAGVRVLATA

SEQ ID NO: 153 - DNA, 261 bp, Chitinophagaceae bacterium PMG_246
atggataaaacccagattgtaggcattgttgcgggcatactcactgcgagcagcctgctgcctcaggtgattaaaaccctaaaagaaaagaaggccgccgaagtaagcattggcatgctgattacgctgatgttaggcgtagcactgtggattgtatatggctttctgcgcgatgatctgccgattattgtaacgaactgcttcagcctgctggtgaatctgaccatgattggactgcggctgaagtataggcatcgctga

SEQ ID NO: 154 - protein, 86 aa, Chitinophagaceae bacterium PMG_246
MDKTQIVGIVAGILTASSLLPQVIKTLKEKKAAEVSIGMLITLMLGVALWIVYGFLRDDLPIIVTNCFSLLVNLTMIGLRLKYRHR

SEQ ID NO: 155 - DNA, 252 bp, Rhizobium sp. PDC82
atgatagatgctgggctcctggtcgggtatttagcgagcatctgcagcgttgcccggaaagtggtaaagaccggggacactgctgcgatcagcgcgcgcatgtatgttttaaccgttattggcttcgccttatggaccgggtttgggctattacgcggcgagtggccgattatcctcaccaatagcatttgcttctgcctgagcggcgtcgtgttatttcgaaaattaaccgcccgcaatcgcgccgggtaa

SEQ ID NO: 156 - protein, 83 aa, Rhizobium sp. PDC82
MIDAGLLVGYLASICSVARKVVKTGDTAAISARMYVLTVIGFALWTGFGLLRGEWPIILTNSICFCLSGVVLFRKLTARNRAG

SEQ ID NO: 157 - DNA, 687 bp, Kineococcus rhizosphaerae DSM 19711
atgccggtgaccaccctaatttcaagcttaggattattagccgcgggattaggcgtgttttgcggtattcctcaggttgtgcgcttagtgcgtgatccggatgcgaccggcctgagctatccgagcgcggtgttaggcgcgttagcgagcgcgacctggttaagctatggcgtggcgctgcgcgacccggcgcagttagtggcgaacgtgccgggtattgtgtgcgcggtgttaaccgtggtgttagcggcgaaacgtctgggcctgccgctgcgcaccgcggcgtatgcggccgccggttgggcgccgttagttgcggccgcgtttgcgttaggtggcgttgtggttgttggcgtgctgggcaccaccgtgagcttagtgaaaatgctgccgcagattttaaccgtggtgcgccgcgatccggtgcatggcctggcgccggcgacctttgtgttaacccaggtgagcgcgaccctgtggaccgcgtatggcctggcgaccgggcagtggagcgttgtggtgtgcagcgcggtgaccgtggtgctggccggtattgtgctgagccgccgctgcccgccgttagcggttgcgcgcgcgctgcatgaaggccgctttggcgtgccgggtcagttattagtgcgcccgtttgtgctggcgcgtcgtgcgggcttaaccctggccgcgtaa

SEQ ID NO: 158 - protein, 228 aa, Kineococcus rhizosphaerae DSM 19711
MPVTTLISSLGLLAAGLGVFCGIPQVVRLVRDPDATGLSYPSAVLGALASATWLSYGVALRDPAQLVANVPGIVCAVLTVVLAAKRLGLPLRTAAYAAAGWAPLVAAAFALGGVVVVGVLGTTVSLVKMLPQILTVVRRDPVHGLAPATFVLTQVSATLWTAYGLATGQWSVVVCSAVTVVLAGIVLSRRCPPLAVARALHEGRFGVPGQLLVRPFVLARRAGLTLAA

SEQ ID NO: 159 - DNA, 279 bp, Morganella morganii IS15
atgtcggatactaaacgtccgccgcacgatcatagccgttttattcgttgtctcggttgggttgccacctttactgccttttgcatgtatgttagctatatcccccagatcatggataacctggccgggcataaaacttcgccgctgcagccgctggctgcggcctttaattgcactctttgggtgatctacggtcttaaagtgaaagatctgccggtcgccgttgcgaacgcgcccggtgttctgttcgggctggccgccatgctgactgccttgtag

SEQ ID NO: 160 - protein, 92 aa, Morganella morganii IS15
MSDTKRPPHDHSRFIRCLGWVATFTAFCMYVSYIPQIMDNLAGHKTSPLQPLAAAFNCTLWVIYGLKVKDLPVAVANAPGVLFGLAAMLTAL

SEQ ID NO: 161 - DNA, 822 bp, Geodermatophilus obscurus
atgacctcaccggtgattcccgctgcggttgcgctgccgctgaccgatgaacgcccggaaccggcgtgtggctgtcccgcgcgccgctggccgcatcccagccagagcaccagaacggagccacgcagcaccatgattgcggcgctgggcagcctggccgcggcgctgagcattaccgtggtgtggccccaggtgtggctgagctgccgccatggccgcaccctgggcctgagcccgacgggcagctggctggccgtgggcctgaacctgtgctggctgaccagcggcctgctggttggcgataccccgcagattgcgacccatgccgtggttggcgcgggcaataccgcggtgctggccgcgctgctgctgacccagccgcatgcgcgcagcgcgcaagtgctgctgcgcaccgccgcgggagccgcgggcctggccgcgctggccgcgggcggcgttgcggccgtggtgctgggagcggatacgactcaggtgacgagcgtgctggcgagcgtgaccaccgtggttggcattgtggccgcgctgcctcagctgctgggcattctgtttgatcgcgcgcaggatctgagcggcatgagcccggcgcgctggtatctgggcgcgggcagctgcgcgagctggaccgcgtatggctggctgctggggcagccgacggtgtggctgagcgcgggctttggcctggtgtgcgcggtgaccacctgcgcggtgctgcgcacccgccgcccggcgccccccgcggctccagtggtgccgctgcgcccggctgctgcgccgcgcagagtgctggccgccgcggcgtaa

SEQ ID NO: 162 - protein, 273 aa, Geodermatophilus obscurus
MTSPVIPAAVALPLTDERPEPACGCPARRWPHPSQSTRTEPRSTMIAALGSLAAALSITVVWPQVWLSCRHGRTLGLSPTGSWLAVGLNLCWLTSGLLVGDTPQIATHAVVGAGNTAVLAALLLTQPHARSAQVLLRTAAGAAGLAALAAGGVAAVVLGADTTQVTSVLASVTTVVGIVAALPQLLGILFDRAQDLSGMSPARWYLGAGSCASWTAYGWLLGQPTVWLSAGFGLVCAVTTCAVLRTRRPAPPAAPVVPLRPAAAPRRVLAAAA

SEQ ID NO: 163 - DNA, 252 bp, Bradyrhizobium sp. BTAi1
atgctgaccaccctgattggcctgggcgcggcgacctgcaccacttgcagctttctgcctcaggtgattaaagcgtggcgaagccgcagcacccaggatattagcgcgggcatgtttgtgctgctgaccaccggcaacgcgatgtggctgctatacggcgcgctgattaacgatctgccgttagtggttgcgaacctgattaccctggcgttagtggcgaccattctgggcctgaaactgcgctacggctga

SEQ ID NO: 164 - protein, 83 aa, Bradyrhizobium sp. BTAi1
MLTTLIGLGAATCTTCSFLPQVIKAWRSRSTQDISAGMFVLLTTGNAMWLLYGALINDLPLVVANLITLALVATILGLKLRYG

SEQ ID NO: 165 - DNA, 261 bp, Bradyrhizobium japonicum USDA 110
atggatccgtttcttattaaactgattggcttcgctgcggcgacctgcaccaccgtggcgtatgcgccgcagtttattaaagtgcttaaaacccgcagcgcgcgcgatattagcctgggcatgtttctggtgatggtgctgggcctggcgctgtggctgatttatggcctgctgagcggcgatgcgccgctgattgcgagcaacgcggtgaccatgctgttagcgggcggcatactggtgatgaaactgagatatggctaa

SEQ ID NO: 166 - protein, 86 aa, Bradyrhizobium japonicum USDA 110
MDPFLIKLIGFAAATCTTVAYAPQFIKVLKTRSARDISLGMFLVMVLGLALWLIYGLLSGDAPLIASNAVTMLLAGGILVMKLRYG

SEQ ID NO: 167 - DNA, 276 bp, Xanthomonas campestris pv. vesicatoria str. 85-10
atgcaatttgttgatgttgtgggctggttagcgagcattattctgattgcgaccttaattcggcagatttataagcaatggcgctcggatgcggcgcaaggcgttagccgctggttatttttaggccagatttcagctagcgtgttatttattttatattcatacctggtaggcaacgcggtgttcattgttagcaatgtgctgattctgctgacggcgctgattggctacgctttgcagcgagttaaacgccgcaggctggaacgcgctgcgtag

SEQ ID NO: 168 - protein, 91 aa, Xanthomonas campestris pv. vesicatoria str. 85-10
MQFVDVVGWLASIILIATLIRQIYKQWRSDAAQGVSRWLFLGQISASVLFILYSYLVGNAVFIVSNVLILLTALIGYALQRVKRRRLERAA

SEQ ID NO: 169 - DNA, 273 bp, Herbaspirillum aquaticum
atgagcgaccagattaccgatttaataggctggggcgccaccttaattttactgctgaccattagcagccaggtgtacgaacagtggcgcagccgcagcacgcaaggcgtgagccattggttattcgcggggcagctagcggcttcagccggctttgtgacctatagcgtgctgcacggtgattgggtatttgtggtgagcaacgtatttctgttattcaccgcgttactgggccaggtgctgtatctgcgcaaccggcgccgccagcagtga

SEQ ID NO: 170 - protein, 90 aa, Herbaspirillum aquaticum
MSDQITDLIGWGATLILLLTISSQVYEQWRSRSTQGVSHWLFAGQLAASAGFVTYSVLHGDWVFVVSNVFLLFTALLGQVLYLRNRRRQQ

SEQ ID NO: 171 - DNA, 261 bp, Flavobacteria bacterium MS024-2A
atgacgcagtttcagattgaactgattggtatgtttgctgcggtattaacgacgagcgcgtttataccgcaagtatataaaatttggaaaacgcgtgtgagcgatggtgtatcactgagcatgtatttgtcattatttataggagtggtgagctggtgtgtgtacggctacctgataggtagcccatcagtgctgatagcgaatattattgcgggaatgtttcagctgatgattatttattttaagctgaaatttaaataa

SEQ ID NO: 172 - protein, 86 aa, Flavobacteria bacterium MS024-2A
MTQFQIELIGMFAAVLTTSAFIPQVYKIWKTRVSDGVSLSMYLSLFIGVVSWCVYGYLIGSPSVLIANIIAGMFQLMIIYFKLKFK

SEQ ID NO: 173 - DNA, 786 bp, Rhizobium sp. Root149
atgctgaatgcgatttggaactatcggtattttattgttaccagcgttcaggccgattttcggaaccgcgttgctcgcagccgcttaggcctgctctggttagtgatcgccccgttagcgcagattgtgacctatgcgttcgtcttaagcagcgttatgagccagcggctgccaggcattgataataccttcgcgtatgcgatttacctgatggccggcttccagggctggctcctgttcgtggaaattattagccgaagcatgaatgtgtttattgaaagcggcaatgtcctgaaaaaaattgcgttcccacgcatcgcgctccccctcgttgtggtgattaccagcgtgataaataacctgattttttttgtcgttctgctggctgtgtttaccttagcgggctttcagatgggatggaatgtgctgtggctcccggtcttaatcgcggtgaatgccgcgttcgccgcgggattaggcgtgaccttaggcgtcctcaacgtgttcatccgcgacgtggggcagattgtaccgattattatccagtttctgttttggatgaccccgattgtgtatgttaaggacatcctgccgccagcgtttcagagcattattaaagcgaatcccctgttttggatggttgagaattatcaccgcgtgatggtgtataataccatgccggacctgaaagttttaggcgttttagggttcctggctctggtctttctcgctttaggcttcaccttatttcgcaaagctagccctgaaatggttgatgttctataa

SEQ ID NO: 174 - protein, 261 aa, Rhizobium sp. Root149
MLNAIWNYRYFIVTSVQADFRNRVARSRLGLLWLVIAPLAQIVTYAFVLSSVMSQRLPGIDNTFAYAIYLMAGFQGWLLFVEIISRSMNVFIESGNVLKKIAFPRIALPLVVVITSVINNLIFFVVLLAVFTLAGFQMGWNVLWLPVLIAVNAAFAAGLGVTLGVLNVFIRDVGQIVPIIIQFLFWMTPIVYVKDILPPAFQSIIKANPLFWMVENYHRVMVYNTMPDLKVLGVLGFLALVFLALGFTLFRKASPEMVDVL

SEQ ID NO: 175 - DNA, 837 bp, Azospirillum brasiliense LMG 04375
atgaccatggccgctagcgctccgcaagtaccgaccggactagcgggcctgtttacgatgccatggcaccatcgcgcgctgctgcgccgcttagtgcgtagggaaattgaggccaggtatcgtggaagcctgctaggcgtggtgtgggcgctcctgaccccggtgttaatgctggccgtgtttacctttgtgtttagcgtggtgtttcaggcgcgctggggcagttcaaccggcggaaaaggggaatttgcgctgattctgtttgctggcctgattgtgtttaatatgtttagcgaagcgctgaaccgtagcccaagcctagtactggaaaacccgagctacgtgaaaaaagtggtgtttccgttagaagtgctgccctgggtgatggtgctaagcagcctgtttcaggttgtgattagcctagcgattctgttagtgttccacctagcggtgtcagggctgcccccgctgagcgtgctgtttctgccgctggtgtggctaccactgctgttcttatgcctgggcaccggcttcctgtttggcgcgttaggcgtatacctgcgcgatctgcgaagcattatgccagtggctagccagttactgatgttcttaagcccgctgttttatccggcgagcgcgctgccggaaagctttcgccgcctgttgcatttaaacccgctgacgggcattattgaaaatacgcgcacggtgctgttcgctggcaccccgccggactggaccgtgttaggcgtgcagattgtggccgggctgttatttgctacctttagctttcgtctgtttcgccatctacgaactggattcgcggatatagtgtaa

SEQ ID NO: 176 - protein, 278 aa, Azospirillum brasiliense LMG 04375
MTMAASAPQVPTGLAGLFTMPWHHRALLRRLVRREIEARYRGSLLGVVWALLTPVLMLAVFTFVFSVVFQARWGSSTGGKGEFALILFAGLIVFNMFSEALNRSPSLVLENPSYVKKVVFPLEVLPWVMVLSSLFQVVISLAILLVFHLAVSGLPPLSVLFLPLVWLPLLFLCLGTGFLFGALGVYLRDLRSIMPVASQLLMFLSPLFYPASALPESFRRLLHLNPLTGIIENTRTVLFAGTPPDWTVLGVQIVAGLLFATFSFRLFRHLRTGFADIV

SEQ ID NO: 177 - DNA, 1233 bp, Rhizobium sp. Root149
atgaccaatgttgtgatgaccgtggacaaagtgggcaaagtcttccggcgctatcggcacgaactgcatcgcgtcttaggctggttctcattaccagtggggcagccggaagagcgctgggtcctgcgggatatttcatttaccgtagaggctgggcaggccctggccatcgtcgggcgcaatggcgccggcaaaagcaccctcttaaaactgatcaccggcaccatgcgcgccgcggaaggcaatattgggttatcaggcaaaattagcgcgattttagaactgggaatgggctttaatccggattataccggccgccagaacgtttttcatgccttaggcttaatgggctaccagcatgccgatatcgcggccgcgatggaaggcattgaggccttcagcgaactgggaagctattttgatcagccgctgcgagtgtatagcagcggcatgcatatgcggttagcttttagcgtggccactgctttccggccagacatcttaattgtggatgaggccttatcagtaggcgacgcgtattttcagcataaaagctttgaccgcattaaagaattccgccagcaggggaccaccctgattttagtcagccacgaccggatggccctgttaagcttatgcgaccgcgcgatcctgctgcacgaaggagcggttgccttagatggggagcccgaaactgtcttagactattataacgcgttattagggcagaaagaagcccaaccgattttaaccgaagagcgagaggatggccgcgtgcagacctcaagcgggagccgccgggtcagcatcgtggaagcccggctggaagatgcccagggcggcccaattgataccttagatgttggggcagaagtggcgatccgcgttaaagcgaaggccaatgaagatgttgaatcattagtgtttggctacgccattaaagaccgctttgggcagaccatgtatggcaccaatacctattatagcgaacaggccctgaccgatgtaaaagctggcgaagagattgagtttagcgcccgctttcgggccgatttaggagtgggcacctattcagttgctctagccctggttgggggcgagaaccatattgaggataattatgaatggcgcgatttagcgattatttttgatgtcgcgaataccagcaaacagacctttgacgggcgaatctatctgccgagcgagttccatttaaagcggaccggtggctag

SEQ ID NO: 178 - protein, 410 aa, Rhizobium sp. Root149
MTNVVMTVDKVGKVFRRYRHELHRVLGWFSLPVGQPEERWVLRDISFTVEAGQALAIVGRNGAGKSTLLKLITGTMRAAEGNIGLSGKISAILELGMGFNPDYTGRQNVFHALGLMGYQHADIAAAMEGIEAFSELGSYFDQPLRVYSSGMHMRLAFSVATAFRPDILIVDEALSVGDAYFQHKSFDRIKEFRQQGTTLILVSHDRMALLSLCDRAILLHEGAVALDGEPETVLDYYNALLGQKEAQPILTEEREDGRVQTSSGSRRVSIVEARLEDAQGGPIDTLDVGAEVAIRVKAKANEDVESLVFGYAIKDRFGQTMYGTNTYYSEQALTDVKAGEEIEFSARFRADLGVGTYSVALALVGGENHIEDNYEWRDLAIIFDVANTSKQTFDGRIYLPSEFHLKRTGG

SEQ ID NO: 179 - DNA, 1416 bp, Azospirillum brasiliense LMG 04375
atgaaggatgataccgctatgcaggatatgccgagcgatggcgatgtgagcattcgtgtaagcggcgtgggcaaatgctaccatatttatgcgcgtccgcaggaccgcttactgcaatttctgctgcgcggccgccgtcagttttatcgtgaattttgggccttaaaagatattgatctgacgattcgccgcggggaaagcgtggcgctgattgggcgcaacggggccggcaagtcaactctactccaggtgattagcggcgtactgcaaccgaccggaggcagcatggatgtgcgcgggcgcattgcgccgctattagagctaggcagctcatttaacccagagtttagcgggatggaaaatattgggctgagcgcgagcgtattaggcttaagcgaggaacagattgcggaacgtcgcgaagcgattattgcgtttgcggatattggcgattttattcaccagccggtgaggacctacagcagcggcatgcaggcgcgcctagcgtttgccgtggctgcgcacgtggatgcggaaattctgattgtggatgaaattctgagcgtgggcgatattgcgtttacccagaaatgcacccgctttattcgcgaatttcgcgagcgcggaacgctgctgtttgtgagccatgacattggcgcgataaccgcgctgtgcgatcgcgcggtgtggctaagcggaggaacggttgtagcggatggcgtgccgcgcgaagtggctcaccactataaagcgtggatggccggacctcagcagcatatgacggcgcaggattttctggaaaccctgaaaaccgcggaagcggaagcgactgaagtggaagcggaagcggaagcggaagccgcggttgaagccgaagttgtggtgacccaaggcgggccggatggcgaaacggcgctagtgcagacgttagatgcccaggtgcagaccgtgtttcgccgcgatgtgctgagcagcggcgaaggcggggcgaaaatattgcaggcgtggattgaagatgcgcgtggcaccccagtgagcatggtgaacggcggcgatttagtgaccctgtgcattgaagcggaagcgcagcagaccctggatagcctgattgtgggctttggcgtgaaagatcgtttaggccagacgatgtttgcgtgggatacgaccaaaaccgacttagtgattccgccggtgaatgtgggcgatcgctttcgcgccgggtttcgctttcattttccatatctgctgggcggcacctatacgaccaatctggcgattgcgaacgggacgagcgatatgtatattcagcaccattggatgtatgatgcgctggaatttcatgtgcaatggagcaccgtggcgcagggcctgtttggcataccaatggatggcgtaagcctggcgattaacccctaa

SEQ ID NO: 180 - protein, 471 aa, Azospirillum brasiliense LMG 04375
MKDDTAMQDMPSDGDVSIRVSGVGKCYHIYARPQDRLLQFLLRGRRQFYREFWALKDIDLTIRRGESVALIGRNGAGKSTLLQVISGVLQPTGGSMDVRGRIAPLLELGSSFNPEFSGMENIGLSASVLGLSEEQIAERREAIIAFADIGDFIHQPVRTYSSGMQARLAFAVAAHVDAEILIVDEILSVGDIAFTQKCTRFIREFRERGTLLFVSHDIGAITALCDRAVWLSGGTVVADGVPREVAHHYKAWMAGPQQHMTAQDFLETLKTAEAEATEVEAEAEAEAAVEAEVVVTQGGPDGETALVQTLDAQVQTVFRRDVLSSGEGGAKILQAWIEDARGTPVSMVNGGDLVTLCIEAEAQQTLDSLIVGFGVKDRLGQTMFAWDTTKTDLVIPPVNVGDRFRAGFRFHFPYLLGGTYTTNLAIANGTSDMYIQHHWMYDALEFHVQWSTVAQGLFGIPMDGVSLAINP

SEQ ID NO: 181 - DNA, 3198 bp, Sinorhizobium medicae WSM419
atgtttctgacacggattagcattaaccacccggtgtttgctaccatgatgatggttatgattttagtgttagggctgttctcatatggacggttaggagtggaccattacccagaaaccgatctgcccgtggtagttgtggcgaccacctacaccggggcaagcccagagagcgtggagagcgaaatttcacggccgatagaagcggcgctgaacacaattggcgggattgataccattaccagcgagagctacgaggggcgaagcattgtggttgttcagtttgaagttgacgtggactcacaagatgcggctcaagaagttcgcgatcgggtagcgcggttagagacaaaattcccagatgctgttgcgaccccccaggtgacccggtacaaaccagagggccaggctattctgtcagtagctgttagcagcaccagccggaccctgccagagattaccaccttagcgacacgcgttattaacaatcgcctgagcgtaataagcggcgtgggccaagtatcactgataggcagcagcgaacggcaagtactggtagtagttgatcccgaccggttaggggcctacggcctggctgtggcgaccgtgattgaggctattcggggcgagaaccaggaccgagcggccgggaccttaacaagcggcattaatcagcggattgtaaccgtggaagggcgcatagcgaatacatcagcgtttaaccgcattattgtggctcaaagaaatggctatccagtgtacttaagcgaagttgctaccatactggatacaggcgccgaagtaaccagcttagctaactaccaaggccagaccacactgggactccatattgtgaaagtgcaaggcgcgaacaccgttgaagtagcgagcgctgtacgccgagaagtaagcgcgctgaacgctgagctgacaaaagataacgtgcaactgaccattacacgggataacagccgcccaatagctagccaggtaagccaagtacagcggaccctggttgagggaggagtactgtcagttctgattgtttttatattcctcaacagctggcgaagcaccgtaattaccggcttaaccctgccaatatcagtaataggcacatttgcggctatatacgctctgggctttaccctgaacattatgacattaatggctctgagcctcagcattggcatactgatagatgacgcgatagtggtacgggagaacattacacgccacctacagatgggcaaagaccccgtacgggccgcgctggacgggaccaacgaaataggcttagctgttctgagcacaacactgtgcattgtggctgttttcctgccggtagcgtttatgggcgggctgattgggcgcttctttttacagttcggcgtaaccgtagcggttgcggtttttataagcctgttcgttagcttcacattagacccgatgctgagcagcatatggcgcgacccacaatcacaaaagacagcgaaacgaggcttttttgggcaactgatagaacggtttgaccaatggtttgaagggttagctagccgctatcggagcgtaatttactttacatttgattaccggaagaccacaatagctatagtgctaggcatgttcgtggtgagcctgctgttagtaccgcgcattgggacagaattcctcccgcccccagatcagggagaagttagcataagcctggaagcgaacgagggcgctagcctggactacatggccgccaaggttgggcagattgagcgggctctgcgggaatttaattatgtgagcagcacctatagcacaattaatagcggggaaatgcgaggctttaacaaagcgctggttgcggttcaattagttcatagctcacaacgacggctgaaaaccgcggagaccctggggcccattcgccgacggttaagccggattgcgggcttagagatttcagtagggcagcggagcgaagttgtggggagcataaaaccgctacaactcagcattctgggagacggggacgaagaactgcgacggattagcgaccaaattaccagcgttttagcggcgataccgggcgctaccgaaattgagtcatcaattgagaaattacggcccaccctggctgtacgggttcgccgcgaagcggcgagcgacttaggcgtgagcattgctaccataggcgacacactgcggtcactagttgcgggagacgctatatcagtatggaacagccccgatggggagagccatgacgtggttgttcgcctgccagcggccggccgggaaaacgcggctcagctgcggaatttacccattgcgaccgctcggatggatgataatggcaaaccgataatggtgctactggaccaggtagccgacgtggttgaaagcaccgcgcccgctcagattacccgcaaagacctgagccgggatatacgcataagcagcaacatagagggacggaccctcggcgatgtagttgcggacctgaaagcggcgatgacaaaaatggacattcccgttgggttccggatatcatttggcggcgacgcggaaaacctgaccgaaagcaccgcgtatgctctgcagagcctggcgatggccgtgatttttatttacataattctcgcttcacagtttggcagctttattcagcccattgcgattattatgacaatgccgctaagcctcatgggagtgctgttaggcttactgttcaccggcagcacactgaatatgtttagcatgattggcattctgatgctgattgggctcgttacaaaaaacgcgattttactggttgattacagcaatctaggcgtacgcgagggcaagagcctgcggcagagcttagctgacgccggggcagtaagactgcggccgattgttatgaccaccctggcgatgatatttgggatgctgcccacagcgctaggcctcggcgagggcggcgcgcagcgcgcgcccatggcgcacgctattattggcgggctcataagcagcaccctgctgtcactcgtgttcgttcccgtggttctgacatatttagacgcgtttgcgcggcgcgtacggcgctgggttccctcacccacagggagcaatgctagcgctcagcatgatgggagcgacaaaacaaagacacccgcttgcgctctgacaagccaggaccagtcagggacaacaattatgtaa

SEQ ID NO: 182 - protein, 1065 aa, Sinorhizobium medicae WSM419
MFLTRISINHPVFATMMMVMILVLGLFSYGRLGVDHYPETDLPVVVVATTYTGASPESVESEISRPIEAALNTIGGIDTITSESYEGRSIVVVQFEVDVDSQDAAQEVRDRVARLETKFPDAVATPQVTRYKPEGQAILSVAVSSTSRTLPEITTLATRVINNRLSVISGVGQVSLIGSSERQVLVVVDPDRLGAYGLAVATVIEAIRGENQDRAAGTLTSGINQRIVTVEGRIANTSAFNRIIVAQRNGYPVYLSEVATILDTGAEVTSLANYQGQTTLGLHIVKVQGANTVEVASAVRREVSALNAELTKDNVQLTITRDNSRPIASQVSQVQRTLVEGGVLSVLIVFIFLNSWRSTVITGLTLPISVIGTFAAIYALGFTLNIMTLMALSLSIGILIDDAIVVRENITRHLQMGKDPVRAALDGTNEIGLAVLSTTLCIVAVFLPVAFMGGLIGRFFLQFGVTVAVAVFISLFVSFTLDPMLSSIWRDPQSQKTAKRGFFGQLIERFDQWFEGLASRYRSVIYFTFDYRKTTIAIVLGMFVVSLLLVPRIGTEFLPPPDQGEVSISLEANEGASLDYMAAKVGQIERALREFNYVSSTYSTINSGEMRGFNKALVAVQLVHSSQRRLKTAETLGPIRRRLSRIAGLEISVGQRSEVVGSIKPLQLSILGDGDEELRRISDQITSVLAAIPGATEIESSIEKLRPTLAVRVRREAASDLGVSIATIGDTLRSLVAGDAISVWNSPDGESHDVVVRLPAAGRENAAQLRNLPIATARMDDNGKPIMVLLDQVADVVESTAPAQITRKDLSRDIRISSNIEGRTLGDVVADLKAAMTKMDIPVGFRISFGGDAENLTESTAYALQSLAMAVIFIYIILASQFGSFIQPIAIIMTMPLSLMGVLLGLLFTGSTLNMFSMIGILMLIGLVTKNAILLVDYSNLGVREGKSLRQSLADAGAVRLRPIVMTTLAMIFGMLPTALGLGEGGAQRAPMAHAIIGGLISSTLLSLVFVPVVLTYLDAFARRVRRWVPSPTGSNASAQHDGSDKTKTPACALTSQDQSGTTIM

SEQ ID NO: 183 - DNA, 1206 bp, Azospirillum brasiliense LMG 04375
atgaggagaacgccaccgcgcatgagcgcggccgctagcgatcgactaccactggcgagcctgctggcgttagcgatggccgcgtttattaccattttaaccgaggcgctgccagcgggactgctgccgcagatggcgcaagggttaggagtgaccgaagcctgggttggccagaccgtgacgatttatgcggccggaagcttagcggccgcgattccgctgaccgcggcgacgcaaggagtgcgccgccgcccgctgctgctggccgcgattgcgggctttgtggttgcgaataccgtgacgaccctgagcggcagctttgcgctgaccatggttgcgcgctttctggccggcgtgagcgcgggcctgatgtgggcgctgctggccggctacgcggcgcgcatggtgccagatcatcagaaaggccgtgcgattgcggttgcgatggttggcgcgccgctagctctgagcttaggcgtaccgaccggcacctttctgggcaatctggttgattggagagtgtgctttggattaatgagcgcgctggcgctggcgttaatgttatgggtgcgcgttgtggtgccggattttgcggggcaagcggccggccgacgtctgagcccagggcgcgtgtttaccctggccggcgtgcgcccggtgctgtttgtggtactggcgtttgtgctggcgcataatattttatatacctatattgcgccgtttctggccgccgcgggcatggaagggcgcacggacctggcgctgctgctgtttggcgtggcgagcctgctgggcatttggattgtgggcgcgctgattgatggccacctgcgtgcgctgacgctggcgagcaccgtgctgtttggactgagcgcgctggtgttaggcgtggctggcgacgcgccggccgtggtgctagcggccgttgcggcgtggggcctggcgtttggcggagcggggacgctgtttcagacggcgctggcgaggacggccggcgatgcggccgacgtggcgcagagcatgctggtgaccgcgtggaatacggcgatagcgggcggcggcttattaggcggcgtgctgttagagcgtttaggcgtgggcgcgtttgcgccggccgtgctggtgctgctggccgcgacgctgttagcggtgtggggagcgaggcgtcatggcttcccagatcccgcggatacggccggagcgtaa

SEQ ID NO: 184 - protein, 401 aa, Azospirillum brasiliense LMG 04375
MRRTPPRMSAAASDRLPLASLLALAMAAFITILTEALPAGLLPQMAQGLGVTEAWVGQTVTIYAAGSLAAAIPLTAATQGVRRRPLLLAAIAGFVVANTVTTLSGSFALTMVARFLAGVSAGLMWALLAGYAARMVPDHQKGRAIAVAMVGAPLALSLGVPTGTFLGNLVDWRVCFGLMSALALALMLWVRVVVPDFAGQAAGRRLSPGRVFTLAGVRPVLFVVLAFVLAHNILYTYIAPFLAAAGMEGRTDLALLLFGVASLLGIWIVGALIDGHLRALTLASTVLFGLSALVLGVAGDAPAVVLAAVAAWGLAFGGAGTLFQTALARTAGDAADVAQSMLVTAWNTAIAGGGLLGGVLLERLGVGAFAPAVLVLLAATLLAVWGARRHGFPDPADTAGA

SEQ ID NO: 185 - DNA, 789 bp, Bradyrhizobium japonicum USDA 110
atggacgacggatacgcgagcgtgatgccggctaatgcgtataattggaccgcggtatggcgacgaaattatcttgcttggcgaaaagtggcgcttgctagccttctgggcaacctggctgacccaataaccaatctgttcggccttggcttcggactgggactgatagtgggacgggtagaaggaactagctatatagcgttccttgcggccggaatggttgcgataagcgctatgactagcgcgaccttcgagactctgtatgctgcgttcgctcggatggacgtgaaacgcacctgggaaggaatactttttactcagctgacccttggcgacattgtactaggagaacttgtgtgggccgcgtcaaaaagcgtactagcgggaactgctattggaatagtggctgcgaccctgggatacgctagctggaccagcgtactttgtgcgataccgactattgcgcttaccggccttgtgtttgcgagcctggctatggttgtgatatcacttgcgcccacttatgactattttgtgttctatcagagcctggtgcttaccccaatggtgtttctttgtggcgcggtgttcccgaccagccaaatgccagattcattccagcatttcgcgggccttcttccgctggctcatagcgtggatcttatacgccccgtgatgcttgaacgcggcgcggataatgcggcgcttcatgtaggcgcgctgtgcgtatatgcggtgcttccattctttgcgagcattgcgctattccggcgccgcctgctgcggtaa

SEQ ID NO: 186 - protein, 262 aa, Bradyrhizobium japonicum USDA 110
MDDGYASVMPANAYNWTAVWRRNYLAWRKVALASLLGNLADPITNLFGLGFGLGLIVGRVEGTSYIAFLAAGMVAISAMTSATFETLYAAFARMDVKRTWEGILFTQLTLGDIVLGELVWAASKSVLAGTAIGIVAATLGYASWTSVLCAIPTIALTGLVFASLAMVVISLAPTYDYFVFYQSLVLTPMVFLCGAVFPTSQMPDSFQHFAGLLPLAHSVDLIRPVMLERGADNAALHVGALCVYAVLPFFASIALFRRRLLR

SEQ ID NO: 187 - DNA, 921 bp, Bradyrhizobium japonicum USDA 110
atgaatatgagcaatatggcgattgatcttgttggcgtgaggaaatcatttggtgataaagtaattgtgaatgatctgagctttagcgtagcgcgcggagaatgctttggactgcttggacccaatggtgctggcaagtcaaccatagctcgcatgctgctgggcatgataagcccagaccgaggcaaaataaccgtgctggacgaacccgtgcccagccgcgctcgggccgcgcgcgtgcgcgtaggcgtggtgccgcagtttgacaaccttgaaccagaatttaccgtgcgggaaaacctgcttgtgttcggccgctactttggcatgagcgctcgcaccattgaggccgtagtgccaagccttcttgaattcgcgcggcttgagagcaaagccgatgtgcgcgtgagccttcttagcggaggaatgaaacggcgactgaccctggcgcgcgctctgattaatgacccccacctactggtgatggatgaaccgaccaccggactggacccgcacgctcgccatctgatttgggagcgcctgcgggctcttcttgcgcgcggcaaaaccattctgcttaccactcattttatggaagaagcggagcgcctttgcgaccggctatgcgtgcttgaatcaggatgcaagattgccgagggcaaacccgatgcgcttattgatgaacatattggctgcaacgtgattgaaatttacggcggagacctagaccaactgcgggaactgattcgaccgtacgcgcggcatattgaagtgtcaggagaaacccttttctgttatgcgcgatgtccggatgagattagcgtgcatctgcgcggacgaaccgatcttcgcgtactgcagcgcccaccgaatctggaagatgtgttccttcggcttaccggacgcgaaatggaaaagtaa

SEQ ID NO: 188 - protein, 306 aa, Bradyrhizobium japonicum USDA 110
MNMSNMAIDLVGVRKSFGDKVIVNDLSFSVARGECFGLLGPNGAGKSTIARMLLGMISPDRGKITVLDEPVPSRARAARVRVGVVPQFDNLEPEFTVRENLLVFGRYFGMSARTIEAVVPSLLEFARLESKADVRVSLLSGGMKRRLTLARALINDPHLLVMDEPTTGLDPHARHLIWERLRALLARGKTILLTTHFMEEAERLCDRLCVLESGCKIAEGKPDALIDEHIGCNVIEIYGGDLDQLRELIRPYARHIEVSGETLFCYARCPDEISVHLRGRTDLRVLQRPPNLEDVFLRLTGREMEK

SEQ ID NO: 189 - DNA, 378 bp, Escherichia coli
atgaaacataaacaacgttgggcgggggcaatctgctgttttgtcctcttcattgtggtgtgcctttttctggcgacgcacatgaaaggcgcttttcgggctgccgggcatcctgaaatcggcttgctatttttcattcttcctggagcagtcgccagcttcttttcacagcgtagagaagtcctgaaacctctgtttggcgcaatgctggcggcaccctgttcgatgctcattatgcggctgtttttttcaccgacgcgctcattctggcaagagctggcatggttactaagcgcggtgttctggtgtgcgctgggggcactgtgtttcttatttatcagtagtttgtttaaaccacagcacagaaaaaatcagtaa

SEQ ID NO: 190 - protein, 125 aa, Escherichia coli
MKHKQRWAGAICCFVLFIVVCLFLATHMKGAFRAAGHPEIGLLFFILPGAVASFFSQRREVLKPLFGAMLAAPCSMLIMRLFFSPTRSFWQELAWLLSAVFWCALGALCFLFISSLFKPQHRKNQ

SEQ ID NO: 191 - DNA, 369 bp, Enterobacteriaceae bacterium ENNIH1
atgaaacataaacgcggactcgctggcataatttgttgttttcttctcttcattgttgtttgttttcttgaagtgacgaatatggaaggcgcgtttcgtgcgtcggggcatcctgaactgggattgttgttttttactcttcctggcgcggtcgcgtctattttttcacgtcgccgtaaagtgatgttgccgctgtttggcgcgatcttagcggcgccgttgtgcttcgttgttatgcgtctgttttttactccggtgcgttcagtgtggcaggaactggcgtggttgttttctgcggtattctggtgtgcgctgggcgctttgtgttatctgtttatcagctctttctttggcaacgggacgcgttaa

SEQ ID NO: 192 - protein, 122 aa, Enterobacteriaceae bacterium ENNIH1
MKHKRGLAGIICCFLLFIVVCFLEVTNMEGAFRASGHPELGLLFFTLPGAVASIFSRRRKVMLPLFGAILAAPLCFVVMRLFFTPVRSVWQELAWLFSAVFWCALGALCYLFISSFFGNGTR

SEQ ID NO: 193 - DNA, 1215 bp, Escherichia coli 113303
atgtcgtatatccgggtcaataacgtcggtaaggcctatcgtcagtatcactcgaagacggggcgactgatcgaatggttatcaccactgaatacgaaacgtcataatttaaaatggatccttcgtgagattaacttcgaagtcgcaccaggtgaggcagtcggtattatcggtatcaatggtgccggtaagtcgacgctgttaaaacttataacggggacttcacgaccaactacgggagaaattgaaatctcaggtcgtgtcgcagccttacttgaattagggatgggatttcattcggatttcacgggtcggcagaacgtttatatgtcggggcaactgttagggttatcatcggagaaaataacggaactgatgcctcaaattgaagagtttgcagagattggggactatatcgatcagccagttcgtgtctactcatccgggatgcaagttcggttagcattttcggtcgccactgcaatccgtccagatgttctaattatcgatgaggccttatcggttggtgatgcctatttccagcataaaagctttgagcgtattcggaaatttcgtcaggaagggacgactctgttactggtctctcatgataaacaagccatccaaagcatttgtgaccgggccattttattaaataaaggtcaaattgaaatggaaggcgaaccagaagccgttatggattattataacgccttactggccgataaacaaaaccagtcaattaaacaagttgagcataacggtaaaactcaaacggtttcgggtacgggtgaagttacgatctccgaagttcatttacttgatgaacagggtaacgttacggaatttgtttcagtcgggcatcgtgtcagcttgcaagtcaatgttgaagtcaaggacgatattccagagttagttgtcgggtatatgattaaggatcggttagggcagccaattttcgggacgaatacttaccatcttaaccagacgcttacgtcactgaaaaaaggggaaaagcgttcattcttattttcgttcgatgcccggttaggagtcggttcatattcggtcgcagtcgccttgcatacgtcatcgactcaccttggtaaaaattatgaatggcgtgatctggccgttgtcttcaatgtcgttaatactgaacaacaagagtttgtcggtgtttcatggttaccaccagaactggaaatttcgtaa

SEQ ID NO: 194 - protein, 404 aa, Escherichia coli 113303
MSYIRVNNVGKAYRQYHSKTGRLIEWLSPLNTKRHNLKWILREINFEVAPGEAVGIIGINGAGKSTLLKLITGTSRPTTGEIEISGRVAALLELGMGFHSDFTGRQNVYMSGQLLGLSSEKITELMPQIEEFAEIGDYIDQPVRVYSSGMQVRLAFSVATAIRPDVLIIDEALSVGDAYFQHKSFERIRKFRQEGTTLLLVSHDKQAIQSICDRAILLNKGQIEMEGEPEAVMDYYNALLADKQNQSIKQVEHNGKTQTVSGTGEVTISEVHLLDEQGNVTEFVSVGHRVSLQVNVEVKDDIPELVVGYMIKDRLGQPIFGTNTYHLNQTLTSLKKGEKRSFLFSFDARLGVGSYSVAVALHTSSTHLGKNYEWRDLAVVFNVVNTEQQEFVGVSWLPPELEIS

SEQ ID NO: 195 - DNA, 795 bp, Escherichia coli 113303
atgcgggatttactaactactatttatcgttatcgaggatttatctggtcgtcggttaaacgtgattttcaggcccgttatcaaacgtcgatgctgggtgccctatggcttgttttgcaaccactttcgatgattctggtctatacgctggttttttcagaagttatgaaggcccggatgcctgataatactgggtcatttgcctattcgatttatctttgctcaggagtcctgacgtggggactctttactgagatgctggataaaggtcagagcgtctttattaataacgcaaacctgatcaagaaactttcgtttccaaaaatctgcctgccaatcatcgttactttatcagccgttctaaacttcgccattattttctcgctgtttctaatttttatcattgtcacgggtaatttccctggttggctttttctttcagttataccagtcctgttattgcaaatcctgtttgccggtgggctgggaatgatcttaggtgtcatgaatgtctttttccgagatgttgggcaactggttggtgttgccctgcaattctggttttggttcacgcctattgtttatgtcctgaactcgttaccagcctgggccaaaaacctgatgatgtataatccaatgacgcggatcatgcaatcgtatcagtcaatcttcgcctatcatctggcccctaattggtattcactatggccagtcttagcacttgccattattttctgcgtcatcggtttccgaatgttccgtaagcatgccgccgatatggttgatgaattataa

SEQ ID NO: 196 - protein, 263 aa, Escherichia coli 113303
MRDLLTTIYRYRGFIWSSVKRDFQARYQTSMLGALWLVLQPLSMILVYTLVFSEVMKARMPDNTGSFAYSIYLCSGVLTWGLFTEMLDKGQSVFINNANLIKKLSFPKICLPIIVTLSAVLNFAIIFSLFLIFIIVTGNFPGWLFLSVIPVLLLQILFAGGLGMILGVMNVFFRDVGQLVGVALQFWFWFTPIVYVLNSLPAWAKNLMMYNPMTRIMQSYQSIFAYHLAPNWYSLWPVLALAIIFCVIGFRMFRKHAADMVDE

SEQ ID NO: 197 - DNA, 1113 bp, Helicobacter pylori
atgtttcagccgctgctggatgcatatgttgaaagcgcaagcattgaaaaaatggcaagcaaaagtccgcctccgctgaaaattgcagttgcaaattggtggggtgatgaagaaatcaaagagtttaaaaacttcgtcctgtactttattctgagccagcgttataccattaccctgcatcagaatccgaatgaattttccgatctggtttttggtaatccgctgggtagcgcacgtaaaattctgagctatcagaatgcaaaacgcgtgttttataccggtgaaaatgaaagcccgaacttcaacctgtttgattatgccattggtttcgatgagctggattttaatgatcgttatctgcgtatgccgctgtattataacgagctgcatatcaaagcagaaagcgttaatgataccaccgcaccgtataaactgaaagataatagcctgtacgcactgaaaaaaccgagccattgctttaaagaaaaacatccgaatctgtgtgccgtggttaatgatgaaagcgatcctctgaaacgtggttttgcaagctttgttgcaagcaatccgaacgcaccgattcgtaatgcattctatgatgcactgaatagcattgaaccggttaccggtggtggtagcgttcgtaataccctgggttataatgtgaaaaacaaaaacgaattcctgagccagtataaattcaatctgtgctttgaaaacacccagggttatggttatgtgaccgaaaaaatcatcgatgcctatttcagccataccattccgatttattggggtagcccgagcgttgcaaaagatttcaatccgaaaagctttgtgaacgtgcacgacttcaaaaactttgatgaagccatcgattatatcaaatacctgcacacccataaaaacgcctatctggatatgctgtatgaaaatccgctgaatacactggatggtaaagcctatttttaccagaacctgagcttcaaaaaaatcctggcctttttcaaaaccatcctggaaaacgataccatctatcacgataacccgtttatcttttgccgtgatctgaatgaaccgctggttaccattgatgatctgcgtgttaattattaa

SEQ ID NO: 198 - protein, 370 aa, Helicobacter pylori
MFQPLLDAYVESASIEKMASKSPPPLKIAVANWWGDEEIKEFKNFVLYFILSQRYTITLHQNPNEFSDLVFGNPLGSARKILSYQNAKRVFYTGENESPNFNLFDYAIGFDELDFNDRYLRMPLYYNELHIKAESVNDTTAPYKLKDNSLYALKKPSHCFKEKHPNLCAVVNDESDPLKRGFASFVASNPNAPIRNAFYDALNSIEPVTGGGSVRNTLGYNVKNKNEFLSQYKFNLCFENTQGYGYVTEKIIDAYFSHTIPIYWGSPSVAKDFNPKSFVNVHDFKNFDEAIDYIKYLHTHKNAYLDMLYENPLNTLDGKAYFYQNLSFKKILAFFKTILENDTIYHDNPFIFCRDLNEPLVTIDDLRVNY

SEQ ID NO: 199 - DNA, 990 bp, Basilea psittacipulmonis JF4266 (DSM 24701)
atgtcagtgctgaaaaaactggtaagaacgctgaaaaagaaaaaagatattcccagcgaaaatcaggaagatattaagccgcaggaatttggacatatcaaacattatcatttctggcctctgtcgaacgaaacgttttttaaccaatttgcgcaggaaaaaaaccttgatctgagccagacggccctaattagctgctttggggaactatcagcgattcctaaaatccctgaacggtataaagtgttttttacgggggaaaatatctatcatccggatcgcatctcatattcggatccggagctctatcgcatggtggatctgtatttaggctttgaataccggacggaaccgaagtatctacgctttccgctgtgggtttggtatttatgcggcctgacgaaaaaaccgcatttttcacatgaatcaattgcggaatttattcggaaaatgaaccaacctgagtttcgcctgcagtcatcaaggaatcgcttttgcagccatattagcagccatgatacgaacggcattcggaaacggatgattgatttaattttaccgattgcgtcagtggattgcgccggcaagtttatgaacaacacggatgagttaaaagcgaagtttaatgatgacaagatcgactatctaaaacagtatcggtttaatctctgtcctgaaaactcagaatcagtcgggtatatcacggaaaagatttttgaatcaattatggccgggtgcattccaatttattggggaggagtcaagcagctttttgtcgaaccggatattttaaatccggaagcatttatttactacgaaaaagggaaagaagagcaattagcgaaacaggttgaagaactttggatatcacctaaacggtatgaagagtttgcagcaattgccccgtttaaagaggacgcagccgaagtgatttatacgtggattgaagaactggaaaaacggctacgggcatttgaaccaaaagcctaa

SEQ ID NO: 200 - protein, 329 aa, Basilea psittacipulmonis JF4266 (DSM 24701)
MSVLKKLVRTLKKKKDIPSENQEDIKPQEFGHIKHYHFWPLSNETFFNQFAQEKNLDLSQTALISCFGELSAIPKIPERYKVFFTGENIYHPDRISYSDPELYRMVDLYLGFEYRTEPKYLRFPLWVWYLCGLTKKPHFSHESIAEFIRKMNQPEFRLQSSRNRFCSHISSHDTNGIRKRMIDLILPIASVDCAGKFMNNTDELKAKFNDDKIDYLKQYRFNLCPENSESVGYITEKIFESIMAGCIPIYWGGVKQLFVEPDILNPEAFIYYEKGKEEQLAKQVEELWISPKRYEEFAAIAPFKEDAAEVIYTWIEELEKRLRAFEPKA

SEQ ID NO: 201 - DNA, 900 bp, Helicobacter pylori
atggcctttaaagttgttcagatttgtggtggtctgggcaatcagatgtttcagtatgcatttgcaaaaagcctgcagaaacatagcaatacaccggttctgctggatattaccagctttgattggagcaatcgtaaaatgcagctggaactgtttccgattgatctgccgtatgcaagcgaaaaagaaattgcaattgccaaaatgcagcatctgccgaaactggttcgtaatgttctgaaatgcatgggttttgatcgtgtgagccaagaaatcgtgtttgaatatgaaccgaaactgctgaaaaccagccgtctgacctatttttatggctattttcaggatccgcgttattttgatgcaattagtccgctgatcaaacagacctttaccctgcctccgcctccggaaaatggtaataacaaaaaaaaagaagaagagtatcatcgtaaactggcactgattctggcagcaaaaaatagcgtgtttgtgcatattcgtcgcggtgattatgttggtattggttgtcagctgggcatcgattatcagaaaaaagcactggaatacatggcaaaacgtgttccgaatatggaactgtttgtgttttgcgaggacctggaatttacccagaatctggatctgggctatccgtttatggatatgaccacccgtgataaagaggaagaggcatattgggatatgctgctgatgcagagctgtaaacatggtattattgccaacagcacctatagttggtgggcagcatatctgattaataacccggaaaaaatcattattggtccgaaacattggctgtttggccatgaaaacatcctgtgtaaagaatgggtgaaaatcgaaagccactttgaagtgaaaagccagaaatataatgcctaa

SEQ ID NO: 202 - protein, 299 aa, Helicobacter pylori
MAFKVVQICGGLGNQMFQYAFAKSLQKHSNTPVLLDITSFDWSNRKMQLELFPIDLPYASEKEIAIAKMQHLPKLVRNVLKCMGFDRVSQEIVFEYEPKLLKTSRLTYFYGYFQDPRYFDAISPLIKQTFTLPPPPENGNNKKKEEEYHRKLALILAAKNSVFVHIRRGDYVGIGCQLGIDYQKKALEYMAKRVPNMELFVFCEDLEFTQNLDLGYPFMDMTTRDKEEEAYWDMLLMQSCKHGIIANSTYSWWAAYLINNPEKIIIGPKHWLFGHENILCKEWVKIESHFEVKSQKYNA

SEQ ID NO: 203 - DNA, 1341 bp, Escherichia coli K12 MG1655
atgatgattactctgcgcaaacttcctctggcggttgccgtcgcagcgggcgtaatgtctgctcaggcaatggctgttgatttccacggctatgcacgttccggtattggttggacaggtagcggcggtgaacaacagtgtttccagactaccggtgctcaaagtaaataccgtcttggcaacgaatgtgaaacttatgctgaattaaaattgggtcaggaagtgtggaaagagggcgataagagcttctatttcgacactaacgtggcctattccgtcgcacaacagaatgactgggaagctaccgatccggccttccgtgaagcaaacgtgcagggtaaaaacctgatcgaatggctgccaggctccaccatctgggcaggtaagcgcttctaccaacgtcatgacgttcatatgatcgacttctactactgggatatttctggtcctggtgccggtctggaaaacatcgatgttggcttcggtaaactctctctggcagcaacccgctcctctgaagctggtggttcttcctctttcgccagcaacaatatttatgactataccaacgaaaccgcgaacgacgttttcgatgtgcgtttagcgcagatggaaatcaacccgggcggcacattagaactgggtgtcgactacggtcgtgccaacttgcgtgataactatcgtctggttgatggcgcatcgaaagacggctggttattcactgctgaacatactcagagtgtcctgaagggctttaacaagtttgttgttcagtacgctactgactcgatgacctcgcagggtaaagggctgtcgcagggttctggcgttgcatttgataacgaaaaatttgcctacaatatcaacaacaacggtcacatgctgcgtatcctcgaccacggtgcgatctccatgggcgacaactgggacatgatgtacgtgggtatgtaccaggatatcaactgggataacgacaacggcaccaagtggtggaccgtcggtattcgcccgatgtacaagtggacgccaatcatgagcaccgtgatggaaatcggctacgacaacgtcgaatcccagcgcaccggcgacaagaacaatcagtacaaaattaccctcgcacaacaatggcaggctggcgacagcatctggtcacgcccggctattcgtgtattcgcaacctacgccaagtgggatgagaaatggggttacgactacaccggtaacgctgataacaacgcgaacttcggcaaagccgttcctgctgatttcaacggcggcagcttcggtcgtggcgacagcgacgagtggaccttcggtgcccagatggaaatctggtggtaa

SEQ ID NO: 204 - protein, 446 aa, Escherichia coli K12 MG1655 [sequence omitted]

SEQ ID NO: 205 - DNA, 1191 bp, Escherichia coli K12 MG1655
atgaaaataaaaacaggtgcacgcatcctcgcattatccgcattaacgacgatgatgttttccgcctcggctctcgccaaaatcgaagaaggtaaactggtaatctggattaacggcgataaaggctataacggtcttgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactcgtatcaccaagtaa

SEQ ID NO: 206 - protein, 396 aa, Escherichia coli K12 MG1655
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 207 - DNA, 1116 bp, Escherichia coli K12 MG1655
atggcgagcgtacagctgcaaaatgtaacgaaagcctggggcgaggtcgtggtatcgaaagatatcaatctcgatatccatgaaggtgaattcgtggtgtttgtcggaccgtctggctgcggtaaatcgactttactgcgcatgattgccgggcttgagactatcaccagcggcgacctgttcatcggtgagaaacggatgaatgacactccgccagcagaacgcggcgttggtatggtgtttcagtcttacgcgctctatccccacctgtcagtagcagaaaacatgtcatttggcctgaaactggctggcgcaaaaaaagaggtgattaaccaacgcgttaaccaggtggcggaagtgctacaactggcgcatttgctggatcgcaaaccgaaagcgctctccggtggtcagcgtcagcgtgtggcgattggccgtacgctggtggccgagccaagcgtatttttgctcgatgaaccgctctccaacctcgatgctgcactgcgtgtgcaaatgcgtatcgaaatctcccgtctgcataaacgcctgggccgcacaatgatttacgtcacccacgatcaggtcgaagcgatgacgctggccgacaaaatcgtggtgctggacgccggtcgcgtggcgcaggttgggaaaccgctggagctgtaccactatccggcagaccgttttgtcgccggatttatcggttcgccaaagatgaacttcctgccggtaaaagtgaccgccaccgcaatcgatcaagtgcaggtggagctgccgatgccaaatcgtcagcaagtctggctgccagttgaaagccgtgatgtccaggttggagccaatatgtcgctgggtattcgcccggaacatctactgccgagtgatatcgctgacgtcatccttgagggtgaagttcaggtcgtcgagcaactcggcaacgaaactcaaatccatatccagatcccttccattcgtcaaaacctggtgtaccgccagaacgacgtggtgttggtagaagaaggtgccacattcgctatcggcctgccgccagagcgttgccatctgttccgtgaggatggcactgcatgtcgtcgactgcataaggagccgggcgtttaa

SEQ ID NO: 208 - protein, 371 aa, Escherichia coli K12 MG1655
MASVQLQNVTKAWGEVVVSKDINLDIHEGEFVVFVGPSGCGKSTLLRMIAGLETITSGDLFIGEKRMNDTPPAERGVGMVFQSYALYPHLSVAENMSFGLKLAGAKKEVINQRVNQVAEVLQLAHLLDRKPKALSGGQRQRVAIGRTLVAEPSVFLLDEPLSNLDAALRVQMRIEISRLHKRLGRTMIYVTHDQVEAMTLADKIVVLDAGRVAQVGKPLELYHYPADRFVAGFIGSPKMNFLPVKVTATAIDQVQVELPMPNRQQVWLPVESRDVQVGANMSLGIRPEHLLPSDIADVILEGEVQVVEQLGNETQIHIQIPSIRQNLVYRQNDVVLVEEGATFAIGLPPERCHLFREDGTACRRLHKEPGV

SEQ ID NO: 209 - DNA, 1947 bp, Escherichia coli K12 MG1655
atgaatattttaggttttttccagcgactcggtagggcgttacagctccctatcgcggtgctgccggtggcggcactgttgctgcgattcggtcagccagatttacttaacgttgcgtttattgcccaggcgggcggtgcgatttttgataacctcgcattaatcttcgccatcggtgtggcatccagctggtcgaaagacagcgctggtgcggcggcgctggcgggtgcggtaggttactttgtgttaaccaaagcgatggtgaccatcaacccagaaattaacatgggtgtactggcgggtatcattaccggtctggttggtggcgcagcctataaccgttggtccgatattaaactgccggacttcctgagcttcttcggcggcaaacgctttgtgccgattgccaccggattcttctgcctggtgctggcggccatttttggttacgtctggccgccggtacagcacgctatccatgcaggcggcgagtggatcgtttctgcgggcgcgctgggttccggtatctttggtttcatcaaccgtctgctgatcccaaccggtctgcatcaggtactgaacaccatcgcctggttccagattggtgaattcaccaacgcggcgggtacggttttccacggtgacattaaccgcttctatgccggtgacggcaccgcggggatgttcatgtccggcttcttcccgatcatgatgttcggtctgccgggtgcggcgctggcgatgtacttcgcagcaccgaaagagcgtcgtccgatggttggcggtatgctgctttctgttgctgttactgcgttcctgaccggtgtgactgagccgctggaattcctgttcatgttccttgctccgctgctgtacctcctgcacgcactgctgaccggtatcagcctgtttgtggcaacgctgctgggtatccacgcgggcttctctttctctgcgggggctatcgactacgcgttgatgtataacctgccggccgccagccagaacgtctggatgctgctggtgatgggcgttatcttcttcgctatctacttcgtggtgttcagtttggttatccgcatgttcaacctgaaaacgccgggtcgtgaagataaagaagatgagatcgttactgaagaagccaacagcaacactgaagaaggtctgactcaactggcaaccaactatattgctgcggttggcggcactgacaacctgaaagcgattgacgcctgtatcacccgtctgcgccttacagtggctgactctgcccgcgttaacgatacgatgtgtaaacgtctgggtgcttctggggtagtgaaactgaacaaacagactattcaggtgattgttggcgcgaaagcagaatccatcggcgatgcgatgaagaaagtcgttgcccgtggtccggtagccgctgcgtcagctgaagcaactccggcaactgccgcgcctgtagcaaaaccgcaggctgtaccaaacgcggtatctatcgcggagctggtatcgccgattaccggtgatgtcgtggcactggatcaggttcctgacgaagcattcgccagcaaagcggtgggtgacggtgtggcggtgaaaccgacagataaaatcgtcgtatcaccagccgcagggacaatcgtgaaaatcttcaacaccaaccacgcgttctgcctggaaaccgaaaaaggcgcggagatcgtcgtccatatgggtatcgacaccgtagcgctggaaggtaaaggctttaaacgtctggtggaagagggtgcgcaggtaagcgcagggcaaccgattctggaaatggatctggattacctgaacgctaacgcccgctcgatgattagcccggtggtttgcagcaatatcgacgatttcagtggcttgatcattaaagctcagggccatattgtggcgggtcaaacaccgctgtatgaaatcaaaaagtaa

SEQ ID NO: 210 - protein, 648 aa, Escherichia coli K12 MG1655
MNILGFFQRLGRALQLPIAVLPVAALLLRFGQPDLLNVAFIAQAGGAIFDNLALIFAIGVASSWSKDSAGAAALAGAVGYFVLTKAMVTINPEINMGVLAGIITGLVGGAAYNRWSDIKLPDFLSFFGGKRFVPIATGFFCLVLAAIFGYVWPPVQHAIHAGGEWIVSAGALGSGIFGFINRLLIPTGLHQVLNTIAWFQIGEFTNAAGTVFHGDINRFYAGDGTAGMFMSGFFPIMMFGLPGAALAMYFAAPKERRPMVGGMLLSVAVTAFLTGVTEPLEFLFMFLAPLLYLLHALLTGISLFVATLLGIHAGFSFSAGAIDYALMYNLPAASQNVWMLLVMGVIFFAIYFVVFSLVIRMFNLKTPGREDKEDEIVTEEANSNTEEGLTQLATNYIAAVGGTDNLKAIDACITRLRLTVADSARVNDTMCKRLGASGVVKLNKQTIQVIVGAKAESIGDAMKKVVARGPVAAASAEATPATAAPVAKPQAVPNAVSIAELVSPITGDVVALDQVPDEAFASKAVGDGVAVKPTDKIVVSPAAGTIVKIFNTNHAFCLETEKGAEIVVHMGIDTVALEGKGFKRLVEEGAQVSAGQPILEMDLDYLNANARSMISPVVCSNIDDFSGLIIKAQGHIVAGQTPLYEIKK

SEQ ID NO: 211 - DNA, 372 bp, Escherichia coli K12 MG1655
atgaccgttatttatcagaccaccatcacccgtatcggcgcgagtgccattgacgccctcagtgaccagatgctcatcacctttcgtgaaggcgcgcctgcggacctcgaagagtattgcttcattcattgccacggcgagttgaaaggtgcactccatcccggtttgcaattttcactcgggcagcatcgctatccggtgaccgctgttggcagcgtggcggaagataaccttcgcgaactgggtcatgtcaccctgcgcttcgatggtttaaacgaagcggaatttccgggcactgtccatgtggcaggccctgtccccgacgatatcgcgccgggatcggttttgaagtttgaatctgttaaggagtaa

SEQ ID NO: 212 - protein, 123 aa, Escherichia coli K12 MG1655
MTVIYQTTITRIGASAIDALSDQMLITFREGAPADLEEYCFIHCHGELKGALHPGLQFSLGQHRYPVTAVGSVAEDNLRELGHVTLRFDGLNEAEFPGTVHVAGPVPDDIAPGSVLKFESVKE

SEQ ID NO: 213 - DNA, 990 bp, Escherichia coli K12 MG1655
atgcacaaatttactaaagccctggcagccattggtctggcagccgttatgtcacaatccgctatggcggagaacctgaagctcggttttctggtgaagcaaccggaagagccgtggttccagaccgaatggaagtttgccgataaagccgggaaggatttagggtttgaggttattaagattgccgtgccggatggcgaaaaaacattgaacgcgatcgacagcctggctgccagtggcgcaaaaggtttcgttatttgtactccggaccccaaactcggctctgccatcgtcgcgaaagcgcgtggctacgatatgaaagtcattgccgtggatgaccagtttgttaacgccaaaggtaagccaatggataccgttccgctggtgatgatggcggcgactaaaattggcgaacgtcagggccaggaactgtataaagagatgcagaaacgtggctgggatgtcaaagaaagcgcggtgatggcgattaccgccaacgaactggataccgcccgccgccgtactacgggatctatggatgcgctgaaagcggccggattcccggaaaaacaaatttatcaggtacctaccaaatctaacgacatcccgggggcatttgacgctgccaactcaatgctggttcaacatccggaagttaaacattggctgatcgtcggtatgaacgacagcaccgtgctgggcggcgtacgcgcgacggaaggtcagggctttaaagcggccgatatcatcggcattggcattaacggtgtggatgcggtgagcgaactgtctaaagcacaggcaaccggcttctacggttccctgctgccaagcccggacgtacatggctataaatccagcgaaatgctttacaactgggtagcaaaagacgttgaaccgccaaaatttaccgaagttaccgacgtggtactgatcacgcgtgacaactttaaagaagaactggagaaaaaaggtttaggcggtaagtaa

SEQ ID NO: 214 - protein, 329 aa, Escherichia coli K12 MG1655
MHKFTKALAAIGLAAVMSQSAMAENLKLGFLVKQPEEPWFQTEWKFADKAGKDLGFEVIKIAVPDGEKTLNAIDSLAASGAKGFVICTPDPKLGSAIVAKARGYDMKVIAVDDQFVNAKGKPMDTVPLVMMAATKIGERQGQELYKEMQKRGWDVKESAVMAITANELDTARRRTTGSMDALKAAGFPEKQIYQVPTKSNDIPGAFDAANSMLVQHPEVKHWLIVGMNDSTVLGGVRATEGQGFKAADIIGIGINGVDAVSELSKAQATGFYGSLLPSPDVHGYKSSEMLYNWVAKDVEPPKFTEVTDVVLITRDNFKEELEKKGLGGK

SEQ ID NO: 215 - DNA, 993 bp, Escherichia coli K12 MG1655
atgaaaataaagaacattctactcaccctttgcacctcactcctgcttaccaacgttgctgcacacgccaaagaagtcaaaataggtatggcgattgatgatctccgtcttgaacgctggcaaaaagatcgagatatctttgtgaaaaaggcagaatctctcggcgcgaaagtatttgtacagtctgcaaatggcaatgaagaaacacaaatgtcgcagattgaaaacatgataaaccggggtgtcgatgttcttgtcattattccgtataacggtcaggtattaagtaacgttgtaaaagaagccaaacaagaaggcattaaagtattagcttacgaccgtatgattaacgatgcggatatcgatttttatatttctttcgataacgaaaaagtcggtgaactgcaggcaaaagccctggtcgatattgttccgcaaggtaattacttcctgatgggcggctcgccggtagataacaacgccaagctgttccgcgccggacaaatgaaagtgttaaaaccttacgttgattccggaaaaattaaagtcgttggtgaccaatgggttgatggctggttaccggaaaacgcattgaaaattatggaaaacgcgctaaccgccaataataacaaaattgatgctgtagttgcctcaaacgatgccaccgcaggtggggcaattcaggcattaagcgcgcaaggtttatcagggaaagtagcaatctccggccaggatgcggatctcgcaggtattaaacgtattgctgccggtacgcaaactatgacggtgtataaacctattacgttgttggcaaatactgccgcagaaattgccgttgagttgggcaatggtcaggaaccaaaagcagataccacactgaataatggcctgaaagatgtcccctcccgcctcctgacaccgatcgatgtgaataaaaacaacatcaaagatacggtaattaaagacggattccacaaagagagcgagctgtaa

SEQ ID NO: 216 - protein, 330 aa, Escherichia coli K12 MG1655
MKIKNILLTLCTSLLLTNVAAHAKEVKIGMAIDDLRLERWQKDRDIFVKKAESLGAKVFVQSANGNEETQMSQIENMINRGVDVLVIIPYNGQVLSNVVKEAKQEGIKVLAYDRMINDADIDFYISFDNEKVGELQAKALVDIVPQGNYFLMGGSPVDNNAKLFRAGQMKVLKPYVDSGKIKVVGDQWVDGWLPENALKIMENALTANNNKIDAVVASNDATAGGAIQALSAQGLSGKVAISGQDADLAGIKRIAAGTQTMTVYKPITLLANTAAEIAVELGNGQEPKADTTLNNGLKDVPSRLLTPIDVNKNNIKDTVIKDGFHKESEL

SEQ ID NO: 217 - DNA, 957 bp, Escherichia coli K12 MG1655
atgtggaaacgcttacttatagtctctgcagtctcggcagccatgtcgtctatggcgttggccgctccattaaccgttggattttcgcaggtcggatcggaatcaggctggcgtgccgcagaaaccaatgtggcgaaaagtgaagccgaaaagcgcggaatcacgttgaaaattgccgatggtcagcaaaagcaggaaaaccagattaaagcggtacgttccttcgttgcacaaggggtggatgcgatctttatcgctccggtggtcgcgacaggttgggaaccggtattaaaagaggcgaaagatgccgaaatcccggtattcttgctcgatcgttccattgatgtgaaagacaaatctctctatatgaccaccgtcactgccgacaacatcctcgaaggcaagttgattggtgactggctggtaaaagaagtgaatggcaaaccatgcaacgtggtggagctgcagggcaccgttggggccagcgtcgccattgaccgtaagaaaggctttgccgaagccattaagaatgcgccaaatatcaaaatcatccgctcgcagtcaggtgacttcacccgcagtaaaggcaaagaagtcatggagagctttatcaaagcggaaaacaacggcaaaaacatctgcatggtttacgcccataacgacgacatggtgattggtgcaattcaggcaattaaagaagcgggcctgaaaccgggcaaagatatcctcacgggttccattgacggtgtaccggacatctacaaagcgatgatggatggcgaagcgaacgccagtgttgaactgacgccgaatatggcaggtcccgccttcgacgcgctggagaaatacaaaaaagacggcaccatgcctgaaaagctgacgttaaccaaatccaccctttacctgcctgataccgcaaaagaagaattagagaagaagaaaaatatggggtattga

SEQ ID NO: 218 - protein, 318 aa, Escherichia coli K12 MG1655
MWKRLLIVSAVSAAMSSMALAAPLTVGFSQVGSESGWRAAETNVAKSEAEKRGITLKIADGQQKQENQIKAVRSFVAQGVDAIFIAPVVATGWEPVLKEAKDAEIPVFLLDRSIDVKDKSLYMTTVTADNILEGKLIGDWLVKEVNGKPCNVVELQGTVGASVAIDRKKGFAEAIKNAPNIKIIRSQSGDFTRSKGKEVMESFIKAENNGKNICMVYAHNDDMVIGAIQAIKEAGLKPGKDILTGSIDGVPDIYKAMMDGEANASVELTPNMAGPAFDALEKYKKDGTMPEKLTLTKSTLYLPDTAKEELEKKKNMGY

SEQ ID NO: 219 - DNA, 936 bp, Helicobacter sp. MIT 01-6242
atgttttcagttcgtttaatggggggcttaggaaaccagatgtttatttatgcttttgcaaaggctataaaggcgcaggggtatccggtgcgtctattttattatgatacggattacaacgtgccgcagacgcacaacattcgtaacttagagatagtggattttggcattgcgatgtgtatagaaactatgtgttatgaagaaccacagatcaaaaaatctttctttgagcgtgcgttaggctttataaaacgtaagttaaaaatacattcaccacattcaagctctttaattagcgatcattgtgagattgctttaactaaagattttcttgatacgttaaacccgaacgctatgtttaacggttatttccagaacgttgtatttttcgatcatcttcgtgaatcattattaagagattttactcttaaacgtccgcttactccggcaaacgaggcgttaaaacatcagatcttacagacgccaaacagctgctttctacatattcggcggggggattatttacaaattccgatatatgttaaacttggtagcacttactacaataacgctatcaaggctcttaaagataagatctcaaagccacatatctttgtttttagcaacgacattgcgtggtgcaaggaatttttcttagatagcttagatcccttagtgattgaaaacgtgacttttagctttatagaaaataacgatgaagggaacgccattgaggaaatggaacttatgcggtcatgccagcacgcaattattgcgaacagcacgtttagctggtgggcggcgtatttaattgacagcgctcagaaactttgcattatgcctaaacactttttcaatgatccccagcaggaagtagcacacaaactaattccaccaccacttcatagcttatctcagactatagtaattggttaa

SEQ ID NO: 220 - protein, 311 aa, Helicobacter sp. MIT 01-6242
MFSVRLMGGLGNQMFIYAFAKAIKAQGYPVRLFYYDTDYNVPQTHNIRNLEIVDFGIAMCIETMCYEEPQIKKSFFERALGFIKRKLKIHSPHSSSLISDHCEIALTKDFLDTLNPNAMFNGYFQNVVFFDHLRESLLRDFTLKRPLTPANEALKHQILQTPNSCFLHIRRGDYLQIPIYVKLGSTYYNNAIKALKDKISKPHIFVFSNDIAWCKEFFLDSLDPLVIENVTFSFIENNDEGNAIEEMELMRSCQHAIIANSTFSWWAAYLIDSAQKLCIMPKHFFNDPQQEVAHKLIPPPLHSLSQTIVIG

(19) United States
(12) Patent Application Publication
(10) Pub. No.: US 2022/0259631 A1
(43) Pub. Date: Aug. 18, 2022

(54) PRODUCTION OF FUCOSYLLACTOSE IN HOST CELLS

(71) Applicant: INBIOSE N.V., Zwijnaarde (BE)

(72) Inventors: Joeri Beauprez, Bredene (BE); Nausicaä Lannoo, Eke-Nazareth (BE); Kristof Vandewalle, Tielt (BE); Annelies Vercauteren, Eke (BE)

(21) Appl. No.: 17/627,088

(22) PCT Filed: Jul. 16, 2020
(86) PCT No.: PCT/EP2020/070209

§ 371 Date: Jan. 13, 2022

(30) Foreign Application Priority Data
Jul. 19, 2019 (EP) 19187404.9

(51) Int. Cl. C12P 19/18; C12N 9/10; C07K 14/245

(52) U.S. Cl. CPC C12P 19/18; C07K 14/245; C12Y 204/01065; C12N 9/1051

(57) ABSTRACT

This disclosure is in the technical field of synthetic biology and metabolic engineering. More particularly, this disclosure is in the technical field of fermentation of metabolically engineered host cells. The disclosure describes a method of producing fucosyllactose by fermentation with a genetically modified cell, as well as to the genetically modified cell used in the method. The genetically modified cell comprises at least one nucleic acid sequence coding for an enzyme involved in fucosyllactose synthesis; more specifically, the cell comprises a nucleic acid sequence coding for a fucosyltransferase, thereby synthesizing fucosyllactose and at least one nucleic acid expressing a membrane protein, more specifically, a nucleic acid sequence expressing a membrane protein enabling fucosyllactose transport.

### CROSS-REFERENCE TO RELATED APPLICATIONS

This application is a national phase entry under 35 U.S.C. § 371 of International Patent Application PCT/EP2020/070209, filed Jul. 16, 2020, designating the United States of America and published as International Patent Publication WO 2021/013708 A1 on Jan. 28, 2021, which claims the benefit under Article 8 of the Patent Cooperation Treaty to European Patent Application Serial No. 19187404.9, filed Jul. 19, 2019.

### TECHNICAL FIELD

This application is in the technical field of synthetic biology and metabolic engineering. More particularly, this disclosure is in the technical field of fermentation of metabolically engineered host cells. The disclosure describes a method of producing fucosyllactose by fermentation with a genetically modified cell, as well as to the genetically modified cell used in the method. The genetically modified cell comprises at least one nucleic acid sequence coding for an enzyme involved in fucosyllactose synthesis, more specifically, the cell comprises a nucleic acid sequence coding for a fucosyltransferase, thereby synthesizing fucosyllactose and at least one nucleic acid expressing a membrane protein, more specifically, a nucleic acid sequence expressing a membrane protein enabling fucosyllactose transport.

### STATEMENT ACCORDING TO 37 C.F.R. § 1.821(c) or (e)—SEQUENCE LISTING SUBMITTED AS A TXT AND PDF FILES

Pursuant to 37 C.F.R. § 1.821(c) or (e), files containing a TXT version and a PDF version of the Sequence Listing have been submitted concomitant with this application, the contents of which are hereby incorporated by reference.

### BACKGROUND

Today, more than 80 compounds belonging to the family of Human Milk Oligosaccharides (HMOs), have been structurally characterized. These HMOs represent a class of complex oligosaccharides that function as prebiotics. Additionally, the structural homology of HMO to epithelial epitopes accounts for protective properties against bacterial pathogens. Within the infant gastrointestinal tract, HMOs selectively nourish the growth of selected bacterial strains and are, thus, priming the development of a unique gut microbiota in breast milk-fed infants.

Some of these Human Milk oligosaccharides require the presence of particular fucosylated structures that most likely exhibit a particular biological activity. Production of these fucosylated oligosaccharides requires the action of a fucosyltransferase. Such fucosyltransferases, which belong to the enzyme family of glycosyltransferases, are widely expressed in vertebrates, invertebrates, plants, fungi, yeasts, and bacteria. They catalyze the transfer of a fucose residue from a donor, generally guanosine-diphosphate fucose (GDP-fucose) to an acceptor, which include oligosaccharides, (glyco)proteins and (glyco)lipids. The thus fucosylated acceptor substrates are involved in a variety of biological and pathological processes.

In microbial fermentative production of fucosyllactose (FL), the FL is in many cases produced intracellularly in the industrial production host. One problem identified in the art as the true difficulty in producing oligosaccharides in cells is the intracellular enrichment of the produced oligosaccharides and their extraction. The intracellular enrichment is deemed to be responsible for the product-inhibitory effect on the production of the desired oligosaccharide. Synthesis may become slow or the desired oligosaccharide may reach cytotoxic concentrations resulting in metabolic arrest or even cell lysis.

### BRIEF SUMMARY

Surprisingly, it has now been found that the membrane proteins used in the disclosure provide for newly identified membrane proteins, more specifically, the disclosure provides for newly identified membrane proteins previously unknown to enable fucosyllactose transport and having a positive effect on fermentative production of fucosyllactose, providing a better yield, productivity, specific productivity and/or growth speed when used to genetically engineer a host cell producing fucosyllactose.

The disclosure also provides methods for producing fucosyllactose. The fucosyllactose is obtained with a host cell comprising the membrane protein of the disclosure.

Provided are tools and methods by means of which fucosyllactose can be produced in an efficient, time and cost-effective way and which yields high amounts of the desired product. Further provided is a method and a cell for the production of fucosyllactose wherein the cell is genetically modified for the production of fucosyllactose and comprises at least one nucleic acid sequence encoding an enzyme involved in fucosyllactose synthesis, more specifically the cell comprises a nucleic acid sequence coding for a fucosyltransferase thereby synthesizing fucosyllactose. The cell furthermore also expresses a membrane protein, more specifically the cell furthermore also expresses a membrane protein previously unknown to enable fucosyllactose transport, according to the disclosure.

### Definitions

The words used in this specification to describe the disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of embodiments of the disclosure described herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the drawings and specification, embodiments of the disclosure have been described, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps.

According to the disclosure, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the disclosure. Moreover, DNAs or RNAs comprising unusual bases, such as inosine, or modified bases, such as tritylated bases, are to be understood to be covered by the term “polynucleotides.” It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” as the term is employed herein. Similarly, a “synthetic” sequence, as the term is used herein, means any sequence that has been generated synthetically and not directly isolated from a natural source. “Synthesized,” as the term is used herein, means any synthetically generated sequence and not directly isolated from a natural source.

“Recombinant” means genetically engineered DNA prepared by transplanting or splicing genes from one species into the cells of a host organism of a different species. Such DNA becomes part of the host's genetic makeup and is replicated.

The term “endogenous,” within the context of the disclosure refers to any polynucleotide, polypeptide or protein sequence that is a natural part of a cell and is occurring at its natural location in the cell chromosome.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “polynucleotide encoding a polypeptide” as used herein encompasses polynucleotides that include a sequence encoding a polypeptide of the disclosure. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

The term “modified expression” of a gene relates to a change in expression compared to the wild-type expression of the gene in any phase of the production process of the fucosyllactose. The modified expression is either a lower or higher expression compared to the wild type, wherein the term “higher expression” is also defined as “overexpression” of the gene in the case of an endogenous gene or “expression” in the case of a heterologous gene that is not present in the wild-type strain. Lower expression is obtained by means of common well-known technologies for a skilled person (such as the usage of siRNA, CrispR, CrispRi, recombineering, homologous recombination, ssDNA mutagenesis, RNAi, miRNA, asRNA, mutating genes, knocking-out genes, transposon mutagenesis, etc.), which are used to change the genes in such a way that they are less-able (i.e., statistically significantly “less-able” compared to a functional wild-type gene) or completely unable (such as knocked-out genes) to produce functional final products. Overexpression or expression is obtained by means of common well-known technologies for a skilled person, wherein the gene is part of an “expression cassette” that relates to any sequence in which a promoter sequence, untranslated region sequence (containing either a ribosome binding sequence or Kozak sequence), a coding sequence (for instance, a membrane protein gene sequence) and optionally a transcription terminator is present, and leading to the expression of a functional active protein. The expression is either constitutive or conditional or regulated.

The term “constitutive expression” is defined as expression that is not regulated by transcription factors other than the subunits of RNA polymerase (e.g., the bacterial sigma factors) under certain growth conditions. Non-limiting examples of such transcription factors are CRP, LacI, ArcA, Cra, IclR in E. coli, or, Aft2p, Crz1p, Skn7 in Saccharomyces cerevisiae, or, DeoR, GntR, Fur in B. subtilis. These transcription factors bind on a specific sequence and may block or enhance expression in certain growth conditions. RNA polymerase binds a specific sequence to initiate transcription, for instance, via a sigma factor in prokaryotic hosts.

The term “regulated expression” is defined as expression that is regulated by transcription factors other than the subunits of RNA polymerase (e.g., bacterial sigma factors) under certain growth conditions. Examples of such transcription factors are described above. Commonly expression regulation is obtained by means of an inducer, such as but not limited to IPTG, arabinose, rhamnose, fucose, allo-lactose or pH shifts, or temperature shifts or carbon depletion or substrates or the produced product.

The term “wild type” refers to the commonly known genetic or phenotypical situation as it occurs in nature.

“Variant(s)” as the term is used herein, is a polynucleotide or polypeptide that differs from a reference polynucleotide or polypeptide respectively but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

In some embodiments, the disclosure contemplates making functional variants by modifying the structure of a membrane protein as used in the disclosure. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, and in the case of the disclosure, to provide better yield, productivity, and/or growth speed than a cell without the variant.

The term “functional homolog” as used herein describes those molecules that have sequence similarity and also share at least one functional characteristic such as a biochemical activity. More specifically, the term “functional homolog” as used herein describes those proteins that have sequence similarity (in other words, homology) and at the same time have at least one functional similarity such as a biochemical activity (Altenhoff et al., PLoS Comput. Biol. 8 (2012) e1002514).

Functional homologs are sometimes referred to as orthologs, where “ortholog” refers to a homologous gene or protein that is the functional equivalent of the referenced gene or protein in another species. Functional homologs will typically give rise to the same characteristics to a similar, but not necessarily the same, degree. Functionally homologous proteins give the same characteristics where the quantitative measurement produced by one homolog is at least 10 percent of the other; more typically, at least 20 percent, between about 30 percent and about 40 percent; for example, between about 50 percent and about 60 percent; between about 70 percent and about 80 percent; or between about 90 percent and about 95 percent; between about 98 percent and about 100 percent, or greater than 100 percent of that produced by the original molecule. Thus, where the molecule has enzymatic activity the functional homolog will have the above-recited percent enzymatic activities compared to the original enzyme. Where the molecule is a DNA-binding molecule (e.g., a polypeptide) the homolog will have the above-recited percentage of binding affinity as measured by weight of bound molecule compared to the original molecule.

A functional homolog and the reference polypeptide may be naturally occurring polypeptides, and the sequence similarity may be due to convergent or divergent evolutionary events.

Functional homologs can be identified by analysis of nucleotide and polypeptide sequence alignments. For example, performing a query on a database of nucleotide or polypeptide sequences can identify homologs of biomass-modulating polypeptides. Sequence analysis can involve BLAST, Reciprocal BLAST, or PSI-BLAST analysis of non-redundant databases using amino acid sequence of a biomass-modulating polypeptide as the reference sequence. Amino acid sequence is, in some instances, deduced from the nucleotide sequence. Typically, those polypeptides in the database that have greater than 40 percent sequence identity are candidates for further evaluation for suitability as a biomass-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in productivity-modulating polypeptides, e.g., conserved functional domains.

“Fragment,” with respect to a polynucleotide, refers to a clone or any part of a polynucleotide molecule, particularly a part of a polynucleotide that retains a usable, functional characteristic. Useful fragments include oligonucleotides and polynucleotides that may be used in hybridization or amplification technologies or in the regulation of replication, transcription, or translation. A “polynucleotide fragment” refers to any subsequence of a polynucleotide, typically, of at least about 9 consecutive nucleotides, for example, at least about 30 nucleotides or at least about 50 nucleotides of any of the sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide.

Fragments may additionally or alternatively include subsequences of polypeptides and protein molecules, or a subsequence of the polypeptide. In some cases, the fragment or domain is a subsequence of the polypeptide that performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, for example, at least about 20 amino acid residues in length, for example, at least about 30 amino acid residues in length. Preferentially a fragment is a functional fragment that has at least one property or activity of the polypeptide from which it is derived, such as, for example, the fragment can include a functional domain or conserved domain of a polypeptide. A domain can be characterized, for example, by a Pfam (https://pfam.xfam.org/) (El-Gebali et al., Nucleic Acids Res. 47 (2019) D427-D432) or Conserved Domain Database (CDD) (https://www.ncbi.nlm.nih.gov/cdd) (Lu et al., Nucleic Acids Res. 48 (2020) D265-D268) designation. The Pfam database as used herein refers to the Pfam database Pfam 32.0 as released in September 2018 and the CDD database as used herein refers to the CDD database v3.17 as released on 3 Apr. 2019.

The terms “fucosyllactose,” “fucosyl lactose” and “FL” as used herein are used interchangeably and refer to an oligosaccharide comprising a fucose residue and a lactose residue. Such fucosyllactose refers to 2′-fucosyllactose, 3-fucosyllactose, or difucosyllactose or any combination thereof, fucosyllactoses refers to a combination of at least any two of 2′-fucosyllactose, 3-fucosyllactose, or difucosyllactose.

The terms “alpha-1,2′-fucosyltransferase,” “alpha 1,2′ fucosyltransferase,” “2′-fucosyltransferase,” “α-1,2′-fucosyltransferase,” “α 1,2′ fucosyltransferase,” “2′fucosyltransferase,” “2′-FT” or “2′FT,” as used in the disclosure, are used interchangeably and refer to a glycosyltransferase that catalyzes the transfer of fucose from the donor substrate GDP-L-fucose, to the acceptor molecule lactose in an alpha-1,2-linkage. A polynucleotide encoding an “alpha-1,2-fucosyltransferase” or any of the above terms, refers to a polynucleotide encoding such glycosyltransferase that catalyzes the transfer of fucose from the donor substrate GDP-L-fucose, to the acceptor molecule lactose in an alpha-1,2-linkage.

The terms “2′-fucosyllactose,” “alpha-1,2′-fucosyllactose,” “alpha 1,2′ fucosyllactose,” “α-1,2′-fucosyllactose,” “α 1,2′ fucosyllactose,” “Fucα1-2Galβ1-4Glc,” “2′FL” or “2′-FL,” as used in the disclosure, are used interchangeably. In a preferred embodiment, these terms refer to the product obtained by the catalysis of the alpha-1,2′-fucosyltransferase transferring the fucose residue from GDP-L-fucose to lactose in an alpha-1,2′-linkage.

The terms “alpha-1,3-fucosyltransferase,” “alpha 1,3 fucosyltransferase,” “3-fucosyltransferase,” “α-1,3-fucosyltransferase,” “α 1,3 fucosyltransferase,” “3 fucosyltransferase,” “3-FT” or “3FT,” as used in the disclosure, are used interchangeably and refer to a glycosyltransferase that catalyzes the transfer of fucose from the donor substrate GDP-L-fucose, to the acceptor molecule lactose in an alpha-1,3-linkage. A polynucleotide encoding an “alpha-1,3-fucosyltransferase” or any of the above terms, refers to a polynucleotide encoding such glycosyltransferase that catalyzes the transfer of fucose from the donor substrate GDP-L-fucose, to the acceptor molecule lactose in an alpha-1,3-linkage.

The terms “3-fucosyllactose,” “alpha-1,3-fucosyllactose,” “alpha 1,3 fucosyllactose,” “α-1,3-fucosyllactose,” “α 1,3 fucosyllactose,”. “Galβ1-4(Fucα1-3)Glc,” “3FL” or “3-FL,” as used in the disclosure, are used interchangeably. In a preferred embodiment, these terms refer to the product obtained by the catalysis of the alpha-1,3-fucosyltransferase transferring the fucose residue from GDP-L-fucose to lactose in an alpha-1,3-linkage.

The terms “difucosyllactose,” “di-fucosyllactose,” “lactodifucotetraose,” “2′,3-difucosyllactose,” “2′,3-difucosyllactose,” “α-2′,3-fucosyllactose,” “α 2′,3 fucosyllactose,” “Fucα1-2Galβ 1-4(Fucα1-3)Glc,” “DFLac,” “2′,3 diFL,” “DFL” or “diFL,” as used in the disclosure, are used interchangeably. In a preferred embodiment, these terms refer to the product obtained by the catalysis of the alpha-1,3-fucosyltransferase transferring the fucose residue to a 2′FL resulting in a 2′,3-difucosyllactose or refer to the product obtained by the catalysis of the alpha-1,2-fucosyltransferase transferring the fucose residue to a 3FL resulting in 2′, 3 difucosyllactose.

“Oligosaccharide” as the term is used herein and as generally understood in the state of the art, refers to a saccharide polymer containing a small number, typically three to ten, of simple sugars, i.e., monosaccharides.

“SET” or “Sugar Efflux Transporter” as used herein refers to membrane proteins of the SET family that are proteins with InterPRO domain IPR001214 as defined by InterPro 75.0 (release date 4 Jul. 2019) and/or are proteins that belong to the eggNOGv4.5 family ENOG410XTE9 as defined by the Eggnogdb 1.0.2 database (release date 3 Nov. 2017). Identification of the InterPro domain can be done by using the online tool on www.ebi.ac.uk/interpro/ or a standalone version of InterProScan (www.ebi.ac.uk/interpro/download.html) using the default values. Identification of the orthology family in eggNOGv4.5 can be done using the online version or a standalone version of eggNOG-mapperv1 (eggnogdb.embl.de/#/app/home).

The term “membrane proteins” as used herein refers to proteins that are part of or interact with the cells membrane and control the flow of molecules and information across the cell. The membrane proteins are thus involved in transport, be it import into or export out of the cell.

Such membrane proteins can be porters, P-P-bond-hydrolysis-driven transporters, β-Barrel Porins, auxiliary transport proteins, putative transport proteins or phosphotransfer-driven group translocators as defined by the Transporter Classification Database that is operated and curated by the Saier Lab Bioinformatics Group available via www.tcdb.org and providing a functional and phylogenetic classification of membrane transport proteins This Transporter Classification Database details a comprehensive IUBMB approved classification system for membrane transport proteins known as the Transporter Classification (TC) system. The TCDB classification searches as described here are defined based on TCDB.org as released on 17 Jun. 2019.

Porters is the collective name of uniporters, symporters, and antiporters that utilize a carrier-mediated process (Saier et al., Nucleic Acids Res. 44 (2016) D372-D379). They belong to the electrochemical potential-driven transporters and are also known as secondary carrier-type facilitators. Membrane proteins are included in this class when they utilize a carrier-mediated process to catalyze uniport when a single species is transported either by facilitated diffusion or in a membrane potential-dependent process if the solute is charged; antiport when two or more species are transported in opposite directions in a tightly coupled process, not coupled to a direct form of energy other than chemiosmotic energy; and/or symport when two or more species are transported together in the same direction in a tightly coupled process, not coupled to a direct form of energy other than chemiosmotic energy, of secondary carriers (Forrest et al., Biochim. Biophys. Acta 1807 (2011) 167-188). These systems are usually stereospecific. Solute:solute counter-transport is a characteristic feature of secondary carriers. The dynamic association of porters and enzymes creates functional membrane transport metabolons that channel substrates typically obtained from the extracellular compartment directly into their cellular metabolism (Moraes and Reithmeier, Biochim. Biophys. Acta 1818 (2012), 2687-2706). Solutes that are transported via this porter system include but are not limited to cations, organic anions, inorganic anions, nucleosides, amino acids, polyols, phosphorylated glycolytic intermediates, osmolites, siderophores.

Membrane proteins are included in the class of P-P-bond hydrolysis-driven transporters if they hydrolyze the diphosphate bond of inorganic pyrophosphate, ATP, or another nucleoside triphosphate, to drive the active uptake and/or extrusion of a solute or solutes (Saier et al., Nucleic Acids Res. 44 (2016) D372-D379). The membrane protein may or may not be transiently phosphorylated, but the substrate is not phosphorylated. Substrates that are transported via the class of P-P-bond hydrolysis-driven transporters include but are not limited to cations, heavy metals, beta-glucan, UDP-glucose, lipopolysaccharides, teichoic acid.

The β-Barrel porins membrane proteins form transmembrane pores that usually allow the energy independent passage of solutes across a membrane. The transmembrane portions of these proteins consist exclusively of β-strands that form a β-barrel (Saier et al., Nucleic Acids Res. 44 (2016) D372-D379). These porin-type proteins are found in the outer membranes of Gram-negative bacteria, mitochondria, plastids, and possibly acid-fast Gram-positive bacteria. Solutes that are transported via these β-Barrel porins membrane proteins include but are not limited to nucleosides, raffinose, glucose, beta-glucosides, and oligosaccharides.

The auxiliary transport proteins are defined to be proteins that facilitate transport across one or more biological membranes but do not themselves participate directly in transport. These membrane proteins always function in conjunction with one or more established transport systems such as but not limited to outer membrane factors (OMFs), polysaccharide (PST) porters, the ATP-binding cassette (ABC)-type transporters. They may provide a function connected with energy coupling to transport, play a structural role in complex formation, serve a biogenic or stability function or function in regulation (Saier et al., Nucleic Acids Res. 44 (2016) D372-D379). Examples of auxiliary transport proteins include but are not limited to the polysaccharide copolymerase family involved in polysaccharide transport, the membrane fusion protein family involved in bacteriocin and chemical toxin transport.

Putative transport protein comprises families that will either be classified elsewhere when the transport function of a member becomes established or will be eliminated from the Transporter Classification system if the proposed transport function is disproven. These families include a member or members for which a transport function has been suggested, but evidence for such a function is not yet compelling (Saier et al., Nucleic Acids Res. 44 (2016) D372-D379). Examples of putative transporters classified in this group under the TCDB system as released on 17 Jun. 2019 include but are not limited to copper transporters.

The phosphotransfer-driven group translocators are also known as the PEP-dependent phosphoryl transfer-driven group translocators of the bacterial phosphoenolpyruvate:sugar phosphotransferase system (PTS). The product of the reaction, derived from extracellular sugar, is a cytoplasmic sugar-phosphate. The enzymatic constituents, catalyzing sugar phosphorylation, are superimposed on the transport process in a tightly coupled process. The PTS system is involved in many different aspects comprising in regulation and chemotaxis, biofilm formation, and pathogenesis (Lengeler, J. Mol. Microbiol. Biotechnol. 25 (2015) 79-93; Saier, J. Mol. Microbiol. Biotechnol. 25 (2015) 73-78). Membrane protein families classified within the phosphotransfer-driven group translocators under the TCDB system as released on 17 Jun. 2019 include PTS systems linked to transport of glucose-glucosides, fructose-mannitol, lactose-N,N′-Diacetylchitobiose-beta-glucoside, glucitol, galactitol, mannose-fructose-sorbose and ascorbate.

It should be understood for those skilled in the art that for the databases used herein, comprising Pfam 32.0 (released September 2018), CDD v3.17 (released 3 Apr. 2019), eggnoddb 1.0.2 (released 3 Nov. 2017), InterPro 75.0 (released 4 Jul. 2019) and TCDB (released 17 Jun. 2019), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

The term “enabling transport” means to introduce the activity of transport of a solute over the cytoplasm membrane and/or the cell wall. The transport may be enabled by introducing and/or increasing the expression of a transporter protein as described in the disclosure. The term “enhancing transport” means to improve the activity of transport of a solute over the cytoplasm membrane and/or the cell wall. The transport may be enhanced by introducing and/or increasing the expression of a transporter protein as described in the disclosure. “Expression” of a transporter protein is defined as “overexpression” of the gene encoding the transporter protein in the case the gene is an endogenous gene or “expression” in the case the gene encoding the transporter protein is a heterologous gene that is not present in the wild-type strain.

### Hybridization

The term “hybridization” as defined herein is a process wherein substantially homologous complementary nucleotide sequences anneal to each other. The hybridization process can occur entirely in solution, i.e., both complementary nucleic acids are in solution. The hybridization process can also occur with one of the complementary nucleic acids immobilized to a matrix such as magnetic beads, Sepharose beads or any other resin. The hybridization process can furthermore occur with one of the complementary nucleic acids immobilized to a solid support such as a nitro-cellulose or nylon membrane or immobilized by e.g., photolithography to, for example, a siliceous glass support (the latter known as nucleic acid arrays or microarrays or as nucleic acid chips). In order to allow hybridization to occur, the nucleic acid molecules are generally thermally or chemically denatured to melt a double strand into two single strands and/or to remove hairpins or other secondary structures from single stranded nucleic acids. The term “stringency” refers to the conditions under which a hybridization takes place. The stringency of hybridization is influenced by conditions such as temperature, salt concentration, ionic strength and hybridization buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridization conditions are typically used for isolating hybridizing sequences that have high sequence similarity to the target nucleic acid sequence. However, nucleic acids may deviate in sequence and still encode a substantially identical polypeptide, due to the degeneracy of the genetic code. Therefore, medium stringency hybridization conditions may sometimes be needed to identify such nucleic acid molecules.

The Tm is the temperature under defined ionic strength and pH, at which 50% of the target sequence hybridizes to a perfectly matched probe. The Tm is dependent upon the solution conditions and the base composition and length of the probe. For example, longer sequences hybridize specifically at higher temperatures. The maximum rate of hybridization is obtained from about 16° C. up to 32° C. below Tm. The presence of monovalent cations in the hybridization solution reduce the electrostatic repulsion between the two nucleic acid strands thereby promoting hybrid formation; this effect is visible for sodium concentrations of up to 0.4 M (for higher concentrations, this effect may be ignored). Formamide reduces the melting temperature of DNA-DNA and DNA-RNA duplexes with 0.6 to 0.7° C. for each % formamide, and addition of 50% formamide allows hybridization to be performed at 30 to 45° C., though the rate of hybridization will be lowered. Base pair mismatches reduce the hybridization rate and the thermal stability of the duplexes. On average and for large probes, the Tm decreases about 1° C. per % base mismatch. The Tm may be calculated using the following equations, depending on the types of hybrids:

1) DNA-DNA hybrids (Meinkoth and Wahl, Anal. Biochem., 138: 267-284, 1984):

Tm=81.5° C.+16.6×(log 10[Na+]a)+0.41×(% [G+Cb]−500×[Lc]−1−0.61×(% formamide)

2) DNA-RNA or RNA-RNA hybrids:

Tm=79.8° C.+18.5×(log 10[Na+]a)+0.58×(% [G+Cb])+11.8×(% [G+Cb])2−820×[Lc]−1

3) oligo-DNA or oligo-RNAd hybrids:

For <20 nucleotides: Tm=2/n

For 20-35 nucleotides: Tm=22+1.46/n

Wherein:
a: or for other monovalent cation, but only accurate in the 0.01-0.4 M range.
b: only accurate for % GC in the 30% to 75% range.
c: L=length of duplex in base pairs,
d: oligo, oligonucleotide,
n: effective length of primer=2×(no. of G+C)+(no. of A+T).

Non-specific binding may be controlled using any one of a number of known techniques such as, for example, blocking the membrane with protein containing solutions, additions of heterologous RNA, DNA, and SDS to the hybridization buffer, and treatment with Rnase. For non-homologous probes, a series of hybridizations may be performed by varying one of (i) progressively lowering the annealing temperature (for example, from 68° C. to 42° C.) or (ii) progressively lowering the formamide concentration (for example, from 50% to 0%). The skilled artisan is aware of various parameters that may be altered during hybridization and that will either maintain or change the stringency conditions.

Besides the hybridization conditions, specificity of hybridization typically also depends on the function of post-hybridization washes. To remove background resulting from nonspecific hybridization, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridization stringency. A positive hybridization gives a signal that is at least twice of that of the background. Generally, suitable stringent conditions for nucleic acid hybridization assays or gene amplification detection procedures are as set forth above. More or less stringent conditions may also be selected. The skilled artisan is aware of various parameters that may be altered during washing and that will either maintain or change the stringency conditions.

For example, typical high stringency hybridization conditions for DNA hybrids longer than 50 nucleotides encompass hybridization at 65° C. in 1×SSC or at 42° C. in 1×SSC and 50% formamide, followed by washing at 65° C. in 0.3×SSC. Examples of medium stringency hybridization conditions for DNA hybrids longer than 50 nucleotides encompass hybridization at 50° C. in 4×SSC or at 40° C. in 6×SSC and 50% formamide, followed by washing at 50° C. in 2×SSC. The length of the hybrid is the anticipated length for the hybridization nucleic acid. When nucleic acids of known sequence are hybridized, the hybrid length may be determined by aligning the sequences and identifying the conserved regions described herein. 1×SSC is 0.15 M NaCl and 15 mM sodium citrate; the hybridized solution and wash solutions may additionally include 5×Denhardt's reagent, 0.5-1.0% SDS, 100 micro g/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 15° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the disclosure are at least about 50%, 55%, 60%, 65%, 70%, 75%, 80% or 85% pure, usually at least about 90°/a, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, e.g., 3-fucosyllactose, purity can be determined using methods such as but not limited to thin layer chromatography, gas chromatography, NMR, HPLC, capillary electrophoresis or mass spectroscopy.

The terms “identical” or percent “identity” or % “identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity can be determined using BLAST and PSI-BLAST (Altschul et al., 1990, J. Mol. Biol. 215:3, 403-410; Altschul et al., 1997, Nucleic Acids Res. 25: 17, 3389-402). For the purposes of this disclosure, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM50.

The term “control sequences” refers to sequences recognized by the host cells transcriptional and translational systems, allowing transcription and translation of a polynucleotide sequence to a polypeptide. Such DNA sequences are thus necessary for the expression of an operably linked coding sequence in a particular host cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. The control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of the polynucleotide to a polypeptide.

Generally, “operably linked” means that the DNA sequences being linked are contiguous, and, in the case of a secretory leader, contiguous and in reading phase. However, enhancers do not have to be contiguous.

As used herein, the term “cell productivity index (CPI)” refers to the mass of the product produced by the recombinant cells divided by the mass of the recombinant cells produced in the culture.

The following drawings and examples will serve as further illustration and clarification of the disclosure and are not intended to be limiting.

### BRIEF DESCRIPTION OF THE DRAWINGS

FIG. 1: CPI in relative percentages (%) obtained in a growth experiment with strains expressing membrane proteins with SEQ ID NO: 58 to SEQ ID NO: 96 (excluding SEQ ID NO: 90) in TU2, with SEQ ID NO: 90 in TU3 or with SEQ ID NO: 02 to SEQ ID NO: 44 in TU10 and expressing an α1,3-fucosyltransferase. Strains with membrane proteins SEQ ID NO: 04 to SEQ ID NO: 34 produce 3-FL from FT1, whereas strains with membrane proteins with SEQ ID NO: 02 and with SEQ ID NO: 40 to SEQ ID NO: 96 produce 3-FL from FT2. The CPI data refer to 3-FL measurements in whole broth samples. The growth experiment was performed in minimal medium supplemented with 20 g/L lactose as precursor for 3-FL. The dashed horizontal line indicates the setpoint to which all adaptations were normalized.

FIG. 2: 3-FL export ratio in relative percentages (%) obtained in a growth experiment with strains expressing membrane proteins with SEQ ID NO: 58 to SEQ ID NO: 104 (excluding SEQ ID NO: 90) in TU2, SEQ ID NO: 90 in TU3 or SEQ ID NO: 02 to SEQ ID NO: 34 in TU10 and expressing an α1,3-fucosyltransferase. Strains with membrane proteins with SEQ ID NO: 08 to SEQ ID NO: 30 produce 3-FL from FT1, whereas strains with membrane proteins with SEQ ID NO: 58 to SEQ ID NO: 104 produce 3-FL from FT2. The strain with the membrane protein with SEQ ID NO: 02 was tested in combination with either FT1 or FT2. The growth experiment was performed in minimal medium supplemented with 20 g/L lactose as precursor for 3-FL. The dashed horizontal line indicates the setpoint to which all adaptations were normalized.

FIG. 3: Growth speed in relative percentages (%) obtained in a growth experiment with strains expressing the membrane proteins with SEQ ID NO: 08, 14, 18 or 22 in TU10 and expressing the α1,3-fucosyl transferase FT1. The growth experiment was performed in minimal medium supplemented with 20 g/L lactose as precursor for 3-FL. The dashed horizontal line indicates the setpoint to which all adaptations were normalized.

FIG. 4: 3-FL export ratio in relative percentages (%) obtained in a growth experiment with a strain expressing the membrane protein with SEQ ID NO: 28 in TU10 and expressing the α1,3-fucosyl transferase FT1. The growth experiment was performed in minimal medium supplemented with 45 g/L lactose as precursor for 3-FL. The dashed horizontal line indicates the setpoint to which all adaptations were normalized.

FIG. 5: CPI in relative percentages (%) obtained in a growth experiment with the strain expressing the membrane protein with SEQ ID NO: 28 in TU10 and expressing the α1,3-fucosyl transferase FT1. The CPI data refer to 3-FL measurements in whole broth samples. The growth experiment was performed in minimal medium supplemented with 90 g/L lactose as precursor for 3-FL. The dashed horizontal line indicates the setpoint to which all adaptations were normalized.

FIG. 6: 3FL export ratio in relative percentages (%) obtained in a growth experiment with strains expressing membrane proteins with SEQ ID NO: 10 or 16 in TU10 and expressing the α1,3-fucosyl transferase FT1. The growth experiment was performed in minimal medium supplemented with 90 g/L lactose as precursor for 3-FL. The dashed horizontal line indicates the setpoint to which all adaptations were normalized.

FIG. 7: CPI in relative percentages (%) obtained in a growth experiment with strains expressing membrane proteins with SEQ ID NO: 10, 16 or 28 in TU10 and expressing the α1,3-fucosyl transferase FT1. The growth experiment was performed in minimal medium supplemented with 100 g/L sucrose and 90 g/L lactose as precursor for 3-FL. The dashed horizontal line indicates the setpoint to which all adaptations were normalized.

FIG. 8: Growth speed in relative percentages (%) obtained in a growth experiment with a strain expressing the membrane protein with SEQ ID NO: 28 in TU10 and the α1,3-fucosyl transferase FT1. The growth experiment was performed in minimal medium supplemented with 100 g/L sucrose and 90 g/L lactose as precursor for 3-FL. The dashed horizontal line indicates the setpoint to which all adaptations were normalized.

FIG. 9: CPI in relative percentages (%) obtained in a growth experiment with the strain expressing the membrane protein with SEQ ID NO: 22 in TU10 and the α1,3-fucosyl transferase FT1. The growth experiment was performed in minimal medium supplemented with 5 g/L lactose as precursor for 3-FL. The dashed horizontal line indicates the setpoint to which all adaptations were normalized.

FIG. 10: CPI in relative percentages (%) obtained in a growth experiment with strains expressing membrane proteins with SEQ ID NO: 02 or 28 in TU10 from the host's genome and expressing an α1,3-fucosyl transferase being either FT1 or FT2 from plasmid. Hereby, the gene with SEQ ID NO: 01 was integrated in the EcLdhA locus, the gene with SEQ ID NO: 27 was integrated in the EcSetA locus. The growth experiment was performed in minimal medium supplemented with 20 g/L lactose as precursor for 3-FL. The dashed horizontal line indicates the setpoint to which all adaptations were normalized.

FIG. 11: 3-FL export ratio in relative percentages (%) obtained in a growth experiment with strains expressing membrane proteins with SEQ ID NO: 02 or 28 in TU10 from the host's genome and expressing the α1,3-fucosyl transferase FT2 from plasmid. Hereby, the gene with SEQ ID NO: 01 was integrated in the EcLdhA locus, the gene with SEQ ID NO: 27 was integrated in the EcSetA locus. The growth experiment was performed in minimal medium supplemented with 20 g/L lactose as precursor for 3-FL. The dashed horizontal line indicates the setpoint to which all adaptations were normalized.

FIG. 12: CPI in relative percentages (%) obtained in a growth experiment with strains expressing membrane proteins with SEQ ID NO: 02, 06, 10, 16, 22, 28, 32, 34, 36, 38, 40, 42, 44 or 50 in different transcriptional units (TU) from plasmid and expressing the α1,3-fucosyl transferase FT2 from plasmid. The growth experiment was performed in minimal medium supplemented with 20 g/L lactose as precursor for 3-FL. The dashed horizontal line indicates the setpoint to which all adaptations were normalized.

FIG. 13: 3-FL export ratio in relative percentages (%) obtained in a growth experiment with strains expressing membrane proteins with SEQ ID NO: 02, 06, 10, 16, 22, 28, 32, 34, 36, 38, 40, 42, 44 or 50 in different transcriptional units (TU) from plasmid, and expressing the α13-fucosyl transferase FT2 from plasmid. The growth experiment was performed in minimal medium supplemented with 20 g/L lactose as precursor for 3-FL. The dashed horizontal line indicates the setpoint to which all adaptations were normalized.

FIG. 14: CPI (left panel) and 3-FL export ratio (right panel) in relative percentages (%) obtained in a growth experiment with strains expressing membrane proteins with SEQ ID NO: 40, 42, 46 or 48, either cloned as single genes in TU10 or cloned in their native transcriptional operon structure containing two membrane protein genes and presented on plasmid, and expressing the α1,3-fucosyl transferase FT2 from plasmid. The growth experiment was performed in minimal medium supplemented with 20 g/L lactose as precursor for 3-FL. The dashed horizontal line indicates the setpoint to which all adaptations were normalized.

FIG. 15: CPI for the hosts with 2′FL (panel A) or DiFL (panel B) production and DiFL export ratio (panel C) in relative percentages (%) obtained in a growth experiment with strains expressing membrane proteins with SEQ ID NO: 02, 06 or 28 in TU10, being integrated in the host's genome in the EcSetA locus (for membrane protein with SEQ ID NO: 28) or in the EcLdhA locus (for membrane protein with SEQ ID NOS: 02 and 06) and expressing the α1,2-fucosyl transferase FT3 from plasmid. The growth experiment was performed in minimal medium supplemented with 20 g/L lactose as precursor for 2′-FL and DiFL. The dashed horizontal line indicates the setpoint to which all adaptations were normalized.

FIG. 16: Productivity parameters enhanced in the batch and fed-batch phase measured in eight independent fermentation runs that were performed with a 3-FL E. coli production host over-expressing the membrane protein with SEQ ID NO: 02 from genome and the α1,3-fucosyl transferase FT2 from plasmid. The dashed horizontal line indicates the setpoint to which all adaptations were normalized. A reference fermentation was performed with an identical strain lacking the over-expression cassette of the membrane protein gene. Fermentations were performed as described in Example 3. CPI, cell performance index (g 3-FL/g biomass); br, whole broth; sn, supernatant; Qp, specific productivity (g 3-FL/g biomass/h); Qs, specific productivity (g sucrose/g biomass/h); Ys, yield on sucrose (g 3-FL/g sucrose); Yx, biomass yield (g biomass/g sucrose); rate, production rate (g 3-FL/L/h); lac_rate, lactose conversion rate (g lactose consumed/h).

FIG. 17: Productivity parameters enhanced in the batch and fed-batch phase measured in a fermentation run that was performed with a 3-FL E. coli production host over-expressing the membrane protein with SEQ ID NO: 06 from a first plasmid and the α1,3-fucosyl transferase FT2 from a second plasmid. The dashed horizontal line indicates the setpoint to which all adaptations were normalized. A specific reference fermentation was performed with an identical strain lacking the membrane protein gene. Fermentations were performed as described in Example 3. CPI, cell performance index (g 3-FL/g biomass); br, whole broth; sn, supernatant; Ys, yield on sucrose (g 3-FL/g sucrose); Yx, biomass yield (g biomass/g sucrose.

FIG. 18: CPI in relative percentages (%) obtained in a growth experiment with strains expressing membrane proteins with SEQ ID NO: 02, 06, 120, 126, 128, 140, 146 or 150 and expressing α1,3-fucosyl transferase FT1 (for membrane proteins with SEQ ID NO: 02 and SEQ ID NO: 06) or FT2 (for the other membrane proteins) from plasmid. Membrane proteins with SEQ ID NO: 02 and SEQ ID NO: 06 were cloned in TU10. The membrane protein with SEQ ID NO: 126 was cloned in TU2. Membrane proteins with SEQ ID NOS: 120, 140 and 150 were cloned in TU3. The membrane proteins with SEQ ID NO: 128 and SEQ ID NO: 146 were cloned either in TU2 (version v1) or TU3 (version v2). The CPI data refer to 3-FL measurements in whole broth samples. The growth experiment was performed in minimal medium supplemented with 20 g/L lactose as precursor for 3-FL. The dashed horizontal line indicates the setpoint to which all adaptations were normalized.

FIG. 19: 3-FL export ratio in relative percentages (%) obtained in a growth experiment with strains expressing membrane proteins with SEQ ID NO: 126 and SEQ ID NO: 128 (version v1) cloned in TU2, SEQ ID NO: 128 (version 2) cloned in TU3 and SEQ ID NO: 02 in TU10 and expressing α1,3-fucosyl transferase FT1 (for strain with membrane protein with SEQ ID NO: 02) or FT2 (for strains with membrane proteins with SEQ ID NOS: 126 and 128) from plasmid. The growth experiment was performed in minimal medium supplemented with 20 g/L lactose as precursor for 3-FL. The dashed horizontal line indicates the setpoint to which all adaptations were normalized.

FIG. 20: Growth speed in relative percentages (%) obtained in a growth experiment with strains expressing membrane proteins with SEQ ID NOS: 120 and 140 in TU3 and expressing the α1,3-fucosyl transferase FT2 from plasmid. The growth experiment was performed in minimal medium supplemented with 20 g/L lactose as precursor for 3-FL. The dashed horizontal line indicates the setpoint to which all adaptations were normalized.

FIG. 21: MATGAT table of Example 20 relating to EcMdfA.

FIG. 22: MATGAT table of Example 20 relating to EcIceT.

FIG. 23: CPI in relative percentages (%) obtained in a growth experiment with strains expressing membrane proteins with SEQ ID NO: 54 cloned in TU1, with SEQ ID NOS: 62, 66, 70, 76, 84, 92, 96, and 104 cloned in TU2, with SEQ ID NOS: 58, 64, 72, 74, and 94 cloned in TU3, with SEQ ID NOS: 184, 204, and 208 cloned in TU11, with SEQ ID NOS: 52, 56, 60, 80, 82, 88, 90, and 98 cloned in TU12 and expressing the α1,3-fucosyltransferase FT2. The CPI data refer to 3-FL measurements in whole broth samples. The growth experiment was performed in minimal medium supplemented with 20 g/L lactose as precursor for 3-FL. The dashed horizontal line indicates the setpoint to which all adaptations were normalized.

FIG. 24: CPI in relative percentages (%) obtained in a growth experiment with strains expressing membrane proteins with SEQ ID NO: 204 or 214 cloned in TU11 and expressing the α1,2-fucosyltransferase FT4. The CPI data refer to 2′-FL measurements in whole broth samples. The growth experiment was performed in minimal medium supplemented with 20 g/L lactose as precursor for 2′-FL. The dashed horizontal line indicates the setpoint to which all adaptations were normalized.

FIG. 25: 2′-FL export ratio in relative percentages (%) obtained in a growth experiment with strains expressing membrane proteins with SEQ ID NOS: 206, 208, 214, 216, and 218 cloned in TU11 and expressing the α1,2-fucosyltransferase FT4. The growth experiment was performed in minimal medium supplemented with 20 g/L lactose as precursor for 2′-FL. The dashed horizontal line indicates the setpoint to which all adaptations were normalized.

### DETAILED DESCRIPTION

In a first embodiment, the disclosure provides a method for the production of fucosyllactose by a genetically modified cell. The method comprises the steps of:
providing a cell capable of producing fucosyllactose, the cell comprising at least one nucleic acid sequence coding for an enzyme involved in fucosyllactose synthesis, more specifically, the cell comprising at least one nucleic acid sequence coding for a fucosyltransferase that transfers a fucose residue from a guanosine-diphosphate fucose (GDP-fucose) donor to a lactose acceptor thereby synthesizing fucosyllactose
the cell further comprising i) a modified expression of an endogenous membrane protein, more specifically an endogenous membrane protein involved in fucosyllactose transport, even more specifically an endogenous membrane protein enabling and/or enhancing fucosyllactose transport, and/or ii) an expression of an heterologous membrane protein, more specifically an heterologous membrane protein involved in fucosyllactose transport, even more specifically an heterologous membrane protein enabling and/or enhancing fucosyllactose transport, and wherein the membrane protein is i) selected from the group of membrane proteins comprising any one of the PFAM domains found by searching the genomic neighborhood of GT10 and GT11 fucosyltransferase families with InterPro number IPR001503 and IPR002516 as defined on InterPro 75.0 as released 4 Jul. 2019 respectively, wherein the genomic neighborhood window size is 14 genes before and 14 genes after the respective fucosyltransferase and wherein the membrane protein is not belonging to the SET family, or ii) selected from the group of membrane proteins comprising SEQ ID NO: 204, 206, 208, 210, 212, 214, 216, or 218, or functional homolog or functional fragment of any one of the membrane proteins comprising SEQ ID NO: 204, 206, 208, 210, 212, 214, 216, or 218 or a sequence having at least 80% sequence identity to any one of the membrane proteins with SEQ ID NO: 204, 206, 208, 210, 212, 214, 216, or 218, and cultivating the cell in a medium under conditions permissive for the production of the desired fucosyllactose. Preferably, the fucosyllactose is separated from the cultivation as explained herein.

In a preferred embodiment of the disclosure, the host cell comprises a membrane protein selected from the group of:
a) porters;
b) P-P-bond-hydrolysis-driven transporters;
c) β-Barrel Porins;
d) Auxiliary transport proteins;
e) Putative transport proteins; and
f) Phosphotransfer-driven group translocators.

Another embodiment provides a method for the production of fucosyllactose by a genetically modified cell, comprising the steps of:
providing a cell capable of producing fucosyllactose, the cell comprising at least one nucleic acid sequence coding for an enzyme involved in fucosyllactose synthesis, more specifically a fucosyltransferase that transfers a fucose residue from a GDP-fucose donor to a lactose acceptor thereby synthesizing fucosyllactose,
the cell further comprising i) a modified expression of an endogenous membrane protein involved in fucosyllactose transport, more specifically enabling and/or enhancing fucosyllactose transport and/or ii) an expression of a heterologous membrane protein involved in fucosyllactose transport, more specifically enabling and/or enhancing fucosyllactose, and wherein the membrane protein is selected from the group of
   a) porters excluding SET;
   b) P-P-bond-hydrolysis-driven transporters;
   c) β-Barrel Porins;
   d) Auxiliary transport proteins;
   e) Putative transport proteins; and
   f) Phosphotransfer-driven group translocators.
cultivating the cell in a medium under conditions permissive for the production of the desired fucosyllactose. Preferably, the produced fucosyllactose is separated from the cultivation as explained herein.

In the method of the disclosure described herein, the membrane protein is either an endogenous protein with a modified expression, preferably, the endogenous protein is overexpressed; or the membrane protein is a heterologous protein, which can be heterologously expressed by the cell. The heterologously expressed membrane protein will then be introduced and expressed, preferably overexpressed. In another embodiment, the endogenous protein can have a modified expression in the cell that also expresses a heterologous membrane protein.

The host cell used herein is preferably genetically modified for the production of fucosyllactose. In a further preferred embodiment, the cell used herein comprises a recombinant fucosyltransferase capable of modifying lactose or an intermediate into fucosyllactose.

The host cell used herein is optionally genetically modified for the production of fucosyllactose, wherein the host cell is modified to express the de novo synthesis of GDP-fucose. The de novo synthesis of GDP-fucose is catalyzed by the enzymes mannose-6-phosphate isomerase, phosphomannomutase encoding gene, mannose-1-phosphate guanosyltransferase, GDP-mannose-4,6-dehydratase and GDP-L-fucose synthase. Preferably, the host cell is further modified to express one or more genes encoding for the enzymes of the de novo synthesis of GDP-fucose.

The host cell used herein is optionally genetically modified to import lactose in the cell, by the introduction and/or overexpression of a lactose permease. The lactose permease is, for example, encoded by the lacY gene or the lac12 gene.

According to a further aspect of the disclosure, the polynucleotide encoding the membrane protein is adapted to the codon usage of the respective cell or expression system.

In a preferred aspect of the above embodiments, the porter is selected from the group of TCDB classes 2.A.1.1, 2.A.1.12, 2.A.1.15, 2.A.1.2, 2.A.1.3, 2.A.1.36, 2.A.1.38, 2.A.1.46, 2.A.1.68, 2.A.1.7, 2.A.1.81, 2.A.123, 2.A.2, 2.A.21, 2.A.58, 2.A.6.3, 2.A.66 and 2.A.7.1; the P-P-bond-hydrolysis-driven transporter is selected from the group of TCDB classes 3.A.1.1, 3.A.1.2, 3.A.1.10, 3.A.1.11 and 3.A.1.5; the β-Barrel Porin is selected from TCDB classes 1.B.18 and 1.B.3.1; the Auxiliary transport protein is selected from TCDB class 8.A.3; the Putative transport protein is selected from the group of TCDB classes 9.B.14 and 9.B.158; or the Phosphotransfer-driven group translocator is selected from the group of TCDB classes 4.A.1.1 and 4.A.4.1. The TCDB classes are classified as defined on TCDB.org as released on 17 Jun. 2019.

In another preferred aspect of the above embodiments, the porter is selected from the group of eggnog families 05BZS, 05C0R, 05C2C, 05CT4, 05CXP, 05CZQ, 05D94, 05DXI, 05E5M, 05E5W, 05E8G, 05EAM, 05EDR, 05EGZ, 05F9N, 05JHE, 05PSV, 05W2Y, 05W3H, 05XJ5, 070Q9, 07CWC, 07QF7, 07QNK, 07RBJ, 07RJ1, 07T5E, 07VQ3, 0814C, 088QT, 08H15, 08N8A, 08SC4, 08Z4Q; the P-P-bond-hydrolysis-driven transporter is selected from the group of eggnog families 05BZ1, 05CJ1, 05DMK, 05DFW, 05EY8, 05HAC, 05MFV, 07FKK, 07R5U, 07V1T, 08IJ9, 08JQ7, 172T7; the β-Barrel Porin is selected from the group of eggnog families 05DAY, 08KDD; the Auxiliary transport protein is selected from the group of eggnog family 07SYR; the Putative transport protein is selected from the group of eggnog families 05CRE, 05GWF, 06N3A; or the Phosphotransfer-driven group translocator protein is selected from the group of eggnog families 05CI1, 05VI0. The eggnog families are classified as defined on eggnogdb 1.0.2 as released on 3 Nov. 2017.

In another preferred aspect of the above embodiments, the porter is chosen from the PFAM list of PF00083, PF00474, PF00873, PF00893, PF01895, PF01943, PF02690, PF03083, PF04193, PF05977, PF07690, PF07690, PF13347, PF13440, PF14667; the P-P-bond-hydrolysis-driven transporter is chosen from PFAM list PF00005, PF00532, PF00664, PF01061, PF08352, PF14524, PF13407, PF13416, PF17912; the β-Barrel Porin is chosen from PFAM list PF02264, PF02563, PF10531, PF18412; the Auxiliary transport protein is chosen from PFAM list PF13807, PF02706 the Putative transport protein is chosen from PFAM list PF01578, PF03932, PF05140, PF11045 and/or the Phosphotransfer-driven group translocator protein is chosen from PFAM list PF00367, PF00358, PF02378, PF03829. The PFAM list is classified as defined on Pfam 32.0 as released on September 2018.

In another preferred aspect of the above embodiments, the porter is chosen from the InterPro list IPR000390, IPR001036, IPR001411, IPR001734, IPR001927, IPR002797, IPR003663, IPR003841, IPR004316, IPR004633, IPR004638, IPR004734, IPR004812, IPR005275, IPR005828, IPR005829, IPR006603, IPR010290, IPR011701, IPR020846, IPR023008, IPR023721, IPR023722, IPR026022, IPR027417, IPR027463, IPR029303, IPR032896, IPR036259, IPR038078, IPR038377, IPR039672; the P-P-bond-hydrolysis-driven transporter is chosen from InterPro list IPR000412, IPR001734, IPR001761, IPR003439, IPR003593, IPR005829, IPR005978, IPR005981, IPR006059, IPR006060, IPR006061, IPR008995, IPR011527, IPR011701, IPR013456, IPR013525, IPR013563, IPR015851, IPR015855, IPR017871, IPR019554, IPR020846, IPR025997, IPR026266, IPR027417, IPR028082, IPR029439, IPR033893, IPR036259, IPR036640, IPR038377, IPR039421, IPR040582; the β-Barrel Porin is chosen from InterPro list IPR003192, IPR003715, IPR019554, IPR023738, IPR036998, IPR040716; the Auxiliary transport protein is chosen from InterPro list IPR003856, IPR020846, IPR027417, IPR032807, IPR036259; the Putative transport protein is chosen from InterPro list IPR002541, IPR003439, IPR003593, IPR004316, IPR005627, IPR006603, IPR007816, IPR017871, IPR020368, IPR020846, IPR023648, IPR027417, IPR036259, IPR036822; or the Phosphotransfer-driven group translocator protein is chosen from InterPro list IPR001127, IPR001996, IPR003352, IPR004716, IPR010974, IPR011055, IPR013013, IPR018113, IPR018454, IPR036665, IPR036878. The InterPro list is classified as defined on InterPro 75.0 as released on 4 Jul. 2019.

In a preferred aspect of the disclosure, the method as described herein uses a host cell expressing a porter membrane protein selected from MdfA from Escherichia coli K12 MG1655 with SEQ ID NO: 02, IceT from Escherichia coli K12 MG1655 with SEQ ID NO: 06, Blon_2331 from B. longum subsp. infantis (strain ATCC 15697) with SEQ ID NO: 40, Blon_2332 from B. longum subsp. infantis (strain ATCC 15697) with SEQ ID NO: 42, wzx-like protein of Chitinophaga sp. CF118 with SEQ ID NO: 58, Prevotella ruminicola (AR32) with SEQ ID NO: 66, Lactococcus raffinolactis (ATCC 43920) with SEQ ID NO: 64, or Dyadobacter soli DSM 25329 with SEQ ID NO: 62, or functional homolog or functional fragment of any of the above porter membrane proteins or a sequence having at least 80% sequence identity to any one of the MdfA, IceT, Blon_2331, Blon_2332 membrane proteins or wzx-like membrane protein with SEQ ID NO: 02, 06, 40, 42, 58, 66, 64 or 62, respectively. In another preferred aspect, the method described herein uses a host cell expressing a P-P-bond-hydrolysis driven transporter membrane protein selected from lmrA from Lactococcus lactis SRCM103457 with SEQ ID NO: 28, LpsE membrane protein from Sporomusa sphaeroides DSM2875 with SEQ ID NOS: 70 and 74 or from Flavobacterium spartansii with SEQ ID NOS: 68 and 72, TolC from Candidatus Planktophila sulfonica with SEQ ID NO: 76, MsbA from Pedobacter ginsengisoli with SEQ ID NO: 82, Verrucomicrobia bacterium CG1_02_43_26 with SEQ ID NO: 84, malE from Escherichia coli K-12 MG1655 with SEQ ID NO: 206, malK from Escherichia coli K-12 MG1655 with SEQ ID NO: 208, araF from Escherichia coli K-12 MG1655 with SEQ ID NO: 214, xylF from Escherichia coli K-12 MG1655 with SEQ ID NO: 216, or ytfQ from Escherichia coli K-12 MG1655 with SEQ ID NO: 218 or functional homolog or functional fragment of any of the above P-P-bond-hydrolysis driven transporter membrane proteins or a sequence having at least 80% sequence identity to any one of the lmrA, LpsE, TolC, MsbA, malE, malK, araF, xylF or ytfQ membrane protein with SEQ ID NO: 28, 70 74, 68, 72, 76, 82, 84, 206, 208, 214, 216 or 218 respectively.

In yet another preferred aspect, the method described herein uses a host cell expressing a β-barrel porin selected from Wza from Escherichia coli K12 MG1655 with SEQ ID NO: 34 or lamB from Escherichia coli K-12 MG1655 with SEQ ID NO: 204 or functional homolog or functional fragment thereof or a sequence having at least 80% sequence identity to the Wza or lamB membrane protein with SEQ ID NO: 34 or 204, respectively.

In an alternative preferred aspect, the method described herein uses a host cell expressing an auxiliary transport protein selected from Wzc from Thermotoga maritima (strain ATCC 43589/MSB8/DSM 3109/JCM 10099) with SEQ ID NO: 88, or functional homolog or functional fragment thereof or a sequence having at least 80% sequence identity to the Wzc membrane protein with SEQ ID NO: 88.

In another alternative preferred aspect, the method described herein uses a host cell expressing a putative transport protein selected from CutC from Clostridium sp. CAG: 1013 with SEQ ID NO: 90, Odoribacter splanchnicus DSM 20712 with SEQ ID NO: 92, Mitsuaria sp. PDC51 with SEQ ID NO: 94 or Prevotella intermedia ATCC 25611 (DSM 20706) with SEQ ID NO: 96, or functional homolog or functional fragment of any one of the CutC membrane proteins or a sequence having at least 80% sequence identity to any one of the CutC membrane protein with SEQ ID NO: 90, 92, 94 or 96, respectively.

In another alternative preferred aspect, the method described herein uses a host cell expressing a phosphotransfer-driven group translocator selected from nagE from Escherichia coli K-12 MG1655 with SEQ ID NO: 210 or srlB from Escherichia coli K-12 MG1655 with SEQ ID NO: 212 or functional homolog or functional fragment thereof or a sequence having at least 80% sequence identity to the nagE or srlB membrane protein with SEQ ID NO: 210 or 212, respectively.

In yet a further alternative preferred aspect, the method described herein uses a host cell expressing a porter membrane protein selected from MdfA from Escherichia coli K12 MG1655 with SEQ ID NO: 02, IceT from Escherichia coli K12 MG1655 with SEQ ID NO: 06, YnfM from Escherichia coli K12 MG1655 with SEQ ID NO: 04, Yhhs from Escherichia coli K12 MG1655 with SEQ ID NO: 08, EmrD from Escherichia coli K12 MG1655 with SEQ ID NO: 10, YdhC from Escherichia coli K12 MG1655 with SEQ ID NO: 12, YbdA from Escherichia coli K12 MG1655 with SEQ ID NO: 14, YdeE from Escherichia coli K12 MG1655 with SEQ ID NO: 16, MhpT from Escherichia coli K12 MG1655 with SEQ ID NO: 18, YebQ from Escherichia coli K12 MG1655 with SEQ ID NO: 20, YjhB from Escherichia coli K12 MG1655 with SEQ ID NO: 22, Bcr from Escherichia coli K12 MG1655 with SEQ ID NO: 24, FucP from Escherichia coli K12 MG1655 with SEQ ID NO: 26, WzxE from Escherichia coli K12 MG1655 with SEQ ID NO: 32, EmrE from Escherichia coli K12 MG1655 with SEQ ID NO: 38, Blon_2331 from Bifidobacterium longum subsp. infantis (strain ATCC 15697) with SEQ ID NO: 40, Blon_2332 from Bifidobacterium longum subsp. infantis (strain ATCC 15697) with SEQ ID NO: 42, Blon_0247 from Bifidobacterium longum subsp. infantis (strain ATCC 15697) with SEQ ID NO: 46, Blon_0245 from Bifidobacterium longum subsp. infantis (strain ATCC 15697) with SEQ ID NO: 48, Blon_0345 from Bifidobacterium longum subsp. infantis (strain ATCC 15697) with SEQ ID NO: 50, CDT2 from Neurospora crassa OR74A with SEQ ID NO: 52, CDT2 from Aspergillus oryzae RIB40 with SEQ ID NO: 54, Wzx from Chitinophaga sp. CF 118 with SEQ ID NO: 58, Wzx from Eubacterium sp. CAG:581 with SEQ ID NO: 60, Wzx from Dyadobacter soli (DSM 25329) with SEQ ID NO: 62, Wzx from Lactococcus raffinolactis (ATCC 43920) with SEQ ID NO: 64, Wzx from Prevotella ruminicola (AR32) with SEQ ID NO: 66, NAPO from Brachyspira hampsonii P280/1 with SEQ ID NO: 86, NAm from Actinobaculum suis (DSM 20639) with SEQ ID NO: 98, NAm from Ruminococcus gnavus with SEQ ID NO: 100, NAm from Curtobacterium sp. 314Chir4.1 with SEQ ID NO: 102, Nap from Niabella drilacis (DSM25811) with SEQ ID NO: 104, Nap from Saccharicrinis fermentans (DSM 9555) with SEQ ID NO: 106, mdtD from Citrobacter freundii MGH152 with SEQ ID NO: 108, mdtD from Citrobacter werkmanii NBRC 105721 with SEQ ID NO: 110, mdtD from Citrobacter amalonaticus with SEQ ID NO: 112, mdtD from Klebsiella oxytoca with SEQ ID NO: 114, mdtD from Escherichia albertii B156 with SEQ ID NO: 116, yegB from Salmonella enterica subsp. salamae with SEQ ID NO: 118, mdtD from Klebsiella pneumoniae 30684/NJST258_2 with SEQ ID NO: 120, Tcr_1_D38215 from Klebsiella pneumoniae with SEQ ID NO: 122, mdtD from Pseudocitrobacter faecalis with SEQ ID NO: 124, Cmr from Yokenella regensburgei (ATCC43003) with SEQ ID NO: 126, MdfA from Cronobacter muytjensii with SEQ ID NO: 128, MdfA from Klebsiella oxytoca with SEQ ID NO: 130, MFS from Citrobacter koseri with SEQ ID NO: 132, MdfA from Escherichia marmotae with SEQ ID NO: 134, Cmr from Shigella flexneri with SEQ ID NO: 136, MdfA from Salmonella enterica subsp. salamae with SEQ ID NO: 138, Cmr from Citrobacter youngae (ATCC 29220) with SEQ ID NO: 140, MdfA from Citrobacter freundii with SEQ ID NO: 142, MdfA from Enterobacter kobei with SEQ ID NO: 144, MdfA from Enterobacter sp. with SEQ ID NO: 146, MdfA from Lelliottia sp. WB101 with SEQ ID NO: 148, MdfA from Enterobacter ludwigii EcWSU1 with SEQ ID NO: 150, Sweet-like protein from Actinoplanes utahensis with SEQ ID NO: 152, Sweet-like protein from Chitinophagaceae bacterium PMG_246 with SEQ ID NO: 154, Sweet-like protein from Rhizobium sp. PDC82 with SEQ ID NO: 156, Sweet-like protein from Kineococcus rhizosphaerae (DSM 19711) with SEQ ID NO: 158, Sweet-like protein from Morganella morganii IS15 with SEQ ID NO: 160, Sweet-like protein from Geodermatophilus obscurus (strain ATCC 25078) with SEQ ID NO: 162, Sweet-like protein from Bradyrhizobium sp. BTAi1 with SEQ ID NO: 164, Sweet-like protein from Bradyrhizobium japonicum USDA 110 with SEQ ID NO: 166, Sweet-like protein from Xanthomonas campestris pv. vesicatoria str. 85-10 with SEQ ID NO: 168, Sweet-like protein from Herbaspirillum aquaticum with SEQ ID NO: 170, Sweet-like protein from Flavobacteria bacterium MS024-2A with SEQ ID NO: 172, rnd-like from Sinorhizobium medicae WSM419 with SEQ ID NO: 182, arabinose efflux from Azospirillum brasiliense LMG 04375 with SEQ ID NO: 184 or functional homolog or functional fragment of any of the above porter membrane proteins or a protein having an amino acid sequence having at least 80% sequence identity to any one of the MdfA, IceT, YnfM, Yhhs, EmrD, YdhC, YbdA, YdeE, MhpT, YebQ, YjhB, Bcr, FucP, WzxE, EmrE, Wzx, Blon_2331, Blon_2232, Blon_0247, Blon_0245, Blon_0345, NAPO, NAm, Nap, mdtD, YegB, Tcr_1_D38215, cmr, MFS, CDT2, rnd, Sweet-like or arabinose efflux membrane proteins with SEQ ID NO: 02, 06, 04, 08, 10, 12, 14, 16, 18, 20, 22, 24, 26, 32, 38, 40, 42, 46, 48, 50, 52, 54, 58, 60, 62, 64, 66, 86, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 182 or 184, respectively; a P-P-bond-hydrolysis-driven transporter is selected from LmrA from Lactococcus lactis strain SRCM103457 with SEQ ID NO: 28, OppF from Escherichia coli strain K12 MG1655 with SEQ ID NO: 30, Wzk from Helicobacter pylori (strain ATCC 700392/26695) with SEQ ID NO: 36, Blon_2475 from Bifidobacterium longum subsp. infantis (strain ATCC 15697) with SEQ ID NO: 44, LpsE from Flavobacterium spartansii with SEQ ID NO: 68 or 72, LpsE from Sporomusa sphaeroides DSM 2875 with SEQ ID NO: 70 or 74, TolC from Candidatus Planktophila sulfonica with SEQ ID NO: 76, TolC from Butyrivibrio hungatei XBD2006 with SEQ ID NO: 78, MsbA from Roseburia intestinalis CAG:13 with SEQ ID NO: 80, MsbA from Pedobacter ginsengisoli with SEQ ID NO: 82, MsbA from Verrucomicrobia bacterium CG1_02_43_26 with SEQ ID NO: 84, Wzm from Rhizobium sp. Root149 with SEQ ID NO: 174, Wzm from Azospirillum brasiliense LMG 04375 with SEQ ID NO: 176, Wzm from Escherichia coli 113303 with SEQ ID NO: 196, Wzt from Rhizobium sp. Root149 with SEQ ID NO: 178, Wzt from Azospirillum brasiliense LMG 04375 with SEQ ID NO: 180, Wzt from Escherichia coli 113303 with SEQ ID NO: 194, Nodj from Bradyrhizobium japonicum USDA 110 with SEQ ID NO: 188 or 190, malE from Escherichia coli K-12 MG1655 with SEQ ID NO: 206, malK from Escherichia coli K-12 MG1655 with SEQ ID NO: 208, araF from Escherichia coli K-12 MG1655 with SEQ ID NO: 214, xylF from Escherichia coli K-12 MG1655 with SEQ ID NO: 216, or ytfQ from Escherichia coli K-12 MG1655 with SEQ ID NO: 218 or functional homolog or functional fragment of any one of the above P-P-bond-hydrolysis-driven transporter membrane protein or a protein having an amino acid sequence having at least 80% sequence identity to any one of the LmrA, OppF, Wzk, Blon_2475, LpsE, TolC, MsbA, Wzm, Wzt or Nodj membrane proteins with SEQ ID NO: 28, 30, 36, 44, 68, 72, 70, 74, 76, 78, 80, 82, 84, 174, 176, 196, 178, 180, 194, 188, 190, 206, 208, 214, 216 or 218, respectively; a putative transport protein is selected from Cytochrome C biogenesis protein from Helicobacter pylori with SEQ ID NO: 56, CutC from Clostridium sp. CAG: 1013 with SEQ ID NO: 90, CutC from Odoribacter splanchnicus DSM 20712 with SEQ ID NO: 92, CutC from Mitsuaria sp. PDC51 with SEQ ID NO: 94, CutC from Prevotella intermedia ATCC 25611 (DSM 20706) with SEQ ID NO: 96, ybjM from Escherichia coli K12 MG1655 with SEQ ID NO: 190, ybjM from Enterobacteriaceae bacterium ENNIH1 with SEQ ID NO: 192 or functional homolog or functional fragment of a polynucleotide encoding any one of the above putative transport proteins; or protein having an amino acid sequence having at least 80% sequence identity to any one of the CytC, CutC or ybjM membrane protein with SEQ ID NO: 56, 90, 92, 94, 96, 190 or 192, respectively.

As used herein, a protein having an amino acid sequence having at least 80% sequence identity to any of the enlisted membrane proteins, is to be understood as that the sequence has 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9% sequence identity to the full length of the amino acid sequence of the respective membrane protein.

The amino acid sequence of such membrane protein can be a sequence chosen from SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 204, 206, 208, 210, 212, 214, 216 or 218 of the attached sequence listing, or an amino acid sequence that has at least 80% sequence identity, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5% sequence identity to the full length amino acid sequence of any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 204, 206, 208, 210, 212, 214, 216 or 218.

In a further aspect of the disclosure, the method as described herein uses a host cell expressing a membrane protein that is a transporter protein involved in transport of compounds across the outer membrane of the cell wall.

In a further preferred aspect the method for the production of fucosyllactose as described herein further comprises at least one of the following steps:
i) Adding to the culture medium a lactose feed comprising at least 50, more preferably at least 75, more preferably at least 100, more preferably at least 120, more preferably at least 150 gram of lactose per liter of initial reactor volume wherein the total reactor volume ranges from 250 mL (milliliter) to 10,000 m³ (cubic meter), preferably in a continuous manner, and preferably, so that the final volume of the culture medium is not more than three-fold, preferably not more than two-fold, more preferably, less than 2-fold of the volume of the culture medium before the addition of the lactose feed;
ii) Adding a lactose feed in a continuous manner to the culture medium over the course of 1 day, 2 days, 3 days, 4 days, 5 days by means of a feeding solution;
iii) Adding a lactose feed in a continuous manner to the culture medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein, preferably, the pH of the solution is set between 3 and 7 and wherein, preferably, the temperature of the feed solution is kept between 20° C. and 80° C.;
   said method resulting in a fucosyllactose concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium.

Preferably, the lactose feed is accomplished by adding lactose from the beginning of the cultivating in a concentration of at least 5 mM, preferably in a concentration of 30, 40, 50, 60, 70, 80, 90, 100, 150 mM, more preferably in a concentration >300 mM.

In another aspect the lactose feed is accomplished by adding lactose to the cultivation medium in a concentration, such that throughout the production phase of the cultivation a lactose concentration of at least 5 mM, preferably 10 mM or 30 mM is obtained.

In a further embodiment of the methods described herein, the host cells are cultivated for at least about 60, 80, 100, or about 120 hours or in a continuous manner.

In another embodiment of the methods described herein, a carbon and energy source, preferably sucrose, glucose, fructose, glycerol, maltose, maltodextrine, trehalose, polyols, starch, succinate, malate, pyruvate, lactate, ethanol, citrate, and/or lactose, is also added, preferably continuously to the culture medium, preferably with the lactose.

In a preferred embodiment, a carbon-based substrate is provided, preferably sucrose, in the culture medium for 3 or more days, preferably up to 7 days; and/or provided, in the culture medium, at least 100, advantageously at least 105, more advantageously at least 110, even more advantageously at least 120 grams of sucrose per liter of initial culture volume in a continuous manner, so that the final volume of the culture medium is not more than three-fold, advantageously not more than two-fold, more advantageously less than two-fold of the volume of the culturing medium before the culturing.

Preferably, when performing the method as described herein, a first phase of exponential cell growth is provided by adding a carbon-based substrate, preferably glucose or sucrose, to the culture medium before the lactose is added to the culture medium in a second phase.

In an alternative preferable embodiment, in the method as described herein, the lactose is added already in the first phase of exponential growth together with the carbon-based substrate.

In another embodiment, the method as described herein produces only one fucosyllactose of the group consisting of 2′-fucosyllactose, 3-fucosyllactose and difucosyllactose.

In an alternative embodiment, the method as described herein is producing a mixture of fucosyllactoses.

Such mixture can comprise at least two of the group consisting of 2′-fucosyllactose, 3-fucosyllactose and difucosyllactose.

In the method described herein the genetically modified cell is selected from the group consisting of microorganism, plant, or animal cells, preferably, the microorganism is a bacterium, fungus or a yeast, preferably, the plant is a rice, cotton, rapeseed, soy, maize or corn plant, preferably, the animal is an insect, fish, bird or non-human mammal, all as described herein.

In a specific exemplary embodiment, the method of the disclosure provides the production of fucosyllactose in high yield. The method comprises the step of culturing or fermenting, an in aqueous culture or fermentation medium containing lactose, a genetically modified cell, preferably an E. coli, more preferably an E. coli cell modified by knocking out the genes lacZ, lacY lacA, glgC, agp, pfkA, pfkB, pgi, arcA, iclR, wcaJ, lon and thyA. Even more preferably, additionally the E. coli lacY gene, a fructose kinase gene (frk) originating from Zymomonas mobilis and a sucrose phosphorylase (SP) originating from Bifidobacterium adolescentis can knocked in into the genome and expressed constitutively. The constitutive promoters originate from the promoter library described by De Mey et al. (BMC Biotechnology, 2007). These genetic modifications are also described in WO2016075243 and WO2012007481. Additionally, the modified E. coli cell has a recombinant gene that encodes a single fucosyltransferase. In an exemplary embodiment, this can be an α-1,3-fucosyltransferase that is capable of modifying lactose to produce 3-fucosyllactose (3-FL). The cell furthermore comprises a recombinant gene that encodes the expression of any one of the membrane proteins as described herein.

Another aspect of the disclosure provides a host cell genetically modified for the production of a fucosyllactose, wherein the host cell comprises at least one nucleic acid sequence coding for an enzyme involved in fucosyllactose synthesis, more specifically a fucosyltransferase that transfers a fucose residue from a GDP-fucose donor to a lactose acceptor thereby synthesizing fucosyllactose, and wherein the cell further comprises i) a modified expression of an endogenous membrane protein involved in fucosyllactose transport, more specifically enabling and/or enhancing fucosyllactose transport and/or ii) an expression of a heterologous membrane protein involved in fucosyllactose transport, more specifically enabling and/or enhancing fucosyllactose transport. The membrane protein is i) selected from the group of membrane proteins comprising any one of the PFAM domains found by searching the genomic neighborhood of GT10 and GT11 fucosyltransferase families with InterPro number IPR001503 and IPR002516 respectively, as defined on InterPro 75.0 as released on 4 Jul. 2019, wherein the genomic neighborhood window size is 14 genes before and 14 genes after the respective fucosyltransferase and wherein the membrane protein is not belonging to the SET family, or ii) selected from the group of membrane proteins comprising SEQ ID NO: 204, 206, 208, 210, 212, 214, 216, or 218, or functional homolog or functional fragment of any one of the membrane proteins comprising SEQ ID NO: 204, 206, 208, 210, 212, 214, 216, or 218, or a sequence having at least 80% sequence identity to any one of the membrane proteins with SEQ ID NO: 204, 206, 208, 210, 212, 214, 216, or 218.

Alternatively or preferably, the membrane protein is selected from the group of
a) porters excluding SET;
b) P-P-bond-hydrolysis-driven transporters;
c) β-Barrel Porins;
d) Auxiliary transport proteins;
e) Putative transport proteins; and
f) Phosphotransfer-driven group translocators.

Alternatively or preferably, the membrane protein is selected from the group of i) porter membrane proteins selected from MdfA from Escherichia coli K12 MG1655 with SEQ ID NO: 02, IceT from Escherichia coli K12 MG1655 with SEQ ID NO: 06, YnfM from Escherichia coli K12 MG1655 with SEQ ID NO: 04, Yhhs from Escherichia coli K12 MG1655 with SEQ ID NO: 08, EmrD from Escherichia coli K12 MG1655 with SEQ ID NO: 10, YdhC from Escherichia coli K12 MG1655 with SEQ ID NO: 12, YbdA from Escherichia coli K12 MG1655 with SEQ ID NO: 14, YdeE from Escherichia coli K12 MG1655 with SEQ ID NO: 16, MhpT from Escherichia coli K12 MG1655 with SEQ ID NO: 18, YebQ from Escherichia coli K12 MG1655 with SEQ ID NO: 20, YjhB from Escherichia coli K12 MG1655 with SEQ ID NO: 22, Bcr from Escherichia coli K12 MG1655 with SEQ ID NO: 24, FucP from Escherichia coli K12 MG1655 with SEQ ID NO: 26, WzxE from Escherichia coli K12 MG1655 with SEQ ID NO: 32, EmrE from Escherichia coli K12 MG1655 with SEQ ID NO: 38, Blon_2331 from Bifidobacterium longum subsp. infantis (strain ATCC 15697) with SEQ ID NO: 40, Blon_2332 from Bifidobacterium longum subsp. infantis (strain ATCC 15697) with SEQ ID NO: 42, Blon_0247 from Bifidobacterium longum subsp. infantis (strain ATCC 15697) with SEQ ID NO: 46, Blon_0245 from Bifidobacterium longum subsp. infantis (strain ATCC 15697) with SEQ ID NO: 48, Blon_0345 from Bifidobacterium longum subsp. infantis (strain ATCC 15697) with SEQ ID NO: 50, CDT2 from Neurospora crassa OR74A with SEQ ID NO: 52, CDT2 from Aspergillus oryzae RIB40 with SEQ ID NO: 54, Wzx from Chitinophaga sp. CF 118 with SEQ ID NO: 58, Wzx from Eubacterium sp. CAG:581 with SEQ ID NO: 60, Wzx from Dyadobacter soli (DSM 25329) with SEQ ID NO: 62, Wzx from Lactococcus raffinolactis (ATCC 43920) with SEQ ID NO: 64, Wzx from Prevotella ruminicola (AR32) with SEQ ID NO: 66, NAPO from Brachyspira hampsonii P280/1 with SEQ ID NO: 86, NAm from Actinobaculum suis (DSM 20639) with SEQ ID NO: 98, NAm from Ruminococcus gnavus with SEQ ID NO: 100, NAm from Curtobacterium sp. 314Chir4.1 with SEQ ID NO: 102, Nap from Niabella drilacis (DSM25811) with SEQ ID NO: 104, Nap from Saccharicrinis fermentans (DSM 9555) with SEQ ID NO: 106, mdtD from Citrobacter freundii MGH152 with SEQ ID: NO 108, mdtD from Citrobacter werkmanii NBRC 105721 with SEQ ID NO: 110, mdtD from Citrobacter amalonaticus with SEQ ID NO: 112, mdtD from Klebsiella oxytoca with SEQ ID NO: 114, mdtD from Escherichia albertii B156 with SEQ ID NO: 116, yegB from Salmonella enterica subsp. salamae with SEQ ID NO: 118, mdtD from Klebsiella pneumoniae 30684/NJST258_2 with SEQ ID NO: 120, Tcr_1_D38215 from Klebsiella pneumoniae with SEQ ID NO: 122, mdtD from Pseudocitrobacter faecalis with SEQ ID NO: 124, Cmr from Yokenella regensburgei (ATCC43003) with SEQ ID NO: 126, MdfA from Cronobacter muytjensii with SEQ ID NO: 128, MdfA from Klebsiella oxytoca with SEQ ID NO: 130, MFS from Citrobacter koseri with SEQ ID NO: 132, MdfA from Escherichia marmotae with SEQ ID NO: 134, Cmr from Shigella flexneri with SEQ ID NO: 136, MdfA from Salmonella enterica subsp. salamae with SEQ ID NO: 138, Cmr from Citrobacter youngae (ATCC 29220) with SEQ ID NO: 140, MdfA from Citrobacter freundii with SEQ ID NO: 142, MdfA from Enterobacter kobei with SEQ ID NO: 144, MdfA from Enterobacter sp. with SEQ ID NO: 146, MdfA from Lelliottia sp. WB101 with SEQ ID NO: 148, MdfA from Enterobacter ludwigii EcWSU1 with SEQ ID NO: 150, Sweet-like protein from Actinoplanes utahensis with SEQ ID NO: 152, Sweet-like protein from Chitinophagaceae bacterium PMG_246 with SEQ ID NO: 154, Sweet-like protein from Rhizobium sp. PDC82 with SEQ ID NO: 156, Sweet-like protein from Kineococcus rhizosphaerae (DSM 19711) with SEQ ID NO: 158, Sweet-like protein from Morganella morganii IS15 with SEQ ID NO: 160, Sweet-like protein from Geodermatophilus obscurus (strain ATCC 25078) with SEQ ID NO: 162, Sweet-like protein from Bradyrhizobium sp. BTAi1 with SEQ ID NO: 164, Sweet-like protein from Bradyrhizobium japonicum USDA 110 with SEQ ID NO: 166, Sweet-like protein from Xanthomonas campestris pv. vesicatoria str. 85-10 with SEQ ID NO: 168, Sweet-like protein from Herbaspirillum aquaticum with SEQ ID NO: 170, Sweet-like protein from Flavobacteria bacterium MS024-2A with SEQ ID NO: 172, rnd-like from Sinorhizobium medicae WSM419 with SEQ ID NO: 182, arabinose efflux from Azospirillum brasiliense LMG 04375 with SEQ ID NO: 184 or functional homolog or functional fragment of any of the above porter membrane protein or a protein having an amino acid sequence having at least 80% sequence identity to any of the MdfA, IceT, YnfM, Yhhs, EmrD, YdhC, YbdA, YdeE, MhpT, YebQ, YjhB, Bcr, FucP, WzxE, EmrE, Wzx, Blon 2331, Blon 2232, Blon 0247, Blon 0245, Blon 0345, NAPO, NAm, Nap, mdtD, YegB, Tcr_1_D38215, cmr, MFS, CDT2, md, Sweet-like or arabinose efflux membrane proteins with SEQ ID NO: 02, 06, 04, 08, 10, 12, 14, 16, 18, 20, 22, 24, 26, 32, 38, 40, 42, 46, 48, 50, 52, 54, 58, 60, 62, 64, 66, 86, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 182 or 184, respectively; ii) P-P-bond-hydrolysis-driven transporters selected from LmrA from Lactococcus lactis strain SRCM103457 with SEQ ID NO: 28, OppF from Escherichia coli strain K12 MG1655 with SEQ ID NO: 30, Wzk from Helicobacter pylori (strain ATCC 700392/26695) with SEQ ID NO: 36, Blon_2475 from Bifidobacterium longum subsp. infantis (strain ATCC 15697) with SEQ ID NO: 44, LpsE from Flavobacterium spartansii with SEQ ID NO: 68 or 72, LpsE from Sporomusa sphaeroides DSM 2875 with SEQ ID NO: 70 or 74, TolC from Candidatus Planktophila sulfonica with SEQ ID NO: 76, TolC from Butyrivibrio hungatei XBD2006 with SEQ ID NO: 78, MsbA from Roseburia intestinalis CAG:13 with SEQ ID NO: 80, MsbA from Pedobacter ginsengisoli with SEQ ID NO: 82, MsbA from Verrucomicrobia bacterium CG1_02_43_26 with SEQ ID NO: 84, Wzm from Rhizobium sp. Root149 with SEQ ID NO: 174, Wzm from Azospirillum brasiliense LMG 04375 with SEQ ID NO: 176, Wzm from Escherichia coli 113303 with SEQ ID NO: 196, Wzt from Rhizobium sp. Root149 with SEQ ID NO: 178, Wzt from Azospirillum brasiliense LMG 04375 with SEQ ID NO: 180, Wzt from Escherichia coli 113303 with SEQ ID NO: 194, Nodj from Bradyrhizobium japonicum USDA 110 with SEQ ID NO: 186 or 188, malE from Escherichia coli K-12 MG1655 with SEQ ID NO: 206, malK from Escherichia coli K-12 MG1655 with SEQ ID NO: 208, araF from Escherichia coli K-12 MG1655 with SEQ ID NO: 214, xylF from Escherichia coli K-12 MG1655 with SEQ ID NO: 216, or ytfQ from Escherichia coli K-12 MG1655 with SEQ ID NO: 218 or functional homolog or functional fragment of any one of the above P-P-bond-hydrolysis-driven transporter membrane protein or a protein having an amino acid sequence having at least 80% sequence identity to any of the LmrA, OppF, Wzk, Blon_2475, LpsE, TolC, MsbA, Wzm, Wzt, Nodj, malE, malK, araF, xylF or ytfQ membrane proteins with SEQ ID NO: 28, 30, 36, 44, 68, 72, 70, 74, 76, 78, 80, 82, 84, 174, 176, 196, 178, 180, 194, 188, 190, 206, 208, 214, 216 or 218, respectively; iii) putative transport proteins selected from Cytochrome C biogenesis protein from Helicobacter pylori with SEQ ID NO: 56, CutC from Clostridium sp. CAG: 1013 with SEQ ID NO: 90, CutC from Odoribacter splanchnicus DSM 20712 with SEQ ID NO: 92, CutC from Mitsuaria sp. PDC51 with SEQ ID NO: 94, CutC from Prevotella intermedia ATCC 25611 (DSM 20706) with SEQ ID NO: 96, ybjM from Escherichia coli K12 MG1655 with SEQ ID NO: 190, ybjM from Enterobacteriaceae bacterium ENNIH1 with SEQ ID NO: 192 or functional homolog or functional fragment of any one of the above putative transport protein or protein having an amino acid sequence having at least 80% sequence identity to any one of the CytC, CutC or ybjM membrane protein with SEQ ID NO: 56, 90, 92, 94, 96, 190 or 192, respectively; iv) β-barrel porins selected from Wza from Escherichia coli K12MG1655 with SEQ ID NO: 34 or lamB from Escherichia coli K12 MG1655 with SEQ ID NO: 204 or functional homolog or functional fragment thereof or a sequence having at least 80% sequence identity to the Wza or lamB membrane protein with SEQ ID NO: 34 or 204, respectively; v) auxiliary transport protein is selected from Wzc from Thermotoga maritima (strain ATCC 43589/MSB8/DSM 3109/JCM 10099) with SEQ ID NO: 88, or functional homolog or functional fragment thereof or a sequence having at least 80% sequence identity to the Wzc membrane protein with SEQ ID NO: 88; vi). phosphotransfer-driven group translocators selected from nagE from Escherichia coli K12 MG1655 with SEQ ID NO: 210 or srlB from Escherichia coli K12 MG1655 with SEQ ID NO: 212, or functional homolog or functional fragment thereof or a sequence having at least 80% sequence identity to the nagE or srlB membrane protein with SEQ ID NO: 210 or 212, respectively.

In a further aspect of the disclosure, the cell as described herein expresses a membrane protein that is a transporter protein involved in transport of compounds across the outer membrane of the cell wall.

Another aspect provides for a cell to be stably cultured in a medium, wherein the medium can be any type of growth medium comprising minimal medium, complex medium or growth medium enriched in certain compounds like, for example, but not limited to, vitamins, trace elements, amino acids.

Preferably, the cell is transformed to comprise at least one nucleic acid sequence encoding a protein selected from the group consisting of lactose transporter, fucose transporter, transporter for a nucleotide-activated sugar.

In the methods described herein the cell can be a cell of any organism. The term “organism” or “cell,” as used herein, refers to a microorganism chosen from the list consisting of a bacterium, a yeast or a fungus, or, refers to a plant cell, animal cell, a mammalian cell, an insect cell and a protozoal cell. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild-type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, preferably, the disclosure specifically relates to a mutated and/or transformed Escherichia coli strain as indicated above wherein the E. coli strain is a K12 strain. More specifically, the disclosure relates to a mutated and/or transformed Escherichia coli strain as indicated above wherein the K12 strain is E. coli MG1655. The latter bacterium belonging to the phylum Firmicutes belongs preferably to the Bacilli, preferably from the species Bacillus. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces, Pichia, Hansunella, Kluyveromyces, Yarrowia, Eremothecium, Zygosaccharomyces or Debaromyces. The latter fungus belongs preferably to the genus Rhizopus, Dictyostelium or Aspergillus. “Plant cells” includes cells of flowering and non-flowering plants, as well as algal cells, for example, Chlamydomonas, Chlorella, etc. Preferably, the plant cell is a tobacco, alfalfa, rice, tomato, corn, maize or soybean cell; the mammalian cell is a CHO cell or a HEK cell; the insect cell is an S. frugiperda cell and the protozoal cell is a L. tarentolae cell.

In a preferred embodiment, the cell is a cell of a microorganism, wherein more preferably, the microorganism is a bacterium or a yeast. In a more preferred embodiment, the microorganism is a bacterium, most preferably Escherichia coli. Examples using such E. coli are described herein.

In another more preferred embodiment, the bacterium is yeast. Examples using yeast for the production of fucosyllactose and useable in the disclosure are, e.g., described by Hollands et al. (Metabolic Engineering 52 (2019) 232-242).

It is generally preferred that the cell's catabolic pathway for selected mono-, di- or oligosaccharides is at least partially inactivated, the mono-, di-, or oligosaccharides being involved in and/or required for the synthesis of fucosyllactose.

In a further embodiment, the disclosure provides a method for the production of fucosyllactose, wherein a cell as described herein is used for culturing in a medium under conditions permissive for the production of the fucosyllactose. The fucosyllactose is then separated from the cultivation. As used herein, conditions permissive for the production are to be understood to be conditions relating to physical or chemical parameters enabling growth of and living cells, including but not limited to temperature, pH, pressure, osmotic pressure and product/educt concentration. Preferably, such permissive conditions may include temperature range of 30+/−20° C., a pH range of 7+/−3.

The cell according to the disclosure produces fucosyllactose. The fucosyllactose is chosen from the group consisting of 2′-fucosyllactose, 3-fucosyllactose and difucosyllactose.

Another aspect of the disclosure provides for the use of a membrane protein selected from the group of membrane proteins as defined herein in the fermentative production of fucosyllactose. The fucosyllactose is chosen from the group consisting of 2′-fucosyllactose, 3-fucosyllactose and difucosyllactose.

In a further aspect, the disclosure provides for the use of a cell as defined herein, in a method for the production of fucosyllactose.

In yet another aspect, the disclosure provides for the use of a cell as defined herein wherein the fucosyllactose is 2′-fucosyllactose, 3-fucosyllactose and/or difucosyllactose.

Furthermore, the disclosure also relates to the fucosyllactose obtained by the methods according to the disclosure, as well as to the use of a polynucleotide, the vector, host cells, microorganisms or the polypeptide as described above for the production of fucosyllactose. The fucosyllactose may be used as food additive, prebiotic, symbiotic, for the supplementation of baby food, adult food or feed, or as either therapeutically or pharmaceutically active compound. With the novel methods, fucosyllactose can easily and effectively be provided, without the need for complicated, time and cost consuming synthetic processes.

As used herein, the term “separating” means harvesting, collecting or retrieving the fucosyllactose from the host cell and/or the medium of its growth as explained herein.

Fucosyllactose can be separated in a conventional manner from the cultivation or aqueous culture medium, in which the mixture was made. In case the fucosyllactose is still present in the cells producing the fucosyllactose, conventional manners to free or to extract the fucosyllactose out of the cells can be used, such as cell destruction using high pH, heat shock, sonication, French press, homogenization, enzymatic hydrolysis, chemical hydrolysis, solvent hydrolysis, detergent, hydrolysis, etc. The culture medium, reaction mixture and/or cell extract, together and separately called fucosyllactose containing mixture or cultivation, can then be further used for separating the fucosyllactose.

Typically oligosaccharides, and fucosyllactose being an oligosaccharide, are purified by first removing macro components, i.e., first the cells and cell debris, then the smaller components, i.e., proteins, endotoxins and other components between 1000 Da and 1000 kDa and then the oligosaccharide is desalted by means of retaining the oligosaccharide with a nanofiltration membrane or with electrodialysis in a first step and ion exchange also known as ion exchange chromatography in a second step, which consists of a cation exchange resin and anion exchange resin, wherein most preferably the cation exchange chromatography is performed before the anion exchange chromatography. These steps do not separate sugars from each other with a small difference in degree of polymerization. The separation is done, for instance, by chromatographical separation.

This preferably involves clarifying the fucosyllactose containing mixtures to remove suspended particulates and contaminants, particularly cells, cell components, insoluble metabolites and debris produced by culturing the genetically modified cell and/or performing the enzymatic reaction. In this step, the fucosyllactose containing mixture can be clarified in a conventional manner. Preferably, the fucosyllactose containing mixture is clarified by centrifugation, flocculation, decantation and/or filtration. A second step of separating the fucosyllactose from the fucosyllactose containing mixture preferably involves removing substantially all the proteins, as well as peptides, amino acids, RNA and DNA and any endotoxins and glycolipids that could interfere with the subsequent separation step, from the fucosyllactose containing mixture, preferably after it has been clarified. In this step, proteins and related impurities can be removed from the fucosyllactose containing mixture in a conventional manner. Preferably, proteins, salts, byproducts, color and other related impurities are removed from the fucosyllactose containing mixture by ultrafiltration, nanofiltration, reverse osmosis, microfiltration, activated charcoal or carbon treatment, tangential flow high-performance filtration, tangential flow ultrafiltration, affinity chromatography, ion exchange chromatography (such as but not limited to cation exchange, anion exchange, mixed bed ion exchange), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane, while fucosyllactose remains in the fucosyllactose containing mixture.

Contaminating compounds with a molecular weight above 1000 Da (dalton) are removed by means of ultrafiltration membranes with a cut-off above 1000 Da to approximately 1000 kDa. The membrane retains the contaminant and the oligosaccharide goes to the filtrate. Typical ultrafiltration principles are well known in the art and are based on Tubular modules, Hollow fiber, spiral-wound or plates. These are used in cross flow conditions or as a dead-end filtration. The membrane composition is well known and available from several vendors, and is composed of PES (Polyethylene sulfone), polyvinylpyrrolidone, PAN (Polyacrylonitrile), PA (Poly-amide), Polyvinylidene difluoride (PVDF), NC (Nitrocellulose), ceramic materials or combinations thereof.

Components smaller than the oligosaccharide, for instance, monosaccharides, salts, disaccharides, acids, bases, medium constituents are separated by means of a nano-filtration or/and electrodialysis. Such membranes have molecular weight cut-offs between 100 Da and 1000 Da. For an oligosaccharide such as 2′-fucosyllactose the optimal cut-off is between 300 Da and 500 Da, minimizing losses in the filtrate. Typical membrane compositions are well known and are, for example, polyamide (PA), TFC, PA-TFC, Polypiperazine-amide, PES, Cellulose Acetate or combinations thereof.

Fucosyllactose is further isolated from the culture medium and/or cell with or without further purification steps by evaporation, lyophilization, crystallization, precipitation, and/or drying, spray drying. The further purification steps allow the formulation of fucosyllactose in combination with other oligosaccharides and/or products, for instance, but not limited to, the co-formulation by means of spray-drying, drying or lyophilization or concentration by means of evaporation in liquid form.

In an even further aspect, the disclosure also provides for a further purification of the fucosyllactose. A further purification of the fucosyllactose may be accomplished, for example, by use of (activated) charcoal or carbon, nanofiltration, ultrafiltration or ion exchange to remove any remaining DNA, protein, LPS, endotoxins, or other impurity. Alcohols, such as ethanol, and aqueous alcohol mixtures can also be used. Another purification step is accomplished by crystallization or precipitation of the product. Another purification step is to spray dry or lyophilize fucosyllactose.

The separated and preferably also purified fucosyllactose can be used as a supplement in infant formulas and for treating various diseases in newborn infants.

As will be shown in the examples herein, the method and the cell of the disclosure provide at least one of the following surprising advantages when using the membrane proteins as defined herein:
Better fucosyllactose titers (enhanced) (g/L),
Better production rate r (g fucosyllactose/L/h),
Better cell performance index CPI (g fucosyllactose/g X),
Better specific productivity Qp (g fucosyllactose/g X/h),
Better yield on sucrose Ys (g fucosyllactose/g sucrose),
Better sucrose uptake/conversion rate Qs (g sucrose/g X/h),
Better lactose conversion/consumption rate rs (g lactose/h),
Enhanced fucosyllactose secretion, and/or
Enhanced growth speed of the production host, when compared to a fucosyllactose production host with an identical genetic background but lacking the expression of the heterologous membrane protein or modulated expression of the endogenous membrane protein.

Moreover, the disclosure relates to the following specific embodiments:

Embodiment 1. Method for the production of fucosyllactose by a genetically modified cell, comprising the steps of: providing a cell capable of producing fucosyllactose, the cell comprising at least one nucleic acid sequence coding for an enzyme involved in fucosyllactose synthesis, the cell further comprising i) a modified expression of an endogenous membrane protein and/or ii) an expression of a heterologous membrane protein, and wherein the membrane protein is selected from the group of membrane proteins comprising any one of the PFAM domains found by searching the genomic neighborhood of GT10 and GT11 fucosyltransferase families with InterPro number IPR001503 and IPR002516 respectively, wherein the genomic neighborhood window size is 14 genes before and 14 genes after the respective fucosyltransferase and wherein the membrane protein is not belonging to the SET family; cultivating the cell in a medium under conditions permissive for the production of the desired fucosyllactose; and preferably separating the fucosyllactose from the cultivation.

Embodiment 2. Method according to embodiment 1 wherein the membrane protein is selected from the group of a) porters; b) P-P-bond-hydrolysis-driven transporters; c) β-Barrel Porins; d) Auxiliary transport proteins; and e) Putative transport proteins.

Embodiment 3. Method for the production of fucosyllactose by a genetically modified cell, comprising the steps of: providing a cell capable of producing fucosyllactose, the cell comprising at least one nucleic acid sequence coding for an enzyme involved in fucosyllactose synthesis, the cell further comprising i) a modified expression of an endogenous membrane protein and/or ii) an expression of a heterologous membrane protein, and wherein the membrane protein is selected from the group of a) porters and wherein the membrane protein is not belonging to the SET family; b) P-P-bond-hydrolysis-driven transporters; c) β-Barrel Porins; d) Auxiliary transport proteins; and e) Putative transport proteins cultivating the cell in a medium under conditions permissive for the production of the desired fucosyllactose, preferably separating the fucosyllactose from the cultivation.

Embodiment 4. Method according to any one of embodiments 2 or 3, wherein the porter is selected from the group of TCDB classes 2.A.1.1, 2.A.1.12, 2.A.1.15, 2.A.1.2, 2.A.1.3, 2.A.1.36, 2.A.1.38, 2.A.1.46, 2.A.1.68, 2.A.1.7, 2.A.1.81, 2.A.123, 2.A.2, 2.A.21, 2.A.58, 2.A.6.3, 2.A.66 and 2.A.7.1.

Embodiment 5. Method according to any one of embodiments 2 or 3, wherein the P-P-bond-hydrolysis-driven transporter is selected from the group of TCDB classes 3.A.1.1, 3.A.1.10, 3.A.1.11 and 3.A.1.5.

Embodiment 6. Method according to any one of embodiments 2 or 3, wherein the β-Barrel Porin is selected from TCDB class 1.B.18.

Embodiment 7. Method according to any one of embodiments 2 or 3, wherein the Auxiliary transport protein is selected from TCDB class 8.A.3.

Embodiment 8. Method according to any one of embodiments 2 or 3, wherein the Putative transport protein is selected from the group of TCDB classes 9.B.14 and 9.B.158.

Embodiment 9. Method according to any one of embodiments 2 or 3, wherein the porter is selected from the group of eggnog families 05BZS, 05C0R, 05C2C, 05CT4, 05CXP, 05CZQ, 05D94, 05DXI, 05E5M, 05E5W, 05E8G, 05EAM, 05EDR, 05EGZ, 05F9N, 05JHE, 05PSV, 05W2Y, 05W3H, 05XJ5, 070Q9, 07CWC, 07QF7, 07QNK, 07RBJ, 07RJ1, 07T5E, 07VQ3, 0814C, 088QT, 08H15, 08N8A, 08SC4, 08Z4Q.

Embodiment 10. Method according to any one of embodiments 2 or 3, wherein the P-P-bond-hydrolysis-driven transporter is selected from the group of eggnog families 05BZ1, 05CJ1, 05EY8, 05HAC, 05MFV, 07V1T, 08IJ9, 08JQ7.

Embodiment 11. Method according to any one of embodiments 2 or 3, wherein the β-Barrel Porin is selected from the group of eggnog family 05DAY.

Embodiment 12. Method according to any one of embodiments 2 or 3, wherein the Auxiliary transport protein is selected from the group of eggnog family 07SYR.

Embodiment 13. Method according to any one of embodiments 2 or 3, wherein the Putative transport protein is selected from the group of eggnog families 05CRE, 05GWF, 06N3A.

Embodiment 14. Method according to any one of embodiments 2 or 3, wherein the porter is chosen from the PFAM list of PF00083, PF00474, PF00873, PF00893, PF01895, PF01943, PF02690, PF03083, PF04193, PF05977, PF07690, PF07690, PF13347, PF13440 and PF14667.

Embodiment 15. Method according to any one of embodiments 2 or 3, wherein the P-P-bond-hydrolysis-driven transporter is chosen from PFAM list PF00005, PF00664, PF01061, PF08352, PF14524 and PF17912.

Embodiment 16. Method according to any one of embodiments 2 or 3, wherein the β-Barrel Porin is chosen from PFAM list PF02563, PF10531 and PF18412.

Embodiment 17. Method according to any one of embodiments 2 or 3, wherein the Auxiliary transport protein is chosen from PFAM list PF13807 and PF02706.

Embodiment 18. Method according to any one of embodiments 2 or 3, wherein the Putative transport protein is chosen from PFAM list PF01578, PF03932, PF05140 and PF11045.

Embodiment 19. Method according to any one of embodiments 2 or 3, wherein the porter is chosen from the InterPro list IPR000390, IPR001036, IPR001411, IPR001734, IPR001927, IPR002797, IPR003663, IPR003841, IPR004316, IPR004633, IPR004638, IPR004734, IPR004812, IPR005275, IPR005828, IPR005829, IPR006603, IPR010290, IPR011701, IPR020846, IPR023008, IPR023721, IPR023722, IPR026022, IPR027417, IPR027463, IPR029303, IPR032896, IPR036259, IPR038078, IPR038377, IPR039672.

Embodiment 20. Method according to any one of embodiments 2 or 3, wherein the P-P-bond-hydrolysis-driven transporter is chosen from InterPro list IPR000412, IPR001734, IPR003439, IPR003593, IPR005829, IPR005978, IPR005981, IPR008995, IPR011527, IPR011701, IPR013525, IPR013563, IPR015851, IPR017871, IPR019554, IPR020846, IPR027417, IPR029439, IPR036259, IPR036640, IPR038377, IPR039421 and IPR040582.

Embodiment 21. Method according to any one of embodiments 2 or 3, wherein the β-Barrel Porin is chosen from InterPro list IPR003715, IPR019554 and IPR040716.

Embodiment 22. Method according to embodiment 2, wherein the Auxiliary transport protein is chosen from InterPro list IPR003856, IPR020846, IPR027417, IPR032807 and IPR036259.

Embodiment 23. Method according to any one of embodiments 2 or 3, wherein the Putative transport protein is chosen from InterPro list IPR002541, IPR003439, IPR003593, IPR004316, IPR005627, IPR006603, IPR007816, IPR017871, IPR020368, IPR020846, IPR023648, IPR027417, IPR036259 and IPR036822.

Embodiment 24. Method according to any one of embodiments 2 or 3, wherein the porter membrane protein is selected from MdfA from Escherichia coli K12 MG1655, IceT from Escherichia coli K12 MG1655, Blon_2331 from B. longum subsp. infantis (strain ATCC 15697), Blon_2332 from B. longum subsp. infantis (strain ATCC 15697), wzx-like protein of Chitinophaga sp. CFJ18, Prevotella ruminicola (AR32), Lactococcus raffinolactis (A ICC 43920), or Dyadobacter soli DSM 25329, or functional homolog or functional fragment of any one of the above porter membrane protein or a sequence having at least 80% sequence identity to any one of the MdfA, IceT, Blon_2331, Blon_2332 membrane proteins or wzx-like membrane protein.

Embodiment 25. Method according to any one of embodiments 2 or 3, wherein the P-P-bond-hydrolysis driven transporter membrane protein is selected from lmrA from Lactococcus lactis subsp. lactis bv. Diacetylactis, LpsE membrane protein from Sporomusa sphaeroides DSM 2875, Flavobacterium spartansii, TolC from Candidatus Planktophila sulfonica, MsbA from Pedobacter ginsengisoli or Verrucomicrobia bacterium CG1_02_43_26, or functional homolog or functional fragment of any of the above P-P-bond-hydrolysis driven transporter membrane protein or a sequence having at least 80% sequence identity to any of the lmrA, LpsE, TolC or MsbA membrane protein.

Embodiment 26. Method according to any one of embodiments 2 or 3, wherein the β-barrel porin is selected from Wza from Escherichia coli K12MG1655 or functional homolog or functional fragment thereof or a sequence having at least 80% sequence identity to the Wza membrane protein.

Embodiment 27. Method according to any one of embodiments 2 or 3, wherein the auxiliary transport protein is selected from Wzc from Thermotoga maritima (strain ATCC 43589/MSB8/DSM 3109/JCM 10099), or functional homolog or functional fragment thereof or a sequence having at least 80% sequence identity to the Wzc membrane protein.

Embodiment 28. Method according to any one of embodiments 2 or 3, wherein the putative transport protein is selected from CutC from Clostridium sp. CAG: 1013, Odoribacter splanchnicus DSM 20712, Mitsuaria sp. PDC51 or Prevotella intermedia ATCC 25611 (DSM 20706), or functional homolog or functional fragment of any of the CutC membrane proteins or a sequence having at least 80% sequence identity to any of the CutC membrane protein.

Embodiment 29. Method according to any one of embodiments 2 or 3, wherein the porter membrane protein is selected from MdfA from Escherichia coli K12 MG1655, IceT from Escherichia coli K12 MG1655, YnfM from Escherichia coli K12 MG1655, Yhhs from Escherichia coli K12 MG1655, EmrD from Escherichia coli K12 MG1655, YdhC from Escherichia coli K12 MG1655, YbdA from Escherichia coli K12 MG1655, YdeE from Escherichia coli K12 MG1655, MhpT from Escherichia coli K12 MG1655, YebQ from Escherichia coli K12 MG1655, YjhB from Escherichia coli K12 MG1655, Bcr from Escherichia coli K12 MG1655, FucP from Escherichia coli K12 MG1655, WzxE from Escherichia coli K12 MG1655, EmrE from Escherichia coli K12 MG1655, Blon_2331 from Bifidobacterium longum subsp. infantis (strain ATCC 15697), Blon_2332 from Bifidobacterium longum subsp. infantis (strain ATCC 15697), Blon_0247 from Bifidobacterium longum subsp. infantis (strain ATCC 15697), Blon_0245 from Bifidobacterium longum subsp. infantis (strain ATCC 15697), Blon_0345 from Bifidobacterium longum subsp. infantis (strain ATCC 15697), CDT2 from Neurospora crassa OR74A, CDT2 from Aspergillus oryzae RIB40, Wzx from Chitinophaga sp. CF 118, Wzx from Eubacterium sp. CAG:581, Wzx from Dyadobacter soli (DSM 25329), Wzx from Lactococcus raffinolactis (ATCC 43920), Wzx from Prevotella ruminicola (AR32), NAPO from Brachyspira hampsonii P280/1, NAm from Actinobaculum suis (DSM 20639), NAm from Ruminococcus gnavus, NAm from Curtobacterium sp. 314Chir4.1, NAm from Planctomycetes bacterium GWF2_42_9, Nap from Niabella drilacis (DSM25811), Nap from Saccharicrinis fermentans (DSM 9555),mdtD from Citrobacter freundii MGH152, mdtD from Citrobacter werkmanii NBRC 105721, mdtD from Citrobacter amalonaticus, mdtD from Klebsiella oxytoca, mdtD from Escherichia albertii B156, yegB from Salmonella enterica subsp. salamae, mdtD from Klebsiella pneumoniae 30684/NJST258_2, Tcr_1_D38215 from Klebsiella pneumoniae, mdtD from Pseudocitrobacter faecalis, Cmr from Yokenella regensburgei (ATCC43003), MdfA from Cronobacter muytjensii, MdfA from Klebsiella oxytoca, MFS from Citrobacter koseri, MdfA from Escherichia marmotae, Cmr from Shigella flexneri, MdfA from Salmonella enterica subsp. salamae, Cmr from Citrobacter youngae (ATCC 29220), MdfA from Citrobacter freundii, MdfA from Enterobacter kobei, MdfA from Enterobacter sp., MdfA from Lelliottia sp. WB101, MdfA from Enterobacter ludwigii EcWSU1, Sweet-like protein from Actinoplanes utahensis, Sweet-like protein from Chitinophagaceae bacterium PMG_246, Sweet-like protein from Rhizobium sp. PDC82, Sweet-like protein from Kineococcus rhizosphaerae (DSM 19711), Sweet-like protein from Morganella morganii IS15, Sweet-like protein from Geodermatophilus obscurus (strain ATCC 25078), Sweet-like protein from Bradyrhizobium sp. BTAi1, Sweet-like protein from Bradyrhizobium japonicum USDA 110, Sweet-like protein from Xanthomonas campestris pv. vesicatoria str. 85-10, Sweet-like protein from Herbaspirillum aquaticum, Sweet-like protein from Flavobacteria bacterium MS024-2A, rnd-like from Sinorhizobium medicae WSM419, arabinose efflux from Azospirillum brasiliense LMG 04375 or functional homolog or functional fragment of any of the above porter membrane protein or a protein having an amino acid sequence having at least 80% sequence identity to any of the MdfA, IceT, YnfM, Yhhs, EmrD, YdhC, YbdA, YdeE, MhpT, YebQ, YjhB, Bcr, FucP, WzxE, EmrE, Wzx, Blon_2331, Blon_2232, Blon_0247, Blon_0245, Blon_0345, NAPO, NAm, Nap, mdtD, YegB, Tcr_1_D38215, cmr, MFS, CDT2, md, Sweet-like or arabinose efflux membrane proteins.

Embodiment 30. Method according to any one of embodiments 2 or 3, wherein the P-P-bond-hydrolysis-driven transporter is selected from LmrA from Lactococcus lactis strain SRCM103457, OppF from Escherichia coli strain K12 MG1655, Wzk from Helicobacter pylori (strain ATCC 700392/26695), Blon_2475 from Bifidobacterium longum subsp. infantis (strain ATCC 15697), LpsE from Flavobacterium spartansii, LpsE from Sporomusa sphaeroides DSM 2875, TolC from Candidatus Planktophila sulfonica, TolC from Butyrivibrio hungatei XBD2006, MsbA from Roseburia intestinalis CAG:13, MsbA from Pedobacter ginsengisoli, MsbA from Verrucomicrobia bacterium CG1_02_43_26, Wzm from Rhizobium sp. Root149, Wzm from Azospirillum brasiliense LMG 04375, Wzm from Escherichia coli 113303, Wzt from Rhizobium sp. Root149, Wzt from Azospirillum brasiliense LMG 04375, Wzt from Escherichia coli 113303, Nodj from Bradyrhizobium japonicum USDA 110 or functional homolog or functional fragment of any one of the above P-P-bond-hydrolysis-driven transporter membrane protein or a protein having an amino acid sequence having at least 80% sequence identity to any of the LmrA, OppF, Wzk, Blon_2475, LpsE, TolC, MsbA, Wzm, Wzt or Nodj membrane proteins.

Embodiment 31. Method according to any one of embodiments 2 or 3, wherein the putative transport protein is selected from Cytochrome C biogenesis protein from Helicobacter pylori, CutC from Clostridium sp. CAG: 1013, CutC from Odoribacter splanchnicus DSM 20712, CutC from Mitsuaria sp. PDC51, CutC from Prevotella intermedia ATCC 25611 (DSM 20706), ybjM from Escherichia coli K12MG1655, ybjM from Enterobacteriaceae bacterium ENNIH1 or functional homolog or functional fragment of any one of the above putative transport protein or protein having an amino acid sequence having at least 80% sequence identity to any one of the CytC, CutC or ybjM membrane protein.

Embodiment 32. Method for the production of fucosyllactose by a genetically modified cell, comprising the steps of: providing a cell capable of producing fucosyllactose, the cell comprising at least one nucleic acid sequence coding for an enzyme involved in fucosyllactose synthesis; the cell further comprising i) a modified expression of an endogenous membrane protein and/or ii) an expression of a heterologous membrane protein, and wherein the membrane protein is selected from the group of membrane proteins consisting of the porter membrane proteins MdfA from Escherichia coli K12 MG1655, IceT from Escherichia coli K12 MG1655, YnfM from Escherichia coli K12 MG1655, Yhhs from Escherichia coli K12 MG1655, EmrD from Escherichia coli K12 MG1655, YdhC from Escherichia coli K12 MG1655, YbdA from Escherichia coli K12 MG1655, YdeE from Escherichia coli K12 MG1655, MhpT from Escherichia coli K12 MG1655, YebQ from Escherichia coli K12 MG1655, YjhB from Escherichia coli K12 MG1655, Bcr from Escherichia coli K12 MG1655, FucP from Escherichia coli K12 MG1655, WzxE from Escherichia coli K12 MG1655, EmrE from Escherichia coli K12 MG1655, Blon_2331 from Bifidobacterium longum subsp. infantis (strain ATCC 15697), Blon_2332 from Bifidobacterium longum subsp. infantis (strain ATCC 15697), Blon_0247 from Bifidobacterium longum subsp. infantis (strain ATCC 15697), Blon_0245 from Bifidobacterium longum subsp. infantis (strain ATCC 15697), Blon_0345 from Bifidobacterium longum subsp. infantis (strain ATCC 15697), CDT2 from Neurospora crassa OR74A, CDT2 from Aspergillus oryzae RIB40, Wzx from Chitinophaga sp. CF1 18, Wzx from Eubacterium sp. CAG:581, Wzx from Dyadobacter soli (DSM 25329), Wzx from Lactococcus raffinolactis (ATCC 43920), Wzx from Prevotella ruminicola (AR32), NAPO from Brachyspira hampsonii P280/1, NAm from Actinobaculum suis (DSM 20639), NAm from Ruminococcus gnavus, NAm from Curtobacterium sp. 314Chir4.1, NAm from Planctomycetes bacterium GWF2_42_9, Nap from Niabella drilacis (DSM25811), Nap from Saccharicrinis fermentans (DSM 9555),mdtD from Citrobacter freundii MGH152, mdtD from Citrobacter werkmanii NBRC 105721, mdtD from Citrobacter amalonaticus, mdtD from Klebsiella oxytoca, mdtD from Escherichia albertii B156, yegB from Salmonella enterica subsp. salamae, mdtD from Klebsiella pneumoniae 30684/NJST258_2, Tcr_1_D38215 from Klebsiella pneumoniae, mdtD from Pseudocitrobacter faecalis, Cmr from Yokenella regensburgei (ATCC43003), MdfA from Cronobacter muytjensii, MdfA from Klebsiella oxytoca, MFS from Citrobacter koseri, MdfA from Escherichia marmotae, Cmr from Shigella flexneri, MdfA from Salmonella enterica subsp. salamae, Cmr from Citrobacter youngae (ATCC 29220), MdfA from Citrobacter freundii, MdfA from Enterobacter kobei, MdfA from Enterobacter sp., MdfA from Lelliottia sp. WB101, MdfA from Enterobacter ludwigii EcWSU1, Sweet-like protein from Actinoplanes utahensis, Sweet-like protein from Chitinophagaceae bacterium PMG_246, Sweet-like protein from Rhizobium sp. PDC82, Sweet-like protein from Kineococcus rhizosphaerae (DSM 19711), Sweet-like protein from Morganella morganii IS15, Sweet-like protein from Geodermatophilus obscurus (strain ATCC 25078), Sweet-like protein from Bradyrhizobium sp. BTAi1, Sweet-like protein from Bradyrhizobium japonicum USDA 110, Sweet-like protein from Xanthomonas campestris pv. vesicatoria str. 85-10, Sweet-like protein from Herbaspirillum aquaticum, Sweet-like protein from Flavobacteria bacterium MS024-2A, rnd-like from Sinorhizobium medicae WSM419, arabinose efflux from Azospirillum brasiliense LMG 04375 or functional homolog or functional fragment of any of the above porter membrane protein or a protein having an amino acid sequence having at least 80% sequence identity to any of the MdfA, IceT, YnfM, Yhhs, EmrD, YdhC, YbdA, YdeE, MhpT, YebQ, YjhB, Bcr, FucP, WzxE, EmrE, Wzx, Blon_2331, Blon_2232, Blon_0247, Blon_0245, Blon_0345, NAPO, NAm, Nap, mdtD, YegB, Tcr_1_D38215, cmr, MFS, CDT2, md, Sweet-like or arabinose efflux membrane proteins; and the P-P-bond-hydrolysis-driven transporters LmrA from Lactococcus lactis strain SRCM103457, OppF from Escherichia coli strain K12 MG1655, Wzk from Helicobacter pylori (strain ATCC 700392/26695), Blon_2475 from Bifidobacterium longum subsp. infantis (strain ATCC 15697), LpsE from Flavobacterium spartansii, LpsE from Sporomusa sphaeroides DSM 2875, TolC from Candidatus Planktophila sulfonica, TolC from Butyrivibrio hungatei XBD2006, MsbA from Roseburia intestinalis CAG:13, MsbA from Pedobacter ginsengisoli, MsbA from Verrucomicrobia bacterium CG1_02_43_26, Wzm from Rhizobium sp. Root149, Wzm from Azospirillum brasiliense LMG 04375, Wzm from Escherichia coli 113303, Wzt from Rhizobium sp. Root149, Wzt from Azospirillum brasiliense LMG 04375, Wzt from Escherichia coli 113303, Nodj from Bradyrhizobium japonicum USDA 110 or functional homolog or functional fragment of any one of the above P-P-bond-hydrolysis-driven transporter membrane protein or a protein having an amino acid sequence having at least 80% sequence identity to any of the LmrA, OppF, Wzk, Blon_2475, LpsE, TolC, MsbA, Wzm, Wzt or Nodj membrane proteins; and a $-barrel porin membrane protein Wza from Escherichia coli K12 MG1655 or functional homolog or functional fragment thereof or a sequence having at least 80% sequence identity to the Wza membrane protein; and auxiliary transport protein Wzc from Thermotoga maritima (strain ATCC 43589/MSB8/DSM 3109/JCM 10099), or functional homolog or functional fragment thereof or a sequence having at least 80% sequence identity to the Wzc membrane protein; and putative transport proteins Cytochrome C biogenesis protein from Helicobacter pylori, CutC from Clostridium sp. CAG: 1013, CutC from Odoribacter splanchnicus DSM 20712, CutC from Mitsuaria sp. PDC51, CutC from Prevotella intermedia ATCC 25611 (DSM 20706), ybjM from Escherichia coli K12 MG1655, ybjM from Enterobacteriaceae bacterium ENNIH1 or functional homolog or functional fragment of any one of the above putative transport protein or protein having an amino acid sequence having at least 80% sequence identity to any one of the CytC, CutC or ybjM membrane protein.

Embodiment 33. Method for the production of fucosyllactose according to any one of the previous embodiments, the method further comprising at least one of the following steps: i) Adding to the culture medium a lactose feed comprising at least 50, more preferably at least 75, more preferably at least 100, more preferably at least 120, more preferably at least 150 gram of lactose per initial reactor volume, preferably in a continuous manner, and preferably so that the final volume of the culture medium is not more than three-fold, preferably not more than two-fold, more preferably less than 2-fold of the volume of the culture medium before the addition of the lactose feed; ii) Adding a lactose feed in a continuous manner to the culture medium over the course of 1 day, 2 days, 3 days, 4 days, 5 days by means of a feeding solution; iii) Adding a lactose feed in a continuous manner to the culture medium over the course of 1 day, 2 days, 3 days, 4 days, 5 days by means of a feeding solution and wherein the concentration of the lactose feeding solution is 50 g/l, preferably 75 g/l, more preferably 100 g/l, more preferably 125 g/l, more preferably 150 g/l, more preferably 175 g/l, more preferably 200 g/l, more preferably 225 g/l, more preferably 250 g/l, more preferably 275 g/l, more preferably 300 g/l, more preferably 325 g/l, more preferably 350 g/l, more preferably 375 g/l, more preferably, 400 g/l, more preferably 450 g/l, more preferably 500 g/l, even more preferably, 550 g/l, most preferably 600 g/l; and wherein preferably the pH of the solution is set between 3 and 7 and wherein preferably, the temperature of the feed solution is kept between 20° C. and 80° C.; iv) the method resulting in a fucosyllactose concentration of at least 50 g/l, preferably at least 75 g/l, more preferably at least 90 g/l, more preferably at least 100 g/l, more preferably at least 125 g/l, more preferably at least 150 g/l, more preferably at least 175 g/l, more preferably at least 200 g/l in the final volume of the culture medium.

Embodiment 34. The method of embodiment 33, wherein the lactose feed is accomplished by adding lactose from the beginning of the cultivating in a concentration of at least 5 mM, preferably in a concentration of 30, 40, 50, 60, 70, 80, 90, 100, 150 mM, more preferably in a concentration >300 mM.

Embodiment 35. The method of any one of the embodiments 33 or 34, wherein the lactose feed is accomplished by adding lactose to the cultivation medium in a concentration, such, that throughout the production phase of the cultivation a lactose concentration of at least 5 mM, preferably 10 mM or 30 mM is obtained.

Embodiment 36. The method of any of the embodiments 33, 34 or 35, wherein the host cells are cultivated for at least about 60, 80, 100, or about 120 hours or in a continuous manner.

Embodiment 37. The method of any one of embodiments 33 to 36, wherein a carbon and energy source, preferably sucrose, glucose, fructose, glycerol, maltose, maltodextrines, trehalose, polyols, starch, succinate, malate, pyruvate, lactate, ethanol, citrate, lactose, is also added, preferably continuously to the culture medium, preferably with the lactose.

Embodiment 38. The method of any one of embodiments 33 to 37, wherein a first phase of exponential cell growth is provided by adding a carbon-based substrate, preferably glucose or sucrose, to the culture medium before the lactose is added to the culture medium in a second phase.

Embodiment 39. Method according to any one of the embodiments 1 to 38, wherein the method is producing a mixture of fucosyllactoses.

Embodiment 40. Method according to any one of embodiments 1 to 39, wherein the fucosyllactose is 2′-fucosyllactose, 3-fucosyllactose and/or difucosyllactose.

Embodiment 41. Method according to any one of embodiment 1 to 40, wherein the genetically modified cell is selected from the group consisting of microorganism, plant, or animal cells, preferably the microorganism is a bacterium, fungus or a yeast, preferably the plant is a rice, cotton, rapeseed, soy, maize or corn plant, preferably the animal is an insect, fish, bird or non-human mammal.

Embodiment 42. Method according to embodiment 41, wherein the cell is an Escherichia coli cell.

Embodiment 43. Host cell genetically modified for the production of a fucosyllactose, wherein the host cell comprises at least one nucleic acid sequence coding for an enzyme involved in fucosyllactose synthesis, the cell further comprising i) a modified expression of an endogenous membrane protein and/or ii) an expression of a heterologous membrane protein, and wherein the membrane protein is selected from the group of membrane proteins comprising any one of the PFAM domains found by searching the genomic neighborhood of GT10 and GT11 fucosyltransferase families with InterPro number IPR001503 and IPR002516 respectively, wherein the genomic neighborhood window size is 14 genes before and 14 genes after the respective fucosyltransferase and wherein the membrane protein is not belonging to the SET family.

Embodiment 44. Host cell according to embodiment 43, wherein the membrane protein is selected from the group of a) porters; b) P-P-bond-hydrolysis-driven transporters; c) $-Barrel Porins; d) Auxiliary transport proteins; and e) Putative transport proteins.

Embodiment 45. Host cell genetically modified for the production of a fucosyllactose, wherein the host cell comprises at least one nucleic acid sequence coding for an enzyme involved in fucosyllactose synthesis, the cell further comprising i) a modified expression of an endogenous membrane protein and/or ii) an expression of a heterologous membrane protein, and wherein the membrane protein is selected from the group of a) porters and wherein the membrane protein is not belonging to the SET family; b) P-P-bond-hydrolysis-driven transporters; c) β-Barrel Porins; d) Auxiliary transport proteins; and e) Putative transport proteins.

Embodiment 46. Host cell according to any one of the embodiments 44 or 45, wherein the membrane protein is chosen from the group of membrane proteins as defined in any one of the embodiments 4 to 32.

Embodiment 47. Host cell genetically modified for the production of a fucosyllactose according to any one of the embodiments 44 or 45, wherein porter membrane protein is selected from MdfA from Escherichia coli K12 MG1655, IceT from Escherichia coli K12 MG1655, YnfM from Escherichia coli K12 MG1655, Yhhs from Escherichia coli K12 MG1655, EmrD from Escherichia coli K12 MG1655, YdhC from Escherichia coli K12 MG1655, YbdA from Escherichia coli K12 MG1655, YdeE from Escherichia coli K12 MG1655, MhpT from Escherichia coli K12 MG1655, YebQ from Escherichia coli K12 MG1655, YjhB from Escherichia coli K12 MG1655, Bcr from Escherichia coli K12 MG1655, FucP from Escherichia coli K12 MG1655, WzxE from Escherichia coli K12 MG1655, EmrE from Escherichia coli K12 MG1655, Blon_2331 from Bifidobacterium longum subsp. infantis (strain ATCC 15697), Blon_2332 from Bifidobacterium longum subsp. infantis (strain ATCC 15697), Blon_0247 from Bifidobacterium longum subsp. infantis (strain ATCC 15697), Blon_0245 from Bifidobacterium longum subsp. infantis (strain ATCC 15697), Blon_0345 from Bifidobacterium longum subsp. infantis (strain ATCC 15697), CDT2 from Neurospora crassa OR74A, CDT2 from Aspergillus oryzae RIB40, Wzx from Chitinophaga sp. CF118, Wzx from Eubacterium sp. CAG:581, Wzx from Dyadobacter soli (DSM 25329), Wzx from Lactococcus raffinolactis (ATCC 43920), Wzx from Prevotella ruminicola (AR32), NAPO from Brachyspira hampsonii P280/1, NAm from Actinobaculum suis (DSM 20639), NAm from Ruminococcus gnavus, NAm from Curtobacterium sp. 314Chir4.1, NAm from Planctomycetes bacterium GWF2_42_9, Nap from Niabella drilacis (DSM25811), Nap from Saccharicrinis fermentans (DSM 9555),mdtD from Citrobacter freundii MGH152, mdtD from Citrobacter werkmanii NBRC 105721, mdtD from Citrobacter amalonaticus, mdtD from Klebsiella oxytoca, mdtD from Escherichia albertii B156, yegB from Salmonella enterica subsp. salamae, mdtD from Klebsiella pneumoniae 30684/NJST258_2, Tcr_1_D38215 from Klebsiella pneumoniae, mdtD from Pseudocitrobacter faecalis, Cmr from Yokenella regensburgei (ATCC43003), MdfA from Cronobacter muytjensii, MdfA from Klebsiella oxytoca, MFS from Citrobacter koseri, MdfA from Escherichia marmotae, Cmr from Shigella flexneri, MdfA from Salmonella enterica subsp. salamae, Cmr from Citrobacter youngae (ATCC 29220), MdfA from Citrobacter freundii, MdfA from Enterobacter kobei, MdfA from Enterobacter sp., MdfA from Lelliottia sp. WB101, MdfA from Enterobacter ludwigli EcWSU1, Sweet-like protein from Actinoplanes utahensis, Sweet-like protein from Chitinophagaceae bacterium PMG_246, Sweet-like protein from Rhizobium sp. PDC82, Sweet-like protein from Kineococcus rhizosphaerae (DSM 19711), Sweet-like protein from Morganella morganii IS15, Sweet-like protein from Geodermatophilus obscurus (strain ATCC 25078), Sweet-like protein from Bradyrhizobium sp. BTAi1, Sweet-like protein from Bradyrhizobium japonicum USDA 110, Sweet-like protein from Xanthomonas campestris pv. vesicatoria str. 85-10, Sweet-like protein from Herbaspirillum aquaticum, Sweet-like protein from Flavobacteria bacterium MS024-2A, rnd-like from Sinorhizobium medicae WSM419, arabinose efflux from Azospirillum brasiliense LMG 04375 or functional homolog or functional fragment of any of the above porter membrane protein or a protein having an amino acid sequence having at least 80% sequence identity to any of the MdfA, IceT, YnfM, Yhhs, EmrD, YdhC, YbdA, YdeE, MhpT, YebQ, YjhB, Bcr, FucP, WzxE, EmrE, Wzx, Blon_2331, Blon_2232, Blon_0247, Blon_0245, Blon_0345, NAPO, NAm, Nap, mdtD, YegB, Tcr_1_D38215, cmr, MFS, CDT2, md, Sweet-like or arabinose efflux membrane proteins.

Embodiment 48. Host cell according to any one of the embodiments 44 or 45, wherein the P-P-bond-hydrolysis-driven transporter is selected from LmrA from Lactococcus lactis strain SRCM103457, OppF from Escherichia coli strain K12 MG1655, Wzk from Helicobacter pylori (strain ATCC 700392/26695), Blon_2475 from Bifidobacterium longum subsp. infantis (strain ATCC 15697), LpsE from Flavobacterium spartansii, LpsE from Sporomusa sphaeroides DSM 2875, TolC from Candidatus Planktophila sulfonica, TolC from Butyrivibrio hungatei XBD2006, MsbA from Roseburia intestinalis CAG:13, MsbA from Pedobacter ginsengisoli, MsbA from Verrucomicrobia bacterium CG1_02_43_26, Wzm from Rhizobium sp. Root149, Wzm from Azospirillum brasiliense LMG 04375, Wzm from Escherichia coli 113303, Wzt from Rhizobium sp. Root149, Wzt from Azospirillum brasiliense LMG 04375, Wzt from Escherichia coli 113303, Nodj from Bradyrhizobium japonicum USDA 110 or functional homolog or functional fragment of any one of the above P-P-bond-hydrolysis-driven transporter membrane protein or a protein having an amino acid sequence having at least 80% sequence identity to any of the LmrA, OppF, Wzk, Blon_2475, LpsE, TolC, MsbA, Wzm, Wzt or Nodj membrane proteins.

Embodiment 49. Host cell according to any one of the embodiments 44 or 45, wherein the putative transport protein is selected from Cytochrome C biogenesis protein from Helicobacter pylori, CutC from Clostridium sp. CAG: 1013, CutC from Odoribacter splanchnicus DSM 20712, CutC from Mitsuaria sp. PDC51, CutC from Prevotella intermedia ATCC 25611 (DSM 20706), ybjM from Escherichia coli K12 MG1655, ybjM from Enterobacteriaceae bacterium ENNIH1 or functional homolog or functional fragment of any one of the above putative transport protein or protein having an amino acid sequence having at least 80% sequence identity to any one of the CytC, CutC or ybjM membrane protein.

Embodiment 50. Host cell according to any one of the embodiments 44 or 45, wherein the β-barrel porin is selected from Wza from Escherichia coli K12 MG1655 or functional homolog or functional fragment thereof or a sequence having at least 80% sequence identity to the Wza membrane protein.

Embodiment 51. Host cell according to any one of the embodiments 44 or 45, wherein the auxiliary transport protein is selected from Wzc from Thermotoga maritima (strain ATCC 43589/MSB8/DSM 3109/JCM 10099), or functional homolog or functional fragment thereof or a sequence having at least 80% sequence identity to the Wzc membrane protein.

Embodiment 52. Cell to be stably cultured in a medium, which cell is adjusted for the production of fucosyllactose, the cell being transformed to comprise at least one nucleic acid sequence coding for an enzyme involved in fucosyllactose synthesis, characterized in that the cell in addition comprises i) a modified expression of an endogenous membrane protein and/or ii) an expression of a heterologous membrane protein and wherein the membrane protein is as defined in any one of the embodiments 1 to 33.

Embodiment 53. Cell according to any one of the embodiments 43 to 52, wherein the cell is selected from the group consisting of microorganism, plant, or animal cells, preferably the microorganism is a bacterium, fungus or a yeast, preferably the plant is a rice, cotton, rapeseed, soy, maize or corn plant, preferably the animal is an insect, fish, bird or non-human mammal.

Embodiment 54. Host cell according to embodiment 53, wherein the cell is an Escherichia coli cell.

Embodiment 55. Cell according to any one of the embodiments 43 to 54 wherein the cell comprises a catabolic pathway for selected mono-, di- or oligosaccharides that is at least partially inactivated, the mono-, di-, or oligosaccharides being involved in and/or required for the synthesis of fucosyllactose.

Embodiment 56. Cell according to any one of the embodiments 43 to 55 wherein the fucosyllactose is 2′-fucosyllactose, 3-fucosyllactose and/or difucosyllactose.

Embodiment 57. Method for the production of fucosyllactose, comprising the steps of: a) providing a cell according to any one of the embodiments 43 to 56, b) culturing the cell in a medium under conditions permissive for the production of the fucosyllactose, c) separating the fucosyllactose from the cultivation.

Embodiment 58. Use of a membrane protein selected from the group membrane proteins as defined in any one of the embodiments 1 to 31 in the fermentative production of fucosyllactose.

Embodiment 59. Use of a cell according to any one of the embodiments 43 to 56, in a method for the production of fucosyllactose.

Embodiment 60. Use of a cell according to embodiment 59 wherein the fucosyllactose is 2′-fucosyllactose, 3-fucosyllactose and/or difucosyllactose.

Moreover, the disclosure relates to the following preferred specific embodiments:

Embodiment 1. Method for the production of fucosyllactose by a genetically modified cell, comprising the steps of: providing a cell capable of producing fucosyllactose, the cell comprising at least one nucleic acid sequence coding for a fucosyltransferase that transfers a fucose residue from a guanosine-diphosphate fucose (GDP-fucose) donor to a lactose acceptor thereby synthesizing fucosyllactose; the cell further comprising i) a modified expression of an endogenous membrane protein enabling and/or enhancing fucosyllactose transport and/or ii) an expression of a heterologous membrane protein enabling and/or enhancing fucosyllactose transport, and wherein the membrane protein is i) selected from the group of membrane proteins comprising any one of the PFAM domains found by searching the genomic neighborhood of GT10 and GT11 fucosyltransferase families with InterPro number IPR001503 and IPR002516 as defined on InterPro 75.0 released on 4 Jul. 2019 respectively, wherein the genomic neighborhood window size is 14 genes before and 14 genes after the respective fucosyltransferase and wherein the membrane protein is not belonging to the SET family, or ii) selected from the group of membrane proteins comprising SEQ ID NO: 204, 206, 208, 210, 212, 214, 216, or 218, or functional homolog or functional fragment of any one of the membrane proteins comprising SEQ ID NO: 204, 206, 208, 210, 212, 214, 216, or 218, or a sequence having at least 80% sequence identity to any one of the membrane proteins with SEQ ID NO: 204, 206, 208, 210, 212, 214, 216, or 218 cultivating the cell in a medium under conditions permissive for the production of the desired fucosyllactose, preferably separating the fucosyllactose from the cultivation.

Embodiment 2. Method according to specific embodiment 1 wherein the membrane protein is selected from the group of a) porters; b) P-P-bond-hydrolysis-driven transporters; c) β-Barrel Porins; d) Auxiliary transport proteins; e) Putative transport proteins; and f) Phosphotransfer-driven group translocators.

Embodiment 3. Method for the production of fucosyllactose by a genetically modified cell, comprising the steps of: providing a cell capable of producing fucosyllactose, the cell comprising at least one nucleic acid sequence coding for a fucosyltransferase that transfers a fucose residue from a GDP-fucose donor to a lactose acceptor thereby synthesizing fucosyllactose, the cell further comprising i) a modified expression of an endogenous membrane protein enabling and/or enhancing fucosyllactose transport and/or ii) an expression of a heterologous membrane protein enabling and/or enhancing fucosyllactose transport, and wherein the membrane protein is selected from the group of a) porters and wherein the membrane protein is not belonging to the SET family; b) P-P-bond-hydrolysis-driven transporters; c) β-Barrel Porins; d) Auxiliary transport proteins; e) Putative transport proteins; and f) Phosphotransfer-driven group translocators cultivating the cell in a medium under conditions permissive for the production of the desired fucosyllactose, preferably separating the fucosyllactose from the cultivation.

Embodiment 4. Method according to any one of specific embodiments 2 or 3, wherein the porter is selected from the group of TCDB classes 2.A.1.1, 2.A.1.12, 2.A.1.15, 2.A.1.2, 2.A.1.3, 2.A.1.36, 2.A.1.38, 2.A.1.46, 2.A.1.68, 2.A.1.7, 2.A.1.81, 2.A.123, 2.A.2, 2.A.21, 2.A.58, 2.A.6.3, 2.A.66 and 2.A.7.1, as defined by TCDB.org as released on 17 Jun. 2019.

Embodiment 5. Method according to any one of specific embodiments 2 or 3, wherein the P-P-bond-hydrolysis-driven transporter is selected from the group of TCDB classes 3.A.1.1, 3.A.1.2, 3.A.1.10, 3.A.1.11 and 3.A.1.5, as defined by TCDB.org as released on 17 Jun. 2019.

Embodiment 6. Method according to any one of specific embodiments 2 or 3, wherein the β-Barrel Porin is selected from TCDB classes 1.B.3.1 and 1.B.18, as defined by TCDB.org as released on 17 Jun. 2019.

Embodiment 7. Method according to any one of specific embodiments 2 or 3, wherein the Auxiliary transport protein is selected from TCDB class 8.A.3, as defined by TCDB.org as released on 17 Jun. 2019.

Embodiment 8. Method according to any one of specific embodiments 2 or 3, wherein the Putative transport protein is selected from the group of TCDB classes 9.B.14 and 9.B.158, as defined by TCDB.org as released on 17 Jun. 2019.

Embodiment 9. Method according to any one of specific embodiments 2 or 3, wherein the phosphotransfer-driven group translocators is selected from the group of TCDB classes 4.A.1.1 and 4.A.4.1, as defined by TCDB.org as released on 17 Jun. 2019.

Embodiment 10. Method according to any one of specific embodiments 2 or 3, wherein the porter is selected from the group of eggnog families 05BZS, 05C0R, 05C2C, 05CT4, 05CXP, 05CZQ, 05D94, 05DXL, 05E5M, 05E5W, 05E8G, 05EAM, 05EDR, 05EGZ, 05F9N, 05JHE, 05PSV, 05W2Y, 05W3H, 05XJ5, 070Q9, 07CWC, 07QF7, 07QNK, 07RBJ, 07RJ1, 07T5E, 07VQ3, 0814C, 088QT, 08H15, 08N8A, 08SC4, 08Z4Q, as defined by eggnogdb 1.0.2 as released on 3 Nov. 2017.

Embodiment 11. Method according to any one of specific embodiments 2 or 3, wherein the P-P-bond-hydrolysis-driven transporter is selected from the group of eggnog families 05BZ1, 05CJ1, 05EY8, 05HAC, 05DMK, 05DFW, 05MFV, 07FKK, 07R5U, 07V1T, 08IJ9, 08JQ7, 172T7, as defined by eggnogdb 1.0.2 as released on 3 Nov. 2017.

Embodiment 12. Method according to any one of specific embodiments 2 or 3, wherein the β-Barrel Porin is selected from the group of eggnog family 05DAY, 08KDD, as defined by eggnogdb 1.0.2 as released on 3 Nov. 2017.

Embodiment 13. Method according to any one of specific embodiments 2 or 3, wherein the Auxiliary transport protein is selected from the group of eggnog family 07SYR, as defined by eggnogdb 1.0.2 as released on 3 Nov. 2017.

Embodiment 14. Method according to any one of specific embodiments 2 or 3, wherein the Putative transport protein is selected from the group of eggnog families 05CRE, 05GWF, 06N3A, as defined by eggnogdb 1.0.2 as released on 3 Nov. 2017.

Embodiment 15. Method according to any one of specific embodiments 2 or 3, wherein the phosphotransfer-driven group translocators is selected from the group of eggnog families 05CI1 and 05VI0, as defined by eggnogdb 1.0.2 as released on 3 Nov. 2017.

Embodiment 16. Method according to any one of specific embodiments 2 or 3, wherein the porter is chosen from the PFAM list of PF00083, PF00474, PF00873, PF00893, PF01895, PF01943, PF02690, PF03083, PF04193, PF05977, PF07690, PF07690, PF13347, PF13440 and PF14667, as defined by Pfam 32.0 as released on September 2018.

Embodiment 17. Method according to any one of specific embodiments 2 or 3, wherein the P-P-bond-hydrolysis-driven transporter is chosen from PFAM list PF00005, PF00532, PF00664, PF01061, PF08352, PF14524, PF13407, PF13416 and PF17912, as defined by Pfam 32.0 as released on September 2018.

Embodiment 18. Method according to any one of specific embodiments 2 or 3, wherein the β-Barrel Porin is chosen from PFAM list PF02264, PF02563, PF10531 and PF18412, as defined by Pfam 32.0 as released on September 2018.

Embodiment 19. Method according to any one of specific embodiments 2 or 3, wherein the Auxiliary transport protein is chosen from PFAM list PF13807 and PF02706, as defined by Pfam 32.0 as released on September 2018.

Embodiment 20. Method according to any one of specific embodiments 2 or 3, wherein the Putative transport protein is chosen from PFAM list PF01578, PF03932, PF05140 and PF11045, as defined by Pfam 32.0 as released on September 2018.

Embodiment 21. Method according to any one of specific embodiments 2 or 3, wherein the phosphotransfer-driven group translocators is chosen from PFAM list PF00367, PF00358, PF02378, PF03829, as defined by Pfam 32.0 as released on September 2018.

Embodiment 22. Method according to any one of specific embodiments 2 or 3, wherein the porter is chosen from the InterPro list IPR000390, IPR001036, IPR001411, IPR001734, IPR001927, IPR002797, IPR003663, IPR003841, IPR004316, IPR004633, IPR004638, IPR004734, IPR004812, IPR005275, IPR005828, IPR005829, IPR006603, IPR010290, IPR011701, IPR020846, IPR023008, IPR023721, IPR023722, IPR026022, IPR027417, IPR027463, IPR029303, IPR032896, IPR036259, IPR038078, IPR038377, IPR039672, as defined by InterPro 75.0 as released on 4 Jul. 2019.

Embodiment 23. Method according to any one of specific embodiments 2 or 3, wherein the P-P-bond-hydrolysis-driven transporter is chosen from InterPro list IPR000412, IPR001734, IPR001761, IPR003439, IPR003593, IPR005829, IPR005978, IPR005981, IPR006059, IPR006060, IPR006061, IPR008995, IPR011527, IPR011701, IPR013456, IPR013525, IPR013563, IPR015851, IPR015855, IPR017871, IPR019554, IPR020846, IPR025997, IPR026266, IPR027417, IPR028082, IPR029439, IPR033893, IPR036259, IPR036640, IPR038377, IPR039421 and IPR040582, as defined by InterPro 75.0 as released on 4 Jul. 2019.

Embodiment 24. Method according to any one of specific embodiments 2 or 3, wherein the β-Barrel Porin is chosen from InterPro list IPR003192, IPR003715, IPR019554, IPR023738, IPR036998 and IPR040716, as defined by InterPro 75.0 as released on 4 Jul. 2019.

Embodiment 25. Method according to any one of specific embodiments 2 or 3, wherein the Auxiliary transport protein is chosen from InterPro list IPR003856, IPR020846, IPR027417, IPR032807 and IPR036259, as defined by InterPro 75.0 as released on 4 Jul. 2019.

Embodiment 26. Method according to any one of specific embodiments 2 or 3, wherein the Putative transport protein is chosen from InterPro list IPR002541, IPR003439, IPR003593, IPR004316, IPR005627, IPR006603, IPR007816, IPR017871, IPR020368, IPR020846, IPR023648, IPR027417, IPR036259 and IPR036822, as defined by InterPro 75.0 as released on 4 Jul. 2019.

Embodiment 27. Method according to any one of specific embodiments 2 or 3, wherein the phosphotransfer-driven group translocators is chosen from InterPro list IPR001127, IPR001996, IPR003352, IPR004716, IPR010974, IPR011055, IPR013013, IPR018113, IPR018454, IPR036665 and IPR036878, as defined by InterPro 75.0 as released on 4 Jul. 2019.

Embodiment 28. Method according to any one of specific embodiments 2 or 3, wherein the porter membrane protein is selected from MdfA from Escherichia coli K12 MG1655 with SEQ ID NO: 02, IceT from Escherichia coli K12MG1655 with SEQ ID NO: 06, Blon_2331 from B. longum subsp. infantis (strain ATCC 15697) with SEQ ID NO: 40, Blon_2332 from B. longum subsp. infantis (strain ATCC 15697) with SEQ ID NO: 42, wzx-like protein of Chitinophaga sp. CF118 with SEQ ID NO: 58, Prevotella ruminicola (AR32) with SEQ ID NO: 66, Lactococcus raffinolactis (ATCC 43920) with SEQ ID NO: 64, or Dyadobacter soli DSM 25329 with SEQ ID NO: 62, or functional homolog or functional fragment of any one of the above porter membrane protein or a sequence having at least 80% sequence identity to any one of the MdfA, IceT, Blon_2331, Blon_2332 membrane proteins or wzx-like membrane protein with SEQ ID NO: 02, 06, 40, 42, 58, 66, 64 or 62, respectively.

Embodiment 29. Method according to any one of specific embodiments 2 or 3, wherein the P-P-bond-hydrolysis driven transporter membrane protein is selected from lmrA from Lactococcus lactis strain SRCM103457 with SEQ ID NO: 28, LpsE membrane protein from Sporomusa sphaeroides DSM 2875 with SEQ ID NO: 70 or 74, Flavobacterium spartansii with SEQ ID NO: 68 or 72, TolC from Candidatus Planktophila sulfonica with SEQ ID NO: 76, MsbA from Pedobacter ginsengisoli with SEQ ID NO: 82 or Verrucomicrobia bacterium CG1_02_43_26 with SEQ ID NO: 84, malE from Escherichia coli K-12 MG1655 with SEQ ID NO: 206, malK from Escherichia coli K-12 MG1655 with SEQ ID NO: 208, araF from Escherichia coli K-12 MG1655 with SEQ ID NO: 214, xylF from Escherichia coli K-12 MG1655 with SEQ ID NO: 216, or ytfQ from Escherichia coli K-12 MG1655 with SEQ ID NO: 218 or functional homolog or functional fragment of any of the above P-P-bond-hydrolysis driven transporter membrane protein or a sequence having at least 80% sequence identity to any of the lmrA, LpsE, TolC, MsbA, malE, malK, araF, xylF or ytfQ membrane protein with SEQ ID NO: 28, 70, 74, 68, 72, 76, 82, 84, 206, 208, 214, 216, or 218, respectively.

Embodiment 30. Method according to any one of specific embodiments 2 or 3, wherein the β-barrel porin is selected from Wza from Escherichia coli K12MG1655 with SEQ ID NO: 34 or lamB from Escherichia coli K12 MG1655 with SEQ ID NO: 204 or functional homolog or functional fragment thereof or a sequence having at least 80% sequence identity to the Wza or lamB membrane proteins with SEQ ID NO: 34 or 204, respectively.

Embodiment 31. Method according to any one of specific embodiments 2 or 3, wherein the auxiliary transport protein is selected from Wzc from Thermotoga maritima (strain ATCC 43589/MSB8/DSM 3109/JCM 10099) with SEQ ID NO: 88, or functional homolog or functional fragment thereof or a sequence having at least 80% sequence identity to the Wzc membrane protein with SEQ ID NO: 88.

Embodiment 32. Method according to any one of specific embodiments 2 or 3, wherein the putative transport protein is selected from CutC from Clostridium sp. CAG: 1013 with SEQ ID NO: 90, Odoribacter splanchnicus DSM 20712 with SEQ ID NO: 92, Mitsuaria sp.

PDC51 with SEQ ID NO: 94 or Prevotella intermedia ATCC 25611 (DSM 20706) with SEQ ID NO: 96, or functional homolog or functional fragment of any of the CutC membrane proteins or a sequence having at least 80% sequence identity to any of the CutC membrane protein with SEQ ID NO: 90, 92, 94 or 96, respectively.

Embodiment 33. Method according to any one of specific embodiments 2 or 3, wherein the phosphotransfer-driven group translocator is selected from nagE from Escherichia coli K12 MG1655 with SEQ ID NO: 210, srlB from Escherichia coli K12 MG1655 with SEQ ID NO: 212 or functional homolog or functional fragment of any of the nagE or srlB membrane protein or a sequence having at least 80% sequence identity to any of the nagE or srlB membrane protein with SEQ ID NO: 210 or 212, respectively.

Embodiment 34. Method according to any one of specific embodiments 2 or 3, wherein the porter membrane protein is selected from MdfA from Escherichia coli K12 MG1655 with SEQ ID NO: 02, IceT from Escherichia coli K12 MG1655 with SEQ ID NO: 06, YnfM from Escherichia coli K12 MG1655 with SEQ ID NO: 04, Yhhs from Escherichia coli K12 MG1655 with SEQ ID NO: 08, EmrD from Escherichia coli K12 MG1655 with SEQ ID NO: 10, YdhC from Escherichia coli K12 MG1655 with SEQ ID NO: 12, YbdA from Escherichia coli K12 MG1655 with SEQ ID NO: 14, YdeE from Escherichia coli K12 MG1655 with SEQ ID NO: 16, MhpT from Escherichia coli K12 MG1655 with SEQ ID NO: 18, YebQ from Escherichia coli K12 MG1655 with SEQ ID NO: 20, YjhB from Escherichia coli K12 MG1655 with SEQ ID NO: 22, Bcr from Escherichia coli K12 MG1655 with SEQ ID NO: 24, FucP from Escherichia coli K12 MG1655 with SEQ ID NO: 26, WzxE from Escherichia coli K12 MG1655 with SEQ ID NO: 32, EmrE from Escherichia coli K12 MG1655 with SEQ ID NO: 38, Blon_2331 from Bifidobacterium longum subsp. infantis (strain ATCC 15697) with SEQ ID NO: 40, Blon_2332 from Bifidobacterium longum subsp. infantis (strain ATCC 15697) with SEQ ID NO: 42, Blon_0247 from Bifidobacterium longum subsp. infantis (strain ATCC 15697) with SEQ ID NO: 46, Blon_0245 from Bifidobacterium longum subsp. infantis (strain ATCC 15697) with SEQ ID NO: 48, Blon_0345 from Bifidobacterium longum subsp. infantis (strain ATCC 15697) with SEQ ID NO: 50, CDT2 from Neurospora crassa OR74A with SEQ ID NO: 52, CDT2 from Aspergillus oryzae RIB40 with SEQ ID NO: 54, Wzx from Chitinophaga sp. CF 118 with SEQ ID NO: 58, Wzx from Eubacterium sp. CAG:581 with SEQ ID NO: 60, Wzx from Dyadobacter soli (DSM 25329) with SEQ ID NO: 62, Wzx from Lactococcus raffinolactis (ATCC 43920) with SEQ ID NO: 64, Wzx from Prevotella ruminicola (AR32) with SEQ ID NO: 66, NAPO from Brachyspira hampsonii P280/1 with SEQ ID NO: 86, NAm from Actinobaculum suis (DSM 20639) with SEQ ID NO: 98, NAm from Ruminococcus gnavus with SEQ ID NO: 100, NAm from Curtobacterium sp. 314Chir4.1 with SEQ ID NO: 102, Nap from Niabella drilacis (DSM25811) with SEQ ID NO: 104, Nap from Saccharicrinis fermentans (DSM 9555) with SEQ ID NO: 106, mdtD from Citrobacter freundii MGH152 with SEQ ID NO: 108, mdtD from Citrobacter werkmanii NBRC 105721 with SEQ ID NO: 110, mdtD from Citrobacter amalonaticus with SEQ ID NO: 112, mdtD from Klebsiella oxytoca with SEQ ID NO: 114, mdtD from Escherichia alberti B156 with SEQ ID NO: 116, yegB from Salmonella enterica subsp. salamae with SEQ ID NO: 118, mdtD from Klebsiella pneumoniae 30684/NJST258_2 with SEQ ID NO: 120, Tcr_1_D38215 from Klebsiella pneumoniae with SEQ ID NO: 122, mdtD from Pseudocitrobacter faecalis with SEQ ID NO: 124, Cmr from Yokenella regensburgei (ATCC43003) with SEQ ID NO: 126, MdfA from Cronobacter muytjensii with SEQ ID NO: 128, MdfA from Klebsiella oxytoca with SEQ ID NO: 130, MFS from Citrobacter koseri with SEQ ID NO: 132, MdfA from Escherichia marmotae with SEQ ID NO: 134, Cmr from Shigella flexneri with SEQ ID NO: 136, MdfA from Salmonella enterica subsp. salamae with SEQ ID NO: 138, Cmr from Citrobacter youngae (ATCC 29220) with SEQ ID NO: 140, MdfA from Citrobacter freundii with SEQ ID NO: 142, MdfA from Enterobacter kobei with SEQ ID NO: 144, MdfA from Enterobacter sp. with SEQ ID NO: 146, MdfA from Lelliottia sp. WB101 with SEQ ID NO: 148, MdfA from Enterobacter ludwigii EcWSU1 with SEQ ID NO: 150, Sweet-like protein from Actinoplanes utahensis with SEQ ID NO: 152, Sweet-like protein from Chitinophagaceae bacterium PMG 246 with SEQ ID NO: 154, Sweet-like protein from Rhizobium sp. PDC82 with SEQ ID NO: 156, Sweet-like protein from Kineococcus rhizosphaerae (DSM 19711) with SEQ ID NO: 158, Sweet-like protein from Morganella morganii IS15 with SEQ ID NO: 160, Sweet-like protein from Geodermatophilus obscurus (strain ATCC 25078) with SEQ ID NO: 162, Sweet-like protein from Bradyrhizobium sp. BTAi1 with SEQ ID NO: 164, Sweet-like protein from Bradyrhizobium japonicum USDA 110 with SEQ ID NO: 166, Sweet-like protein from Xanthomonas campestris pv. vesicatoria str. 85-10 with SEQ ID NO: 168, Sweet-like protein from Herbaspirillum aquaticum with SEQ ID NO: 170, Sweet-like protein from Flavobacteria bacterium MS024-2A with SEQ ID NO: 172, rnd-like from Sinorhizobium medicae WSM419 with SEQ ID NO: 182, arabinose efflux from Azospirillum brasiliense LMG 04375 with SEQ ID NO: 184 or functional homolog or functional fragment of any of the above porter membrane protein or a protein having an amino acid sequence having at least 80% sequence identity to any of the MdfA, IceT, YnfM, Yhhs, EmrD, YdhC, YbdA, YdeE, MhpT, YebQ, YjhB, Bcr, FucP, WzxE, EmrE, Wzx, Blon_2331, Blon_2232, Blon_0247, Blon_0245, Blon_0345, NAPO, NAm, Nap, mdtD, YegB, Tcr_1_D38215, cmr, MFS, CDT2, md, Sweet-like or arabinose efflux membrane proteins with SEQ ID NO: 02, 06, 04, 08, 10, 12, 14, 16, 18, 20, 22, 24, 26, 32, 38, 40, 42, 46, 48, 50, 52, 54, 58, 60, 62, 64, 66, 86, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 182 or 184, respectively.

Embodiment 35. Method according to any one of specific embodiments 2 or 3, wherein the P-P-bond-hydrolysis-driven transporter is selected from LmrA from Lactococcus lactis strain SRCM103457 with SEQ ID NO: 28, OppF from Escherichia coli strain K12 MG1655 with SEQ ID NO: 30, Wzk from Helicobacter pylori (strain ATCC 700392/26695) with SEQ ID NO: 36, Blon_2475 from Bifidobacterium longum subsp. infantis (strain ATCC 15697) with SEQ ID NO: 44, LpsE from Flavobacterium spartansii with SEQ ID NO: 68 or 72, LpsE from Sporomusa sphaeroides DSM 2875 with SEQ ID NO: 70 or 74, TolC from Candidatus Planktophila sulfonica with SEQ ID NO: 76, TolC from Butyrivibrio hungatei XBD2006 with SEQ ID NO: 78, MsbA from Roseburia intestinalis CAG:13 with SEQ ID NO: 80, MsbA from Pedobacter ginsengisoli with SEQ ID NO: 82, MsbA from Verrucomicrobia bacterium CG1_02_43_26 with SEQ ID NO: 84, Wzm from Rhizobium sp. Root149 with SEQ ID NO: 174, Wzm from Azospirillum brasiliense LMG 04375 with SEQ ID NO: 176, Wzm from Escherichia coli 113303 with SEQ ID NO: 196, Wzt from Rhizobium sp. Root149 with SEQ ID NO: 178, Wzt from Azospirillum brasiliense LMG 04375 with SEQ ID NO: 180, Wzt from Escherichia coli 113303 with SEQ ID NO: 194, Nodj from Bradyrhizobium japonicum USDA 110 with SEQ ID NO: 188 or 190, malE from Escherichia coli K-12 MG1655 with SEQ ID NO: 206, malK from Escherichia coli K-12 MG1655 with SEQ ID NO: 208, araF from Escherichia coli K-12 MG1655 with SEQ ID NO: 214, xylF from Escherichia coli K-12 MG1655 with SEQ ID NO: 216, or ytfQ from Escherichia coli K-12 MG1655 with SEQ ID NO: 218 or functional homolog or functional fragment of any one of the above P-P-bond-hydrolysis-driven transporter membrane protein or a protein having an amino acid sequence having at least 80% sequence identity to any of the LmrA, OppF, Wzk, Blon_2475, LpsE, TolC, MsbA, Wzm, Wzt, Nodj, malE, malK, araF, xylF or ytfQ membrane proteins with SEQ ID NO: 28, 30, 36, 44, 68, 72, 70, 74, 76, 78, 80, 82, 84, 174, 176, 196, 178, 180, 194, 188, 190, 206, 208, 214, 216 or 218, respectively.

Embodiment 36. Method according to any one of specific embodiments 2 or 3, wherein the putative transport protein is selected from Cytochrome C biogenesis protein from Helicobacter pylori with SEQ ID NO: 56, CutC from Clostridium sp. CAG: 1013 with SEQ ID NO: 90, CutC from Odoribacter splanchnicus DSM 20712 with SEQ ID NO: 92, CutC from Mitsuaria sp. PDC51 with SEQ ID NO: 94, CutC from Prevotella intermedia ATCC 25611 (DSM 20706) with SEQ ID NO: 96, ybjM from Escherichia coli K12 MG1655 with SEQ ID NO: 190, ybjM from Enterobacteriaceae bacterium ENNIH1 with SEQ ID NO: 192 or functional homolog or functional fragment of any one of the above putative transport protein or protein having an amino acid sequence having at least 80% sequence identity to any one of the CytC, CutC or ybjM membrane protein with SEQ ID NO: 56, 90, 92, 94, 96, 190 or 192, respectively.

Embodiment 37. Method for the production of fucosyllactose by a genetically modified cell, comprising the steps of: providing a cell capable of producing fucosyllactose, the cell comprising at least one nucleic acid sequence coding for a fucosyltransferase that transfers a fucose residue from a GDP-fucose donor to a lactose acceptor thereby synthesizing fucosyllactose, the cell further comprising i) a modified expression of an endogenous membrane protein enabling and/or enhancing fucosyllactose transport and/or ii) an expression of a heterologous membrane protein enabling and/or enhancing fucosyllactose transport, and wherein the membrane protein is selected from the group of membrane proteins consisting of the porter membrane proteins MdfA from Escherichia coli K12 MG1655 with SEQ ID NO: 02, IceT from Escherichia coli K12 MG1655 with SEQ ID NO: 06, YnfM from Escherichia coli K12 MG1655 with SEQ ID NO: 04, Yhhs from Escherichia coli K12 MG1655 with SEQ ID NO: 08, EmrD from Escherichia coli K12 MG1655 with SEQ ID NO: 10, YdhC from Escherichia coli K12 MG1655 with SEQ ID NO: 12, YbdA from Escherichia coli K12 MG1655 with SEQ ID NO: 14, YdeE from Escherichia coli K12 MG1655 with SEQ ID NO: 16, MhpT from Escherichia coli K12 MG1655 with SEQ ID NO: 18, YebQ from Escherichia coli K12 MG1655 with SEQ ID NO: 20, YjhB from Escherichia coli K12 MG1655 with SEQ ID NO: 22, Bcr from Escherichia coli K12 MG1655 with SEQ ID NO: 24, FucP from Escherichia coli K12 MG1655 with SEQ ID NO: 26, WzxE from Escherichia coli K12 MG1655 with SEQ ID NO: 32, EmrE from Escherichia coli K12 MG1655 with SEQ ID NO: 38, Blon_2331 from Bifidobacterium longum subsp. infantis (strain ATCC 15697) with SEQ ID NO: 40, Blon_2332 from Bifidobacterium longum subsp. infantis (strain ATCC 15697) with SEQ ID NO: 42, Blon_0247 from Bifidobacterium longum subsp. infantis (strain ATCC 15697) with SEQ ID NO: 46, Blon_0245 from Bifidobacterium longum subsp. infantis (strain ATCC 15697) with SEQ ID NO: 48, Blon_0345 from Bifidobacterium longum subsp. infantis (strain ATCC 15697) with SEQ ID NO: 50, CDT2 from Neurospora crassa OR74A with SEQ ID NO: 52, CDT2 from Aspergillus oryzae RIB40 with SEQ ID NO: 54, Wzx from Chitinophaga sp. CF 118 with SEQ ID NO: 58, Wzx from Eubacterium sp. CAG:581 with SEQ ID NO: 60, Wzx from Dyadobacter soli (DSM 25329) with SEQ ID NO: 62, Wzx from Lactococcus raffinolactis (ATCC 43920) with SEQ ID NO: 64, Wzx from Prevotella ruminicola (AR32) with SEQ ID NO: 66, NAPO from Brachyspira hampsonii P280/1 with SEQ ID NO: 86, NAm from Actinobaculum suis (DSM 20639) with SEQ ID NO: 98, NAm from Ruminococcus gnavus with SEQ ID NO: 100, NAm from Curtobacterium sp. 314Chir4.1 with SEQ ID NO: 102, Nap from Niabella drilacis (DSM25811) with SEQ ID NO: 104, Nap from Saccharicrinis fermentans (DSM 9555) with SEQ ID NO: 106, mdtD from Citrobacter freundii MGH152 with SEQ ID NO: 108, mdtD from Citrobacter werkmanii NBRC 105721 with SEQ ID NO: 110, mdtD from Citrobacter amalonaticus with SEQ ID NO: 112, mdtD from Klebsiella oxytoca with SEQ ID NO: 114, mdtD from Escherichia alberti B156 with SEQ ID NO: 116, yegB from Salmonella enterica subsp. salamae with SEQ ID NO: 118, mdtD from Klebsiella pneumoniae 30684/NJST258_2 with SEQ ID NO: 120, Tcr_1_D38215 from Klebsiella pneumoniae with SEQ ID NO: 122, mdtD from Pseudocitrobacter faecalis with SEQ ID NO: 124, Cmr from Yokenella regensburgei (ATCC43003) with SEQ ID NO: 126, MdfA from Cronobacter muytjensii with SEQ ID NO: 128, MdfA from Klebsiella oxytoca with SEQ ID NO: 130, MFS from Citrobacter koseri with SEQ ID NO: 132, MdfA from Escherichia marmotae with SEQ ID NO: 134, Cmr from Shigella flexneri with SEQ ID NO: 136, MdfA from Salmonella enterica subsp. salamae with SEQ ID NO: 138, Cmr from Citrobacter youngae (ATCC 29220) with SEQ ID NO: 140, MdfA from Citrobacter freundii with SEQ ID NO: 142, MdfA from Enterobacter kobei with SEQ ID NO: 144, MdfA from Enterobacter sp. with SEQ ID NO: 146, MdfA from Lelliottia sp. WB101 with SEQ ID NO: 148, MdfA from Enterobacter ludwigii EcWSU1 with SEQ ID NO: 150, Sweet-like protein from Actinoplanes utahensis with SEQ ID NO: 152, Sweet-like protein from Chitinophagaceae bacterium PMG 246 with SEQ ID NO: 154, Sweet-like protein from Rhizobium sp. PDC82 with SEQ ID NO: 156, Sweet-like protein from Kineococcus rhizosphaerae (DSM 19711) with SEQ ID NO: 158, Sweet-like protein from Morganella morganii IS15 with SEQ ID NO: 160, Sweet-like protein from Geodermatophilus obscurus (strain ATCC 25078) with SEQ ID NO: 162, Sweet-like protein from Bradyrhizobium sp. BTAi1 with SEQ ID NO: 164, Sweet-like protein from Bradyrhizobium japonicum USDA 110 with SEQ ID NO: 166, Sweet-like protein from Xanthomonas campestris pv. vesicatoria str. 85-10 with SEQ ID NO: 168, Sweet-like protein from Herbaspirillum aquaticum with SEQ ID NO: 170, Sweet-like protein from Flavobacteria bacterium MS024-2A with SEQ ID NO: 172, rnd-like from Sinorhizobium medicae WSM419 with SEQ ID NO: 182, arabinose efflux from Azospirillum brasiliense LMG 04375 with SEQ ID NO: 184 or functional homolog or functional fragment of any of the above porter membrane protein or a protein having an amino acid sequence having at least 80% sequence identity to any of the MdfA, IceT, YnfM, Yhhs, EmrD, YdhC, YbdA, YdeE, MhpT, YebQ, YjhB, Bcr, FucP, WzxE, EmrE, Wzx, Blon_2331, Blon_2232, Blon_0247, Blon_0245, Blon_0345, NAPO, NAm, Nap, mdtD, YegB, Tcr_1_D38215, cmr, MFS, CDT2, md, Sweet-like or arabinose efflux membrane proteins with SEQ ID NO: 02, 06, 04, 08, 10, 12, 14, 16, 18, 20, 22, 24, 26, 32, 38, 40, 42, 46, 48, 50, 52, 54, 58, 60, 62, 64, 66, 86, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 182 or 184, respectively; and the P-P-bond-hydrolysis-driven transporters LmrA from Lactococcus lactis strain SRCM103457 with SEQ ID NO: 28, OppF from Escherichia coli strain K12 MG1655 with SEQ ID NO: 30, Wzk from Helicobacter pylori (strain ATCC 700392/26695) with SEQ ID NO: 36, Blon_2475 from Bifidobacterium longum subsp. infantis (strain ATCC 15697) with SEQ ID NO: 44, LpsE from Flavobacterium spartansii with SEQ ID NO: 68 or 72, LpsE from Sporomusa sphaeroides DSM 2875 with SEQ ID NO: 70 or 74, TolC from Candidatus Planktophila sulfonica with SEQ ID NO: 76, TolC from Butyrivibrio hungatei XBD2006 with SEQ ID NO: 78, MsbA from Roseburia intestinalis CAG:13 with SEQ ID NO: 80, MsbA from Pedobacter ginsengisoli with SEQ ID NO: 82, MsbA from Verrucomicrobia bacterium CG1_02_43_26 with SEQ ID NO: 84, Wzm from Rhizobium sp. Root149 with SEQ ID NO: 174, Wzm from Azospirillum brasiliense LMG 04375 with SEQ ID NO: 176, Wzm from Escherichia coli 113303 with SEQ ID NO: 196, Wzt from Rhizobium sp. Root149 with SEQ ID NO: 178, Wzt from Azospirillum brasiliense LMG 04375 with SEQ ID NO: 180, Wzt from Escherichia coli 113303 with SEQ ID NO: 194, Nodj from Bradyrhizobium japonicum USDA 110 with SEQ ID NO: 188 or 190, malE from Escherichia coli K-12 MG1655 with SEQ ID NO: 206, malK from Escherichia coli K-12 MG1655 with SEQ ID NO: 208, araF from Escherichia coli K-12 MG1655 with SEQ ID NO: 214, xylF from Escherichia coli K-12 MG1655 with SEQ ID NO: 216, or ytfQ from Escherichia coli K-12 MG1655 with SEQ ID NO: 218 or functional homolog or functional fragment of any one of the above P-P-bond-hydrolysis-driven transporter membrane protein or a protein having an amino acid sequence having at least 80% sequence identity to any of the LmrA, OppF, Wzk, Blon_2475, LpsE, TolC, MsbA, Wzm, Wzt, Nodj, malE, malK, araF, xylF or ytfQ membrane proteins with SEQ ID NO: 28, 30, 36, 44, 68, 72, 70, 74, 76, 78, 80, 82, 84, 174, 176, 196, 178, 180, 194, 188, 190, 206, 208, 214, 216, or 218, respectively; and a β-barrel porin membrane protein Wza from Escherichia coli K12 MG1655 with SEQ ID NO: 34 or lamB from Escherichia coli K12 MG1655 with SEQ ID NO: 204 or functional homolog or functional fragment of any one of the Wza or lamB protein or a sequence having at least 80% sequence identity to any one of the Wza or lamB membrane protein with SEQ ID NO: 34 or 204, respectively; and auxiliary transport protein Wzc from Thermotoga maritima (strain ATCC 43589/MSB8/DSM 3109/JCM 10099) with SEQ ID NO: 88, or functional homolog or functional fragment thereof or a sequence having at least 80% sequence identity to the Wzc membrane protein with SEQ ID NO: 88; putative transport proteins Cytochrome C biogenesis protein from Helicobacter pylori with SEQ ID NO: 56, CutC from Clostridium sp. CAG: 1013 with SEQ ID NO: 90, CutC from Odoribacter splanchnicus DSM 20712 with SEQ ID NO: 92, CutC from Mitsuaria sp. PDC51 with SEQ ID NO: 94, CutC from Prevotella intermedia ATCC 25611 (DSM 20706) with SEQ ID NO: 96, ybjM from Escherichia coli K12 MG1655 with SEQ ID NO: 190, ybjM from Enterobacteriaceae bacterium ENNIH1 with SEQ ID NO: 192 or functional homolog or functional fragment of any one of the above putative transport protein or protein having an amino acid sequence having at least 80% sequence identity to any one of the CytC, CutC or ybjM membrane protein with SEQ ID NO: 56, 90, 92, 94, 96, 190 or 192, respectively; and phosphotransfer-driven group translocators nagE from Escherichia coli K12 MG1655 with SEQ ID NO: 210, srlB from Escherichia coli K12 MG1655 with SEQ ID NO: 212 or functional homolog or functional fragment of any of the nagE or srlB membrane protein or a sequence having at least 80% sequence identity to any one of the nagE or srlB membrane protein with SEQ ID NO: 210 or 212, respectively.

Embodiment 38. Method for the production of fucosyllactose according to any one of the previous specific embodiments, wherein the membrane protein is a transporter protein involved in transport of compounds across the outer membrane of the cell wall.

Embodiment 39. Method for the production of fucosyllactose according to any one of the previous specific embodiments, the method further comprising at least one of the following steps: i) adding to the culture medium a lactose feed comprising at least 50, more preferably at least 75, more preferably at least 100, more preferably at least 120, more preferably at least 150 gram of lactose per liter of initial reactor volume wherein the reactor volume ranges from 250 mL to 10,000 m³, preferably in a continuous manner, and preferably so that the final volume of the culture medium is not more than three-fold, preferably not more than two-fold, more preferably less than two-fold of the volume of the culture medium before the addition of the lactose feed; ii) adding a lactose feed in a continuous manner to the culture medium over the course of 1 day, 2 days, 3 days, 4 days, 5 days by means of a feeding solution; iii) adding a lactose feed in a continuous manner to the culture medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein, preferably, the pH of the solution is set between 3 and 7 and wherein, preferably, the temperature of the feed solution is kept between 20° C. and 80° C.; the method resulting in a fucosyllactose concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium.

Embodiment 40. The method of specific embodiment 39, wherein the lactose feed is accomplished by adding lactose from the beginning of the cultivating in a concentration of at least 5 mM, preferably in a concentration of 30, 40, 50, 60, 70, 80, 90, 100, 150 mM, more preferably in a concentration >300 mM.

Embodiment 41. The method of any one of the specific embodiments 39 or 40, wherein the lactose feed is accomplished by adding lactose to the cultivation medium in a concentration, such, that throughout the production phase of the cultivation a lactose concentration of at least 5 mM, preferably 10 mM or 30 mM is obtained.

Embodiment 42. The method of any of the specific embodiments 39, 40 or 41, wherein the host cells are cultivated for at least about 60, 80, 100, or about 120 hours or in a continuous manner.

Embodiment 43. The method of any one of specific embodiments 39 to 42, wherein a carbon and energy source, preferably sucrose, glucose, fructose, glycerol, maltose, maltodextrines, trehalose, polyols, starch, succinate, malate, pyruvate, lactate, ethanol, citrate, lactose, is also added, preferably continuously to the culture medium, preferably with the lactose.

Embodiment 44. The method of any one of specific embodiments 39 to 43, wherein a first phase of exponential cell growth is provided by adding a carbon-based substrate, preferably glucose or sucrose, to the culture medium before the lactose is added to the culture medium in a second phase.

Embodiment 45. Method according to any one of the specific embodiments 1 to 44, wherein the method is producing a mixture of fucosyllactoses.

Embodiment 46. Method according to any one of specific embodiments 1 to 45, wherein the fucosyllactose is 2′-fucosyllactose, 3-fucosyllactose and/or difucosyllactose.

Embodiment 47. Method according to any one of specific embodiment 1 to 46, wherein the genetically modified cell is selected from the group consisting of microorganism, plant, or animal cells, preferably the microorganism is a bacterium, fungus or a yeast, preferably the plant is a rice, cotton, rapeseed, soy, maize or corn plant, preferably the animal is an insect, fish, bird or non-human mammal.

Embodiment 48. Method according to specific embodiment 47, wherein the cell is an Escherichia coli cell.

Embodiment 49. Host cell genetically modified for the production of a fucosyllactose, wherein the host cell comprises at least one nucleic acid sequence coding for a fucosyltransferase that transfers a fucose residue from a GDP-fucose donor to a lactose acceptor thereby synthesizing fucosyllactose, the cell further comprising i) a modified expression of an endogenous membrane protein enabling and/or enhancing fucosyllactose transport and/or ii) an expression of a heterologous membrane protein enabling and/or enhancing fucosyllactose transport, and wherein the membrane protein is i) selected from the group of membrane proteins comprising any one of the PFAM domains found by searching the genomic neighborhood of GT10 and GT11 fucosyltransferase families with InterPro number IPR001503 and IPR002516 as defined on InterPro 75.0 released on 4 Jul. 2019 respectively, wherein the genomic neighborhood window size is 14 genes before and 14 genes after the respective fucosyltransferase and wherein the membrane protein is not belonging to the SET family, or ii) selected from the group of membrane proteins comprising SEQ ID NO: 204, 206, 208, 210, 212, 214, 216, or 218, or functional homolog or functional fragment of any one of the membrane proteins comprising SEQ ID NO: 204, 206, 208, 210, 212, 214, 216, or 218, or a sequence having at least 80% sequence identity to any one of the membrane proteins with SEQ ID NO: 204, 206, 208, 210, 212, 214, 216, or 218.

Embodiment 50. Host cell according to specific embodiment 49, wherein the membrane protein is selected from the group of a) porters; b) P-P-bond-hydrolysis-driven transporters; c) β-Barrel Porins; d) Auxiliary transport proteins; e) Putative transport proteins; and f) Phosphotransfer-driven group translocators.

Embodiment 51. Host cell genetically modified for the production of a fucosyllactose, wherein the host cell comprises at least one nucleic acid sequence coding for a fucosyltransferase that transfers a fucose residue from a GDP-fucose donor to a lactose acceptor thereby synthesizing fucosyllactose, the cell further comprising i) a modified expression of an endogenous membrane protein enabling and/or enhancing fucosyllactose transport and/or ii) an expression of a heterologous membrane protein enabling and/or enhancing fucosyllactose transport, and wherein the membrane protein is selected from the group of a) porters and wherein the membrane protein is not belonging to the SET family; b) P-P-bond-hydrolysis-driven transporters; c) β-Barrel Porins; d) Auxiliary transport proteins; e) Putative transport proteins; and f) Phosphotransfer-driven group translocators.

Embodiment 52. Host cell according to any one of the specific embodiments 50 or 51, wherein the membrane protein is chosen from the group of membrane proteins as defined in any one of the specific embodiments 4 to 38.

Embodiment 53. Host cell genetically modified for the production of a fucosyllactose according to any one of the specific embodiments 50 or 51, wherein porter membrane protein is selected from MdfA from Escherichia coli K12 MG1655 with SEQ ID NO: 02, IceT from Escherichia coli K12 MG1655 with SEQ ID NO: 06, YnfM from Escherichia coli K12 MG1655 with SEQ ID NO: 04, Yhhs from Escherichia coli K12 MG1655 with SEQ ID NO: 08, EmrD from Escherichia coli K12 MG1655 with SEQ ID NO: 10, YdhC from Escherichia coli K12 MG1655 with SEQ ID NO: 12, YbdA from Escherichia coli K12 MG1655 with SEQ ID NO: 14, YdeE from Escherichia coli K12 MG1655 with SEQ ID NO: 16, MhpT from Escherichia coli K12 MG1655 with SEQ ID NO: 18, YebQ from Escherichia coli K12 MG1655 with SEQ ID NO: 20, YjhB from Escherichia coli K12 MG1655 with SEQ ID NO: 22, Bcr from Escherichia coli K12 MG1655 with SEQ ID NO: 24, FucP from Escherichia coli K12 MG1655 with SEQ ID NO: 26, WzxE from Escherichia coli K12 MG1655 with SEQ ID NO: 32, EmrE from Escherichia coli K12 MG1655 with SEQ ID NO: 38, Blon_2331 from Bifidobacterium longum subsp. infantis (strain ATCC 15697) with SEQ ID NO: 40, Blon_2332 from Bifidobacterium longum subsp. infantis (strain ATCC 15697) with SEQ ID NO: 42, Blon_0247 from Bifidobacterium longum subsp. infantis (strain ATCC 15697) with SEQ ID NO: 46, Blon_0245 from Bifidobacterium longum subsp. infantis (strain ATCC 15697) with SEQ ID NO: 48, Blon_0345 from Bifidobacterium longum subsp. infantis (strain ATCC 15697) with SEQ ID NO: 50, CDT2 from Neurospora crassa OR74A with SEQ ID NO: 52, CDT2 from Aspergillus oryzae RIB40 with SEQ ID NO: 54, Wzx from Chitinophaga sp. CF 118 with SEQ ID NO: 58, Wzx from Eubacterium sp. CAG:581 with SEQ ID NO: 60, Wzx from Dyadobacter soli (DSM 25329) with SEQ ID NO: 62, Wzx from Lactococcus raffinolactis (ATCC 43920) with SEQ ID NO: 64, Wzx from Prevotella ruminicola (AR32) with SEQ ID NO: 66, NAPO from Brachyspira hampsonii P280/1 with SEQ ID NO: 86, NAm from Actinobaculum suis (DSM 20639) with SEQ ID NO: 98, NAm from Ruminococcus gnavus with SEQ ID NO: 100, NAm from Curtobacterium sp. 314Chir4.1 with SEQ ID NO: 102, Nap from Niabella drilacis (DSM25811) with SEQ ID NO: 104, Nap from Saccharicrinis fermentans (DSM 9555) with SEQ ID NO: 106, mdtD from Citrobacter freundii MGH152 with SEQ ID NO: 108, mdtD from Citrobacter werkmanii NBRC 105721 with SEQ ID NO: 110, mdtD from Citrobacter amalonaticus with SEQ ID NO: 112, mdtD from Klebsiella oxytoca with SEQ ID NO: 114, mdtD from Escherichia alberti B156 with SEQ ID NO: 116, yegB from Salmonella enterica subsp. salamae with SEQ ID NO: 118, mdtD from Klebsiella pneumoniae 30684/NJST258_2 with SEQ ID NO: 120, Tcr_1_D38215 from Klebsiella pneumoniae with SEQ ID NO: 122, mdtD from Pseudocitrobacter faecalis with SEQ ID NO: 124, Cmr from Yokenella regensburgei (ATCC43003) with SEQ ID NO: 126, MdfA from Cronobacter muytjensii with SEQ ID NO: 128, MdfA from Klebsiella oxytoca with SEQ ID NO: 130, MFS from Citrobacter koseri with SEQ ID NO: 132, MdfA from Escherichia marmotae with SEQ ID NO: 134, Cmr from Shigella flexneri with SEQ ID NO: 136, MdfA from Salmonella enterica subsp. salamae with SEQ ID NO: 138, Cmr from Citrobacter youngae (ATCC 29220) with SEQ ID NO: 140, MdfA from Citrobacter freundii with SEQ ID NO: 142, MdfA from Enterobacter kobei with SEQ ID NO: 144, MdfA from Enterobacter sp. with SEQ ID NO: 146, MdfA from Lelliottia sp. WB101 with SEQ ID NO: 148, MdfA from Enterobacter ludwigii EcWSU1 with SEQ ID NO: 150, Sweet-like protein from Actinoplanes utahensis with SEQ ID NO: 152, Sweet-like protein from Chitinophagaceae bacterium PMG 246 with SEQ ID NO: 154, Sweet-like protein from Rhizobium sp. PDC82 with SEQ ID NO: 156, Sweet-like protein from Kineococcus rhizosphaerae (DSM 19711) with SEQ ID NO: 158, Sweet-like protein from Morganella morganii IS15 with SEQ ID NO: 160, Sweet-like protein from Geodermatophilus obscurus (strain ATCC 25078) with SEQ ID NO: 162, Sweet-like protein from Bradyrhizobium sp. BTAi1 with SEQ ID NO: 164, Sweet-like protein from Bradyrhizobium japonicum USDA 110 with SEQ ID NO: 166, Sweet-like protein from Xanthomonas campestris pv. vesicatoria str. 85-10 with SEQ ID NO: 168, Sweet-like protein from Herbaspirillum aquaticum with SEQ ID NO: 170, Sweet-like protein from Flavobacteria bacterium MS024-2A with SEQ ID NO: 172, rnd-like from Sinorhizobium medicae WSM419 with SEQ ID NO: 182, arabinose efflux from Azospirillum brasiliense LMG 04375 with SEQ ID NO: 184 or functional homolog or functional fragment of any of the above porter membrane protein or a protein having an amino acid sequence having at least 80% sequence identity to any of the MdfA, IceT, YnfM, Yhhs, EmrD, YdhC, YbdA, YdeE, MhpT, YebQ, YjhB, Bcr, FucP, WzxE, EmrE, Wzx, Blon_2331, Blon_2232, Blon_0247, Blon_0245, Blon_0345, NAPO, NAm, Nap, mdtD, YegB, Tcr_1_D38215, cmr, MFS, CDT2, md, Sweet-like or arabinose efflux membrane proteins with SEQ ID NOS 02, 06, 04, 08, 10, 12, 14, 16, 18, 20, 22, 24, 26, 32, 38, 40, 42, 46, 48, 50, 52, 54, 58, 60, 62, 64, 66, 86, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 182 or 184, respectively.

Embodiment 54. Host cell according to any one of the specific embodiments 50 or 51, wherein the P-P-bond-hydrolysis-driven transporter is selected from LmrA from Lactococcus lactis strain SRCM103457 with SEQ ID NO: 28, OppF from Escherichia coli strain K12 MG1655 with SEQ ID NO: 30, Wzk from Helicobacter pylori (strain ATCC 700392/26695) with SEQ ID NO: 36, Blon_2475 from Bifidobacterium longum subsp. infantis (strain ATCC 15697) with SEQ ID NO: 44, LpsE from Flavobacterium spartansii with SEQ ID NO: 68 or 72, LpsE from Sporomusa sphaeroides DSM 2875 with SEQ ID NO: 70 or 74, TolC from Candidatus Planktophila sulfonica with SEQ ID NO: 76, TolC from Butyrivibrio hungatei XBD2006 with SEQ ID NO: 78, MsbA from Roseburia intestinalis CAG:13 with SEQ ID NO: 80, MsbA from Pedobacter ginsengisoli with SEQ ID NO: 82, MsbA from Verrucomicrobia bacterium CG1_02_43_26 with SEQ ID NO: 84, Wzm from Rhizobium sp. Root149 with SEQ ID NO: 174, Wzm from Azospirillum brasiliense LMG 04375 with SEQ ID NO: 176, Wzm from Escherichia coli 113303 with SEQ ID NO: 196, Wzt from Rhizobium sp. Root149 with SEQ ID NO: 178, Wzt from Azospirillum brasiliense LMG 04375 with SEQ ID NO: 180, Wzt from Escherichia coli 113303 with SEQ ID NO: 194, Nodj from Bradyrhizobium japonicum USDA 110 with SEQ ID NO: 188 or 190, malE from Escherichia coli K-12 MG1655 with SEQ ID NO: 206, malK from Escherichia coli K-12 MG1655 with SEQ ID NO: 208, araF from Escherichia coli K-12 MG1655 with SEQ ID NO: 214, xylF from Escherichia coli K-12 MG1655 with SEQ ID NO: 216, or ytfQ from Escherichia coli K-12 MG1655 with SEQ ID NO: 218 or functional homolog or functional fragment of any one of the above P-P-bond-hydrolysis-driven transporter membrane protein or a protein having an amino acid sequence having at least 80% sequence identity to any of the LmrA, OppF, Wzk, Blon_2475, LpsE, TolC, MsbA, Wzm, Wzt, Nodj, malE, malK, araF, xylF or ytfQ membrane proteins with SEQ ID NO: 28, 30, 36, 44, 68, 72, 70, 74, 76, 78, 80, 82, 84, 174, 176, 196, 178, 180, 194, 188, 190, 206, 208, 214, 216, or 218 respectively.

Embodiment 55. Host cell according to any one of the specific embodiments 50 or 51, wherein the putative transport protein is selected from Cytochrome C biogenesis protein from Helicobacter pylori with SEQ ID NO: 56, CutC from Clostridium sp. CAG: 1013 with SEQ ID NO: 90, CutC from Odoribacter splanchnicus DSM 20712 with SEQ ID NO: 92, CutC from Mitsuaria sp. PDC51 with SEQ ID NO: 94, CutC from Prevotella intermedia ATCC 25611 (DSM 20706) with SEQ ID NO: 96, ybjM from Escherichia coli K12 MG1655 with SEQ ID NO: 190, ybjM from Enterobacteriaceae bacterium ENNIH1 with SEQ ID NO: 192 or functional homolog or functional fragment of any one of the above putative transport protein or protein having an amino acid sequence having at least 80% sequence identity to any one of the CytC, CutC or ybjM membrane protein with SEQ ID NO: 56, 90, 92, 94, 96, 190 or 192, respectively.

Embodiment 56. Host cell according to any one of the specific embodiments 50 or 51, wherein the β-barrel porin is selected from Wza from Escherichia coli K12 MG1655 with SEQ ID NO: 34 or lamB from Escherichia coli K12 MG1655 with SEQ ID NO: 204 or functional homolog or functional fragment to any one of the Wza or lamB protein or a sequence having at least 80% sequence identity to any one of the Wza or lamB membrane protein with SEQ ID NO: 34 or 204, respectively.

Embodiment 57. Host cell according to any one of the specific embodiments 50 or 51, wherein the auxiliary transport protein is selected from Wzc from Thermotoga maritima (strain ATCC 43589/MSB8/DSM 3109/JCM 10099) with SEQ ID NO: 88, or functional homolog or functional fragment thereof or a sequence having at least 80% sequence identity to the Wzc membrane protein with SEQ ID NO: 88.

Embodiment 58. Host cell according to any one of the specific embodiments 50 or 51, wherein the phosphotransfer-driven group translocators is selected from nagE from Escherichia coli K12 MG1655 with SEQ ID NO: 210 or srlB from Escherichia coli K12 MG1655 with SEQ ID NO: 212 or functional homolog or functional fragment of any of the nagE or srlB membrane protein or a sequence having at least 80% sequence identity to any one of the nagE or srlB membrane protein with SEQ ID NO: 210 or 212, respectively.

Embodiment 59. Cell according to any one of the previous specific embodiments 49 to 58, wherein the membrane protein is a transporter protein involved in transport of compounds across the outer membrane of the cell wall.

Embodiment 60. Cell according to any one of the specific embodiments 49 to 59, wherein the cell is stably cultured in a medium.

Embodiment 61. Cell according to any one of the specific embodiments 49 to 60, wherein the cell is selected from the group consisting of microorganism, plant, or animal cells, preferably the microorganism is a bacterium, fungus or a yeast, preferably the plant is a rice, cotton, rapeseed, soy, maize or corn plant, preferably the animal is an insect, fish, bird or non-human mammal.

Embodiment 62. Host cell according to specific embodiment 61, wherein the cell is an Escherichia coli cell.

Embodiment 63. Cell according to any one of the specific embodiments 49 to 62 wherein the cell comprises a catabolic pathway for selected mono-, di- or oligosaccharides that is at least partially inactivated, the mono-, di-, or oligosaccharides being involved in and/or required for the synthesis of fucosyllactose.

Embodiment 64. Cell according to any one of the specific embodiments 49 to 63 wherein the fucosyllactose is 2′-fucosyllactose, 3-fucosyllactose or difucosyllactose.

Embodiment 65. Method for the production of fucosyllactose, comprising the steps of: a) providing a cell according to any one of the specific embodiments 49 to 64, b) culturing the cell in a medium under conditions permissive for the production of the fucosyllactose, c) separating the fucosyllactose from the cultivation.

Embodiment 66. Use of a membrane protein selected from the group of membrane proteins as defined in any one of the specific embodiments 1 to 38, for fucosyllactose transport, in the fermentative production of fucosyllactose.

Embodiment 67. Use of a cell according to any one of the specific embodiments 49 to 64, for the production of fucosyllactose.

Embodiment 68. Use of a cell according to specific embodiment 67 wherein the fucosyllactose is 2′-fucosyllactose, 3-fucosyllactose or difucosyllactose.

### EXAMPLES

### Example 1: Identification of Membrane Protein Families

An HMM is a probabilistic model called profile hidden Markov models. It characterizes a set of aligned proteins into a position-specific scoring system. Amino acids are given a score at each position in the sequence alignment according to the frequency by which they occur (Eddy, S. R. 1998, Profile hidden Markov models. Bioinformatics 14: 755-63). HMMs have wide utility, as is clear from the numerous databases that use this method for protein classification, including Pfam, InterPro, SMART, TIGRFAM, PIRSF, PANTHER, SFLD, Superfamily and Gene3D.

HMMsearch from the HMMER package 3.2.1 (http://hmmer.org/_) as released on 13 Jun. 2019 can use this HMM to search sequence databases for sequence homologs. Sequence databases that can be used are, for example, but not limited to: the NCBI nr Protein Database (NR; https://www.ncbi.nlm.nih.gov/protein), UniProt Knowledgebase (UniProtKB, https://www.uniprot.org/help/uniprotkb) and the SWISS-PROT database (https://web.expasy.org/docs/swiss-prot_guideline.html).

Membrane protein families were classified based on the eggNOG database 1.0.2 (www.ncbi.nlm.nih.gov/pmc/articles/PMC6324079/; eggnog.embl.de/#/app/home) as released on 3 Nov. 2017, the TCDB database (www.tcdb.org/public/tcdb) as released on 17 Jun. 2019, InterPro 75.0 (www.ebi.ac.uk/interpro/) as released on 4 Jul. 2019 and PFAM domains using Pfam 32.0 (pfam.xfam.org/) as released on September 2018. The eggNOG database is a public database of orthology relationships, gene evolutionary histories and functional annotations. The Transporter Classification DataBase (TCDB) is analogous to the Enzyme Commission (EC) system for classifying enzymes and incorporates both functional and phylogenetic information. The Pfam and InterPro databases are a large collection of protein families. Other protein domains like SMART (smart.embl-heidelberg.de/), TIGRFAM (https://www.jcvi.org/tigrfams), PIRSF (proteininformationresource.org/pirwww/dbinfo/pirsf.shtml), PANTHER (pantherdb.org/), SFLD (sfld.rbvi.ucsf.edu/archive/django/index.html), Superfamily (supfam.org/) and Gene3D (gene3d.biochem.ucl.ac.uk/Gene3D/), NCBI Conserved Domains (www.ncbi.nlm.nih.gov/Structure/cdd/wrpsb.cgi) can also be used.

Identification of eggNOG families was done by using a standalone version of eggNOG-mapper (github.com/eggnogdb/eggnog-mapper) based on eggnogdb 1.0.2 as released on 3 Nov. 2017. For each of the eggNOG families an HMM can be downloaded on the eggNOG website and can be used for HMMsearch to the protein databases.

Identification of the TCDB family was done by blasting (blastp) to the TCDB database as released on 17 Jun. 2019. New members of the obtained family can be retrieved on the website (www.tcdb.org/download.php). Fasta files can be used as input in blastp to the protein databases.

Identification of the PFAM domains was done by an online search on pfam.xfam.org/search#tabview=tab1 as released on September 2018. The HMM for the obtained family was downloaded in “Curation & model.” HMMsearches with this model to the protein databases will identify new family members. Sequences comprising the InterPro hit can also be downloaded from the PFAM website.

Identification of the InterPro (super)families, domains and sites was done by using the online tools on www.ebi.ac.uk/interpro/ or a standalone version of InterProScan (www.ebi.ac.uk/interpro/download.html), both based on InterPro 75.0 as released on 4 Jul. 2019.

InterPro is a composite database combining the information of many databases of protein motifs and domains. The HMM of the InterPro domain and/or (super)families can be obtained from InterProScan and can be used to identify new family members in the protein databases. Sequences comprising the InterPro hit can also be downloaded from the InterPro website (“Protein Matched”) or can be queried on the UniProt website (www.uniprot.org).

### Example 2: Identification of Membrane Proteins or Protein Sequences Useful in the Methods of the Disclosure

A first set of membrane proteins or protein sequences were found by identifying the PFAM domains of the membrane proteins found in the neighborhood of fucosyltransferases and selecting membrane proteins having any one of the PFAM domains identified, as exemplified in Example 1. Protein identifiers belonging to fucosyltransferase families IPR001053 (GT10) and IPR002516 (GT11) were extracted from UniProtKB/trembl, as defined by InterPro 75.0 as released on 4 Jul. 2019. These identifiers were used as input in the genome neighborhood tool efi.igb.illinois.edu/efi-gnt/ as released on 19 Jun. 2019. EFI-GNT (EFI Genome Neighborhood Tool) allows exploration of the genome neighborhoods and is focused on placing protein families and superfamilies into a genomic context. A sequence similarity network (SSN) is used as an input. Each sequence within an SSN is used as a query for interrogation of its genome neighborhood. EFI-GNT enables exploration of the genome neighborhoods for sequences in SSN clusters in order to facilitate their assignment of function.

A neighborhood window size of 14 was selected. Neighboring genes were classified based on their PFAM domain. Membrane proteins with the following PFAM domains are present near GT10 (IPR001503) and GT11 (IPR002516) fucosyltransferases: PF00005, PF00006, PF00023, PF00083, PF00092, PF00115, PF00116, PF00122, PF00209, PF00213, PF00230, PF00231, PF00254, PF00359, PF00375, PF00381, PF00391, PF00401, PF00403, PF00474, PF00484, PF00520, PF00528, PF00529, PF00543, PF00571, PF00593, PF00625, PF00654, PF00664, PF00689, PF00690, PF00702, PF00860, PF00873, PF00892, PF00893, PF00902, PF00909, PF00916, PF00939, PF00999, PF01032, PF01061, PF01103, PF01203, PF01235, PF01384, PF01496, PF01544, PF01547, PF01554, PF01566, PF01578, PF01614, PF01618, PF01656, PF01699, PF01740, PF01741, PF01758, PF01810, PF01813, PF01891, PF01895, PF01899, PF01943, PF02026, PF02080, PF02133, PF02136, PF02225, PF02254, PF02277, PF02302, PF02321, PF02355, PF02378, PF02386, PF02417, PF02447, PF02501, PF02563, PF02632, PF02652, PF02653, PF02690, PF02706, PF02874, PF02896, PF03030, PF03083, PF03186, PF03222, PF03412, PF03459, PF03471, PF03544, PF03547, PF03548, PF03567, PF03605, PF03606, PF03610, PF03616, PF03814, PF03840, PF03865, PF03932, PF04193, PF04277, PF04389, PF04966, PF05134, PF05140, PF05524, PF05552, PF05977, PF06251, PF06826, PF06835, PF07264, PF07549, PF07660, PF07670, PF07685, PF07690, PF07715, PF07885, PF07969, PF08239, PF08279, PF08334, PF08352, PF08402, PF08479, PF10531, PF11356, PF11612, PF12156, PF12399, PF12796, PF12822, PF12848, PF12974, PF13306, PF13347, PF13409, PF13410, PF13416, PF13417, PF13440, PF13442, PF13462, PF13466, PF13473, PF13499, PF13505, PF13520, PF13531, PF13599, PF13609, PF13637, PF13807, PF13855, PF14524, PF14667, PF16327, PF17912 and PF18412.

### Example 3: Materials and Methods Escherichia coli

### Media

The Luria Broth (LB) medium consisted of 1% tryptone peptone (Difco, Erembodegem, Belgium), 0.5% yeast extract (Difco) and 0.5% sodium chloride (VWR. Leuven, Belgium). The minimal medium used in the cultivation experiments in 96-well plates or in shake flasks contained 2.00 g/L NH4Cl, 5.00 g/L (NH4)2SO4, 2.993 g/L KH2PO4, 7.315 g/L K2HPO4, 8.372 g/L MOPS, 0.5 g/L NaCl, 0.5 g/L MgSO4.7H2O, 30 g/L sucrose or another carbon source when specified in the examples, 1 ml/L vitamin solution, 100 μL/L molybdate solution, and 1 mL/L selenium solution. As specified in the respective examples, 20 or 45 g/L lactose was additionally added to the medium as precursor. The medium was set to a pH of 7 with 1 M KOH. Vitamin solution consisted of 3.6 g/L FeCl2.4H2O, 5 g/L CaCl2.2H2O, 1.3 g/L MnCl2.2H2O, 0.38 g/L CuCl2.2H2O, 0.5 g/L CoCl2.6H2O, 0.94 g/L ZnCl2, 0.0311 g/L H3BO4, 0.4 g/L Na2EDTA.2H2O and 1.01 g/L thiamine.HCl. The molybdate solution contained 0.967 g/L NaMoO4.2H2O. The selenium solution contained 42 g/L Seo2.

The minimal medium for fermentations contained 6.75 g/L NH4Cl, 1.25 g/L (NH4)2SO4, 2.93 g/L KH2PO4 and 7.31 g/L KH2PO4, 0.5 g/L NaCl, 0.5 g/L MgSO4.7H2O, 30 g/L sucrose, 1 mL/L vitamin solution, 100 μL/L molybdate solution, and 1 mL/L selenium solution with the same composition as described above. As specified in the respective examples, 20 g/L lactose was additionally added to the medium as precursor.

Complex medium was sterilized by autoclaving (121° C., 21′) and minimal medium by filtration (0.22 μm Sartorius). When necessary, the medium was made selective by adding an antibiotic (e.g., chloramphenicol (20 mg/L), carbenicillin (100 mg/L), spectinomycin (40 mg/L) and/or kanamycin (50 mg/L)).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007).

Plasmids for membrane protein and for fucosyltransferase expression were constructed in a pSC101 on (Rep10-v3) and pMB1 on containing backbone vector, respectively, using Golden Gate assembly. All membrane protein and fucosyltransferase encoding genes were synthetically synthetized at Twist Biosciences (San Francisco, USA). Polynucleotide sequences of the membrane proteins and the corresponding membrane protein polypeptides are shown in SEQ ID NOS: 1 to 196 and SEQ ID NOS: 204 to 218 and enlisted in Table 1. The fucosyltransferases used in the examples are 3-fucosyltransferases FT1 with nucleic acid and protein sequence SEQ ID NOS: 197 and 198 respectively and FT2 with SEQ ID NOS: 199 and 200. The 2-fucosyltransferases used are HpFutC with SEQ ID NOS: 201 and 202, referred to here as FT3, and FT4 with nucleic acid and protein sequence SEQ ID NOS: 219 and 220, respectively. Both membrane protein and fucosyltransferase genes were expressed in different transcriptional units (TUs) using specific promoter, UTR and terminator combinations as enlisted in Table 2. The genes were expressed using promoters MutalikP5 (“PROM0005_MutalikP5”), MutalikP12 (“PROM0012_MutalikP12”), apFAB146 (“PROM0032”) and MutalikP10 (“PROM0010_MutalikP10”) (as described by Mutalik t al. (Nat. Methods 2013, No. 10, 354-360)) and promoters p22 (PROM015_p22) and p14 (PROM0016p14) both as described by De Mey et al. (BMC Biotechnology 2007, 7:34)). UTR's used comprise Gene-LeuAB-BCD2 (“UTR0002_Gene10-LeuAB-BCD2”), BCD1 (“UTR003_BCD1”), Gene10_LeuL (“UTR0011_Gene10_LeuL”), ThrA_BCD2 (“UTR0013_ThrA_BCD2”), GalE_LeuAB (“UTR0014_GalE_LeuAB”), GalE_lptFG (“UTR0038_GalE_IptFG”) and uspF_iptFG (“UTR55_uspF_iptFG”) (as described by Mutalik et al. (Nat. Methods 2013, No. 10, 354-360)). Terminator used in the examples is TER0010_T7 Early as described by Dunn et al. (Nucleic Acids Res. 1980, 8(10), 2119-32). Table 3 shows the overview of the transcriptional units used in the examples by combination of the above promoter UTRs and terminator. Expression could be further facilitated by optimizing the codon usage to the codon usage of the expression host. Genes were optimized using the tools of the supplier.

TABLE 1

| SEQ | | | | Country of origin |
|---|---|---|---|---|
| ID NO: | Name/ | | | of digital sequence |
| (protein) | TCDB group | Organism | Origin | information |
| 2 | EcMdfA | E. coli K12 MG1655 | Synthetic | USA |
| 4 | EcYnfM | E. coli K12 MG1655 | Synthetic | USA |
| 6 | EcIceT | E. coli K12 MG1655 | Synthetic | USA |
| 8 | EcYhhs | E. coli K12 MG1655 | Synthetic | USA |
| 10 | EcEmrD | E. coli K12 MG1655 | Synthetic | USA |
| 12 | EcYdhC | E. coli K12 MG1655 | Synthetic | USA |
| 14 | EcYbdA | E. coli K12 MG1655 | Synthetic | USA |
| 16 | EcYdeE | E. coli K12 MG1655 | Synthetic | USA |
| 18 | EcMhpT | E. coli K12 MG1655 | Synthetic | USA |
| 20 | EcYebQ | E. coli K12 MG1655 | Synthetic | USA |
| 22 | EcYjhB | E. coli K12 MG1655 | Synthetic | USA |
| 24 | EcBcr | E. coli K12 MG1655 | Synthetic | USA |
| 26 | EcFucP | E. coli K12 MG1655 | Synthetic | USA |
| 28 | LllmrA | Lactococcus lactis | Synthetic | South Korea |
| | | strain SRCM103457 | | |
| 30 | EcOppF | E. coli strain K12 | Synthetic | USA |
| | | (MG1655) | | |
| 32 | EcWzxE | E. coli strain K12 | Synthetic | USA |
| | | (MG1655) | | |
| 34 | EcWza | E. coli strain K12 | Synthetic | USA |
| | | (MG1655) | | |
| 36 | HpWzk | Helicobacter pylori | Synthetic | United Kingdom |
| | | strain ATCC 700392/ | | |
| | | 26695 | | |
| 38 | EcEmrE | E. coli strain K12 | Synthetic | USA |
| 40 | Blon_2331 | B. longum subsp. | Synthetic | Germany |
| | | Infantis (strain | | |
| | | ATCC 15697) | | |
| 42 | Blon_2332 | B. longum subsp. | Synthetic | Germany |
| | | Infantis (strain | | |
| | | ATCC 15697) | | |
| 44 | Blon_2475 | B. longum subsp. | Synthetic | Germany |
| | | Infantis (strain | | |
| | | ATCC 15697) | | |
| 46 | Blon_0247 | B. longum subsp. | Synthetic | Germany |
| | | Infantis (strain | | |
| | | ATCC 15697) | | |
| 48 | Blon_0245 | B. longum subsp. | Synthetic | Germany |
| | | Infantis (strain | | |
| | | ATCC 15697) | | |
| 50 | Blon_0345 | B. longum subsp. | Synthetic | Germany |
| | | Infantis (strain | | |
| | | ATCC 15697) | | |
| 52 | NcCDT2 | Neurospora crassa | Synthetic | USA |
| | | OR74A | | |
| 54 | AoCDT2 | Aspergillus oryzae | Synthetic | Japan |
| | | RIB40 | | |
| 56 | HpCytC | Helicobacter pylori | Synthetic | United Kingdom |
| | | (Campylobacter pylori) | | |
| 58 | ChWzx | Chitinophaga sp. CF118 | Synthetic | USA |
| 60 | EuWzx | Eubacterium sp. | Synthetic | Denmark |
| | | CAG:581 | | |
| 62 | DsWzx | Dyadobacter soli DSM | Synthetic | South Korea |
| | | 25329 contig | | |
| | | Ga0069981_102 | | |
| 64 | LrWzx | Lactococcus | Synthetic | Unknown |
| | | raffinolactis | | |
| | | (ATCC 43920) | | |
| 66 | PrWzx | Prevotella ruminicola | Synthetic | USA |
| | | (AR32) | | |
| 68 | FsLpsE | Flavobacterium | Synthetic | USA |
| | | spartansii | | |
| 70 | SsLpsE | Sporomusa sphaeroides | Synthetic | Germany |
| | | DSM 2875 | | |
| 72 | FsLpsE | Flavobacterium | Synthetic | USA |
| | | spartansii | | |
| 74 | SsLpsE | Sporomusa sphaeroides | Synthetic | Germany |
| | | DSM 2875 | | |
| 76 | PsTolC | Candidatus Planktophila | Synthetic | Switzerland |
| | | sulfonica | | |
| 78 | BhTolC | Butyrivibrio hungatei | Synthetic | USA |
| | | XBD2006 | | |
| 80 | RiMsbA | Roseburia intestinalis | Synthetic | Denmark |
| | | CAG:13 | | |
| 82 | PgMsbA | Pedobacter ginsengisoli | Synthetic | South Korea |
| 84 | VbMsbA | Verrucomicrobia | Synthetic | USA |
| | | bacterium | | |
| | | CG1_02_43_26 | | |
| 86 | BhNAPO | Brachyspira hampsonii | Synthetic | UK |
| | | P280/1 | | |
| 88 | TmWzc | Thermotoga maritima | Synthetic | Italy |
| | | (strain ATCC 43589/ | | |
| | | MSB8/DSM 3109/ | | |
| | | JCM 10099) | | |
| 90 | ClCutC | Clostridium sp. | Synthetic | Denmark |
| | | CAG:1013 | | |
| 92 | OsCutC | Odoribacter | Synthetic | Unknown |
| | | splanchnicus | | |
| | | DSM 20712 | | |
| 94 | MiCutC | Mitsuaria sp. PDC51 | Synthetic | USA |
| 96 | PiCutC | Prevotella intermedia | Synthetic | USA |
| | | ATCC 25611 | | |
| | | (DSM 20706) | | |
| 98 | AsNAm | Actinobaculum suis | Synthetic | Unknown |
| | | DSM 20639 | | |
| 100 | RgNAm | Ruminococcus gnavus | Synthetic | USA |
| 102 | CuNAm | Curtobacterium sp. | Synthetic | USA |
| | | 314Chir4.1 | | |
| 104 | NdNap | Niabella drilacis | Synthetic | Germany |
| | | DSM25811 | | |
| 106 | SfNap | Saccharicrinis | Synthetic | unknown |
| | | fermentans DSM | | |
| | | 9555 = JCM 21142 | | |
| 108 | mdtD | Citrobacter freundii | Synthetic | USA |
| | | MGH152 | | |
| 110 | mdtD | Citrobacter werkmanii | Synthetic | Belgium, not further |
| | | NBRC 105721 | | specified |
| 112 | mdtD | Citrobacter amalonaticus | Synthetic | USA |
| 114 | mdtD | Klebsiella oxytoca | Synthetic | United Kingdom |
| 116 | mdtD | Escherichia albertii | Synthetic | USA |
| | | B156 | | |
| 118 | yegB | Salmonella enterica | Synthetic | United Kingdom |
| | | subsp. Salamae | | |
| 120 | mdtD | Klebsiella pneumoniae | Synthetic | USA |
| | | 30684/NJST258_2 | | |
| 122 | Tcr_1_D38215 | Klebsiella pneumoniae | Synthetic | USA |
| 124 | mdtD | Pseudocitrobacter | Synthetic | USA |
| | | faecalis | | |
| 126 | Cmr | Yokenella regensburgei | Synthetic | United Kingdom |
| | | ATCC43003 | | |
| 128 | MdfA | Cronobacter muytjensii | Synthetic | USA |
| 130 | MdfA | Klebsiella oxytoca | Synthetic | Sweden |
| 132 | MFS | Citrobacter koseri | Synthetic | USA |
| | | (Citrobacter diversus) | | |
| 134 | MdfA | Escherichia marmotae | Synthetic | USA |
| 136 | Cmr | Shigella flexneri | Synthetic | United Kingdom |
| 138 | MdfA | Salmonella enterica | Synthetic | Denmark |
| | | subsp. Salamae | | |
| 140 | Cmr/MdfA | Citrobacter youngae | Synthetic | USA |
| | | ATCC 29220 | | |
| 142 | MdfA | Citrobacter freundii | Synthetic | USA |
| 144 | MdfA/DHA1/MFS | Enterobacter kobei | Synthetic | USA |
| 146 | MdfA | Enterobacter sp. | Synthetic | Australia |
| 148 | MdfA | Lelliottia sp. WB101 | Synthetic | USA |
| 150 | MdfA | Enterobacter ludwigii | Synthetic | USA |
| | | EcWSU1 | | |
| 152 | Sweet-like | Actinoplanes utahensis | Synthetic | USA |
| 154 | Sweet-like | Chitinophagaceae | Synthetic | United Kingdom |
| | | bacterium PMG 246 | | |
| 156 | Sweet-like | Rhizobium sp. PDC82 | Synthetic | USA |
| 158 | Sweet-like | Kineococcus | Synthetic | South-Korea |
| | | rhizosphaerae | | |
| | | DSM 19711 | | |
| 160 | Sweet-like | Morganella morganii | Synthetic | Austria |
| | | IS15 | | |
| 162 | Sweet-like | Geodermatophilus | Synthetic | USA |
| | | obscurus (strain ATCC | | |
| | | 25078/DSM 43160/ | | |
| | | JCM 3152/G-20) | | |
| 164 | Sweet-like | Bradyrhizobium sp. | Synthetic | USA |
| | | BTAi1 | | |
| 166 | Sweet-like | Bradyrhizobium | Synthetic | USA |
| | | japonicum USDA 110 | | |
| 168 | Sweet-like | Xanthomonas | Synthetic | South Korea |
| | | campestris pv. | | |
| | | vesicatoria str. 85-10 | | |
| 170 | Sweet-like | Herbaspirillum | Synthetic | USA |
| | | aquaticum | | |
| 172 | Sweet-like | Flavobacteria bacterium | Synthetic | USA |
| | | MS024-2A | | |
| 174 | wzm | Rhizobium sp. Root149 | Synthetic | Germany |
| 176 | wzm | Azospirillum brasiliense | Synthetic | Belgium, Flanders |
| | | LMG 04375 | | |
| 178 | wzt | Rhizobium sp. Rootl49 | Synthetic | Germany |
| 180 | wzt | Azospirillum brasiliense | Synthetic | Belgium, Flanders |
| | | LMG 4375 | | |
| 182 | rnd-like | Sinorhizobium medicae | Synthetic | USA |
| | | WSM419 | | |
| 184 | arabinose efflux | Azospirillum brasiliense | Synthetic | Belgium, Flanders |
| | | LMG 4375 | | |
| 186 | nodj | Bradyrhizobium | Synthetic | USA |
| | | japonicum USDA 110 | | |
| 188 | nodi | Bradyrhizobium | Synthetic | USA |
| | | japonicum USDA 110 | | |
| 190 | ybjM | E. coli K12 MG1655 | Synthetic | USA |
| 192 | ybjM-like | Enterobacteriaceae | Synthetic | USA |
| | | bacterium ENNIH1 | | |
| 194 | wzt | Escherichia coli 113303 | Synthetic | USA |
| 196 | wzm | Escherichia coli 113303 | Synthetic | USA |
| 198 | FT1 | Helicobacter pylori | Synthetic | Australia |
| 200 | FT2 | Basilea psittacipulmonis | Synthetic | Switzerland |
| | | JF4266 (DSM 24701) | | |
| 202 | HpFutC (FT3) | Helicobacter pylori | Synthetic | Australia |
| 204 | lamB | E. coli K12 MG1655 | Synthetic | USA |
| 206 | malE | E. coli K12 MG1655 | Synthetic | USA |
| 208 | malK | E. coli K12 MG1655 | Synthetic | USA |
| 210 | nagE | E. coli K12 MG1655 | Synthetic | USA |
| 212 | srlB | E. coli K12 MG1655 | Synthetic | USA |
| 214 | araF | E. coli K12 MG1655 | Synthetic | USA |
| 216 | xylF | E. coli K12 MG1655 | Synthetic | USA |
| 218 | ytfQ | E. coli K12 MG1655 | Synthetic | USA |
| 220 | FT4 | Helicobacter sp. MIT | Synthetic | USA |
| | | 01-6242 | | |

TABLE 2

| SEQ | | | | | |
|---|---|---|---|---|---|
| ID NO: | | | | | |
| (protein) | Family | EggNOG | PFAM | InterPro | TCDB |
| 2 | porters | 07QF7 | PF07690 | IPR011701 | 2.A.1.2 |
| | | | | IPR020846 | |
| | | | | IPR036259 | |
| | | | | IPR005829 | |
| 4 | porters | 05D94 | PF07690 | IPR011701 | 2.A.1.36 |
| | | | | IPR020846 | |
| | | | | IPR036259 | |
| | | | | IPR005829 | |
| 6 | porters | 05C0R | PF07690 | IPR004638 | 2.A.1.3 |
| | | | | IPR011701 | |
| | | | | IPR020846 | |
| | | | | IPR036259 | |
| | | | | IPR023721 | |
| | | | | IPR001411 | |
| 8 | porters | 05F9N | PF07690 | IPR011701 | 2.A.1.46 |
| | | | | IPR020846 | |
| | | | | IPR036259 | |
| | | | | IPR023008 | |
| 10 | porters | 05E5M | PF07690 | IPR011701 | 2.A.1.2 |
| | | | | IPR020846 | |
| | | | | IPR036259 | |
| | | | | IPR004734 | |
| 12 | porters | 05CZQ | PF07690 | IPR004812 | 2.A.1.2 |
| | | | | IPR011701 | |
| | | | | IPR020846 | |
| | | | | IPR036259 | |
| 14 | porters | 05E8G | PF05977 | IPR023722 | 2.A.1.38 |
| | | | | IPR020846 | |
| | | | | IPR036259 | |
| | | | | IPR010290 | |
| 16 | porters | 05E5W | PF07690 | IPR011701 | 2.A.1.2 |
| | | | | IPR020846 | |
| | | | | IPR036259 | |
| 18 | porters | 08SC4 | PF07690 | IPR011701 | 2.A.1.15 |
| | | | | IPR020846 | |
| | | | | IPR036259 | |
| | | | | IPR005829 | |
| 20 | porters | 08H15 | PF07690 | IPR011701 | 2.A.1.3 |
| | | | | IPR020846 | |
| | | | | IPR036259 | |
| | | | | IPR001411 | |
| 22 | porters | 05JHE | PF07690 | IPR011701 | 2.A.1.12 |
| | | | | IPR020846 | |
| | | | | IPR036259 | |
| 24 | porters | 05C2C | PF07690 | IPR004812 | 2.A.1.2 |
| | | | | IPR011701 | |
| | | | | IPR020846 | |
| | | | | IPR036259 | |
| | | | | IPR004734 | |
| 26 | porters | 05EDR | PF07690 | IPR005275 | 2.A.1.7 |
| | | | | IPR011701 | |
| | | | | IPR020846 | |
| | | | | IPR036259 | |
| 28 | P—P-bond- | 05BZ1 | PF00664 | IPR003593 | 3.A.1.11 |
| | hydrolysis-driven | | PF00005 | IPR011527 | |
| | transporters | | | IPR036640 | |
| | | | | IPR003439 | |
| | | | | IPR017871 | |
| | | | | IPR027417 | |
| | | | | IPR039421 | |
| 30 | P—P-bond- | 08JQ7 | PF00005 | IPR003593 | 3.A.1.5 |
| | hydrolysis-driven | | PF08352 | IPR003439 | |
| | transporters | | | IPR017871 | |
| | | | | IPR013 563 | |
| | | | | IPR027417 | |
| 32 | porters | 05EGZ | PF01943 | IPR002797 | 2.A.66 |
| | | | | IPR032896 | |
| 34 | β-Barrel Porins | 05DAY | PF02563 | IPR003715 | 1.B.18 |
| | | | PF10531 | IPR0195 54 | |
| | | | PF18412 | IPR040716 | |
| 36 | P—P-bond- | 05BZ1 | PF00005 | IPR003593 | 3.A.1.10 |
| | hydrolysis-driven | | | IPR011527 | |
| | transporters | | | IPR036640 | |
| | | | | IPR003439 | |
| | | | | IPR017871 | |
| | | | | IPR027417 | |
| | | | | IPR039421 | |
| 38 | porters | 0814C | PF00893 | IPR000390 | 2.A.7.1 |
| 40 | porters | 07RBJ | PF13347 | IPR039672 | 2.A.2 |
| | | | | IPR036259 | |
| | | | | IPR001927 | |
| 42 | porters | 07RBJ | PF13347 | IPR039672 | 2.A.2 |
| | | | | IPR036259 | |
| | | | | IPR001927 | |
| 44 | P—P-bond- | 08IJ9 | PF00005 | IPR003593 | 3.A.1.1 |
| | hydrolysis-driven | | PF17912 | IPR003439 | |
| | transporters | | | IPR017871 | |
| | | | | IPR008995 | |
| | | | | IPR040582 | |
| | | | | IPR027417 | |
| 46 | porters | 08N8A | PF07690 | IPR011701 | 2.A.1.68 |
| | | | | IPR020846 | |
| | | | | IPR036259 | |
| 48 | porters | 05DXI | PF07690 | IPR011701 | 2.A.1.81 |
| | | | | IPR020846 | |
| | | | | IPR036259 | |
| 50 | porters | 08N8A | PF07690 | IPR011701 | 2.A.1.68 |
| | | | | IPR020846 | |
| | | | | IPR036259 | |
| 52 | porters | 07QNK | PF00083 | IPR020846 | 2.A.1.1 |
| | | | | IPR005828 | |
| | | | | IPR036259 | |
| | | | | IPR003663 | |
| | | | | IPR005829 | |
| 54 | porters | 07QNK | PF00083 | IPR020846 | 2.A.1.1 |
| | | | | IPR005828 | |
| | | | | IPR036259 | |
| 56 | Putative transport | 05CRE | PF01578 | IPR002541 | 9.B.14 |
| | proteins | | PF05140 | IPR007816 | |
| 58 | porters | 07T5E | PF14667 | IPR029303 | 2.A.66 |
| | | | PF13440 | | |
| 60 | porters | 07VQ3 | PF01943 | IPR029303 | 2.A.66 |
| | | | PF14667 | IPR002797 | |
| 62 | porters | 08Z4Q | PF13440 | | 2.A.66 |
| 64 | porters | 07VQ3 | PF01943 | IPR002797 | 2.A.66 |
| 66 | porters | 05PSV | PF13440 | | 2.A.66 |
| 68 | P—P-bond- | 05EY8 | PF00005 | IPR003593 | 3.A.1.10 |
| | hydrolysis-driven | | PF14524 | IPR003439 | |
| | transporters | | | IPR027417 | |
| | | | | IPR029439 | |
| 70 | P—P-bond- | 05EY8 | PF00005 | IPR003593 | 3.A.1.10 |
| | hydrolysis-driven | | | IPR003439 | |
| | transporters | | | IPR027417 | |
| 72 | P—P-bond- | 07V1T | PF01061 | IPR013525 | 3.A.1.10 |
| | hydrolysis-driven | | | IPR000412 | |
| | transporters | | | | |
| 74 | P—P-bond- | 07V1T | PF01061 | IPR013525 | 3.A.1.10 |
| | hydrolysis-driven | | | IPR000412 | |
| | transporters | | | | |
| 76 | P—P-bond- | 05BZ1 | PF00005 | IPR003593 | 3.A.1.11 |
| | hydrolysis-driven | | | IPR011527 | |
| | transporters | | | IPR036640 | |
| | | | | IPR003439 | |
| | | | | IPR017871 | |
| | | | | IPR027417 | |
| | | | | IPR039421 | |
| 78 | P—P-bond- | 05BZ1 | PF00664 | IPR003593 | 3.A.1.10 |
| | hydrolysis-driven | | PF00005 | IPR011527 | |
| | transporters | | | IPR036640 | |
| | | | | IPR003439 | |
| | | | | IPR017871 | |
| | | | | IPR027417 | |
| | | | | IPR039421 | |
| 80 | P—P-bond- | 05BZ1 | PF00664 | IPR003593 | 3.A.1.10 |
| | hydrolysis-driven | | PF00005 | IPR011527 | |
| | transporters | | | IPR036640 | |
| | | | | IPR003439 | |
| | | | | IPR017871 | |
| | | | | IPR027417 | |
| | | | | IPR039421 | |
| 82 | P—P-bond- | 05BZ1 | PF00664 | IPR003593 | 3.A.1.10 |
| | hydrolysis-driven | | PF00005 | IPR011527 | |
| | transporters | | | IPR036640 | |
| | | | | IPR003439 | |
| | | | | IPR017871 | |
| | | | | IPR027417 | |
| 84 | P—P-bond- | 05BZ1 | PF00664 | IPR003593 | 3.A.1.10 |
| | hydrolysis-driven | | PF00005 | IPR011527 | |
| | transporters | | | IPR036640 | |
| | | | | IPR003439 | |
| | | | | IPR017871 | |
| | | | | IPR027417 | |
| 86 | porters | 05EAM | PF02690 | IPR003841 | 2.A.58 |
| | | | PF01895 | IPR004633 | |
| | | | | IPR038078 | |
| | | | | IPR026022 | |
| 88 | Auxiliary | 07SYR | PF13807 | IPR032807 | 8.A.3 |
| | transport proteins | | PF02706 | IPR003856 | |
| | | | | IPR027417 | |
| 90 | Putative transport | 06N3A | PF03932 | IPR005627 | 9.B.158 |
| | proteins | | | IPR023648 | |
| | | | | IPR036822 | |
| 92 | Putative transport | 06N3A | PF03932 | IPR005627 | 9.B.158 |
| | proteins | | | IPR023648 | |
| | | | | IPR036822 | |
| 94 | Putative transport | 06N3A | PF03932 | IPR005627 | 9.B.158 |
| | proteins | | | IPR023648 | |
| | | | | IPR036822 | |
| 96 | Putative transport | 06N3A | PF03932 | IPR005627 | 9.B.158 |
| | proteins | | | IPR023648 | |
| | | | | IPR036822 | |
| 98 | porters | 05CT4 | PF13347 | IPR039672 | 2.A.2 |
| | | | | IPR020846 | |
| | | | | IPR036259 | |
| 100 | porters | 088QT | PF13347 | IPR039672 | 2.A.2 |
| | | | | IPR020846 | |
| | | | | IPR036259 | |
| | | | | IPR001927 | |
| 102 | porters | 05C0R | PF07690 | IPR004638 | 2.A.1.3 |
| | | | | IPR011701 | |
| | | | | IPR020846 | |
| | | | | IPR036259 | |
| | | | | IPR001411 | |
| 104 | porters | 07CWC | PF00474 | IPR038377 | 2.A.21 |
| | | | | IPR001734 | |
| 106 | porters | 07RJ1 | PF00474 | IPR038377 | 2.A.21 |
| | | | | IPR001734 | |
| 108 | porters | 05C0R | PF07690 | IPR011701 | 2.A.1.3 |
| | | | | IPR020846 | |
| | | | | IPR036259 | |
| | | | | IPR023721 | |
| | | | | IPR001411 | |
| 110 | porters | 05C0R | PF07690 | IPR004638 | 2.A.1.3 |
| | | | | IPR011701 | |
| | | | | IPR020846 | |
| | | | | IPR036259 | |
| | | | | IPR023721 | |
| | | | | IPR001411 | |
| 112 | porters | 05C0R | PF07690 | IPR011701 | 2.A.1.3 |
| | | | | IPR020846 | |
| | | | | IPR036259 | |
| | | | | IPR023721 | |
| | | | | IPR001411 | |
| 114 | porters | 05C0R | PF07690 | IPR004638 | 2.A.1.3 |
| | | | | IPR011701 | |
| | | | | IPR020846 | |
| | | | | IPR036259 | |
| | | | | IPR023721 | |
| | | | | IPR001411 | |
| 116 | porters | 05C0R | PF07690 | IPR004638 | 2.A.1.3 |
| | | | | IPR011701 | |
| | | | | IPR020846 | |
| | | | | IPR036259 | |
| | | | | IPR023721 | |
| | | | | IPR001411 | |
| 118 | porters | 05C0R | PF07690 | IPR011701 | 2.A.1.3 |
| | | | | IPR020846 | |
| | | | | IPR036259 | |
| | | | | IPR023721 | |
| | | | | IPR001411 | |
| 120 | porters | 05C0R | PF07690 | IPR011701 | 2.A.1.3 |
| | | | | IPR020846 | |
| | | | | IPR036259 | |
| | | | | IPR023721 | |
| | | | | IPR001411 | |
| 122 | porters | 05C0R | PF07690 | IPR004638 | 2.A.1.3 |
| | | | | IPR011701 | |
| | | | | IPR020846 | |
| | | | | IPR036259 | |
| | | | | IPR023721 | |
| | | | | IPR001411 | |
| 124 | porters | 05C0R | PF07690 | IPR004638 | 2.A.1.3 |
| | | | | IPR011701 | |
| | | | | IPR020846 | |
| | | | | IPR036259 | |
| | | | | IPR023721 | |
| | | | | IPR001411 | |
| 126 | porters | 07QF7 | PF07690 | IPR011701 | 2.A.1.2 |
| | | | PF07690 | IPR011701 | |
| | | | | IPR020846 | |
| | | | | IPR020846 | |
| | | | | IPR036259 | |
| | | | | IPR036259 | |
| | | | | IPR005829 | |
| | | | | IPR005829 | |
| 128 | porters | 07QF7 | PF07690 | IPR011701 | 2.A.1.2 |
| | | | | IPR020846 | |
| | | | | IPR036259 | |
| | | | | IPR005829 | |
| 130 | porters | 07QF7 | PF07690 | IPR011701 | 2.A.1.2 |
| | | | | IPR020846 | |
| | | | | IPR036259 | |
| | | | | IPR005829 | |
| 132 | porters | 07QF7 | PF07690 | IPR011701 | 2.A.1.2 |
| | | | | IPR020846 | |
| | | | | IPR036259 | |
| | | | | IPR005829 | |
| 134 | porters | 07QF7 | PF07690 | IPR011701 | 2.A.1.2 |
| | | | | IPR020846 | |
| | | | | IPR036259 | |
| | | | | IPR005829 | |
| 136 | porters | 07QF7 | PF07690 | IPR011701 | 2.A.1.2 |
| | | | | IPR020846 | |
| | | | | IPR036259 | |
| | | | | IPR005829 | |
| 138 | porters | 07QF7 | PF07690 | IPR011701 | 2.A.1.2 |
| | | | | IPR020846 | |
| | | | | IPR036259 | |
| | | | | IPR005829 | |
| 140 | porters | 07QF7 | PF07690 | IPR011701 | 2.A.1.2 |
| | | | | IPR020846 | |
| | | | | IPR036259 | |
| | | | | IPR005829 | |
| 142 | porters | 07QF7 | PF07690 | IPR011701 | 2.A.1.2 |
| | | | | IPR020846 | |
| | | | | IPR036259 | |
| | | | | IPR005829 | |
| 144 | porters | 07QF7 | PF07690 | IPR011701 | 2.A.1.2 |
| | | | | IPR020846 | |
| | | | | IPR036259 | |
| | | | | IPR005829 | |
| 146 | porters | 07QF7 | PF07690 | IPR011701 | 2.A.1.2 |
| | | | | IPR020846 | |
| | | | | IPR036259 | |
| | | | | IPR005829 | |
| 148 | porters | 07QF7 | PF07690 | IPR011701 | 2.A.1.2 |
| | | | | IPR020846 | |
| | | | | IPR036259 | |
| | | | | IPR005829 | |
| 150 | porters | 07QF7 | PF07690 | IPR011701 | 2.A.1.2 |
| | | | | IPR020846 | |
| | | | | IPR036259 | |
| | | | | IPR005829 | |
| 152 | porters | 070Q9 | PF04193 | IPR006603 | 2.A.123 |
| 154 | porters | 05W3H | PF04193 | IPR006603 | 2.A.123 |
| 156 | porters | 05W3H | PF03083 | IPR004316 | 2.A.123 |
| 158 | porters | 05W3H | PF03083 | IPR006603 | 2.A.123 |
| | | | PF04193 | IPR004316 | |
| 160 | porters | 05W2Y | PF03083 | IPR004316 | 2.A.123 |
| 162 | porters | 070Q9 | PF04193 | IPR006603 | 2.A.123 |
| 164 | porters | 05W3H | PF04193 | IPR006603 | 2.A.123 |
| 166 | porters | 05W3H | PF04193 | IPR006603 | 2.A.123 |
| 168 | porters | 05XJ5 | PF04193 | IPR006603 | 2.A.123 |
| 170 | porters | 05XJ5 | PF03083 | IPR004316 | 2.A.123 |
| 172 | porters | 05W3H | PF04193 | IPR006603 | 2.A.123 |
| 174 | P—P-bond- | 05MFV | PF01061 | IPR013525 | 3.A.1.10 |
| | hydrolysis-driven | | | IPR000412 | |
| | transporters | | | | |
| 176 | P—P-bond- | 05MFV | PF01061 | IPR013525 | 3.A.1.10 |
| | hydrolysis-driven | | | IPR000412 | |
| | transporters | | | | |
| 178 | P—P-bond- | 05EY8 | PF00005 | IPR003593 | 3.A.1.10 |
| | hydrolysis-driven | | PF14524 | IPR003439 | |
| | transporters | | | IPR027417 | |
| | | | | IPR029439 | |
| 180 | P—P-bond- | 05EY8 | PF14524 | IPR003593 | 3.A.1.10 |
| | hydrolysis-driven | | | IPR003439 | |
| | transporters | | | IPR017871 | |
| | | | | IPR027417 | |
| | | | | IPR029439 | |
| 182 | porters | 05BZS | PF00873 | IPR027463 | 2.A.6.3 |
| | | | | IPR001036 | |
| 184 | porters | 05CXP | PF07690 | IPR011701 | 2.A.1.2 |
| | | | | IPR020846 | |
| | | | | IPR036259 | |
| 186 | P—P-bond- | 05HAC | PF01061 | IPR013525 | 3.A.1.10 |
| | hydrolysis-driven | | | IPR000412 | |
| | transporters | | | IPR005981 | |
| 188 | P—P-bond- | 05CJ1 | PF00005 | IPR003593 | 3.A.1.10 |
| | hydrolysis-driven | | | IPR003439 | |
| | transporters | | | IPR017871 | |
| | | | | IPR015851 | |
| | | | | IPR005978 | |
| | | | | IPR027417 | |
| 190 | Putative transport | 05GWF | PF11045 | IPR020368 | N/A |
| | proteins | | | | |
| 192 | Putative transport | 05GWF | PF11045 | IPR020368 | N/A |
| | proteins | | | | |
| 194 | P—P-bond- | 05EY8 | PF00005 | IPR003593 | 3.A.1.10 |
| | hydrolysis-driven | | PF14524 | IPR003439 | |
| | transporters | | | IPR017871 | |
| | | | | IPR027417 | |
| | | | | IPR029439 | |
| 196 | P—P-bond- | 05MFV | PF01061 | IPR013525 | 3.A.1.10 |
| | hydrolysis-driven | | | IPR000412 | |
| | transporters | | | | |
| 204 | β-Barrel Porins | 08KDD | PF02264 | IPR003192 | 1.B.3.1 |
| | | | | IPR023738 | |
| | | | | IPR036998 | |
| 206 | P—P-bond- | 07FKK | PF13416 | IPR006059 | 3.A.1.1 |
| | hydrolysis-driven | | | IPR006060 | |
| | transporters | | | IPR006061 | |
| 208 | P—P-bond- | 172T7 | PF00005 | IPR003439 | 3.A.1.1 |
| | hydrolysis-driven | | PF17912 | IPR003593 | |
| | transporters | | | IPR008995 | |
| | | | | IPR015855 | |
| | | | | IPR017871 | |
| | | | | IPR027417 | |
| | | | | IPR040582 | |
| 210 | Phosphotransfer- | 05CI1 | PF00367 | IPR001127 | 4.A.1.1 |
| | driven Group | | PF00358 | IPR001996 | |
| | Translocators | | PF02378 | IPR003352 | |
| | | | | IPR010974 | |
| | | | | IPR011055 | |
| | | | | IPR013013 | |
| | | | | IPR018113 | |
| | | | | IPR036878 | |
| 212 | Phosphotransfer- | 05VI0 | PF03829 | IPR004716 | 4.A.4.1 |
| | driven Group | | | IPR018454 | |
| | Translocators | | | IPR036665 | |
| 214 | P—P-bond- | 05DMK | PF00532 | IPR001761 | 3.A.1.2 |
| | hydrolysis-driven | | | IPR026266 | |
| | transporters | | | IPR028082 | |
| 216 | P—P-bond- | 05DFW | PF13407 | IPR013456 | 3.A.1.2 |
| | hydrolysis-driven | | | IPR025997 | |
| | transporters | | | IPR028082 | |
| 218 | P—P-bond- | 07R5U | PF13407 | IPR025997 | 3.A.1.2 |
| | hydrolysis-driven | | | IPR028082 | |
| | transporters | | | IPR033893 | |

TABLE 3
Transcriptional units for expression of the membrane protein genes

| TU number | Promoter part | UTR part | Terminator part |
|---|---|---|---|
| TU1 | PROM0005_MutalikP5 | UTR0014_GalE_LeuAB | TER0010_T7early |
| TU2 | PROM0005_MutalikP5 | UTR0038_GalE_IptFG | TER0010_T7early |
| TU3 | PROM0005_MutalikP5 | UTR0055_uspF_iptFG | TER0010_T7early |
| TU4 | PROM0012_MutalikP12 | UTR0014_GalE_LeuAB | TER0010_T7early |
| TU5 | PROM0012_MutalikP12 | UTR0038_GalE_IptFG | TER0010_T7early |
| TU6 | PROM0012_MutalikP12 | UTR0055_uspF_iptFG | TER0010_T7early |
| TU7 | PROM0032_apFAB146 | UTR0014_GalE_LeuAB | TER0010_T7early |
| TU8 | PROM0032_apFAB146 | UTR0038_GalE_IptFG | TER0010_T7early |
| TU9 | PROM0032_apFAB146 | UTR0055_uspF_iptFG | TER0010_T7early |
| TU10 | PROM0015_p22 | UTR0003_BCD1 | TER0010_T7early |
| TU11 | PROM0010_MutalikP10 | UTR0013_ThrA_BCD2 | TER0010_T7early |
| TU12 | PROM0005_MutalikP5 | UTR0011_Gene10_LeuL | TER0010_T7early |
| TU13 | PROM0016_p14 | UTR0002_Gene10-LeuAB-BCD2 | TER0010_T7early |

Plasmids were maintained in the host E. coli DH5alpha (F⁻, phi80dlacZdeItaM15, delta(lacZYAargF) U169, deoR, recA1, endA1, hsdR17(rk⁻, mk⁺), phoA, supE44, lambda⁻, thi-1, gyrA96, relA1) bought from Invitrogen.

### Strains and Mutations

Escherichia coli K12 MG1655 [lambda⁻, F⁻, rph-1] was obtained from the Coli Genetic Stock Center (US), CGSC Strain #: 7740, in March 2007. Gene disruptions as well as gene introductions were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain.

Transformants carrying a Red helper plasmid pKD46 were grown in 10 ml LB media with ampicillin, (100 mg/L) and L-arabinose (10 mM) at 30° C. to an OD^{6oonm} of 0.6. The cells were made electrocompetent by washing them with 50 ml of ice-cold water, a first time, and with 1 ml ice cold water, a second time. Then, the cells were resuspended in 50 μl of ice-cold water. Electroporation was done with 50 μl of cells and 10-100 ng of linear double-stranded-DNA product by using a Gene Pulser™ (BioRad) (600 Ω, 25 μFD, and 250 volts).

After electroporation, cells were added to 1 ml LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity.

The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with Dpnl, repurified from an agarose gel, and suspended in elution buffer (5 mM Tris, pH 8.0).

The selected mutants (chloramphenicol or kanamycin resistant) were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock ins are checked with control primers (Fw/Rv-gene-out).

A mutant strain derived from E. coli K12 MG1655 was created by knocking out the genes lacZ, lacY lacA, glgC, agp, pfkA, pfkB, pgi, arcA, iclR, wcaJ, lon and thyA. Additionally, the E. coli lacY gene, a fructose kinase gene (frk) originating from Zymomonas mobilis, an E. coli W sucrose transporter (cscB) and a sucrose phosphorylase (SP) originating from Bifidobacterium adolescentis were knocked in into the genome and expressed constitutively. The constitutive promoters originate from the promoter library described by De Mey et al. (BMC Biotechnology, 2007). These genetic modifications are also described in WO2016075243 and WO2012007481. The α1,3- or α1,2-fucosyltransferase genes were presented to the mutant strain from a plasmid as described in the same. All membrane protein genes were evaluated in this mutant strain derived from E. coli K12 MG1655. Membrane protein genes were evaluated either present on plasmid or integrated in the host's genome (in the setA or IdhA locus). All strains are stored in cryovials at −80° C. (overnight LB culture mixed in a 1:1 ratio with 70% glycerol).

An alternative mutant strain can be derived from E. coli K12 JM109 wherein the genes lacZ, resA and wcaJ are knocked out. α1,3- or α1,2-fucosyltransferase genes are presented to the mutant strain from a plasmid as described above resulting in the production of 2′fucosyllactose, 3-fucosyllactose or 2′,3-difucosyllactose. Membrane protein genes are evaluated in the same way as described above. The strain is enabled to internalize lactose by means of allo-lactose or IPTG, inducing the lactose permease gene lacY.

Another alternative mutant strain can be derived from E coli BL21. The genes lacZ, fucI, fucK and wzxC-wcaJ are knocked out in the strain. In order to improve the synthesis of GDP-fucose in the mutant strain the genes encoding for phosphomannomutase (manB), mannose-1-phosphate guanosyltransferase (manC), GDP-mannose-4,6-dehydratase (gmd), and GDP-L-fucose synthase (wcaG) from E. coli K12 were overexpressed in a similar way as described above. Intracellular lactose synthesis is accomplished by overexpression of the gene encoding for beta-1,4-galactosyltransferase encoded by the gene lgtB. To enhance the synthesis of UDP-galactose the operon encoding for galETKM is knocked out and the gene encoding for UDP-glucose epimerase is overexpressed. α1,3- or α1,2-fucosyltransferase genes are presented to the mutant strain from a plasmid as described above resulting in the production of 2′fucosyllactose, 3-fucosyllactose or 2′,3-difucosyllactose. Membrane protein genes are evaluated in the same way as described above.

Another alternative mutant strain can be derived from E. coli K12. The genes lacZ, fucI, fucK and wzxC-wcaJ are knocked out in the strain. In order to improve the synthesis of GDP-fucose in the mutant strain the genes encoding for phosphomannomutase (manB), mannose-1-phosphate guanosyltransferase (manC), GDP-mannose-4,6-dehydratase (gmd), and GDP-L-fucose synthase (wcaG) from E. coli K12 were overexpressed in a similar way as described above. In addition, the strain is modified with genomic knock-ins of the fucose permease (fucP) gene from E. coli and the bifunctional fucose kinase/fucose-1-phosphate guanylyltransferase (fkp) gene from Bacteroides fragilis. α1,3- or α1,2-fucosyltransferase genes are presented to the mutant strain from a plasmid as described above resulting in the production of 2′fucosyllactose, 3-fucosyllactose or 2′,3-difucosyllactose. Membrane protein genes are evaluated in the same way as described above. The strain is enabled to internalize lactose by means of allo-lactose or IPTG, inducing the lactose permease gene lacY.

Another alternative mutant strain can be derived from E. coli K12. The genes lacZ, and wzxC-wcaJ are knocked out in the strain. In order to improve the synthesis of GDP-fucose in the mutant strain the genes encoding for phosphomannomutase (manB), mannose-1-phosphate guanosyltransferase (manC), GDP-mannose-4,6-dehydratase (gmd), and GDP-L-fucose synthase (wcaG) from E. coli K12 were overexpressed in a similar way as described above. To improve the formation of fructose-6-phosphate from gluconeogenic substrates such as glycerol, acetate, lactate, ethanol, succinate, pyruvate, the genes encoding for phosphofructokinase (pfkA and pfkB) are knocked out and the genes encoding for fructose-1,6-bisphosphate aldolase (fbaB) and a heterologous fructose-1,6-bisphosphate phosphatase (fbpase) from Pisum sativum were overexpressed. α1,3- or α1,2-fucosyltransferase genes are presented to the mutant strain from a plasmid as described above resulting in the production of 2′fucosyllactose, 3-fucosyllactose or 2′,3-difucosyllactose. Membrane protein genes are evaluated in the same way as described above.

### Cultivation Conditions

A preculture of 96-well microtiter plate experiments was started from a cryovial, in 150 μL LB and was incubated overnight at 37° C. on an orbital shaker at 800 rpm. This culture was used as inoculum for a 96-well square microtiter plate, with 400 μL minimal medium by diluting 400×. Each strain was grown in multiple wells of the 96-well plate as biological replicates.

These final 96-well culture plates were then incubated at 37° C. on an orbital shaker at 800 rpm for 72 hours, or shorter, or longer. At the end of the cultivation experiment samples were taken from each well to measure sugar concentrations in the broth supernatant (extracellular sugar concentrations, after 5 spinning down the cells), or by boiling the culture broth for 15 min at 90° C. before spinning down the cells (=whole broth measurements, average of intra- and extracellular sugar concentrations).

Also, a dilution of the cultures was made to measure the optical density at 600 nm. The cell performance index or CPI was determined by dividing the fucosyllactose concentrations measured in the whole broth by the biomass, in relative percentages compared to the reference strain. The biomass is empirically determined to be approximately ⅓^{rd} of the optical density measured at 600 nm. The fucosyllactose export ratio was determined by dividing the fucosyllactose concentrations measured in the supernatant by the fucosyllactose concentrations measured in the whole broth, in relative percentages compared to the reference strain.

A preculture for the bioreactor was started from an entire 1 mL cryovial of a certain strain, inoculated in 250 mL or 500 mL of minimal medium in a 1 L or 2.5 L shake flask and incubated for 24 hours at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing conditions were set to 37° C., and maximal stirring; pressure gas flow rates were dependent on the strain and bioreactor. The pH was controlled at 6.8 using 0.5 M H2SO4 and 20% NH40H. The exhaust gas was cooled. 10% solution of silicone antifoaming agent was added when foaming raised during the fermentation.

### Optical Density

Cell density of the cultures was frequently monitored by measuring optical density at 600 nm (Implen Nanophotometer NP80, Westburg, Belgium or with a Spark 1OM microplate reader, Tecan, Switzerland).

### Productivity

The specific productivity Qp is the specific production rate of the fucosyllactose product, typically expressed in mass units of product per mass unit of biomass per time unit (=g fucosyllactose/g biomass/h). The Qp value has been determined for each phase of the fermentation runs, i.e., Batch and Fed-Batch phase, by measuring both the amount of product and biomass formed at the end of each phase and the time frame each phase lasted.

The specific productivity Qs is the specific consumption rate of the substrate, e.g., sucrose, typically expressed in mass units of substrate per mass unit of biomass per time unit (=g sucrose/g biomass/h). The Qs value has been determined for each phase of the fermentation runs, i.e., Batch and Fed-Batch phase, by measuring both the total amount of sucrose consumed and biomass formed at the end of each phase and the time frame each phase lasted.

The yield on sucrose Ys is the fraction of product that is made from substrate and is typically expressed in mass unit of product per mass unit of substrate (=g fucosyllactose/g sucrose). The Ys has been determined for each phase of the fermentation runs, i.e., Batch and Fed-Batch phase, by measuring both the total amount of fucosyllactose produced and total amount of sucrose consumed at the end of each phase.

The yield on biomass Yx is the fraction of biomass that is made from substrate and is typically expressed in mass unit of biomass per mass unit of substrate (=g biomass/g sucrose). The Yp has been determined for each phase of the fermentation runs, i.e., Batch and Fed-Batch phase, by measuring both the total amount of biomass produced and total amount of sucrose consumed at the end of each phase.

The rate is the speed by which the product is made in a fermentation run, typically expressed in concentration of product made per time unit (=g fucosyllactose/L h). The rate is determined by measuring the concentration of fucosyllactose that has been made at the end of the Fed-Batch phase and dividing this concentration by the total fermentation time.

The lactose conversion rate is the speed by which lactose is consumed in a fermentation run, typically expressed in mass units of lactose per time unit (=g lactose consumed/h). The lactose conversion rate is determined by measurement of the total lactose that is consumed during a fermentation run, divided by the total fermentation time.

### Growth Rate/Speed Measurement

The maximal growth rate (μMax) was calculated based on the observed optical densities at 600 nm using the R package grofit.

### Liquid Chromatography

Standards for 2′-fucosyllactose, 3-fucosyllactose and 2′, 3-difucosyllactose were synthetized in house. Other standards such as but not limited to lactose, sucrose, glucose, glycerol, fructose were purchased from Sigma. Carbohydrates were analyzed via a HPLC-RI (Waters, USA) method, whereby RI (Refractive Index) detects the change in the refraction index of a mobile phase when containing a sample. The sugars were separated in an isocratic flow using an X-Bridge column (Waters X-bridge HPLC column, USA) and a mobile phase containing 75 ml acetonitrile and 25 ml Ultrapure water and 0.15 ml triethylamine. The column size was 4.6×150 mm with 3.5 μm particle size. The temperature of the column was set at 35° C. and the pump flow rate was 1 mL/min.

### Normalization of the Data

For all types of cultivation conditions, data obtained from the mutant strains was normalized against data obtained in identical cultivation conditions with reference strains having an identical genetic background as the mutant strains but lacking the membrane protein expression cassettes. The dashed horizontal line on each plot that is shown in the examples, indicates the setpoint to which all adaptations were normalized. All data is given in relative percentages to that setpoint.

### Example 4: Membrane Proteins Identified that Enhance 3-Fucosyllactose (3-FL) Production in an E. coli Host Cultivated 72 Hours in a Growth Experiment in Minimal Media Supplemented with 20 g/L Lactose

An experiment was set up to evaluate membrane proteins for their ability to enhance fucosyllactose production of a host cell growing in minimal media supplemented with 20 g/L lactose. The membrane proteins with SEQ ID NOS: 02, 04, 06, 18, 20, 22, 26, 28, 30, 32, 34, 40, 42, 44, 58, 62, 64, 66, 70, 72, 74, 82, 84, 90, 92, 94 and 96 showed that they are able to enhance 3-FL production that is being produced in a 3-FL production host expressing the α1,3-fucosyl transferases FT1 or FT2. Candidate genes were combined in transcriptional unit TU2, TU3 or TU10 and presented to the 3-FL production hosts on a pSC101 plasmid. A growth experiment was performed according to the cultivation conditions provided in Example 3. FIG. 1 presents the CPI of the strains, in relative percentages compared to the respective reference strain.

### Example 5: Membrane Proteins Identified that Enhance 3-FL Secretion in an E. coli Host Cultivated 72 Hours in a Growth Experiment in Minimal Media Supplemented with 20 g/L Lactose

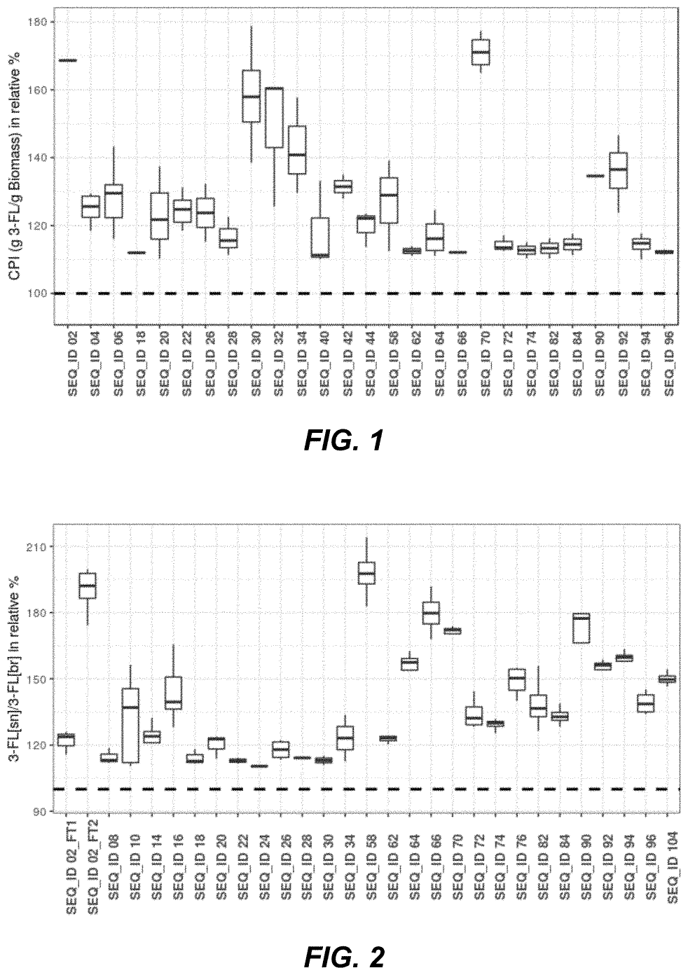

An experiment was set up to evaluate membrane proteins for their ability to enhance fucosyllactose secretion of a host cell growing in minimal media supplemented with 20 g/L lactose. The membrane proteins with SEQ ID NOS: 02, 08, 10, 14, 16, 18, 20, 22, 24, 26, 28, 30, 34, 58, 62, 64, 66, 70, 72, 74, 76, 82, 84, 90, 92, 94, 96 and 104 showed that they are able to enhance secretion of 3-FL that is being produced intracellularly in a 3-FL bacterial production host expressing the α1,3-fucosyl transferases FT1 or FT2. Candidate genes were combined in transcriptional unit TU2, TU3 or TU10 and presented to the 3-FL production hosts on a pSC101 plasmid. A growth experiment was performed according to the cultivation conditions provided in Example 3. FIG. 2 demonstrates the export ratio of 3-FL in the strains, in relative percentages compared to the respective reference strain.

### Example 6: Membrane Proteins Identified that Enhance Growth Speed in an E. coli Host Cultivated 72 Hours in a Growth Experiment in Minimal Media Supplemented with 20 g/L Lactose

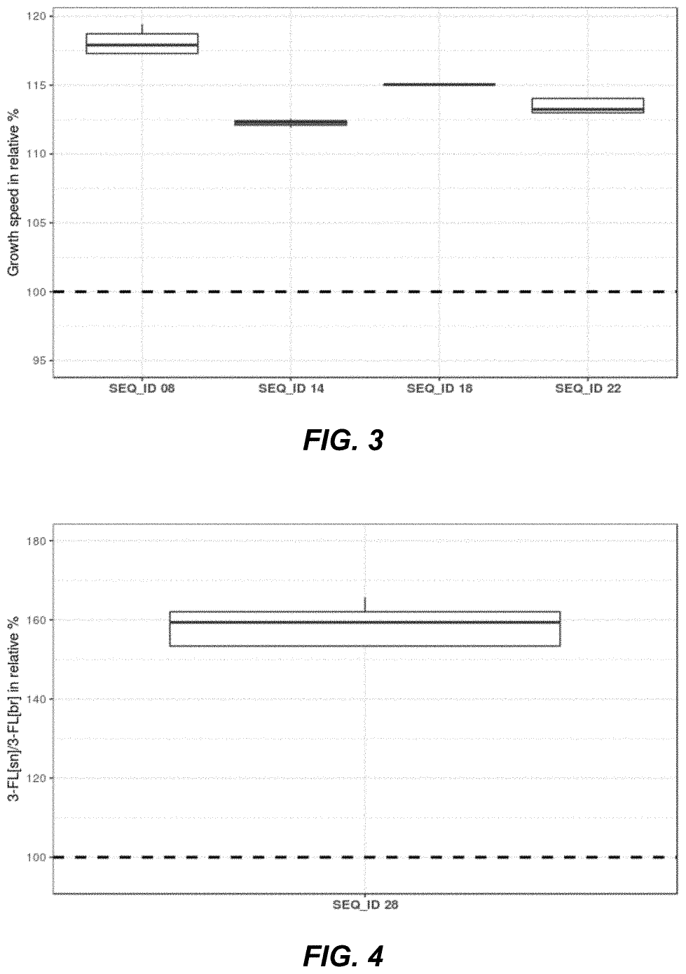

An experiment was set up to evaluate membrane proteins for their ability to influence growth speed of a host cell growing in minimal media supplemented with 20 g/L lactose. Membrane proteins with SEQ ID NOS: 08, 14, 18 and 22 showed to be able to enhance the growth speed of a 3-FL production host expressing the α1,3-fucosyl transferase FT1 or FT2. Candidate genes were combined in transcriptional unit TU2, TU3 or TU10 and presented to the 3-FL production hosts on a pSC101 plasmid. A growth experiment was performed according to the cultivation conditions provided in Example 3. FIG. 3 demonstrates the growth speed of the strains, in relative percentages compared to the respective reference strain.

### Example 7: Membrane Protein Identified that Enhances 3-FL Secretion in an E. coli Host Cultivated 72 Hours in a Growth Experiment in Minimal Media Supplemented with 45 g/L Lactose

An experiment was set up to evaluate the effectiveness of a range of identified membrane proteins to enhance fucosyllactose secretion by a host cell growing in minimal media supplemented with 45 g/L lactose. Membrane protein with SEQ ID NO: 28 showed to be able to enhance 3-FL secretion in a 3-FL production host expressing the α1,3-fucosyl transferase enzyme FT1. The gene with SEQ ID NO: 27 was combined in transcriptional unit TU10 and presented to the 3-FL production host on a pSC101 plasmid. A growth experiment was performed according to the cultivation conditions provided in Example 3 using minimal medium supplemented with 45 g/L lactose. FIG. 4 shows the CPI of the strain, in relative percentages compared to the respective reference strain.

### Example 8: Membrane Protein Identified that Enhances 3-FL Production in an E. coli Host Cultivated 72 Hours in a Growth Experiment in Minimal Media Supplemented with 90 g/L Lactose

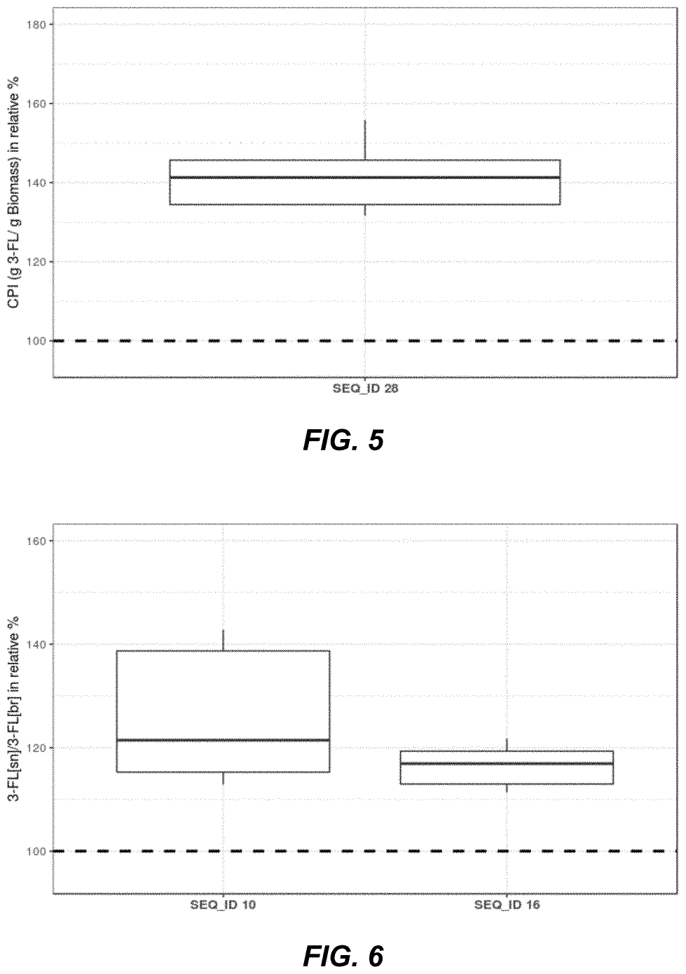

An experiment was set up to evaluate the ability of a range of membrane proteins to enhance fucosyllactose production in minimal media supplemented with 90 g/L lactose. The membrane protein with SEQ ID NO: 28 showed to be able to enhance 3-FL production in a 3-FL production host expressing the α1,3-fucosyl transferase enzyme FT1. The gene with SEQ ID NO: 27 was combined in transcriptional unit TU10 and presented to the 3-FL production host on a pSC101 plasmid. A growth experiment was performed according to the cultivation conditions provided in Example 3 using minimal medium supplemented with 90 g/L lactose. FIG. 5 shows the CPI, in relative percentages compared to the respective reference strain.

### Example 9: Membrane Proteins Identified that Increase 3-FL Secretion in an E. coli Host Cultivated 72 Hours in a Growth Experiment in Minimal Media Supplemented with 90 g/L Lactose

An experiment was set up to evaluate the ability of a range of membrane proteins to increase fucosyllactose secretion by a host cell growing in minimal media supplemented with 90 g/L lactose. The membrane proteins with SEQ ID NOS: 10 and 16 showed to be able to enhance 3-FL secretion in a 3-FL production host expressing the α1,3-fucosyl transferase enzyme FT1. The candidate genes were combined in transcriptional unit TU10 and presented to the 3-FL production host on a pSC101 plasmid. A growth experiment was performed according to the cultivation conditions provided in Example 3 using minimal medium supplemented with 90 g/L lactose. FIG. 6 demonstrates the export ratio of 3-FL, in relative percentages compared to the respective reference strain.

### Example 10: Membrane Proteins Identified that Increase 3-FL Production in an E. coli Host Cultivated 72 Hours in a Growth Experiment in Minimal Media Supplemented with 100 g/L Sucrose and 90 g/L Lactose

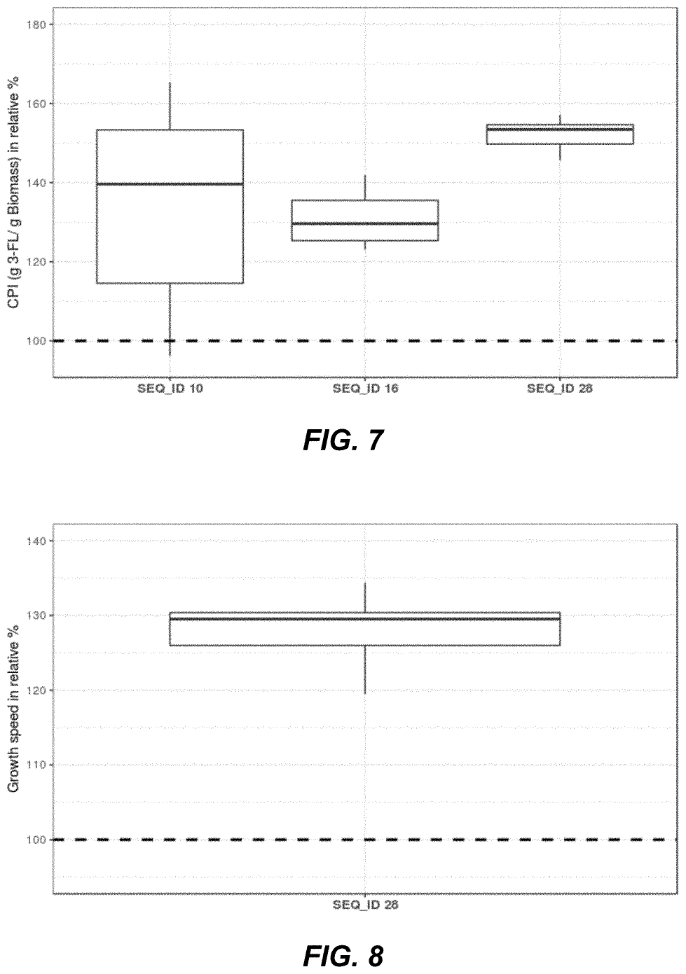

An experiment was set up to evaluate the ability of a range of membrane proteins for their ability to increase fucosyllactose production in a host cell growing in minimal media supplemented with 100 g/L sucrose and 90/L of lactose. The membrane proteins with SEQ ID NOS: 10, 16 and 28 showed to be able to enhance 3-FL production in a 3-FL production host expressing the α1,3-fucosyl transferase enzyme FT1. All candidate genes were combined in transcriptional unit TU10 and presented to the 3-FL production host on a pSC101 plasmid. A growth experiment was performed according to the cultivation conditions provided in Example 3 using minimal medium supplemented with 100 g/L sucrose and 90 g/L lactose. FIG. 7 demonstrates the CPI, in relative percentages compared to the respective reference strain.

### Example 11: Membrane Protein Identified that Increases the Growth Speed of E. coli Hosts when Cultivated 72 Hours in a Growth Experiment in Minimal Media Supplemented with 100 g/L Sucrose and 90 g/L Lactose

An experiment was set up to evaluate membrane proteins for their ability to influence growth speed of a host cell growing in minimal media supplemented with 100 g/L sucrose and 90/L of lactose. The membrane protein with SEQ ID NO: 28 showed to be able to enhance the growth speed of a 3-FL production host expressing the α1,3-fucosyl transferase enzyme FT1. The gene with SEQ ID NO: 27 was combined in transcriptional unit TU10 and presented to the 3-FL production host on a pSC101 plasmid. A growth experiment was performed according to the cultivation conditions provided in Example 3 using minimal medium supplemented with 100 g/L sucrose and 90 g/L lactose. FIG. 8 demonstrates the growth speed, in relative percentages compared to the respective reference strain.

### Example 12: Membrane Protein Identified that Increases 3-FL Production in an E. coli Host Cultivated 72 Hours in a Growth Experiment in Minimal Media Supplemented with 5 g/L Lactose

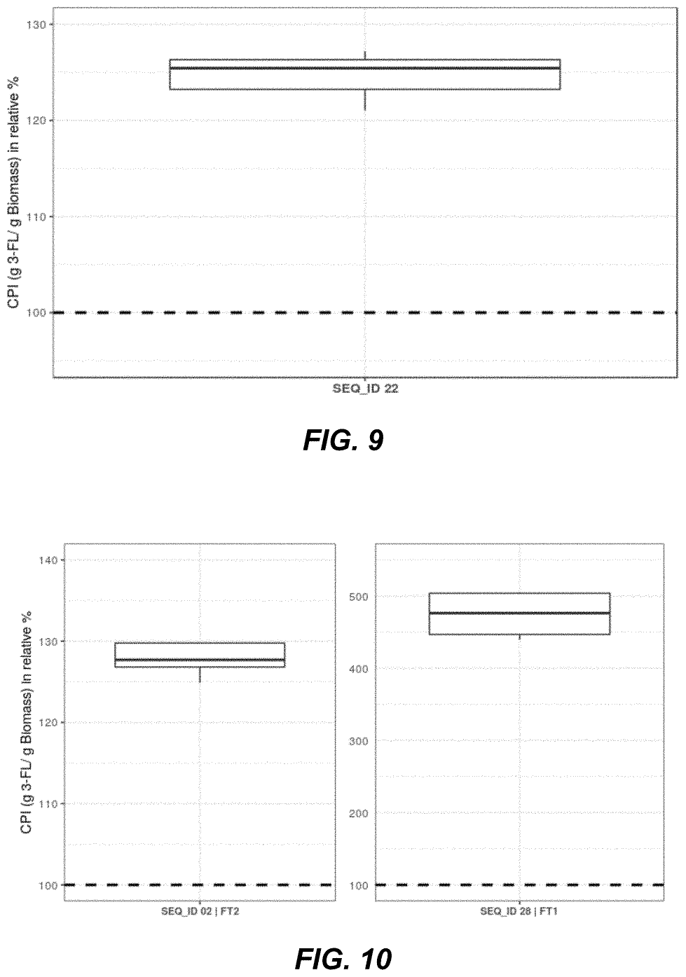

An experiment was set up to evaluate the ability of membrane proteins for their ability to influence fucosyllactose production by a host cell growing in minimal media supplemented with 5 g/L lactose. The membrane protein with SEQ ID NO: 22 showed that it is able to enhance 3-FL production in a 3-FL production host expressing the α1,3-fucosyl transferase enzyme FT1. The gene with SEQ ID NO: 21 was combined in transcriptional unit TU10 and presented to the 3-FL production host on a pSC101 plasmid. A growth experiment was performed according to the cultivation conditions provided in Example 3 using minimal medium supplemented with 5 g/L lactose. FIG. 9 demonstrates the CPI, in relative percentages compared to the respective reference strain.

### Example 13: Membrane Proteins Identified that, when Integrated in the Host's Genome. Increase 3-FL Production and/or Secretion in an E. coli Host Cultivated 72 Hours in a Growth Experiment in Minimal Media Supplemented with 20 g/L Lactose

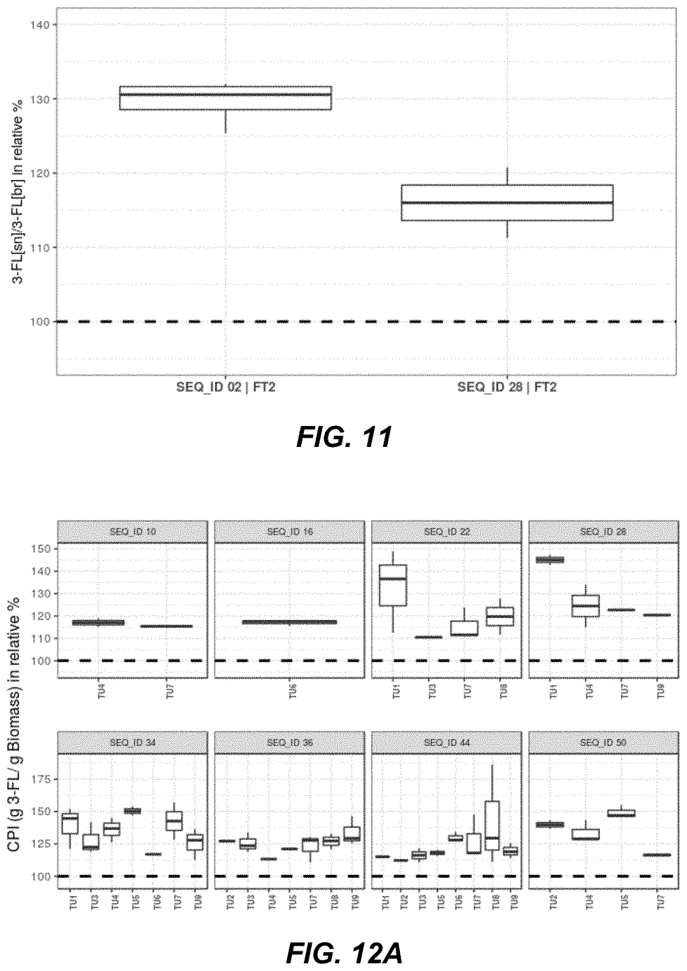

Another series of experiments was set up to evaluate the ability of membrane proteins integrated in the genome to increase fucosyllactose production and/or secretion in/by a host cell cultivated for 72 hours in minimal media supplemented with 20 g/L lactose. The membrane proteins with SEQ ID NOS: 02 and 28 showed that they are able to enhance 3-FL production and/or secretion of 3-FL that is being produced intracellularly in a 3-FL production host either expressing the α1,3-fucosyl transferase enzymes FT1 or FT2. The genes with SEQ ID NOS: 01 and 27 were combined in the transcriptional unit TU10 and presented to the genome of the 3-FL production hosts as genomic KI in the EcLdhA or the EcSetA locus, respectively. A growth experiment was performed according to the cultivation conditions provided in Example 3. FIG. 10 demonstrates the CPI whereas FIG. 11 shows the 3-FL export, both times in relative percentages compared to the respective reference strain.

### Example 14: Membrane Proteins that Independently from the Transcriptional Unit they are Cloned in, Enhance 3-FL Production and/or 3-FL Secretion in an E. coli Host

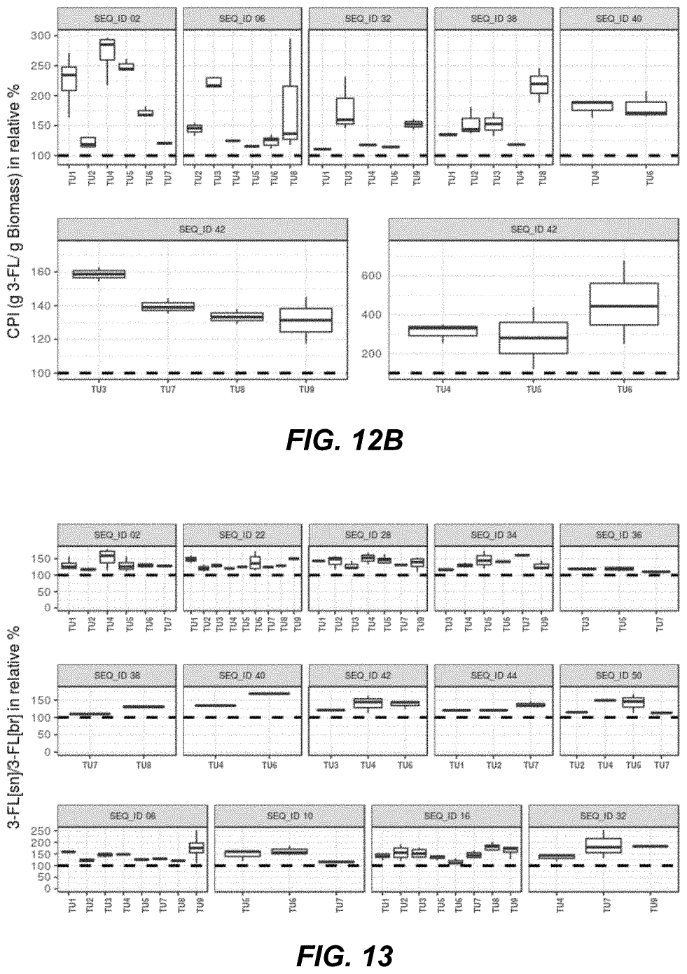

Another series of experiments was set up to evaluate the ability of membrane proteins to influence fucosyllactose production and/or secretion, of a host cell. In this example, several transcriptional units were also used for cloning. The membrane proteins with SEQ ID NOS: 02, 06, 10, 16, 22, 28, 32, 34, 36, 38, 40, 42, 44 and 50 showed that they are able to enhance 3-FL production and/or secretion of 3-FL that is being produced intracellularly in a 3-FL production host by the α1,3-fucosyl transferase enzyme FT2. The exporter genes were cloned in different transcriptional units and presented as clonal vector (pSC101 ori) to the 3-FL production host. A growth experiment was performed according to the cultivation conditions provided in Example 3. FIG. 12 demonstrates the CPI whereas FIG. 13 shows the 3-FL export, both times in relative percentages compared to the respective reference strain.

### Example 15: Membrane Proteins Identified that, when Cloned in their Native Transcriptional Operon Structure, Enhance 3-FL Production and/or 3-FL Secretion in an E. coli Host Cultivated 72 Hours in a Growth Experiment in Minimal Media Supplemented with 20 g/L Lactose

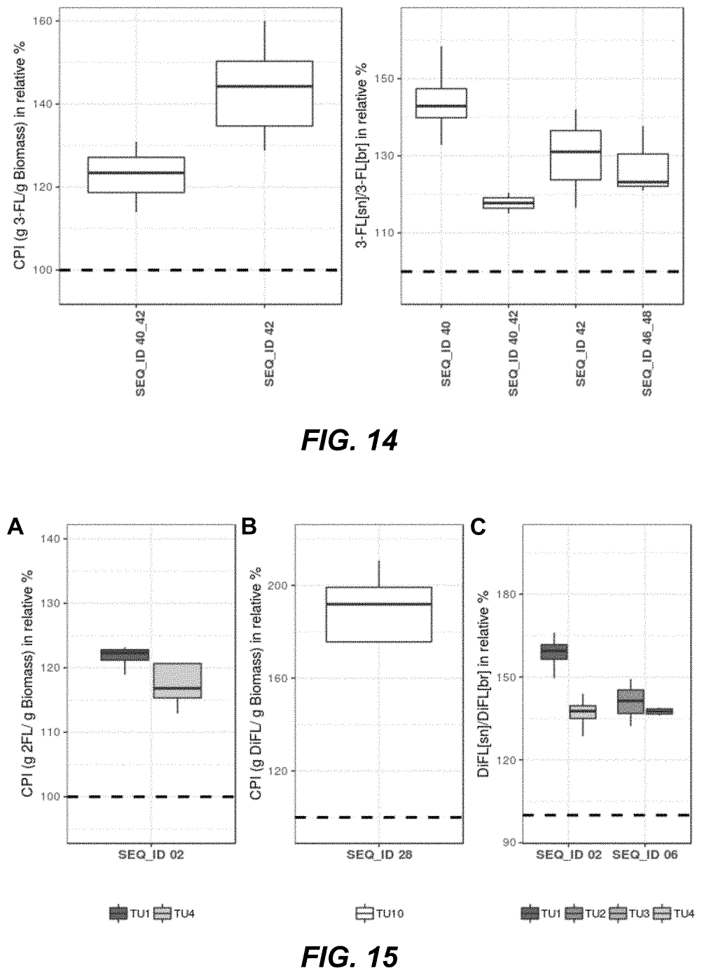

An experiment was set up to evaluate the ability of membrane proteins to enhance fucosyllactose production and/or secretion of a host cell cultivated for 72 hours in minimal media supplemented with 20 g/L lactose. This time the membrane proteins where cloned in their native transcriptional operon structure. The membrane proteins with SEQ ID NOS: 40, 42, 46 and 48 showed to be able to enhance 3-FL production and/or 3-FL secretion in a 3-FL production host expressing the α1,3-fucosyl transferase enzyme FT2. All candidate exporters genes were cloned either in TU10 as single genes or in their native transcriptional operon structure containing two membrane protein genes and presented to the 3-FL production host on a pSC101 plasmid. A growth experiment was performed according to the cultivation conditions provided in Example 3. FIG. 14 shows the CPI (left panel) and the 3-FL export ratio (right panel), in relative percentages compared to the respective reference strain.

### Example 16: Membrane Proteins Identified that Enhance 2′-FL and/or DiFL Production. And/or DiFL Secretion in a 2′-FL E. coli Host Cultivated 72 Hours in a Growth Experiment in Minimal Media Supplemented with 20 g/L Lactose

An experiment was set up to evaluate the ability of a range of membrane proteins to enhance fucosyllactose production and/or secretion of a host cell cultivated for 72 hours in minimal media supplemented with 20 g/L lactose. This time the membrane proteins where tested for 2′-FL and/or diFL production. The membrane proteins with SEQ ID NOS: 02, 06 and 28 showed to be able to enhance 2′FL and/or DiFL production and/or DiFL secretion in a 2′-FL production host expressing the α1,2-fucosyltransferase FT3. Candidate genes were cloned in different TUs and presented on the genome of the 2′-FL production host, using either the SetA locus (for the gene with SEQ ID NO: 27) or the ldhA locus (for the gene with SEQ ID NOS: 01 and 05). A growth experiment was performed according to the cultivation conditions provided in Example 3. FIG. 15 shows the CPI for the hosts with 2′FL (panel A) or DiFL (panel B) production and the DiFL export ratio (panel C), every time in relative percentages compared to the respective reference strain.

### Example 17: The Membrane Protein MdfA Enhances the Productivity of a 3-FL Production E. coli Host in (30 L) Fermentation Runs

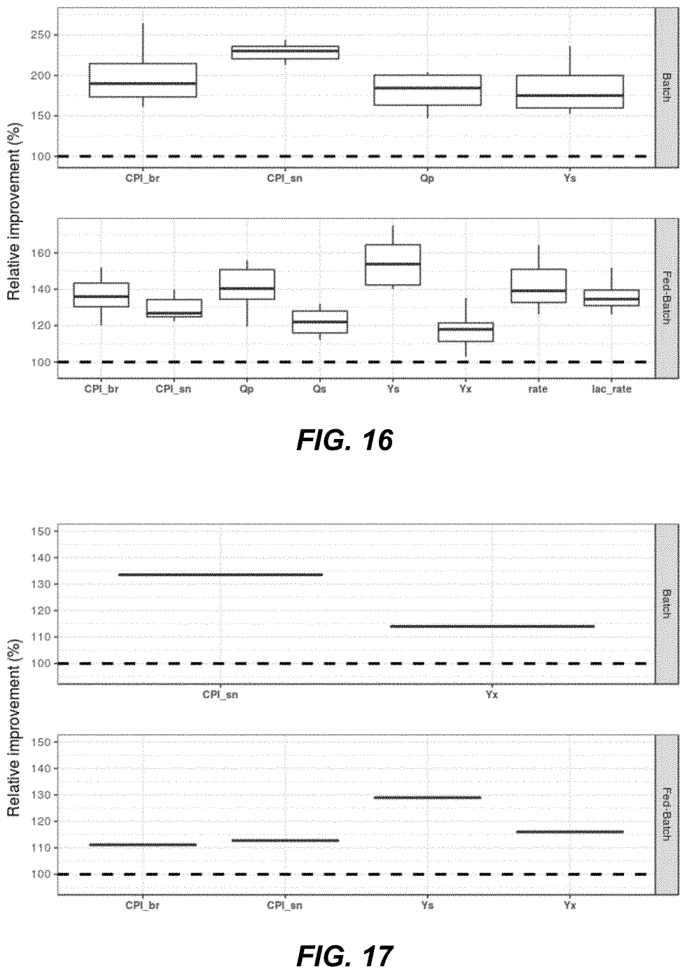

A 3-FL producing E. coli host having the membrane protein gene with SEQ ID NO: 01 expressed in TU1 and presented in the host's genome in the EcldhA locus, and expressing the α1,3-fucosyl transferase FT2 from plasmid was evaluated for its productivity in bioreactor settings. Eight fermentation runs were performed according to the conditions provided in Example 3. Also, a reference strain identical to the 3-FL production host but lacking the membrane protein gene was analyzed in identical fermentation settings. FIG. 16 demonstrates the enhanced productivity of the strain over-expressing the membrane protein EcMdfA with SEQ ID NO: 02 in the eight different fermentation runs, relatively compared to this reference strain.

### Example 18: The Membrane Protein IceT Enhances the Productivity of a 3-FL Production E. coli Host in (30 L) Fermentation Runs

Another 3-FL production E. coli host expressing a membrane protein was evaluated for its productivity in 30 L bioreactors. The 3-FL strain had the membrane protein gene EcIceT with SEQ ID NO: 05 expressed in TU3 from a first plasmid and the α1,3-fucosyl transferase FT2 expressed from a second plasmid. A specific reference strain identical to the 3-FL production hosts but lacking the membrane protein gene construct was used to analyze the 3-FL productivity in identical fermentation settings. FIG. 17 demonstrates the enhanced productivity of the strain over-expressing the membrane protein relatively compared to the specific reference strain.

### Example 19: Membrane Proteins Homologous to EcMdfA or EcIceT Enhance 3-FL Production. And/or 3-FL Secretion. And/or Growth Speed in a 3-FL E. coli Host Cultivated 72 Hours in a Growth Experiment in Minimal Media Supplemented with 20 g/L Lactose

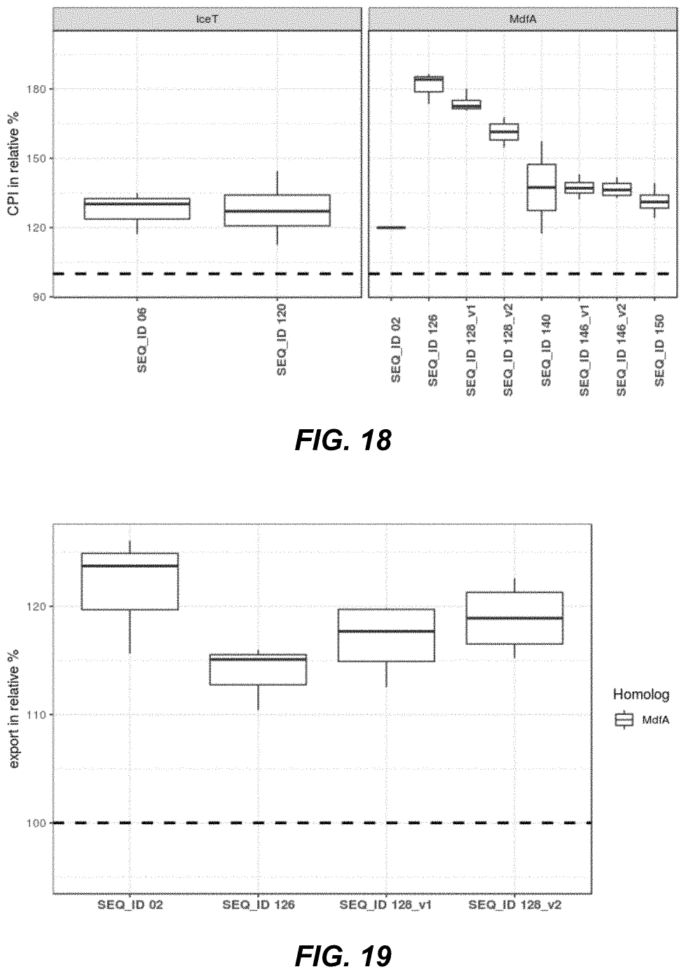

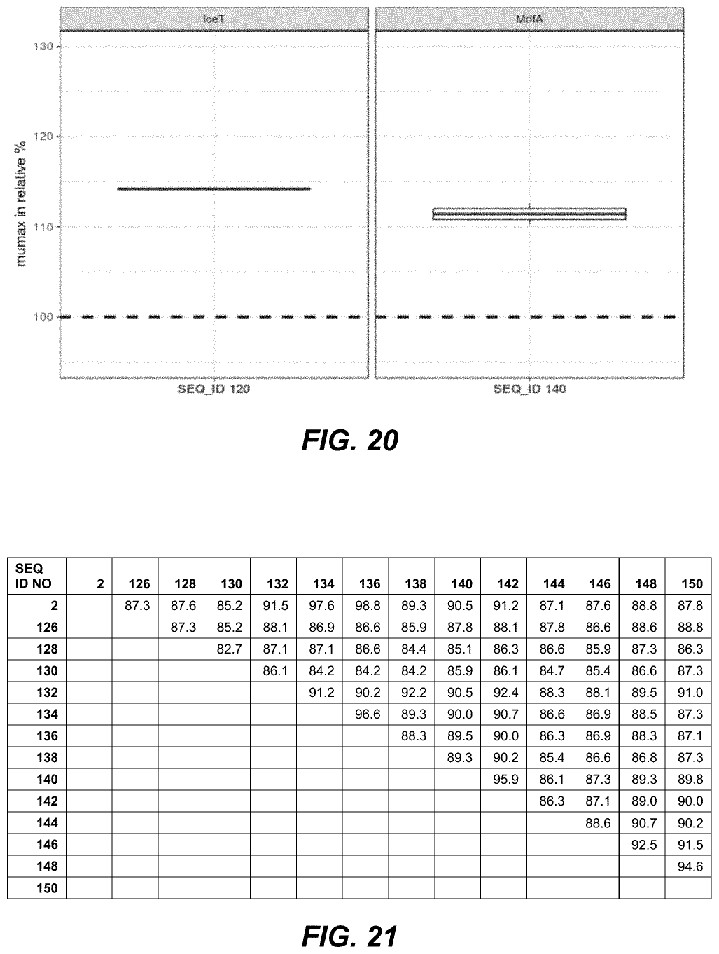

An experiment was set up to evaluate the membrane protein with SEQ ID NO: 120 (homologous to EcIceT with SEQ ID NO: 06), and membrane proteins with SEQ ID NOS: 126, 128, 140, 146 and 150 (homologous to membrane protein EcMdfA with SEQ ID NO: 02) that are able to enhance 3-FL production and/or secretion of 3-FL that is being produced intracellularly in a 3-FL production host by the α1,3-fucosyl transferase enzymes FT1 or FT2 and/or that improve growth speed of the 3-FL E. coli host. The exporter genes were cloned in different transcriptional units and presented as clonal vector (pSC101 ori) to the 3-FL production host. A growth experiment was performed according to the cultivation conditions provided in Example 3. FIG. 18 demonstrates the CPI whereas FIG. 19 shows the 3-FL export and FIG. 20 presents the strains with elevated growth speed, each time in relative percentages compared to the respective reference strains.

### Example 20: Calculation of Global Percentage Identity Between Polypeptide Sequences

Methods for the alignment of sequences for comparison are well known in the art, such methods include GAP, BESTFIT, BLAST, FASTA and TFASTA. GAP uses the algorithm of Needleman and Wunsch ((1970) J. Mol. Biol. 48: 443-453) to find the global (i.e., spanning the complete sequences) alignment of two sequences that maximizes the number of matches and minimizes the number of gaps. The BLAST algorithm (Altschul et al. (1990) J. Mol. Biol. 215: 403-10) calculates percentage sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics, 2003 Jul. 10; 4:29, MatGAT: an application that generates similarity/identity matrices using protein or DNA sequences). Minor manual editing may be performed to optimize alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T. F., Waterman M. S. (1981) J. Mol. Biol. 147(1); 195-7).

Global percentages of similarity and identity between full length polypeptide sequences useful in performing the methods of the disclosure were determined using MatGAT (Matrix Global Alignment Tool) software (BMC Bioinformatics. 2003 4:29. MatGAT: an application that generates similarity/identity matrices using protein or DNA sequences. Campanella J. J., Bitincka L., S. malley J.; software hosted by Ledion Bitincka). MatGAT generates similarity/identity matrices for DNA or protein sequences without needing pre-alignment of the data. The program performs a series of pair-wise alignments using the Myers and Miller global alignment algorithm, calculates similarity and identity, and then places the results in a distance matrix.

1. CYP704-Like Polypeptides.

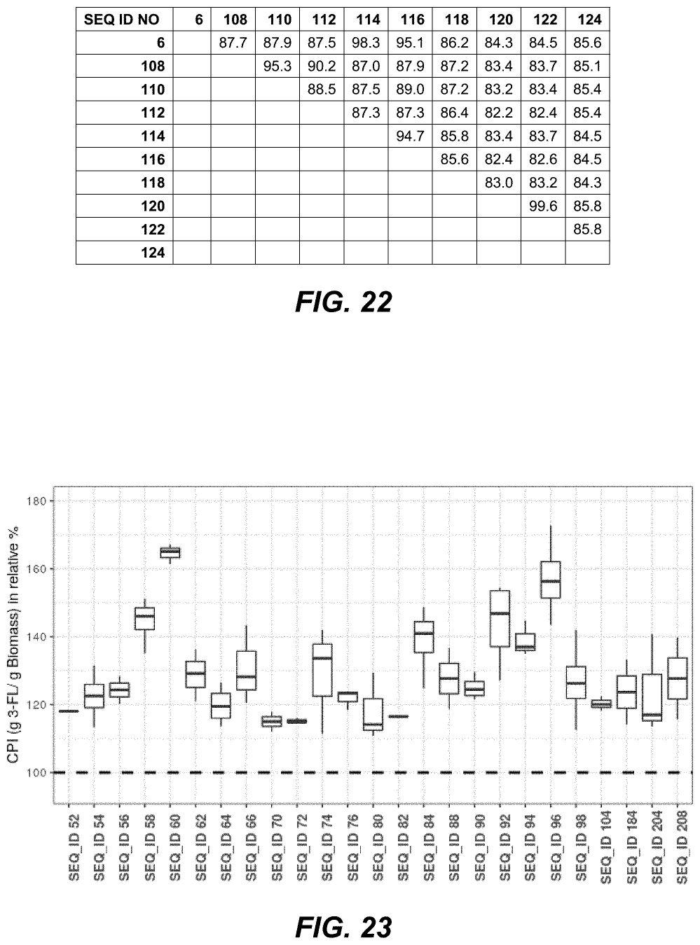

Results of an exemplary analysis are shown in FIGS. 21 and 22 for the global identity over the full length of the polypeptide sequences relating to EcMdfA (SEQ ID NO: 2) and EcIceT (SEQ ID NO: 6). Sequence identity is shown in the top half of the diagonal dividing line. Parameters used in the comparison were: Scoring matrix: Blosum62, First Gap: 12, Extending Gap: 2. The sequence identity (in percent) between the EcMdfA membrane protein and its homologs useful in performing the methods of the disclosure is generally higher than 80%. The sequence identity in percent between the EcIceT membrane protein and its homologs useful in performing the methods of the disclosure is generally higher than 80%.

### Example 21: Membrane Proteins Identified that Enhance 3-Fucosyllactose (3-FL) Production in an E. coli Host Cultivated 72 Hours in a Growth Experiment in Minimal Media Supplemented with 20 g/L Lactose

An experiment was set up to evaluate membrane proteins for their ability to enhance fucosyllactose production of a host cell growing in minimal media supplemented with 20 g/L lactose. The membrane proteins with SEQ ID NOS: 52, 54, 56, 58, 60, 62, 64, 66, 70, 72, 74, 76, 80, 82, 84, 88, 90, 92, 94, 96, 98, 104, 184, 204 and 208 showed that they are able to enhance 3-FL production that is being produced in a 3-FL production host expressing the α1,3-fucosyltransferase FT2. Candidate genes were combined in transcriptional unit TU1, TU2, TU3, TU11 or TU12 and presented to the 3-FL production hosts on a pSC101 plasmid. A growth experiment was performed according to the cultivation conditions provided in Example 3. FIG. 23 presents the CPI of the strains, in relative percentages compared to the respective reference strain.

### Example 22: Membrane Proteins Identified that Enhance 2′-Fucosyllactose (2′-FL) Production in an E. coli Host Cultivated 72 Hours in a Growth Experiment in Minimal Media Supplemented with 20 g/L Lactose

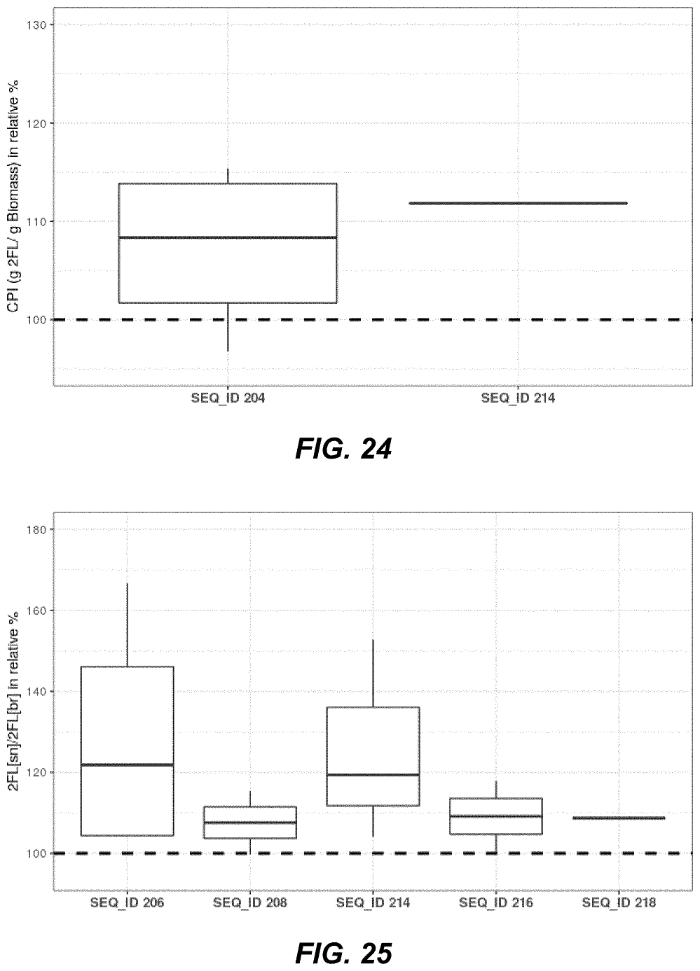

An experiment was set up to evaluate membrane proteins for their ability to enhance fucosyllactose production of a host cell growing in minimal media supplemented with 20 g/L lactose. The membrane proteins with SEQ ID NOS: 204 and 214 showed that they are able to enhance 2′-FL production that is being produced in a 2′-FL production host expressing the α1,2-fucosyltransferase FT4. Candidate genes were combined in transcriptional unit TU11 and presented to the 2′-FL production hosts on a pSC101 plasmid. A growth experiment was performed according to the cultivation conditions provided in Example 3. FIG. 24 presents the CPI of the strains, in relative percentages compared to the respective reference strain.

### Example 23: Membrane Proteins Identified that Enhance 2′-FL Secretion in an E. coli Host Cultivated 72 Hours in a Growth Experiment in Minimal Media Supplemented with 20 g/L Lactose

An experiment was set up to evaluate membrane proteins for their ability to enhance fucosyllactose secretion of a host cell growing in minimal media supplemented with 20 g/L lactose. The membrane proteins with SEQ ID NOS: 206, 208, 214, 216 and 218 showed that they are able to enhance secretion of 2′-FL that is being produced intracellularly in a 2′-FL bacterial production host expressing the α1,2-fucosyltransferase FT4. Candidate genes were combined in transcriptional unit TU11 and presented to the 2′-FL production hosts on a pSC101 plasmid. A growth experiment was performed according to the cultivation conditions provided in Example 3. FIG. 25 demonstrates the export ratio of 2′-FL in the strains, in relative percentages compared to the respective reference strain.

1. A method of producing fucosyllactose by a genetically modified cell, the method comprising:
providing a cell capable of producing fucosyllactose, the cell comprising at least one nucleic acid sequence coding for a fucosyltransferase that transfers a fucose residue from a guanosine-diphosphate fucose (GDP-fucose) donor to a lactose acceptor, thereby synthesizing fucosyllactose;
the cell further comprising i) a modified expression of an endogenous membrane protein enabling and/or enhancing fucosyllactose transport and/or ii) an expression of a heterologous membrane protein enabling and/or enhancing fucosyllactose transport, and wherein the membrane protein is i) selected from the group consisting of membrane proteins comprising any one of the PFAM domains found by searching the genomic neighborhood of GT10 and GT11 fucosyltransferase families with InterPro number IPR001503 and IPR002516 as defined on InterPro 75.0 released on 4 Jul. 2019, respectively, wherein the genomic neighborhood window size is 14 genes before and 14 genes after the respective fucosyltransferase and wherein the membrane protein does not belong to the SET family, or ii) selected from the group consisting of membrane proteins comprising SEQ ID NO: 204, 206, 208, 210, 212, 214, 216, and 218, or functional homolog or functional fragment of any one of the membrane proteins comprising SEQ ID NO: 204, 206, 208, 210, 212, 214, 216, and 218 or a polypeptide having at least 80% sequence identity to any one of the membrane proteins with SEQ ID NO: 204, 206, 208, 210, 212, 214, 216, or 218;
cultivating the cell in a medium under conditions permissive for the production of the desired fucosyllactose; and
separating the fucosyllactose from the cultivation.

2. The method according to claim 1, wherein the membrane protein is selected from the group consisting of:
a) porters;
b) P-P-bond-hydrolysis-driven transporters;
c) β-Barrel Porins;
d) Auxiliary transport proteins;
e) Putative transport proteins; and
f) Phosphotransfer-driven group translocators.

3. A method of producing fucosyllactose by a genetically modified cell, the method comprising:
providing a cell capable of producing fucosyllactose, the cell comprising at least one nucleic acid sequence coding for a fucosyltransferase that transfers a fucose residue from a guanosine-diphosphate fucose (GDP-fucose) donor to a lactose acceptor, thereby synthesizing fucosyllactose;
the cell further comprising i) a modified expression of an endogenous membrane protein enabling and/or enhancing fucosyllactose transport and/or ii) an expression of a heterologous membrane protein enabling and/or enhancing fucosyllactose transport, and wherein the membrane protein is selected from the group consisting of:
a) porters and wherein the membrane protein does not belonging to the SET family;
b) P-P-bond-hydrolysis-driven transporters;
c) β-Barrel Porins;
d) Auxiliary transport proteins;
e) Putative transport proteins; and
f) Phosphotransfer-driven group translocators;
cultivating the cell in a medium under conditions permissive for the production of the desired fucosyllactose; and
optionally separating the fucosyllactose from the cultivation.

4. The method according to claim 2, wherein the membrane protein is selected from the group consisting of TCDB classes 2.A.1.1, 2.A.1.12, 2.A.1.15, 2.A.1.2, 2.A.1.3, 2.A.1.36, 2.A.1.38, 2.A.1.46, 2.A.1.68, 2.A.1.7, 2.A.1.81, 2.A.123, 2.A.2, 2.A.21, 2.A.58, 2.A.6.3, 2.A.66 and 2.A.7.1, as defined by TCDB.org as released on 17 Jun. 2019.

5. The method according to claim 2, wherein the membrane protein is selected from the group consisting of TCDB classes 3.A.1.1, 3.A.1.2, 3.A.1.10, 3.A.1.11 and 3.A.1.5, as defined by TCDB.org as released on 17 Jun. 2019.

6. The method according to claim 2, wherein the membrane protein is selected from TCDB classes 1.B.3.1 and 1.B.18, as defined by TCDB.org as released on 17 Jun. 2019.

7. The method according to claim 2, wherein the membrane protein is selected from TCDB class 8.A.3, as defined by TCDB.org as released on 17 Jun. 2019.

8. The method according to claim 2, wherein the membrane protein is selected from the group consisting of TCDB classes 9.B.14 and 9.B.158, as defined by TCDB.org as released on 17 Jun. 2019.

9. The method according to claim 2, wherein the membrane protein is selected from the group consisting of TCDB classes 4.A.1.1 and 4.A.4.1, as defined by TCDB.org as released on 17 Jun. 2019.

10. The method according to claim 2, wherein the membrane protein is selected from the group consisting of eggnog families 05BZS, 05C0R, 05C2C, 05CT4, 05CXP, 05CZQ, 05D94, 05DXL, 05E5M, 05E5W, 05E8G, 05EAM, 05EDR, 05EGZ, 05F9N, 05JHE, 05PSV, 05W2Y, 05W3H, 05XJ5, 070Q9, 07CWC, 07QF7, 07QNK, 07RBJ, 07RJ1, 07T5E, 07VQ3, 0814C, 088QT, 08H15, 08N8A, 08SC4, and 08Z4Q, as defined by eggnogdb 1.0.2 as released on 3 Nov. 2017.

11. The method according to claim 2, wherein the membrane protein is selected from the group consisting of eggnog families 05BZ1, 05CJ1, 05EY8, 05HAC, 05DMK, 05DFW, 05MFV, 07FKK, 07R5U, 07V1T, 08IJ9, 08JQ7, and 172T7, as defined by eggnogdb 1.0.2 as released on 3 Nov. 2017.

12. The method according to claim 2, wherein the membrane protein is selected from the group consisting of eggnog family 05DAY, and 08KDD, as defined by eggnogdb 1.0.2 as released on 3 Nov. 2017.

13. The method according to claim 2, wherein the membrane protein is selected from the group consisting of eggnog family 07SYR, as defined by eggnogdb 1.0.2 as released on 3 Nov. 2017.

14. The method according to claim 2, wherein the membrane protein is selected from the group consisting of eggnog families 05CRE, 05GWF, and 06N3A, as defined by eggnogdb 1.0.2 as released on 3 Nov. 2017.

15. The method according to claim 2, wherein the membrane protein is selected from the group consisting of eggnog families 05CI1 and 05VI0, as defined by eggnogdb 1.0.2 as released on 3 Nov. 2017.

16. The method according to claim 2, wherein the membrane protein is chosen from the PFAM list of PF00083, PF00474, PF00873, PF00893, PF01895, PF01943, PF02690, PF03083, PF04193, PF05977, PF07690, PF07690, PF13347, PF13440 and PF14667, as defined by Pfam 32.0 as released on September 2018.

17. The method according to claim 2, wherein the membrane protein is a P-P-bond-hydrolysis-driven transporter chosen from PFAM list PF00005, PF00532, PF00664, PF01061, PF08352, PF14524, PF13407, PF13416 and PF17912, as defined by Pfam 32.0 as released on September 2018.

18. The method according to claim 2, wherein the membrane protein is a β-Barrel Porin chosen from PFAM list PF02264, PF02563, PF10531 and PF18412, as defined by Pfam 32.0 as released on September 2018.

19. The method according to claim 2, wherein the membrane protein is an Auxiliary transport protein chosen from PFAM list PF13807 and PF02706, as defined by Pfam 32.0 as released on September 2018.

20. The method according to claim 2, wherein the membrane protein is a Putative transport protein chosen from PFAM list PF01578, PF03932, PF05140 and PF11045, as defined by Pfam 32.0 as released on September 2018.

21. The method according to claim 2, wherein the membrane protein is a phosphotransfer-driven group translocator chosen from PFAM list PF00367, PF00358, PF02378, and PF03829, as defined by Pfam 32.0 as released on September 2018.

22. The method according to claim 2, wherein the membrane protein is a porter chosen from the InterPro list IPR000390, IPR001036, IPR001411, IPR001734, IPR001927, IPR002797, IPR003663, IPR003841, IPR004316, IPR004633, IPR004638, IPR004734, IPR004812, IPR005275, IPR005828, IPR005829, IPR006603, IPR010290, IPR011701, IPR020846, IPR023008, IPR023721, IPR023722, IPR026022, IPR027417, IPR027463, IPR029303, IPR032896, IPR036259, IPR038078, IPR038377, and IPR039672, as defined by InterPro 75.0 as released on 4 Jul. 2019.

23. The method according to claim 2, wherein the membrane protein is a P-P-bond-hydrolysis-driven transporter chosen from InterPro list IPR000412, IPR001734, IPR001761, IPR003439, IPR003593, IPR005829, IPR005978, IPR005981, IPR006059, IPR006060, IPR006061, IPR008995, IPR011527, IPR011701, IPR013456, IPR013525, IPR013563, IPR015851, IPR015855, IPR017871, IPR019554, IPR020846, IPR025997, IPR026266, IPR027417, IPR028082, IPR029439, IPR033893, IPR036259, IPR036640, IPR038377, IPR039421 and IPR040582, as defined by InterPro 75.0 as released on 4 Jul. 2019.

24. The method according to claim 2, wherein the membrane protein is a β-Barrel Porin chosen from InterPro list IPR003192, IPR003715, IPR019554, IPR023738, IPR036998 and IPR040716, as defined by InterPro 75.0 as released on 4 Jul. 2019.

25. The method according to claim 2, wherein the membrane protein is an Auxiliary transport protein chosen from InterPro list IPR003856, IPR020846, IPR027417, IPR032807 and IPR036259, as defined by InterPro 75.0 as released on 4 Jul. 2019.

26. The method according to claim 2, wherein the membrane protein is a Putative transport protein chosen from InterPro list IPR002541, IPR003439, IPR003593, IPR004316, IPR005627, IPR006603, IPR007816, IPR017871, IPR020368, IPR020846, IPR023648, IPR027417, IPR036259 and IPR036822, as defined by InterPro 75.0 as released on 4 Jul. 2019.

27. The method according to claim 2, wherein the membrane protein is a phosphotransfer-driven group translocator chosen from InterPro list IPR001127, IPR001996, IPR003352, IPR004716, IPR010974, IPR011055, IPR013013, IPR018113, IPR018454, IPR036665 and IPR036878, as defined by InterPro 75.0 as released on 4 Jul. 2019.

28. The method according to claim 2, wherein the membrane protein is i) a porter membrane protein selected from the group consisting of MdfA from Escherichia coli K12 MG1655 with SEQ ID NO: 02, IceT from Escherichia coli K12 MG1655 with SEQ ID NO: 06, Blon_2331 from Bifidobacterium longum subsp. infantis (strain ATCC 15697) with SEQ ID NO: 40, Blon_2332 from B. longum subsp. infantis (strain ATCC 15697) with SEQ ID NO: 42, wzx-like protein of Chitinophaga sp. CF118 with SEQ ID NO: 58, Prevotella ruminicola (AR32) with SEQ ID NO: 66, Lactococcus raffinolactis (ATCC 43920) with SEQ ID NO: 64, and Dyadobacter soli DSM 25329 with SEQ ID NO: 62, ii) a functional homolog or functional fragment of any one of the above porter membrane proteins, or iii) a polypeptide having at least 80% sequence identity to any one of the MdfA, IceT, Blon_2331, Blon_2332 or wzx-like membrane proteins with SEQ ID NO: 02, 06, 40, 42, 58, 66, 64 or 62, respectively.

29. The method according to claim 2, wherein the membrane protein is i) a P-P-bond-hydrolysis driven transporter membrane protein selected from the group consisting of lmrA from Lactococcus lactis strain SRCM103457 with SEQ ID NO: 28, LpsE membrane protein from Sporomusa sphaeroides DSM 2875 with SEQ ID NO: 70 or 74, Flavobacterium spartansii with SEQ ID NO: 68 or 72, TolC from Candidatus Planktophila sulfonica with SEQ ID NO: 76, MsbA from Pedobacter ginsengisoli with SEQ ID NO: 82 or Verrucomicrobia bacterium CG1_02_43_26 with SEQ ID NO: 84, malE from Escherichia coli K-12 MG1655 with SEQ ID NO: 206, malK from Escherichia coli K-12 MG1655 with SEQ ID NO: 208, araF from Escherichia coli K-12 MG1655 with SEQ ID NO: 214, xylF from Escherichia coli K-12 MG1655 with SEQ ID NO: 216, or ytfQ from Escherichia coli K-12 MG1655 with SEQ ID NO: 218, ii) a functional homolog or functional fragment of any of the above P-P-bond-hydrolysis driven transporter membrane proteins or iii) a polypeptide having at least 80% sequence identity to any of the lmrA, LpsE, TolC, MsbA, malE, malK, araF, xylF or ytfQ membrane proteins with SEQ ID NO: 28, 70, 74, 68, 72, 76, 82, 84, 206, 208, 214, 216, or 218, respectively.

30. The method according to claim 2, wherein the membrane protein is i) a β-barrel porin selected from the group consisting of Wza from Escherichia coli K12 MG1655 with SEQ ID NO: 34 and lamB from Escherichia coli K12 MG1655 with SEQ ID NO: 204, ii) a functional homolog or functional fragment of any of the β-barrel porins, or iii) polypeptide having at least 80% sequence identity to the Wza or lamB membrane proteins with SEQ ID NO: 34 or 204, respectively.

31. The method according to claim 2, wherein the membrane protein is i) the auxiliary transport protein Wzc from Thermotoga maritima (strain ATCC 43589/MSB8/DSM 3109/JCM 10099) with SEQ ID NO: 88, ii) a functional homolog or functional fragment of the auxiliary transport protein, or iii) a polypeptide having at least 80% sequence identity to the Wzc membrane protein with SEQ ID NO: 88.

32. The method according to claim 2, wherein the membrane protein is i) a putative transport protein selected from the group consisting of CutC from Clostridium sp. CAG: 1013 with SEQ ID NO: 90, Odoribacter splanchnicus DSM 20712 with SEQ ID NO: 92, Mitsuaria sp. PDC51 with SEQ ID NO: 94, and Prevotella intermedia ATCC 25611 (DSM 20706) with SEQ ID NO: 96, ii) a functional homolog or functional fragment of any of the CutC membrane proteins, or iii) a polypeptide having at least 80% sequence identity to any of the CutC membrane proteins with SEQ ID NO: 90, 92, 94 or 96, respectively.

33. The method according to claim 2, wherein the membrane protein is i) a phosphotransfer-driven group translocator selected from the group consisting of nagE from Escherichia coli K12 MG1655 with SEQ ID NO: 210 and srlB from Escherichia coli K12 MG1655 with SEQ ID NO: 212, ii) a functional homolog or functional fragment of any of the nagE or srlB membrane protein, or iii) a polypeptide having at least 80% sequence identity to any of the nagE or srlB membrane proteins with SEQ ID NO: 210 or 212, respectively.

34. The method according to claim 2, wherein the membrane protein is i) a porter membrane protein selected from the group consisting of MdfA from Escherichia coli K12 MG1655 with SEQ ID NO: 02, IceT from Escherichia coli K12 MG1655 with SEQ ID NO: 06, YnfM from Escherichia coli K12 MG1655 with SEQ ID NO: 04, Yhhs from Escherichia coli K12 MG1655 with SEQ ID NO: 08, EmrD from Escherichia coli K12 MG1655 with SEQ ID NO: 10, YdhC from Escherichia coli K12 MG1655 with SEQ ID NO: 12, YbdA from Escherichia coli K12 MG1655 with SEQ ID NO: 14, YdeE from Escherichia coli K12 MG1655 with SEQ ID NO: 16, MhpT from Escherichia coli K12 MG1655 with SEQ ID NO: 18, YebQ from Escherichia coli K12 MG1655 with SEQ ID NO: 20, YjhB from Escherichia coli K12 MG1655 with SEQ ID NO: 22, Bcr from Escherichia coli K12 MG1655 with SEQ ID NO: 24, FucP from Escherichia coli K12 MG1655 with SEQ ID NO: 26, WzxE from Escherichia coli K12 MG1655 with SEQ ID NO: 32, EmrE from Escherichia coli K12 MG1655 with SEQ ID NO: 38, Blon_2331 from Bifidobacterium longum subsp. infantis (strain ATCC 15697) with SEQ ID NO: 40, Blon_2332 from Bifidobacterium longum subsp. infantis (strain ATCC 15697) with SEQ ID NO: 42, Blon_0247 from Bifidobacterium longum subsp. infantis (strain ATCC 15697) with SEQ ID NO: 46, Blon_0245 from Bifidobacterium longum subsp. infantis (strain ATCC 15697) with SEQ ID NO: 48, Blon_0345 from Bifidobacterium longum subsp. infantis (strain ATCC 15697) with SEQ ID NO: 50, CDT2 from Neurospora crassa OR74A with SEQ ID-NA NO: 52, CDT2 from Aspergillus oryzae RIB40 with SEQ ID NO: 54, Wzx from Chitinophaga sp. CF 118 with SEQ ID NO: 58, Wzx from Eubacterium sp. CAG:581 with SEQ ID NO: 60, Wzx from Dyadobacter soli (DSM 25329) with SEQ ID NO: 62, Wzx from Lactococcus raffinolactis (ATCC 43920) with SEQ ID NO: 64, Wzx from Prevotella ruminicola (AR32) with SEQ ID NO: 66, NAPO from Brachyspira hampsonii P280/1 with SEQ ID NO: 86, NAm from Actinobaculum suis (DSM 20639) with SEQ ID NO: 98, NAm from Ruminococcus gnavus with SEQ ID NO: 100, NAm from Curtobacterium sp. 314Chir4.1 with SEQ ID NO: 102, Nap from Niabella drilacis (DSM25811) with SEQ ID NO: 104, Nap from Saccharicrinis fermentans (DSM 9555) with SEQ ID NO: 106, mdtD from Citrobacter freundii MGH152 with SEQ ID NO: 108, mdtD from Citrobacter werkmanii NBRC 105721 with SEQ ID NO: 110, mdtD from Citrobacter amalonaticus with SEQ ID NO: 112, mdtD from Klebsiella oxytoca with SEQ ID NO: 114, mdtD from Escherichia albertii B156 with SEQ ID NO: 116, yegB from Salmonella enterica subsp. salamae with SEQ ID NO: 118, mdtD from Klebsiella pneumoniae 30684/NJST258_2 with SEQ ID NO: 120, Tcr_1_D38215 from Klebsiella pneumoniae with SEQ ID NO: 122, mdtD from Pseudocitrobacter faecalis with SEQ ID NO: 124, Cmr from Yokenella regensburgei (ATCC43003) with SEQ ID NO: 126, MdfA from Cronobacter muytjensii with SEQ ID NO: 128, MdfA from Klebsiella oxytoca with SEQ ID NO: 130, MFS from Citrobacter koseri with SEQ ID NO: 132, MdfA from Escherichia marmotae with SEQ ID NO: 134, Cmr from Shigella flexneri with SEQ ID NO: 136, MdfA from Salmonella enterica subsp. salamae with SEQ ID NO: 138, Cmr from Citrobacter youngae (ATCC 29220) with SEQ ID NO: 140, MdfA from Citrobacter freundii with SEQ ID NO: 142, MdfA from Enterobacter kobei with SEQ ID NO: 144, MdfA from Enterobacter sp. with SEQ ID NO: 146, MdfA from Lelliottia sp. WB101 with SEQ ID NO: 148, MdfA from Enterobacter ludwigii EcWSU1 with SEQ ID NO: 150, Sweet-like protein from Actinoplanes utahensis with SEQ ID NO: 152, Sweet-like protein from Chitinophagaceae bacterium PMG_246 with SEQ ID NO: 154, Sweet-like protein from Rhizobium sp. PDC82 with SEQ ID NO: 156, Sweet-like protein from Kineococcus rhizosphaerae (DSM 19711) with SEQ ID NO: 158, Sweet-like protein from Morganella morganii IS15 with SEQ ID NO: 160, Sweet-like protein from Geodermatophilus obscurus (strain ATCC 25078) with SEQ ID NO: 162, Sweet-like protein from Bradyrhizobium sp. BTAi1 with SEQ ID NO: 164, Sweet-like protein from Bradyrhizobium japonicum USDA 110 with SEQ ID NO: 166, Sweet-like protein from Xanthomonas campestris pv. vesicatoria str. 85-10 with SEQ ID NO: 168, Sweet-like protein from Herbaspirillum aquaticum with SEQ ID NO: 170, Sweet-like protein from Flavobacteria bacterium MS024-2A with SEQ ID NO: 172, rnd-like from Sinorhizobium medicae WSM419 with SEQ ID NO: 182 and arabinose efflux from Azospirillum brasiliense LMG 04375 with SEQ ID NO: 184, ii) a functional homolog or functional fragment of any of the above porter membrane proteins, or iii) a polypeptide having an amino acid sequence having at least 80% sequence identity to any of the MdfA, IceT, YnfM, Yhhs, EmrD, YdhC, YbdA, YdeE, MhpT, YebQ, YjhB, Bcr, FucP, WzxE, EmrE, Wzx, Blon_2331, Blon_2232, Blon_0247, Blon_0245, Blon_0345, NAPO, NAm, Nap, mdtD, YegB, Tcr_1_D38215, cmr, MFS, CDT2, rnd, Sweet-like or arabinose efflux membrane proteins with SEQ ID NO: 02, 06, 04, 08, 10, 12, 14, 16, 18, 20, 22, 24, 26, 32, 38, 40, 42, 46, 48, 50, 52, 54, 58, 60, 62, 64, 66, 86, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 182 or 184, respectively.

35. The method according to claim 2, wherein the membrane protein is i) a P-P-bond-hydrolysis-driven transporter selected from the group consisting of LmrA from Lactococcus lactis strain SRCM103457 with SEQ ID NO: 28, OppF from Escherichia coli strain K12 MG1655 with SEQ ID NO: 30, Wzk from Helicobacter pylori (strain ATCC 700392/26695) with SEQ ID NO: 36, Blon_2475 from Bifidobacterium longum subsp. infantis (strain ATCC 15697) with SEQ ID NO: 44, LpsE from Flavobacterium spartansii with SEQ ID NO: 68 or 72, LpsE from Sporomusa sphaeroides DSM 2875 with SEQ ID NO: 70 or 74, TolC from Candidatus Planktophila sulfonica with SEQ ID NO: 76, TolC from Butyrivibrio hungatei XBD2006 with SEQ ID NO: 78, MsbA from Roseburia intestinalis CAG:13 with SEQ ID NO: 80, MsbA from Pedobacter ginsengisoli with SEQ ID G-NO: 82, MsbA from Verrucomicrobia bacterium CG1_02_43_26 with SEQ ID NO: 84, Wzm from Rhizobium sp. Root149 with SEQ ID NO: 174, Wzm from Azospirillum brasiliense LMG 04375 with SEQ ID NO: 176, Wzm from Escherichia coli 113303 with SEQ ID NO: 196, Wzt from Rhizobium sp. Root149 with SEQ ID NO: 178, Wzt from Azospirillum brasiliense LMG 04375 with SEQ ID NO: 180, Wzt from Escherichia coli 113303 with SEQ ID NO: 194, Nodj from Bradyrhizobium japonicum USDA 110 with SEQ ID NO: 188 or 190, malE from Escherichia coli K-12 MG1655 with SEQ ID NO: 206, malK from Escherichia coli K-12 MG1655 with SEQ ID NO: 208, araF from Escherichia coli K-12 MG1655 with SEQ ID NO: 214, xylF from Escherichia coli K-12 MG1655 with SEQ ID NO: 216, and ytfQ from Escherichia coli K-12 MG1655 with SEQ ID NO: 218, ii) a functional homolog or functional fragment of any one of the above P-P-bond-hydrolysis-driven transporter membrane proteins, or iii) a polypeptide having an amino acid sequence having at least 80% sequence identity to any of the LmrA, OppF, Wzk, Blon_2475, LpsE, TolC, MsbA, Wzm, Wzt, Nodj, malE, malK, araF, xylF or ytfQ membrane proteins with SEQ ID NO: 28, 30, 36, 44, 68, 72, 70, 74, 76, 78, 80, 82, 84, 174, 176, 196, 178, 180, 194, 188, 190, 206, 208, 214, 216 or 218, respectively.

36. The method according to claim 2, wherein the membrane protein is i) a putative transport protein selected from the group consisting of Cytochrome C biogenesis protein from Helicobacter pylori with SEQ ID NO: 56, CutC from Clostridium sp. CAG: 1013 with SEQ ID NO: 90, CutC from Odoribacter splanchnicus DSM 20712 with SEQ ID NO: 92, CutC from Mitsuaria sp. PDC51 with SEQ ID NO: 94, CutC from Prevotella intermedia ATCC 25611 (DSM 20706) with SEQ ID NO: 96, ybjM from Escherichia coli K12 MG1655 with SEQ ID-N9 NO: 190- and ybjM from Enterobacteriaceae bacterium ENNIH1 with SEQ ID NO: 192, ii) a functional homolog or functional fragment of any one of the above putative transport proteins, or iii) a polypeptide having an amino acid sequence having at least 80% sequence identity to any one of the CytC, CutC or ybjM membrane proteins with SEQ ID NO: 56, 90, 92, 94, 96, 190 or 192, respectively.

37. A method for producing fucosyllactose by a genetically modified cell, the method comprising:
providing a cell capable of producing fucosyllactose, the cell comprising at least one nucleic acid sequence coding for a fucosyltransferase that transfers a fucose residue from a GDP-fucose donor to a lactose acceptor, thereby synthesizing fucosyllactose
the cell further comprising i) a modified expression of an endogenous membrane protein enabling and/or enhancing fucosyllactose transport and/or ii) an expression of a heterologous membrane protein enabling and/or enhancing fucosyllactose transport, and wherein membrane protein is i) selected from the group of membrane proteins consisting of the porter membrane proteins MdfA from Escherichia coli K12 MG1655 with SEQ ID NO: 02, IceT from Escherichia coli K12 MG1655 with SEQ ID NO: 06, YnfM from Escherichia coli K12 MG1655 with SEQ ID NO: 04, Yhhs from Escherichia coli K12 MG1655 with SEQ ID NO: 08, EmrD from Escherichia coli K12 MG1655 with SEQ ID NO: 10, YdhC from Escherichia coli K12 MG1655 with SEQ ID NO: 12, YbdA from Escherichia coli K12 MG1655 with SEQ ID NO: 14, YdeE from Escherichia coli K12 MG1655 with SEQ ID NO: 16, MhpT from Escherichia coli K12 MG1655 with SEQ ID NO: 18, YebQ from Escherichia coli K12 MG1655 with SEQ ID NO: 20, YjhB from Escherichia coli K12 MG1655 with SEQ ID NO: 22, Bcr from Escherichia coli K12 MG1655 with SEQ ID NO: 24, FucP from Escherichia coli K12 MG1655 with SEQ ID-NO: 26, WzxE from Escherichia coli K12 MG1655 with SEQ ID NO: 32, EmrE from Escherichia coli K12 MG1655 with SEQ ID NO: 38, Blon_2331 from Bifidobacterium longum subsp. infantis (strain ATCC 15697) with SEQ ID NO: 40, Blon_2332 from Bifidobacterium longum subsp. infantis (strain ATCC 15697) with SEQ ID NO: 42, Blon_0247 from Bifidobacterium longum subsp. infantis (strain ATCC 15697) with SEQ ID NO: 46, Blon_0245 from Bifidobacterium longum subsp. infantis (strain ATCC 15697) with SEQ ID NO: 48, Blon_0345 from Bifidobacterium longum subsp. infantis (strain ATCC 15697) with SEQ ID NO: 50, CDT2 from Neurospora crassa OR74A with SEQ ID NO: 52, CDT2 from Aspergillus oryzae RIB40 with SEQ ID NO-_NO 0.54, Wzx from Chitinophaga sp. CF118 with SEQ ID NO: 58, Wzx from Eubacterium sp. CAG:581 with SEQ ID NO: 60, Wzx from Dyadobacter soli (DSM 25329) with SEQ ID NO: 62, Wzx from Lactococcus raffinolactis (ATCC 43920) with SEQ ID NO: 64, Wzx from Prevotella ruminicola (AR32) with SEQ ID NO: 66, NAPO from Brachyspira hampsonii P280/1 with SEQ ID NO: 86, NAm from Actinobaculum suis (DSM 20639) with SEQ ID NO: 98, NAm from Ruminococcus gnavus with SEQ ID NO: 100, NAm from Curtobacterium sp. 314Chir4.1 with SEQ ID NO: 102, Nap from Niabella drilacis (DSM25811) with SEQ ID NO: 104, Nap from Saccharicrinis fermentans (DSM 9555) with SEQ ID NO: 106, mdtD from Citrobacter freundii MGH152 with SEQ ID NO: 108, mdtD from Citrobacter werkmanii NBRC 105721 with SEQ ID NO: 110, mdtD from Citrobacter amalonaticus with SEQ ID NO: 112, mdtD from Klebsiella oxytoca with SEQ ID NO: 114, mdtD from Escherichia albertii B156 with SEQ ID NO: 116, yegB from Salmonella enterica subsp. salamae with SEQ ID NO: 118, mdtD from Klebsiella pneumoniae 30684/NJST258_2 with SEQ ID NO: 120, Tcr_1_D38215 from Klebsiella pneumoniae with SEQ ID NO: 122, mdtD from Pseudocitrobacter faecalis with SEQ ID NO: 124, Cmr from Yokenella regensburgei (ATCC43003) with SEQ ID NO: 126, MdfA from Cronobacter muytjensii with SEQ ID NO: 128, MdfA from Klebsiella oxytoca with SEQ ID NO: 130, MFS from Citrobacter koseri with SEQ ID NO: 132, MdfA from Escherichia marmotae with SEQ ID NO: 134, Cmr from Shigella flexneri with SEQ ID NO: 136, MdfA from Salmonella enterica subsp. salamae with SEQ ID NO: 138, Cmr from Citrobacter youngae (ATCC 29220) with SEQ ID NO: 140, MdfA from Citrobacter freundii with SEQ ID NO: 142, MdfA from Enterobacter kobei with SEQ ID NO: 144, MdfA from Enterobacter sp. with SEQ ID NO: 146, MdfA from Lelliottia sp. WB101 with SEQ ID NO: 148, MdfA from Enterobacter ludwigii EcWSU1 with SEQ ID NO: 150, Sweet-like protein from Actinoplanes utahensis with SEQ ID NO: 152, Sweet-like protein from Chitinophagaceae bacterium PMG_246 with SEQ ID NO: 154, Sweet-like protein from Rhizobium sp. PDC82 with SEQ ID NO: 156, Sweet-like protein from Kineococcus rhizosphaerae (DSM 19711) with SEQ ID NO: 158, Sweet-like protein from Morganella morganii IS15 with SEQ ID NO: 160, Sweet-like protein from Geodermatophilus obscurus (strain ATCC 25078) with SEQ ID NO: 162, Sweet-like protein from Bradyrhizobium sp. BTAi1 with SEQ ID NO: 164, Sweet-like protein from Bradyrhizobium japonicum USDA 110 with SEQ ID NO: 166, Sweet-like protein from Xanthomonas campestris pv. vesicatoria str. 85-10 with SEQ ID NO: 168, Sweet-like protein from Herbaspirillum aquaticum with SEQ ID NO: 170, Sweet-like protein from Flavobacteria bacterium MS024-2A with SEQ ID NO: 172, rnd-like from Sinorhizobium medicae WSM419 with SEQ ID NO: 182 and arabinose efflux from Azospirillum brasiliense LMG 04375 with SEQ ID NO: 184, ii) a functional homolog or functional fragment of any of the above porter membrane proteins, iii) a polypeptide having an amino acid sequence having at least 80% sequence identity to any of the MdfA, IceT, YnfM, Yhhs, EmrD, YdhC, YbdA, YdeE, MhpT, YebQ, YjhB, Bcr, FucP, WzxE, EmrE, Wzx, Blon_2331, Blon_2232, Blon_0247, Blon_0245, Blon_0345, NAPO, NAm, Nap, mdtD, YegB, Tcr_1_D38215, cmr, MFS, CDT2, rnd, Sweet-like or arabinose efflux membrane proteins with SEQ ID NO: 02, 06, 04, 08, 10, 12, 14, 16, 18, 20, 22, 24, 26, 32, 38, 40, 42, 46, 48, 50, 52, 54, 58, 60, 62, 64, 66, 86, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 182 or 184, respectively; iv) selected from the group of membraned proteins consisting of P-P-bond-hydrolysis-driven transporters LmrA from Lactococcus lactis strain SRCM103457 with SEQ ID NO: 28, OppF from Escherichia coli strain K12 MG1655 with SEQ ID NO: 30, Wzk from Helicobacter pylori (strain ATCC 700392/26695) with SEQ ID NO: 36, Blon_2475 from Bifidobacterium longum subsp. infantis (strain ATCC 15697) with SEQ ID NO: 44, LpsE from Flavobacterium spartansii with SEQ ID NO: 68 or 72, LpsE from Sporomusa sphaeroides DSM 2875 with SEQ ID NO: 70 or 74, TolC from Candidatus Planktophila sulfonica with SEQ ID NO: 76, TolC from Butyrivibrio hungatei XBD2006 with SEQ ID NO: 78, MsbA from Roseburia intestinalis CAG:13 with SEQ ID NO: 80, MsbA from Pedobacter ginsengisoli with SEQ ID NO: 82, MsbA from Verrucomicrobia bacterium CG1_02_43_26 with SEQ ID NO: 84, Wzm from Rhizobium sp. Root149 with SEQ ID NO: 174, Wzm from Azospirillum brasiliense LMG 04375 with SEQ ID NO: 176, Wzm from Escherichia coli 113303 with SEQ ID NO: 196, Wzt from Rhizobium sp. Root149 with SEQ ID NO: 178, Wzt from Azospirillum brasiliense LMG 04375 with SEQ ID NO: 180, Wzt from Escherichia coli 113303 with SEQ ID NO: 194, Nodj from Bradyrhizobium japonicum USDA 110 with SEQ ID NO: 188 or 190, malE from Escherichia coli K-12 MG1655 with SEQ ID NO: 206, malK from Escherichia coli K-12 MG1655 with SEQ ID NO: Q208, araF from Escherichia coli K-12 MG1655 with SEQ ID NO: 214, xylF from Escherichia coli K-12 MG1655 with SEQ ID NO: 216, and ytfQ from Escherichia coli K-12 MG1655 with SEQ ID NO: 218, v) a functional homolog or functional fragment of any one of the P-P-bond-hydrolysis-driven transporter membrane proteins, vi) a polypeptide having an amino acid sequence having at least 80% sequence identity to any of the LmrA, OppF, Wzk, Blon_2475, LpsE, TolC, MsbA, Wzm, Wzt, Nodj, malE, malK, araF, xylF or ytfQ membrane proteins with SEQ ID NO: 28, 30, 36, 44, 68, 72, 70, 74, 76, 78, 80, 82, 84, 174, 176, 196, 178, 180, 194, 188, 190, 206, 208, 214, 216, or 218, respectively; vii) selected from the group of membrane proteins consisting of β-barrel porin membrane proteins Wza from Escherichia coli K12 MG1655 with SEQ ID NO: 34 and lamB from Escherichia coli K12 MG1655 with SEQ ID NO: 204, viii) a functional homolog or functional fragment of any one of the Wza or lamB protein, ix) a polypeptide having at least 80% sequence identity to any one of the Wza or lamB membrane protein with SEQ ID NO: 34 or 204, respectively; x) the auxiliary transport protein Wzc from Thermotoga maritima (strain ATCC 43589/MSB8/DSM 3109/JCM 10099) with SEQ ID NO: 88, xi) a functional homolog or functional fragment of the auxiliary transport protein, xii) polypeptide having at least 80% sequence identity to the Wzc membrane protein with SEQ ID NO: 88; xiii) selected from the group of membrane proteins consisting of the putative transport proteins Cytochrome C biogenesis protein from Helicobacter pylori with SEQ ID NO: 56, CutC from Clostridium sp. CAG: 1013 with SEQ ID NO: 90, CutC from Odoribacter splanchnicus DSM 20712 with SEQ ID NO: 92, CutC from Mitsuaria sp. PDC51 with SEQ ID NO: 94, CutC from Prevotella intermedia ATCC 25611 (DSM 20706) with SEQ ID NO: 96, ybjM from Escherichia coli K12 MG1655 with SEQ ID NO: 190 and ybjM from Enterobacteriaceae bacterium ENNIH1 with SEQ ID NO: 192, xiv) a functional homolog or functional fragment of any one of the putative transport protein, xv) a polypeptide having an amino acid sequence having at least 80% sequence identity to any one of the CytC, CutC or ybjM membrane proteins with SEQ ID NO: 56, 90, 92, 94, 96, 190 or 192, respectively, xvi) selected from the group of membrane proteins consisting of the phosphotransfer-driven group translocators nagE from Escherichia coli K12 MG1655 with SEQ ID NO: 210, and srlB from Escherichia coli K12 MG1655 with SEQ ID NO: 212, xviii a functional homolog or functional fragment of any of the nagE or srlB membrane proteins, or xviii) a polypeptide having at least 80% sequence identity to any one of the nagE or srlB membrane proteins with SEQ ID NO: 210 or 212, respectively.

38. The method according to claim 1, wherein the membrane protein is a transporter protein involved in transport of compounds across the outer membrane of the cell wall.

39. The method according to claim 1, the method further comprising:
i) adding to the culture medium a lactose feed comprising at least 50 grams of lactose per liter of initial reactor volume wherein the reactor volume ranges from 250 mL to 10 000 m³;
ii) adding a lactose feed in a continuous manner to the culture medium over the course of 1 to 5 days by means of a feeding solution; and
iii) adding a lactose feed in a continuous manner to the culture medium over the course of 1 to 5 days by means of a feeding solution and wherein the concentration of the lactose feeding solution is 50 g/L to 600 g/L;
the method resulting in a fucosyllactose concentration of at least 50 g/L in the final volume of the culture medium.

40. The method of claim 39, wherein the lactose feed is accomplished by adding lactose from the beginning of the cultivation in a concentration of at least 5 mM.

41. The method of claim 39, wherein the lactose feed is accomplished by adding lactose to the cultivation medium in a concentration, such that throughout the production phase of the cultivation a lactose concentration of at least 5 mM is obtained.

42. The method of claim 39, wherein the host cells are cultivated for at least about 60 hours.

43. The method of claim 39, wherein a carbon and energy source is also added to the culture medium.

44. The method of claim 39, wherein a first phase of exponential cell growth is provided by adding a carbon-based substrate to the culture medium before the lactose is added to the culture medium in a second phase.

45. The method according to claim 1, wherein the method is producing a mixture of fucosyllactoses.

46. The method according to claim 1, wherein the fucosyllactose is 2′-fucosyllactose, 3-fucosyllactose and/or difucosyllactose.

47. The method according to claim 1, wherein the genetically modified cell is selected from the group consisting of microorganism, plant, and animal cells.

48. The method according to claim 47, wherein the cell is an Escherichia coli cell.

49. A host cell genetically modified for the production of a fucosyllactose, wherein the host cell comprises at least one nucleic acid sequence coding for a fucosyltransferase that transfers a fucose residue from a GDP-fucose donor to a lactose acceptor thereby synthesizing fucosyllactose the cell further comprising i) a modified expression of an endogenous membrane protein enabling and/or enhancing fucosyllactose transport and/or ii) an expression of a heterologous membrane protein enabling and/or enhancing fucosyllactose transport, and wherein the membrane protein is i) selected from the group consisting of membrane proteins comprising any one of the PFAM domains found by searching the genomic neighborhood of GT10 and GT11 fucosyltransferase families with InterPro number IPR001503 and IPR002516 as defined on InterPro 75.0 released on 4 Jul. 2019 respectively, wherein the genomic neighborhood window size is 14 genes before and 14 genes after the respective fucosyltransferase and wherein the membrane protein is not belonging to the SET family, ii) selected from the group consisting of membrane proteins comprising SEQ ID NO: 204, 206, 208, 210, 212, 214, 216, and 218, iii) a functional homolog or functional fragment of any one of the membrane proteins comprising SEQ ID NO: 204, 206, 208, 210, 212, 214, 216, and 218, or iv) a polypeptide having at least 80% sequence identity to any one of the membrane proteins with SEQ ID NO: 204, 206, 208, 210, 212, 214, 216, or 218.

50. The host cell according to claim 49, wherein the membrane protein is selected from the group consisting of:
a) porters;
b) P-P-bond-hydrolysis-driven transporters;
c) β-Barrel Porins;
d) Auxiliary transport proteins;
e) Putative transport proteins; and
f) Phosphotransfer-driven group translocators.

51. A host cell genetically modified for production of a fucosyllactose, wherein the host cell comprises at least one nucleic acid sequence coding for a fucosyltransferase that transfers a fucose residue from a GDP-fucose donor to a lactose acceptor, thereby synthesizing fucosyllactose,
the cell further comprising i) a modified expression of an endogenous membrane protein enabling and/or enhancing fucosyllactose transport and/or ii) an expression of a heterologous membrane protein enabling and/or enhancing fucosyllactose transport, and wherein the membrane protein is selected from the group consisting of:
a) porters and wherein the membrane protein does not belonging to the SET family;
b) P-P-bond-hydrolysis-driven transporters;
c) β-Barrel Porins;
d) Auxiliary transport proteins;
e) Putative transport proteins; and
f) Phosphotransfer-driven group translocators.

52. The host cell of claim 50, wherein the membrane protein is selected from the group consisting of TCDB classes 2.A.1.1, 2.A.1.12, 2.A.1.15, 2.A.1.2, 2.A.1.3, 2.A.1.36, 2.A.1.38, 2.A.1.46, 2.A.1.68, 2.A.1.7, 2.A.1.81, 2.A.123, 2.A.2, 2.A.21, 2.A.58, 2.A.6.3, 2.A.66, and 2.A.7.1, as defined by TCDB.org as released on 17 Jun. 2019.

53. The host cell of claim 50, wherein the membrane protein is i) a porter membrane protein selected from the group consisting of MdfA from Escherichia coli K12 MG1655 with SEQ ID NO: 02, IceT from Escherichia coli K12 MG1655 with SEQ ID NO: 06, YnfM from Escherichia coli K12 MG1655 with SEQ ID NO: 04, Yhhs from Escherichia coli K12 MG1655 with SEQ ID NO: 08, EmrD from Escherichia coli K12 MG1655 with SEQ ID NO: 10, YdhC from Escherichia coli K12 MG1655 with SEQ ID NO: 12, YbdA from Escherichia coli K12 MG1655 with SEQ ID NO: 14, YdeE from Escherichia coli K12 MG1655 with SEQ ID NO: 16, MhpT from Escherichia coli K12 MG1655 with SEQ ID NO: 18, YebQ from Escherichia coli K12 MG1655 with SEQ ID NO: 20, YjhB from Escherichia coli K12 MG1655 with SEQ ID NO: 22, Bcr from Escherichia coli K12 MG1655 with SEQ ID NO: 24, FucP from Escherichia coli K12 MG1655 with SEQ ID NO: 26, WzxE from Escherichia coli K12 MG1655 with SEQ ID NO: 32, EmrE from Escherichia coli K12 MG1655 with SEQ ID NO: 38, Blon_2331 from Bifidobacterium longum subsp. infantis (strain ATCC 15697) with SEQ ID NO: 40, Blon_2332 from Bifidobacterium longum subsp. infantis (strain ATCC 15697) with SEQ ID NO: 42, Blon_0247 from Bifidobacterium longum subsp. infantis (strain ATCC 15697) with SEQ ID NO: 46, Blon_0245 from Bifidobacterium longum subsp. infantis (strain ATCC 15697) with SEQ ID NO:48, Blon_0345 from Bifidobacterium longum subsp. infantis (strain ATCC 15697) with SEQ ID NO: 50, CDT2 from Neurospora crassa OR74A with SEQ ID NO: 52, CDT2 from Aspergillus oryzae RIB40 with SEQ ID NO: 54, Wzx from Chitinophaga sp. CF 118 with SEQ ID-ONO:58, Wzx from Eubacterium sp. CAG:581 with SEQ ID NO: 60, Wzx from Dyadobacter soli (DSM 25329) with SEQ ID NO: 62, Wzx from Lactococcus raffinolactis (ATCC 43920) with SEQ ID NO: 64, Wzx from Prevotella ruminicola (AR32) with SEQ ID NO: 66, NAPO from Brachyspira hampsonii P280/1 with SEQ ID NO: 86, NAm from Actinobaculum suis (DSM 20639) with SEQ ID NO: 98, NAm from Ruminococcus gnavus with SEQ ID NO: 100, NAm from Curtobacterium sp. 314Chir4.1 with SEQ ID NO: 102, Nap from Niabella drilacis (DSM25811) with SEQ ID NO: 104, Nap from Saccharicrinis fermentans (DSM 9555) with SEQ ID NO: 106, mdtD from Citrobacter freundii MGH152 with SEQ ID NO: 108, mdtD from Citrobacter werkmanii NBRC 105721 with SEQ ID NO: 110, mdtD from Citrobacter amalonaticus with SEQ ID NO: 112, mdtD from Klebsiella oxytoca with SEQ ID NO: 114, mdtD from Escherichia albertii B156 with SEQ ID NO: 116, yegB from Salmonella enterica subsp. salamae with SEQ ID NO: 118, mdtD from Klebsiella pneumoniae 30684/NJST258_2 with SEQ ID NO: 120, Tcr_1_D38215 from Klebsiella pneumoniae with SEQ ID NO: 122, mdtD from Pseudocitrobacter faecalis with SEQ ID NO: 124, Cmr from Yokenella regensburgei (ATCC43003) with SEQ ID NO: 126, MdfA from Cronobacter muytjensii with SEQ ID NO: 128, MdfA from Klebsiella oxytoca with SEQ ID NO: 130, MFS from Citrobacter koseri with SEQ ID NO: 132, MdfA from Escherichia marmotae with SEQ ID NO: 134, Cmr from Shigella flexneri with SEQ ID NO: 136, MdfA from Salmonella enterica subsp. salamae with SEQ ID NO: 138, Cmr from Citrobacter youngae (ATCC 29220) with SEQ ID NO: 140, MdfA from Citrobacter freundii with SEQ ID NO: 142, MdfA from Enterobacter kobei with SEQ ID NO: 144, MdfA from Enterobacter sp. with SEQ ID NO: 146, MdfA from Lelliottia sp. WB101 with SEQ ID NO: 148, MdfA from Enterobacter ludwigii EcWSU1 with SEQ ID NO: 150, Sweet-like protein from Actinoplanes utahensis with SEQ ID NO: 152, Sweet-like protein from Chitinophagaceae bacterium PMG_246 with SEQ ID NO: 154, Sweet-like protein from Rhizobium sp. PDC82 with SEQ ID NO: 156, Sweet-like protein from Kineococcus rhizosphaerae (DSM 19711) with SEQ ID NO: 158, Sweet-like protein from Morganella morganii IS15 with SEQ ID NO: 160, Sweet-like protein from Geodermatophilus obscurus (strain ATCC 25078) with SEQ ID NO: 162, Sweet-like protein from Bradyrhizobium sp. BTAi1 with SEQ ID NO: 164, Sweet-like protein from Bradyrhizobium japonicum USDA 110 with SEQ ID NO: 166, Sweet-like protein from Xanthomonas campestris pv. vesicatoria str. 85-10 with SEQ ID NO: 168, Sweet-like protein from Herbaspirillum aquaticum with SEQ ID NO: 170, Sweet-like protein from Flavobacteria bacterium MS024-2A with SEQ ID NO: 172, rnd-like from Sinorhizobium medicae WSM419 with SEQ ID NO: 182 and arabinose efflux from Azospirillum brasiliense LMG 04375 with SEQ ID NO: 184, ii) a functional homolog or functional fragment of any of the above porter membrane proteins, or iii) a protein-polypeptide having an amino acid sequence having at least 80% sequence identity to any of the MdfA, IceT, YnfM, Yhhs, EmrD, YdhC, YbdA, YdeE, MhpT, YebQ, YjhB, Bcr, FucP, WzxE, EmrE, Wzx, Blon_2331, Blon_2232, Blon_0247, Blon_0245, Blon_0345, NAPO, NAm, Nap, mdtD, YegB, Tcr_1_D38215, cmr, MFS, CDT2, md, Sweet-like or arabinose efflux membrane proteins with SEQ ID NO: 02, 06, 04, 08, 10, 12, 14, 16, 18, 20, 22, 24, 26, 32, 38, 40, 42, 46, 48, 50, 52, 54, 58, 60, 62, 64, 66, 86, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 182 or 184, respectively.

54. The host cell of claim 50, wherein the membrane protein is i) a P-P-bond-hydrolysis-driven transporter is selected from the group consisting of LmrA from Lactococcus lactis strain SRCM103457 with SEQ ID NO: 28, OppF from Escherichia coli strain K12 MG1655 with SEQ ID NO: 30, Wzk from Helicobacter pylori (strain ATCC 700392/26695) with SEQ ID NO: 36, Blon_2475 from Bifidobacterium longum subsp. infantis (strain ATCC 15697) with SEQ ID NO: 44, LpsE from Flavobacterium spartansii with SEQ ID NO: 68 or 72, LpsE from Sporomusa sphaeroides DSM 2875 with SEQ ID NO: 70 or 74, TolC from Candidatus Planktophila sulfonica with SEQ ID NO: 76, TolC from Butyrivibrio hungatei XBD2006 with SEQ ID NO: 78, MsbA from Roseburia intestinalis CAG:13 with SEQ ID NO: 80, MsbA from Pedobacter ginsengisoli with SEQ ID NO: 82, MsbA from Verrucomicrobia bacterium CG1_02_43_26 with SEQ ID NO: 84, Wzm from Rhizobium sp. Root149 with SEQ ID NO: 174, Wzm from Azospirillum brasiliense LMG 04375 with SEQ ID NO: 176, Wzm from Escherichia coli 113303 with SEQ ID NO: 196, Wzt from Rhizobium sp. Root149 with SEQ ID NO: 178, Wzt from Azospirillum brasiliense LMG 04375 with SEQ ID NO: 180, Wzt from Escherichia coli 113303 with SEQ ID NO NO: 194, Nodj from Bradyrhizobium japonicum USDA 110 with SEQ ID NO: 188 or 190, malE from Escherichia coli K-12 MG1655 with SEQ ID NO: 206, malK from Escherichia coli K-12 MG1655 with SEQ ID NO: 208, araF from Escherichia coli K-12 MG1655 with SEQ ID NO: 214, xylF from Escherichia coli K-12 MG1655 with SEQ ID NO: 216, and ytfQ from Escherichia coli K-12 MG1655 with SEQ ID NO: 218, ii) a functional homolog or functional fragment of any one of the above P-P-bond-hydrolysis-driven transporter membrane proteins, or iii) a polypeptide having an amino acid sequence having at least 80% sequence identity to any of the LmrA, OppF, Wzk, Blon_2475, LpsE, TolC, MsbA, Wzm, Wzt, Nodj, malE, malK, araF, xylF or ytfQ membrane proteins with SEQ ID NO: 28, 30, 36, 44, 68, 72, 70, 74, 76, 78, 80, 82, 84, 174, 176, 196, 178, 180, 194, 188, 190, 206, 208, 214, 216, or 218, respectively.

55. The host cell of claim 50, wherein the membrane protein is i) a putative transport protein selected from the group consisting of Cytochrome C biogenesis protein from Helicobacter pylori with SEQ ID NO: 56, CutC from Clostridium sp. CAG: 1013 with SEQ ID NO: 90, CutC from Odoribacter splanchnicus DSM 20712 with SEQ ID NO: 92, CutC from Mitsuaria sp. PDC51 with SEQ ID NO: 94, CutC from Prevotella intermedia ATCC 25611 (DSM 20706) with SEQ ID NO: 96, ybjM from Escherichia coli K12 MG1655 with SEQ ID NO: 190, and ybjM from Enterobacteriaceae bacterium ENNIH1 with SEQ ID NO: 192, ii) a functional homolog or functional fragment of any one of the above putative transport proteins, or iii) a polypeptide having an amino acid sequence having at least 80% sequence identity to any one of the CytC, CutC or ybjM membrane proteins with SEQ NO: 56, 90, 92, 94, 96, 190 or 192, respectively.

56. The host cell of claim 50, wherein the membrane protein is i) a β-barrel porin selected from the group consisting of Wza from Escherichia coli K12 MG1655 with SEQ ID NO: 34 and lamB from Escherichia coli K12 MG1655 with SEQ ID NO: 204, ii) a functional homolog or functional fragment to any one of the Wza or lamB proteins, or iii) a polypeptide having at least 80% sequence identity to any one of the Wza or lamB membrane proteins with SEQ ID NO: 34 or 204, respectively.

57. The host cell of claim 50, wherein the membrane protein is i) auxiliary transport protein is Wzc from Thermotoga maritima (strain ATCC 43589/MSB8/DSM 3109/JCM 10099) with SEQ ID NO: 88, ii) a functional homolog or functional fragment thereof or iii) a polypeptide having at least 80% sequence identity to the Wzc membrane protein with SEQ ID NO: 88.

58. The host cell of claim 50, wherein the membrane protein is i) a phosphotransfer-driven group translocator selected from the group consisting of nagE from Escherichia coli K12 MG1655 with SEQ ID NO: 210 and srlB from Escherichia coli K12 MG1655 with SEQ ID NO: 212, ii) a functional homolog or functional fragment of any of the nagE or srlB membrane proteins, or iii) a polypeptide having at least 80% sequence identity to any one of the nagE or srlB membrane proteins with SEQ ID NO: 210 or 212, respectively.

59. The host cell of claim 49, wherein the membrane protein is a transporter protein involved in transport of compounds across the outer membrane of the cell wall.

60. The host cell of claim 49, wherein the cell is stably cultured in a medium.

61. The host cell of claim 49, wherein the cell is selected from the group consisting of microorganism, plant, and animal cells.

62. The host cell according to claim 61, wherein the cell is an Escherichia coli cell.

63. The host cell of claim 49, wherein the cell comprises a catabolic pathway for selected mono-, di- or oligosaccharides that is at least partially inactivated, the mono-, di-, or oligosaccharides being involved in and/or required for the synthesis of fucosyllactose.

64. The host cell of claim 49, wherein the fucosyllactose is 2′-fucosyllactose, 3-fucosyllactose or difucosyllactose.

65. A method of producing fucosyllactose, the method comprising:
culturing the host cell of claim 49 in a medium under conditions permissive for the production of the fucosyllactose; and
separating the fucosyllactose from the cultivation.

66. (canceled)

67. A method of using the host cell of claim 49 for the production of fucosyllactose, the method comprising cultivating the host cell.

68. The method according to claim 67, wherein the fucosyllactose is 2′-fucosyllactose, 3-fucosyllactose or difucosyllactose.

69. The method according to claim 39, wherein the host cells are cultivated in a continuous manner.

70. The method according to claim 42, wherein the host cells are cultivated for at least about 80 hours.

71. The method according to claim 42, wherein the host cells are cultivated for at least about 100 hours.

72. The method according to claim 42, wherein the host cells are cultivated for at least about 120 hours.
